SEQUENCE LISTING (1103 sequences)

SEQ ID NO: 1 - protein, 17 aa, synthetic construct
AGLQFPVGRIHRHLKTR

SEQ ID NO: 2 - protein, 20 aa, synthetic construct
GLVGKLVKGGLKLIGHVANG

SEQ ID NO: 3 - protein, 25 aa, synthetic construct
GLLGKIVGGGLKLIGNHVANHLANG

SEQ ID NO: 4 - protein, 8 aa, synthetic construct
FPFPFGRR

SEQ ID NO: 5 - protein, 18 aa, synthetic construct
LLICALCPPGAARSLRKR

SEQ ID NO: 6 - protein, 25 aa, synthetic construct
HTGLHLCIYCCNCCRNKGCGYCCRT

SEQ ID NO: 7 - protein, 23 aa, synthetic construct
SRMIRRPPGFSPFRVAPASSLRR

SEQ ID NO: 8 - protein, 26 aa, synthetic construct
AGYSRMIRRPPGFSPFRVAPASSLRR

SEQ ID NO: 9 - protein, 23 aa, synthetic construct
SWHGRGGGRRGGSGRRGGRGGGR

SEQ ID NO: 10 - protein, 23 aa, synthetic construct
FCSQIGCRIGKRRLSSSVHRERR

SEQ ID NO: 11 - protein, 15 aa, synthetic construct
FLPIVGKLLSGLLGK

SEQ ID NO: 12 - protein, 29 aa, synthetic construct
GILDTLKQLGKAAVQGLLSKAACKLAKTC

SEQ ID NO: 13 - protein, 22 aa, synthetic construct
RDRSLCRRRFPVLLVPELRTRR

SEQ ID NO: 14 - protein, 27 aa, synthetic construct
ISEHVEKIKIGYEKHSLSKHFKFVHNK

SEQ ID NO: 15 - protein, 29 aa, synthetic construct
GLWTTIKEGVKNFSVGVLDKIRCKITGGC

SEQ ID NO: 16 - protein, 24 aa, synthetic construct
KEERGAPKGCWTKSYPPKPCSGKR

SEQ ID NO: 17 - protein, 15 aa, synthetic construct
GILTLKYPCRAWYHH

SEQ ID NO: 18 - protein, 9 aa, synthetic construct
LLWGHRLQR

SEQ ID NO: 19 - protein, 15 aa, synthetic construct
KVWCQIFSMSSQSKR

SEQ ID NO: 20 - protein, 17 aa, synthetic construct
LPPPHLWRRAMNCLMGR

SEQ ID NO: 21 - protein, 6 aa, synthetic construct
LVWVKK

SEQ ID NO: 22 - protein, 20 aa, synthetic construct
GILTLKVSNMIWVIFSRLAL

SEQ ID NO: 23 - protein, 16 aa, synthetic construct
HRAKTVQTFLEKRHRR

SEQ ID NO: 24 - protein, 29 aa, synthetic construct
GVFDTLKKIGKTVGKVALGVAKNYLNSQK

SEQ ID NO: 25 - protein, 28 aa, synthetic construct
GFWSHLKDKFIETGKAIKRVWKNFFSSG

SEQ ID NO: 26 - protein, 23 aa, synthetic construct
FLGTALKLGKEKKMRRKRKKMKK

SEQ ID NO: 27 - protein, 28 aa, synthetic construct
FLGTALKLGKAFAKTIVPMISDAFHKNQ

SEQ ID NO: 28 - protein, 28 aa, synthetic construct
FLGTAIKLGKAFAKTIVPMISDALRKNQ

SEQ ID NO: 29 - protein, 12 aa, synthetic construct
GRKKPKLGTFWG

SEQ ID NO: 30 - protein, 20 aa, synthetic construct
GVFDTLKKIRKRRMKNFMKR

SEQ ID NO: 31 - protein, 29 aa, synthetic construct
GVFDTLKKIGKTVVKVALGVAKNYLNSQK

SEQ ID NO: 32 - protein, 21 aa, synthetic construct
GLLGKILGVGKKVLCGVSGLC

SEQ ID NO: 33 - protein, 14 aa, synthetic construct
GILLNTLKGAAKNV

SEQ ID NO: 34 - protein, 15 aa, synthetic construct
VSMLIPVTSRIRVKR

SEQ ID NO: 35 - protein, 21 aa, synthetic construct
SGKTLEKTLAKKNPKLGKTKF

SEQ ID NO: 36 - protein, 19 aa, synthetic construct
GWLDIAKNVASIVAGLGKR

SEQ ID NO: 37 - protein, 19 aa, synthetic construct
GWLDIAKKVASVVAGIVKR

SEQ ID NO: 38 - protein, 19 aa, synthetic construct
GWLDIAKKVASVVAGIGKR

SEQ ID NO: 39 - protein, 17 aa, synthetic construct
GLFDLVTSVLGGLGKRR

SEQ ID NO: 40 - protein, 19 aa, synthetic construct
GWLDIAKKVASVVAGLGKR

SEQ ID NO: 41 - protein, 23 aa, synthetic construct
GWLDIAKKSLQLLQASEKEVKRR

SEQ ID NO: 42 - protein, 17 aa, synthetic construct
GLFDLVTGLLGNLGKRR

SEQ ID NO: 43 - protein, 19 aa, synthetic construct
RIQEHLRSFSKYVPVPVRR

SEQ ID NO: 44 - protein, 28 aa, synthetic construct
SNRRPCTGRNCRPRRPGVFTPIAKPLKK

SEQ ID NO: 45 - protein, 9 aa, synthetic construct
RIHRHLKTR

SEQ ID NO: 46 - protein, 19 aa, synthetic construct
GWSDIAKKVASAVAGLGKR

SEQ ID NO: 47 - protein, 25 aa, synthetic construct
GWSDIAKKVASAVAGLGKRIIFFPG

SEQ ID NO: 48 - protein, 25 aa, synthetic construct
GSSRMIRRPPGFSPFRVAPASSLKR

SEQ ID NO: 49 - protein, 23 aa, synthetic construct
GMQKNGRCANVNVMCVSVRSNKK

SEQ ID NO: 50 - protein, 25 aa, synthetic construct
FFRVLCTSIRRRLGSKDLMCKDKKK

SEQ ID NO: 51 - protein, 25 aa, synthetic construct
HTGLHLCIYCCKCCRYKGCGYCCRT

SEQ ID NO: 52 - protein, 23 aa, synthetic construct
SNIKGTYRAVAVVKKIKYKRHIP

SEQ ID NO: 53 - protein, 27 aa, synthetic construct
GIIDSVMGQVCVKMNYKLPFCARFKPK

SEQ ID NO: 54 - protein, 22 aa, synthetic construct
AITRMIRRPPGFTPFRIAPAIV

SEQ ID NO: 55 - protein, 21 aa, synthetic construct
GIFGKILGVGKKVLCGLSGLC

SEQ ID NO: 56 - protein, 18 aa, synthetic construct
VVGNLISTIVNLVKKLGK

SEQ ID NO: 57 - protein, 15 aa, synthetic construct
AAPRGCWTKSIPPKP

SEQ ID NO: 58 - protein, 28 aa, synthetic construct
EQERFLPLLAGLAANFLPKLFCKITRKC

SEQ ID NO: 59 - protein, 21 aa, synthetic construct
GLLSGLLGAGKKIVCGLSGMC

SEQ ID NO: 60 - protein, 29 aa, synthetic construct
GLFTLIKGAAKLIGKTVAKEAGKTVAKEA

SEQ ID NO: 61 - protein, 21 aa, synthetic construct
GLLSGILGAGKNRVCGLSGLC

SEQ ID NO: 62 - protein, 15 aa, synthetic construct
GILSGLLGAGKKIVC

SEQ ID NO: 63 - protein, 12 aa, synthetic construct
GLISGILGAGKK

SEQ ID NO: 64 - protein, 21 aa, synthetic construct
GLLRGILGAGKKIVCGLSGLC

SEQ ID NO: 65 - protein, 30 aa, synthetic construct
GLFTLIKGAAKLIGKTVAKEAGKTGLEFMF

SEQ ID NO: 66 - protein, 26 aa, synthetic construct
ATAWGIPPNGIPPIVAVRIRPLCGTV

SEQ ID NO: 67 - protein, 29 aa, synthetic construct
GFMDTAKSVAKNVAVTLLDNLKCKITKAC

SEQ ID NO: 68 - protein, 15 aa, synthetic construct
GLISGILGAGKKVLC

SEQ ID NO: 69 - protein, 29 aa, synthetic construct
GFMDTAKNVAKNVAVTLLYNLKCKITKAC

SEQ ID NO: 70 - protein, 13 aa, synthetic construct
GFMDTAKNVAKKV

SEQ ID NO: 71 - protein, 21 aa, synthetic construct
GLLSGILGAGKKIVCGFSGLC

SEQ ID NO: 72 - protein, 11 aa, synthetic construct
FLPPWLRPSKK

SEQ ID NO: 73 - protein, 21 aa, synthetic construct
FLPPWLRPSKTTKRQLELKQY

SEQ ID NO: 74 - protein, 20 aa, synthetic construct
CYSFSFLGPSPYLSVRKSKR

SEQ ID NO: 75 - protein, 15 aa, synthetic construct
GIFGKILGVGKKVLC

SEQ ID NO: 76 - protein, 22 aa, synthetic construct
GVLGTVKNLLIGAGKSAAQSVR

SEQ ID NO: 77 - protein, 23 aa, synthetic construct
AGYTRMIRRPPGFSPFRIAPAIV

SEQ ID NO: 78 - protein, 17 aa, synthetic construct
GLLDSFKNLALNAAKRC

SEQ ID NO: 79 - protein, 16 aa, synthetic construct
TVWGFRPTNPKPKSPG

SEQ ID NO: 80 - protein, 18 aa, synthetic construct
GLRSGILGAGKKVLCGLS

SEQ ID NO: 81 - protein, 27 aa, synthetic construct
ATALGLSSRGLLPIGFMFKDTIRCRKD

SEQ ID NO: 82 - protein, 26 aa, synthetic construct
AGYTRMIRRPPGFSPFRVAPASSLKR

SEQ ID NO: 83 - protein, 24 aa, synthetic construct
FFPLFASLAANFLPKLFCKIARKC

SEQ ID NO: 84 - protein, 7 aa, synthetic construct
RPAKSRR

SEQ ID NO: 85 - protein, 24 aa, synthetic construct
GVLGTVKNLPGKGLRICSSKGSST

SEQ ID NO: 86 - protein, 18 aa, synthetic construct
GLLSGILGAGKKLLKWKR

SEQ ID NO: 87 - protein, 30 aa, synthetic construct
GIFTLIKGAAKLIGKTVAKEAGKTGLEFMF

SEQ ID NO: 88 - protein, 12 aa, synthetic construct
GLLSGILGAGKR

SEQ ID NO: 89 - protein, 24 aa, synthetic construct
VIPFVARVAAEMMQHVYCAASKRC

SEQ ID NO: 90 - protein, 30 aa, synthetic construct
DDPQERFLPFLAGLAANFLPKLFCKITRKC

SEQ ID NO: 91 - protein, 28 aa, synthetic construct
ERERFLPFLAGLAANFLPKLFCKITRKC

SEQ ID NO: 92 - protein, 26 aa, synthetic construct
EQARFLPLLAGLAANFLPKLFCKITR

SEQ ID NO: 93 - protein, 24 aa, synthetic construct
KQERCVDMGFSPTGKRPPFCPYPG

SEQ ID NO: 94 - protein, 28 aa, synthetic construct
EQERFLPLLAGLAANFLPKIFCKITKKC

SEQ ID NO: 95 - protein, 28 aa, synthetic construct
EQERFFPLFASLAANFLPKLFCKIARKC

SEQ ID NO: 96 - protein, 28 aa, synthetic construct
EQERFLPFLAGLAANFLPKLFCKITRKC

SEQ ID NO: 97 - protein, 24 aa, synthetic construct
KQERWVDTGFSHTGKRPPFCPYPG

SEQ ID NO: 98 - protein, 9 aa, synthetic construct
LPHLHPWRR

SEQ ID NO: 99 - protein, 23 aa, synthetic construct
IIKGLSPLRKIEFGPGGSIRSKR

SEQ ID NO: 100 - protein, 25 aa, synthetic construct
NSHLSICIYCCKCCKRKGCGMCCKT

SEQ ID NO: 101 - protein, 24 aa, synthetic construct
FFPLFGSLVANFLPKLICKIARKC

SEQ ID NO: 102 - protein, 21 aa, synthetic construct
GIFGGLLKVGKKIACGLSGLC

SEQ ID NO: 103 - protein, 28 aa, synthetic construct
GIFGGLLKVGKKIACGSVFTLVKSSLDC

SEQ ID NO: 104 - protein, 25 aa, synthetic construct
GLFTIIKGAAKLIGKAVAKEAGKSG

SEQ ID NO: 105 - protein, 21 aa, synthetic construct
AVRLRGCWTKSLPPQPCYGKR

SEQ ID NO: 106 - protein, 25 aa, synthetic construct
DIFGGLLKVGKKIACGLSGLCQSLN

SEQ ID NO: 107 - protein, 12 aa, synthetic construct
GIFGGLLKVGKK

SEQ ID NO: 108 - protein, 29 aa, synthetic construct
GIFGGLLKVGKKIPRFISSILATSSSLFS

SEQ ID NO: 109 - protein, 21 aa, synthetic construct
GIFGGLLKVGMKIACGLSGLC

SEQ ID NO: 110 - protein, 18 aa, synthetic construct
GIFGGLLKVGKKIACGLS

SEQ ID NO: 111 - protein, 27 aa, synthetic construct
ATAWDLGPHGLRPIRPIRIRPLCGDRS

SEQ ID NO: 112 - protein, 22 aa, synthetic construct
GFLDTAKNAAKNVAKNVAVTLL

SEQ ID NO: 113 - protein, 27 aa, synthetic construct
GIFGLLRKKYFLQFLNFQKTVIKKLQL

SEQ ID NO: 114 - protein, 19 aa, synthetic construct
GIFGGRGKLFIEHAPPRPL

SEQ ID NO: 115 - protein, 14 aa, synthetic construct
ALKFPFRCKARIRR

SEQ ID NO: 116 - protein, 21 aa, synthetic construct
AAPLRGCWTKSLPPQPCYGKR

SEQ ID NO: 117 - protein, 23 aa, synthetic construct
GIFGGLLKVGKKKEMKKLLKWKR

SEQ ID NO: 118 - protein, 28 aa, synthetic construct
ATAWDLGPHGLRPIRPIRIRPLCGKDNS

SEQ ID NO: 119 - protein, 18 aa, synthetic construct
GIFGGLLKVGKKLLKWKR

SEQ ID NO: 120 - protein, 26 aa, synthetic construct
AGYSRMIRRPPGFSPFRVAPPSSLKR

SEQ ID NO: 121 - protein, 25 aa, synthetic construct
GLFTIIKGAAKLIGKAGQRSRQEWA

SEQ ID NO: 122 - protein, 18 aa, synthetic construct
GIFPKMFTLKKPLLLIVL

SEQ ID NO: 123 - protein, 15 aa, synthetic construct
GFLDTAKNAAKNAAK

SEQ ID NO: 124 - protein, 18 aa, synthetic construct
GIFGGLLKMGKKIACGLS

SEQ ID NO: 125 - protein, 10 aa, synthetic construct
GIFGGLLKVR

SEQ ID NO: 126 - protein, 19 aa, synthetic construct
FKCRIPLVKPLLNNKRQKR

SEQ ID NO: 127 - protein, 23 aa, synthetic construct
AGYSRMIRRPPGFTPFRIAPEMG

SEQ ID NO: 128 - protein, 19 aa, synthetic construct
GIFGGLLKVGKKIACGLSG

SEQ ID NO: 129 - protein, 21 aa, synthetic construct
GIFGGILKVGKKIACGLSGLC

SEQ ID NO: 130 - protein, 21 aa, synthetic construct
GIFGGLLKVGKKIACGLTGLC

SEQ ID NO: 131 - protein, 20 aa, synthetic construct
GIFGGLLKVGKKIACGLSGL

SEQ ID NO: 132 - protein, 22 aa, synthetic construct
SADQTGMNKAALSPIKKKKKSV

SEQ ID NO: 133 - protein, 14 aa, synthetic construct
GIFGGLLKVGKKIA

SEQ ID NO: 134 - protein, 8 aa, synthetic construct
RWVPFGKR

SEQ ID NO: 135 - protein, 7 aa, synthetic construct
WVPFGKR

SEQ ID NO: 136 - protein, 23 aa, synthetic construct
RRYKKSNSLGLGIVSSSPCLRKR

SEQ ID NO: 137 - protein, 23 aa, synthetic construct
GIFGGLLKVGKKIACGLSGLCQS

SEQ ID NO: 138 - protein, 13 aa, synthetic construct
GCSRWIIGSRKKR

SEQ ID NO: 139 - protein, 21 aa, synthetic construct
GIFGGHLKVGKKIACGLSGLC

SEQ ID NO: 140 - protein, 28 aa, synthetic construct
GIFGGLLKVGKKIACGLNTDVSVIYNMN

SEQ ID NO: 141 - protein, 21 aa, synthetic construct
GIFGGLLKEGKKIACGLSGLC

SEQ ID NO: 142 - protein, 9 aa, synthetic construct
KLMIPRKKR

SEQ ID NO: 143 - protein, 13 aa, synthetic construct
GIFGGLLKVGKKI

SEQ ID NO: 144 - protein, 17 aa, synthetic construct
GLLGTVKNLLIGAGKSA

SEQ ID NO: 145 - protein, 12 aa, synthetic construct
GILTLKYPRSSL

SEQ ID NO: 146 - protein, 18 aa, synthetic construct
GIFGGLLKVGKKIACRLS

SEQ ID NO: 147 - protein, 23 aa, synthetic construct
GIFGGLLKVKKKKEMKKLLKWKR

SEQ ID NO: 148 - protein, 19 aa, synthetic construct
GILSVFKNALPGIMKFITG

SEQ ID NO: 149 - protein, 21 aa, synthetic construct
GIFGGLLKVRKKIACGLSVLC

SEQ ID NO: 150 - protein, 26 aa, synthetic construct
APAWGIPPNGIPRMVAVRIRPLCGAV

SEQ ID NO: 151 - protein, 16 aa, synthetic construct
GIFGGLLKVGKKIACG

SEQ ID NO: 152 - protein, 26 aa, synthetic construct
VIPFVASVAAEMMQHVYCAASRRCKN

SEQ ID NO: 153 - protein, 21 aa, synthetic construct
GILSGLLGAGKKLACGLIGLC

SEQ ID NO: 154 - protein, 21 aa, synthetic construct
GIFGGLLKVGKKIACGLSTAR

SEQ ID NO: 155 - protein, 19 aa, synthetic construct
GILSVIKNALPGIMRFIAG

SEQ ID NO: 156 - protein, 25 aa, synthetic construct
GIFGGLLKVGKKIACGLSGQRIGGE

SEQ ID NO: 157 - protein, 16 aa, synthetic construct
NAISMIADLARKLLGK

SEQ ID NO: 158 - protein, 13 aa, synthetic construct
ALKFPFRCKAAFC

SEQ ID NO: 159 - protein, 22 aa, synthetic construct
GIFSKITGKGIKNLLFKGIKNV

SEQ ID NO: 160 - protein, 28 aa, synthetic construct
ATAWDLGPHGLRPIRPIRIRPLCGKDRS

SEQ ID NO: 161 - protein, 26 aa, synthetic construct
APVWGIPPNGIPRMVAVRIRPLCGAV

SEQ ID NO: 162 - protein, 25 aa, synthetic construct
ATAWDLGPHGLRPIRPIRIRPLCGK

SEQ ID NO: 163 - protein, 26 aa, synthetic construct
SGYSRMIRRPPGFSPFRVAPASSLKR

SEQ ID NO: 164 - protein, 29 aa, synthetic construct
GSLEVQRARCRHPLLRSSLRCRHLAQMRT

SEQ ID NO: 165 - protein, 14 aa, synthetic construct
FLPPWLRPPKKTKR

SEQ ID NO: 166 - protein, 16 aa, synthetic construct
RRYWILRRLESGVERR

SEQ ID NO: 167 - protein, 26 aa, synthetic construct
VIPFVASVAAEMMQNVYCAASRRCKN

SEQ ID NO: 168 - protein, 29 aa, synthetic construct
GSLEVQRARCRHPLYRSSLRCRHLAQMRT

SEQ ID NO: 169 - protein, 21 aa, synthetic construct
AAPLRGCWTKNLPPQPCYGKR

SEQ ID NO: 170 - protein, 19 aa, synthetic construct
SILSVIKNALPGIMKLIAG

SEQ ID NO: 171 - protein, 17 aa, synthetic construct
GIFGGLLKVGKKIACGL

SEQ ID NO: 172 - protein, 16 aa, synthetic construct
TIWGFRPTNPKPKPPG

SEQ ID NO: 173 - protein, 12 aa, synthetic construct
GIFGGLLKKKKK

SEQ ID NO: 174 - protein, 19 aa, synthetic construct
SILSVIKNALPGIMKFIAG

SEQ ID NO: 175 - protein, 30 aa, synthetic construct
GIFGGLLKVGKKIACFISSILATSSSLFSS

SEQ ID NO: 176 - protein, 20 aa, synthetic construct
GIFSKITGKGIKNLLFKGIK

SEQ ID NO: 177 - protein, 20 aa, synthetic construct
GLFTIIKGAAKLIGKAVAKR

SEQ ID NO: 178 - protein, 28 aa, synthetic construct
EEERFFPLFGSLVANFLPKLICKIARKC

SEQ ID NO: 179 - protein, 24 aa, synthetic construct
QIGSRKKRGCSRWIIGINGPVCRD

SEQ ID NO: 180 - protein, 24 aa, synthetic construct
SNRGIFGGLLKVGKKIACGLSGLC

SEQ ID NO: 181 - protein, 27 aa, synthetic construct
AGYSRMIRRPPGFSPFRVAPASSLKRA

SEQ ID NO: 182 - protein, 15 aa, synthetic construct
RWVPFGKRWVPFGKR

SEQ ID NO: 183 - protein, 14 aa, synthetic construct
WVPFGKRWVPFGKR

SEQ ID NO: 184 - protein, 22 aa, synthetic construct
RWVPFGKRWVPFGKRWVPFGKR

SEQ ID NO: 185 - protein, 21 aa, synthetic construct
WVPFGKRWVPFGKRWVPFGKR

SEQ ID NO: 186 - protein, 16 aa, synthetic construct
VPFGKRWVPFGKRQIE

SEQ ID NO: 187 - protein, 17 aa, synthetic construct
WVPFGKRWVPFGKRQIE

SEQ ID NO: 188 - protein, 22 aa, synthetic construct
GSRKKRGCSRWIIGINGPVCRD

SEQ ID NO: 189 - protein, 30 aa, synthetic construct
AGYSRMIRRPPGFSPFRVAPASSLKRAGYS

SEQ ID NO: 190 - protein, 30 aa, synthetic construct
KLMIPRKKRGIFGGLLKVGKKIACGLSGLC

SEQ ID NO: 191 - protein, 29 aa, synthetic construct
QDERNAEEEKRGIFGGLLKVGKKIACRLS

SEQ ID NO: 192 - protein, 30 aa, synthetic construct
QDGRNAEEEKRGIFGGLLKVGKKIACGLSG

SEQ ID NO: 193 - protein, 23 aa, synthetic construct
IPNGCSIRAEPPVPPPKPSLKKR

SEQ ID NO: 194 - protein, 21 aa, synthetic construct
SRSGRGGRGGRGGRGGRGGRG

SEQ ID NO: 195 - protein, 24 aa, synthetic construct
SSATWLWTLKMKWLLLPLPLPLKR

SEQ ID NO: 196 - protein, 20 aa, synthetic construct
GWACSIGVRLLPQWFGLFLR

SEQ ID NO: 197 - protein, 23 aa, synthetic construct
QTNLLGKVSKVANDALKILPIQV

SEQ ID NO: 198 - protein, 25 aa, synthetic construct
HTVLGLCIYCCKCCRYKGCGYCCRT

SEQ ID NO: 199 - protein, 23 aa, synthetic construct
GLWTTIKEGVKNFSVGVLDKIRC

SEQ ID NO: 200 - protein, 27 aa, synthetic construct
ATAWKVPPGLQPIRPIRIRPLCGNDKS

SEQ ID NO: 201 - protein, 19 aa, synthetic construct
RRPPGFTPFRVAPASSLKR

SEQ ID NO: 202 - protein, 25 aa, synthetic construct
KSVMSGRGKGGKSKSSGKVKSRSSR

SEQ ID NO: 203 - protein, 16 aa, synthetic construct
GRGKRGLVTNLLSTVL

SEQ ID NO: 204 - protein, 16 aa, synthetic construct
GRGKRGIVKNLLSTVL

SEQ ID NO: 205 - protein, 25 aa, synthetic construct
HTGLHLCTYCCKCCRNKGCGYCCRT

SEQ ID NO: 206 - protein, 23 aa, synthetic construct
DTLFGPCGHIATCSLCSPRVKKC

SEQ ID NO: 207 - protein, 21 aa, synthetic construct
FLKKTWKKLKKAAKSILKNTG

SEQ ID NO: 208 - protein, 25 aa, synthetic construct
RAKNTFIVESEKTVLSICRKAIKKR

SEQ ID NO: 209 - protein, 29 aa, synthetic construct
FLKKTWKKLKKAAKSILKNTGPVTYQHKF

SEQ ID NO: 210 - protein, 18 aa, synthetic construct
GLLGKILGVGKKVLCGVS

SEQ ID NO: 211 - protein, 18 aa, synthetic construct
GIFSKLAGKIPLFVSLPS

SEQ ID NO: 212 - protein, 29 aa, synthetic construct
GIFSKLAGKKIKNPLISGLKNIGKEVGMA

SEQ ID NO: 213 - protein, 26 aa, synthetic construct
VILFVASVAAEMMHHVYCAASKRCKN

SEQ ID NO: 214 - protein, 30 aa, synthetic construct
GILTDTLKGAAKNLAGVLLDKLKCKITGGC

SEQ ID NO: 215 - protein, 24 aa, synthetic construct
GIFSLIKGAAKVVAKGLGKEEVKR

SEQ ID NO: 216 - protein, 24 aa, synthetic construct
GIFSKLAGKMIKNLLISGLKNIGK

SEQ ID NO: 217 - protein, 21 aa, synthetic construct
GLLGKIPGVGKKVLCGVSGLC

SEQ ID NO: 218 - protein, 12 aa, synthetic construct
GIFSKLAGKKIK

SEQ ID NO: 219 - protein, 29 aa, synthetic construct
GIFSLIKGAAKVVAKGLGREVQEEEVKRG

SEQ ID NO: 220 - protein, 21 aa, synthetic construct
GLLGKILGLGKKVLCGVSGLC

SEQ ID NO: 221 - protein, 29 aa, synthetic construct
GLWTTIKEGVKNFSVGVLDKIRCKITGEC

SEQ ID NO: 222 - protein, 21 aa, synthetic construct
GLLGKILGVGKKVLCGGSGLC

SEQ ID NO: 223 - protein, 20 aa, synthetic construct
GVPLIYKRPGIYVTKRPRGK

SEQ ID NO: 224 - protein, 24 aa, synthetic construct
GLLGKILGVRKKEEMSLMKGMLKR

SEQ ID NO: 225 - protein, 14 aa, synthetic construct
GILLNTLKGAAKNG

SEQ ID NO: 226 - protein, 26 aa, synthetic construct
VIPFVASVAAEMMHRVYCAASKRCKN

SEQ ID NO: 227 - protein, 17 aa, synthetic construct
GIFSKLAGKRNKKRYFL

SEQ ID NO: 228 - protein, 22 aa, synthetic construct
GLLLDTVKGAAKNVAGKLQMKK

SEQ ID NO: 229 - protein, 29 aa, synthetic construct
RLHHNLSRMIRRPPGFTPFRVAPASSLKR

SEQ ID NO: 230 - protein, 29 aa, synthetic construct
FDQGYGHFCRQGALQNLLKMASCKLDKTC

SEQ ID NO: 231 - protein, 15 aa, synthetic construct
FIPLVSGLFSRLLGK

SEQ ID NO: 232 - protein, 30 aa, synthetic construct
GILTDTLKGAAKNVAGVLLDKLKCKITGGY

SEQ ID NO: 233 - protein, 23 aa, synthetic construct
GIFSLIKGAAKVVAKGLGKEVKR

SEQ ID NO: 234 - protein, 15 aa, synthetic construct
GLLGKILGVGKKVLC

SEQ ID NO: 235 - protein, 20 aa, synthetic construct
GIFSLIKGAAKVVAKPLATT

SEQ ID NO: 236 - protein, 15 aa, synthetic construct
GIFSLIKGAAKNVAG

SEQ ID NO: 237 - protein, 18 aa, synthetic construct
GIFPKLAGKKIKNLLISG

SEQ ID NO: 238 - protein, 30 aa, synthetic construct
GILTDTSKGAAKNVAGVLLDKLKCKITGGC

SEQ ID NO: 239 - protein, 11 aa, synthetic construct
GRPRWSHRSRR

SEQ ID NO: 240 - protein, 22 aa, synthetic construct
GLLGKILGVGKKVLTPHSTFFP

SEQ ID NO: 241 - protein, 19 aa, synthetic construct
SNRDFFKVNIFGFGRFSWC

SEQ ID NO: 242 - protein, 14 aa, synthetic construct
GIFSKLAGKKRYFL

SEQ ID NO: 243 - protein, 23 aa, synthetic construct
GIFSLIKGAAKVVAKGLGKEVGG

SEQ ID NO: 244 - protein, 21 aa, synthetic construct
GPLGKILGVGKKVLCGVSGLC

SEQ ID NO: 245 - protein, 27 aa, synthetic construct
GIFSKLAGKKIKKREAKQKEVFSLNWR

SEQ ID NO: 246 - protein, 10 aa, synthetic construct
GAPKGCWTKS

SEQ ID NO: 247 - protein, 19 aa, synthetic construct
FLPSSPWNEGTYVLKKLKT

SEQ ID NO: 248 - protein, 20 aa, synthetic construct
GIFSKLAGKKIKNPLMSRFL

SEQ ID NO: 249 - protein, 14 aa, synthetic construct
GIFSKLAGKKIKNL

SEQ ID NO: 250 - protein, 17 aa, synthetic construct
GILLNTLKGAAKNGRGF

SEQ ID NO: 251 - protein, 30 aa, synthetic construct
GILLNTLKGAAKNVAGLLLDKLKCKITGGC

SEQ ID NO: 252 - protein, 24 aa, synthetic construct
FLPLLAGLAANFLPTIFCKISRKC

SEQ ID NO: 253 - protein, 26 aa, synthetic construct
GIFSKLAGKKIKNLLISGLKNLLISG

SEQ ID NO: 254 - protein, 25 aa, synthetic construct
GIFSKLAGKKIKNLLISGLKNIGAR

SEQ ID NO: 255 - protein, 30 aa, synthetic construct
GIFSLIKGAAKVVAKGLGKFGLDLMACKVT

SEQ ID NO: 256 - protein, 14 aa, synthetic construct
GIFSKLAGKKIKSL

SEQ ID NO: 257 - protein, 20 aa, synthetic construct
GALKGCWTKSYPPKPCSGKR

SEQ ID NO: 258 - protein, 27 aa, synthetic construct
GIFSKLAGKKIKNLLISGLKNYPPKDV

SEQ ID NO: 259 - protein, 15 aa, synthetic construct
GIFSKLLGKRSRTCS

SEQ ID NO: 260 - protein, 19 aa, synthetic construct
GIFKVNIFGRVVLSIFSAL

SEQ ID NO: 261 - protein, 30 aa, synthetic construct
GILLNTLKGAAKNVAGDLLDKLKCKITGGC

SEQ ID NO: 262 - protein, 28 aa, synthetic construct
GAPKGCWTKSYPPKPCSATLGRTSFYFF

SEQ ID NO: 263 - protein, 21 aa, synthetic construct
GIFSKLAGKKMFTLKKPLLLI

SEQ ID NO: 264 - protein, 30 aa, synthetic construct
GIFSKLAGKKIKNLLISGLKNIGKEVGKFG

SEQ ID NO: 265 - protein, 29 aa, synthetic construct
GIFTKLAGKKIKNLLISGLKNIGRFLIFF

SEQ ID NO: 266 - protein, 12 aa, synthetic construct
VLLGKILGVGKK

SEQ ID NO: 267 - protein, 18 aa, synthetic construct
TPSWVFPISYCTSVSLKR

SEQ ID NO: 268 - protein, 26 aa, synthetic construct
GAPKGCWTKSYPPQPCSGRSKKRCAQ

SEQ ID NO: 269 - protein, 20 aa, synthetic construct
GASKGCWTKSYPPKPCSGKR

SEQ ID NO: 270 - protein, 30 aa, synthetic construct
GLLGKILGVGKKALTPHSTFFPTPRILPNR

SEQ ID NO: 271 - protein, 30 aa, synthetic construct
GLLLDTVKGAAKNVAGVLLDKLKCKITGGC

SEQ ID NO: 272 - protein, 25 aa, synthetic construct
GSSRMIRRPPGFTPFRIAPASSLKR

SEQ ID NO: 273 - protein, 30 aa, synthetic construct
GIFSKLAGKKIKNLLISGPKNIGKEVGMDV

SEQ ID NO: 274 - protein, 28 aa, synthetic construct
GIFSKLAGKKIKNLLISGLKNIGKEAGP

SEQ ID NO: 275 - protein, 25 aa, synthetic construct
GIFSKLAGKKIKNLLISGLKNIGLP

SEQ ID NO: 276 - protein, 12 aa, synthetic construct
GLLGKILGVEKR

SEQ ID NO: 277 - protein, 26 aa, synthetic construct
GIFSKLAGKKIKNLLISGLKNIGAPP

SEQ ID NO: 278 - protein, 25 aa, synthetic construct
GIFSKLAGKKIKNLLISGLKNIGPQ

SEQ ID NO: 279 - protein, 24 aa, synthetic construct
GLLGKVLGVGKKEEMSLMKGMLKR

SEQ ID NO: 280 - protein, 16 aa, synthetic construct
GIFSLIKGAAKVVAKG

SEQ ID NO: 281 - protein, 12 aa, synthetic construct
GLLGKVLGVGKK

SEQ ID NO: 282 - protein, 29 aa, synthetic construct
GIFSKLAGKKIKNLLISGLKNIGKNLLIS

SEQ ID NO: 283 - protein, 26 aa, synthetic construct
GIFSKLAGKKIKNFLAYVLESAYEQV

SEQ ID NO: 284 - protein, 30 aa, synthetic construct
GILLYTLKGAAKNVAEVLLDKLKCKITGGC

SEQ ID NO: 285 - protein, 27 aa, synthetic construct
GIFSLIKGAAKVVAKGLGKEGKFRRKK

SEQ ID NO: 286 - protein, 12 aa, synthetic construct
GLLGKILGVGKR

SEQ ID NO: 287 - protein, 15 aa, synthetic construct
GFSPFRIAPASSLKR

SEQ ID NO: 288 - protein, 28 aa, synthetic construct
GIFSKLAGKKIKNLLISGQPGPSAPWDV

SEQ ID NO: 289 - protein, 25 aa, synthetic construct
GIFSKLAGKKIKNLLISGLKNIGSP

SEQ ID NO: 290 - protein, 24 aa, synthetic construct
GIFSKLAGKKIKSLLISGLKNIGE

SEQ ID NO: 291 - protein, 21 aa, synthetic construct
FLPSSPWNEGTYVLKKLKRKK

SEQ ID NO: 292 - protein, 21 aa, synthetic construct
GLLGKILGVGKKVQCGVSGLC

SEQ ID NO: 293 - protein, 12 aa, synthetic construct
GILLNTLKGAAR

SEQ ID NO: 294 - protein, 29 aa, synthetic construct
GIFSKLAGKKIKNPLISGLKNIGKEVGMD

SEQ ID NO: 295 - protein, 21 aa, synthetic construct
GLLGKILGVGRKVLCGVSGLC

SEQ ID NO: 296 - protein, 30 aa, synthetic construct
GIFSLIKGAAKVVAKGLGKEVGKFGQGSGQ

SEQ ID NO: 297 - protein, 21 aa, synthetic construct
GLLGKILGVGKKALCGVSGLC

SEQ ID NO: 298 - protein, 13 aa, synthetic construct
KWGKPRWASSLKR

SEQ ID NO: 299 - protein, 23 aa, synthetic construct
GLLGKILGVGKKVLCGVSGFVKA

SEQ ID NO: 300 - protein, 20 aa, synthetic construct
GLLGKILGVGKKVLCGVSGL

SEQ ID NO: 301 - protein, 18 aa, synthetic construct
RRCNTKWGKPRWASSLKR

SEQ ID NO: 302 - protein, 18 aa, synthetic construct
GILLNTLKGAAKNWPGFC

SEQ ID NO: 303 - protein, 24 aa, synthetic construct
GLLGKILGVGKKEEMSLMKGMLKR

SEQ ID NO: 304 - protein, 29 aa, synthetic construct
GIFSLIKGAAKVVAKGLGKEVGKFGQGSG

SEQ ID NO: 305 - protein, 28 aa, synthetic construct
GIFSLIKGAAKVVAKGLGKEVQEEEVKR

SEQ ID NO: 306 - protein, 29 aa, synthetic construct
GTMSTNKGVSKNVAVALLDRLQCKITGTC

SEQ ID NO: 307 - protein, 17 aa, synthetic construct
RCNTKWGKPRWASSLKR

SEQ ID NO: 308 - protein, 10 aa, synthetic construct
RPRWSHRSRR

SEQ ID NO: 309 - protein, 30 aa, synthetic construct
GIFSKLAGKKIKNLLISGLKNMGKEVGKFG

SEQ ID NO: 310 - protein, 29 aa, synthetic construct
GIFSKLAGKKIKNLLISGLKNIGKEVGKR

SEQ ID NO: 311 - protein, 27 aa, synthetic construct
GLWTTIKEGVKNFSVGVLDKIRCKITG

SEQ ID NO: 312 - protein, 21 aa, synthetic construct
GLLGKILGVRKKVLCGVSGLC

SEQ ID NO: 313 - protein, 22 aa, synthetic construct
GIFSKLAGKKIKNLLISGLKNK

SEQ ID NO: 314 - protein, 10 aa, synthetic construct
GIFSKLAGKK

SEQ ID NO: 315 - protein, 11 aa, synthetic construct
GIFSKLARRKR

SEQ ID NO: 316 - protein, 26 aa, synthetic construct
GIFSKLAGKKKIKNLLISGLKNIGKE

SEQ ID NO: 317 - protein, 18 aa, synthetic construct
VVPGFTPFRIAPASSLKR

SEQ ID NO: 318 - protein, 20 aa, synthetic construct
GLLGKVLGVGKKVLTPHSTF

SEQ ID NO: 319 - protein, 22 aa, synthetic construct
ATAWKVPPGLQPIRPIRIRPLC

SEQ ID NO: 320 - protein, 29 aa, synthetic construct
GIFSKLAGKKIKSLLVSGLKNIGKEVGMD

SEQ ID NO: 321 - protein, 13 aa, synthetic construct
GIFSLIKGAAKVV

SEQ ID NO: 322 - protein, 15 aa, synthetic construct
GIFSKLAVKKIKNLL

SEQ ID NO: 323 - protein, 25 aa, synthetic construct
GIFSLIKGAAKVVAKGLGKEAGKFG

SEQ ID NO: 324 - protein, 15 aa, synthetic construct
GIFSKLAGKKIKNLP

SEQ ID NO: 325 - protein, 24 aa, synthetic construct
GIFSKLAGKKIKNLPMFLSPLMSR

SEQ ID NO: 326 - protein, 24 aa, synthetic construct
GIFSKLAGEAIKNLLISGLKNIGK

SEQ ID NO: 327 - protein, 25 aa, synthetic construct
EQKRGLLGKILGVGKKVLCGVSGLC

SEQ ID NO: 328 - protein, 29 aa, synthetic construct
LAHRELRRGLLGKILGVGKKVLCGVSGLC

SEQ ID NO: 329 - protein, 25 aa, synthetic construct
EQERGLLGKIPGVGKKVLCGVSGLC

SEQ ID NO: 330 - protein, 20 aa, synthetic construct
KRSKRVIPIVSGLLSSLLGK

SEQ ID NO: 331 - protein, 19 aa, synthetic construct
RKKRVIPIVSGLLSSLLGK

SEQ ID NO: 332 - protein, 30 aa, synthetic construct
EQKRGIFSKLAGKKIKNLLISGLKNLLISG

SEQ ID NO: 333 - protein, 27 aa, synthetic construct
EQKRGIFSKLAGKKIKNLLISGLKNIG

SEQ ID NO: 334 - protein, 25 aa, synthetic construct
EQKRGIFSKLAGKKIKNLLISGLKN

SEQ ID NO: 335 - protein, 25 aa, synthetic construct
ELERGLLGKILGVGKKVLCGVSGLC

SEQ ID NO: 336 - protein, 30 aa, synthetic construct
KEERGAPKGCWTKSYPPQPCSGRSKKRCAQ

SEQ ID NO: 337 - protein, 24 aa, synthetic construct
KEERGASKGCWTKSYPPKPCSGKR

SEQ ID NO: 338 - protein, 29 aa, synthetic construct
EQKRGIFSKLAGKKIKNLLISGLKNIGPQ

SEQ ID NO: 339 - protein, 25 aa, synthetic construct
EQERGLLGKILGVGKKVLCGVSGLC

SEQ ID NO: 340 - protein, 29 aa, synthetic construct
DANGEERRGLLGKILGVGKKVLCGVSGLC

SEQ ID NO: 341 - protein, 24 aa, synthetic construct
NEEERRGLLGKILGVGKKVLCGVS

SEQ ID NO: 342 - protein, 30 aa, synthetic construct
EQKRGIFSKLAGKKKIKNLLISGLKNIGKE

SEQ ID NO: 343 - protein, 26 aa, synthetic construct
GIFSLIKGAAKVVAKGLGKEVDKFGL

SEQ ID NO: 344 - protein, 30 aa, synthetic construct
GIFSLIKGAAKVVAKGLGKGGEVQEEEVKR

SEQ ID NO: 345 - protein, 22 aa, synthetic construct
VIPIVSGLLSCLLEKKKKILKL

SEQ ID NO: 346 - protein, 16 aa, synthetic construct
GILLNTLKGAAKNVAG

SEQ ID NO: 347 - protein, 26 aa, synthetic construct
GLWTTIKEGVKNFSVGVLDKIRSKKR

SEQ ID NO: 348 - protein, 18 aa, synthetic construct
GIFSLIKGLGKEVGKFGL

SEQ ID NO: 349 - protein, 22 aa, synthetic construct
VIPIVSGLLSSLLKKKQKILKL

SEQ ID NO: 350 - protein, 18 aa, synthetic construct
GVPLIYKRPGLLVTYIPG

SEQ ID NO: 351 - protein, 24 aa, synthetic construct
SLLDSIKSMAISAGKGALQNLLKM

SEQ ID NO: 352 - protein, 29 aa, synthetic construct
GIVSLIKGAAKVVAKGLGKEVGKFGLDLM

SEQ ID NO: 353 - protein, 25 aa, synthetic construct
GSSRMIRRPPGFTPFRVAPASSLKR

SEQ ID NO: 354 - protein, 28 aa, synthetic construct
GIFSLIKGAAKVVAKGLGREVQEEEVKR

SEQ ID NO: 355 - protein, 27 aa, synthetic construct
GIFSKLAGKKIKRKYLFLFRFPLLHHH

SEQ ID NO: 356 - protein, 24 aa, synthetic construct
GIFSLIKGAAKVVAKGSGGRSEKR

SEQ ID NO: 357 - protein, 15 aa, synthetic construct
GIFSLIKGAAKVVAK

SEQ ID NO: 358 - protein, 23 aa, synthetic construct
VIPFQHPLHRDHLFFLPHRRLSL

SEQ ID NO: 359 - protein, 24 aa, synthetic construct
GLLGKVLGVGKKVLCGVTGRERCQ

SEQ ID NO: 360 - protein, 20 aa, synthetic construct
GIFSKLAGKKIKNRQCLSQF

SEQ ID NO: 361 - protein, 18 aa, synthetic construct
RPPGFPPFRVAPASSLKR

SEQ ID NO: 362 - protein, 28 aa, synthetic construct
GICSLIKGAAKVVAKGLGKEVGKFGLDL

SEQ ID NO: 363 - protein, 21 aa, synthetic construct
GLLGKVLGVGKKVLCGVTGLC

SEQ ID NO: 364 - protein, 22 aa, synthetic construct
GIFSLIKGAAKVVAKGLGKEVE

SEQ ID NO: 365 - protein, 12 aa, synthetic construct
GLLGKVLGVGKR

SEQ ID NO: 366 - protein, 20 aa, synthetic construct
GVPLIYKRPGIYVTKRPRGS

SEQ ID NO: 367 - protein, 22 aa, synthetic construct
RMIRRPPGFTPFRVAPASSLKR

SEQ ID NO: 368 - protein, 18 aa, synthetic construct
RPPGFPPFRIAPASSLKR

SEQ ID NO: 369 - protein, 16 aa, synthetic construct
GIFKVNIFGRVVLCNA

SEQ ID NO: 370 - protein, 11 aa, synthetic construct
GILLNTLKRCR

SEQ ID NO: 371 - protein, 18 aa, synthetic construct
RPPGCSPFRIAPASSLKR

SEQ ID NO: 372 - protein, 30 aa, synthetic construct
GIFSLIKGAAKVVAKGLGKEVGKFGLGNTS

SEQ ID NO: 373 - protein, 15 aa, synthetic construct
GFSPFRVAPASFLKR

SEQ ID NO: 374 - protein, 29 aa, synthetic construct
GIFSLIKGAAKVVAKGLGRGSSGGRSEKR

SEQ ID NO: 375 - protein, 24 aa, synthetic construct
GIFSLIKGAAKVVAKSSGGRSEKR

SEQ ID NO: 376 - protein, 29 aa, synthetic construct
GIFSLIKGLGKEVGKFGLDLMACKVTNQC

SEQ ID NO: 377 - protein, 29 aa, synthetic construct
GIFSLIKGAAKVVAKGLISEKIPLFTSSS

SEQ ID NO: 378 - protein, 21 aa, synthetic construct
FLPSSPWNEGTYVLKKLKWKK

SEQ ID NO: 379 - protein, 11 aa, synthetic construct
GIFSKLAGKKR

SEQ ID NO: 380 - protein, 27 aa, synthetic construct
SIISKLAGKKIKNLLISGLKNVGKEVG

SEQ ID NO: 381 - protein, 14 aa, synthetic construct
GLLGKVLGVGNKAQ

SEQ ID NO: 382 - protein, 24 aa, synthetic construct
GIFSKLAGKKIKNLAYVLESAYEQ

SEQ ID NO: 383 - protein, 18 aa, synthetic construct
GVPLIYKRLGLLVTYIPG

SEQ ID NO: 384 - protein, 18 aa, synthetic construct
GLLGKVLGVGKKVLCGVT

SEQ ID NO: 385 - protein, 16 aa, synthetic construct
SSCRTLSRYEAKRSLR

SEQ ID NO: 386 - protein, 21 aa, synthetic construct
VIPFQHPLHRDHLFFLPHRRL

SEQ ID NO: 387 - protein, 17 aa, synthetic construct
PPGFSPFRVAPASFLKR

SEQ ID NO: 388 - protein, 30 aa, synthetic construct
GIFSLIKGAAKVVAKGLGKVVAKGLGKEVG

SEQ ID NO: 389 - protein, 30 aa, synthetic construct
GIFSLIKGAAKVVAKGLGKEVAKGLGKEVG

SEQ ID NO: 390 - protein, 17 aa, synthetic construct
ATAWKVPPGLQPIRPIR

SEQ ID NO: 391 - protein, 25 aa, synthetic construct
GIFSLIKGAAKVVPKLAHFLAQTFG

SEQ ID NO: 392 - protein, 14 aa, synthetic construct
GLLGKVLGVGKKVL

SEQ ID NO: 393 - protein, 30 aa, synthetic construct
GIFSLIKGAAKVVARFGQGSGQVWAGPYGL

SEQ ID NO: 394 - protein, 17 aa, synthetic construct
VIPIVSGLLSCLLEKKK

SEQ ID NO: 395 - protein, 21 aa, synthetic construct
GLLGKVLGVGKKVLCVVSGLC

SEQ ID NO: 396 - protein, 26 aa, synthetic construct
VIPFVASVAAEMMHHMYCAASKRCKN

SEQ ID NO: 397 - protein, 26 aa, synthetic construct
EQERVIPIVSGLLSCLLEKKKKILKL

SEQ ID NO: 398 - protein, 26 aa, synthetic construct
EQERVIPIVSGLLSSLLKKKQKILKL

SEQ ID NO: 399 - protein, 17 aa, synthetic construct
GIFSKLAGKKRGIFSKL

SEQ ID NO: 400 - protein, 27 aa, synthetic construct
GIFSPIKGAAKVVAKGLGKEVGKFGLD

SEQ ID NO: 401 - protein, 18 aa, synthetic construct
VPPVFTPFRIAPASSLKR

SEQ ID NO: 402 - protein, 24 aa, synthetic construct
FLPAIIGMAANVLPTLLCKITKKC

SEQ ID NO: 403 - protein, 15 aa, synthetic construct
ILPFLAGLFSKILGK

SEQ ID NO: 404 - protein, 24 aa, synthetic construct
FLPALIGMAANVLPTLICKITKKC

SEQ ID NO: 405 - protein, 27 aa, synthetic construct
GIFSLIKGAAKVVAKGLGKEVGKFGVD

SEQ ID NO: 406 - protein, 29 aa, synthetic construct
GLWTTIKEGVKNVSVGVLDKIRCKITGGC

SEQ ID NO: 407 - protein, 15 aa, synthetic construct
GFSPFRVAPASSLKR

SEQ ID NO: 408 - protein, 24 aa, synthetic construct
GLWTTIKEGVKNFSVGSYRRSKKR

SEQ ID NO: 409 - protein, 12 aa, synthetic construct
GLWTTIKEGVKK

SEQ ID NO: 410 - protein, 29 aa, synthetic construct
FLKKTWKKLKKAAKSILKNTGPVTYQQSF

SEQ ID NO: 411 - protein, 30 aa, synthetic construct
GILTDTLKGAAKNVAGVLLDKLRCKITGGC

SEQ ID NO: 412 - protein, 28 aa, synthetic construct
GIFSLIKGAAKVVAKGLGKEVGKFRRKK

SEQ ID NO: 413 - protein, 12 aa, synthetic construct
FLKKTWKKLKKA

SEQ ID NO: 414 - protein, 20 aa, synthetic construct
GALKGCWTKSYPPQPCSGKR

SEQ ID NO: 415 - protein, 14 aa, synthetic construct
GIFSLIKGAAKVVA

SEQ ID NO: 416 - protein, 26 aa, synthetic construct
GLCTTIKEGVNNFSVGVLDKIRCKIT

SEQ ID NO: 417 - protein, 24 aa, synthetic construct
FLPAILGMAAKLVPTLLCKITKKC

SEQ ID NO: 418 - protein, 15 aa, synthetic construct
GLLGKVLGVGKKMWS

SEQ ID NO: 419 - protein, 26 aa, synthetic construct
VIPFVASVATEMMHHVYCAASRRKKR

SEQ ID NO: 420 - protein, 18 aa, synthetic construct
RLPGFSPFRVAPASSLKR

SEQ ID NO: 421 - protein, 10 aa, synthetic construct
GIFSKLSGKK

SEQ ID NO: 422 - protein, 24 aa, synthetic construct
FFPAILRMAAKVVPTVFCQISKKC

SEQ ID NO: 423 - protein, 18 aa, synthetic construct
VPPGFTAFRIAPASSLKR

SEQ ID NO: 424 - protein, 15 aa, synthetic construct
GLLGKVLGVGKKVLC

SEQ ID NO: 425 - protein, 26 aa, synthetic construct
VIPFGASVAAEMMHHVYCAASKRCKK

SEQ ID NO: 426 - protein, 14 aa, synthetic construct
GIFSKLAGKKIKDL

SEQ ID NO: 427 - protein, 29 aa, synthetic construct
VPPGFTPFRIAPASSLKRVPPGFTPFRIA

SEQ ID NO: 428 - protein, 29 aa, synthetic construct
VPPVFTPFRIAPASSLKRVPPGFTPFRIA

SEQ ID NO: 429 - protein, 24 aa, synthetic construct
KEERGALKGCWTKSYPPQPCSGKR

SEQ ID NO: 430 - protein, 28 aa, synthetic construct
EEERFLPAILGMAAKLVPTLLCKITKKC

SEQ ID NO: 431 - protein, 27 aa, synthetic construct
GSSGGRSEKRGIFSLIKGAAKVVAKGL

SEQ ID NO: 432 - protein, 30 aa, synthetic construct
VPPGFTPFRIAPASSLKRVPPGFTPFRIAP

SEQ ID NO: 433 - protein, 30 aa, synthetic construct
EQKRGIFSKLAGKKIKNLLISGLKNIGKEV

SEQ ID NO: 434 - protein, 21 aa, synthetic construct
GLLGKILGVGKRGLLGKILGV

SEQ ID NO: 435 - protein, 25 aa, synthetic construct
RNKKRYFLCEQKRGIFSLSLCEQKR

SEQ ID NO: 436 - protein, 18 aa, synthetic construct
GLLGKIVGGGLKLIGNHV

SEQ ID NO: 437 - protein, 22 aa, synthetic construct
LIDGYMKPLVSVIFSKGCATKY

SEQ ID NO: 438 - protein, 20 aa, synthetic construct
GLLTPIGDRGFCKINNSCRN

SEQ ID NO: 439 - protein, 18 aa, synthetic construct
HLERMKKSADVIKGIRKY

SEQ ID NO: 440 - protein, 24 aa, synthetic construct
IKDHIAPLYKHLTTTALKACGHRA

SEQ ID NO: 441 - protein, 18 aa, synthetic construct
CYSFSSLGPSTYLSVRKR

SEQ ID NO: 442 - protein, 29 aa, synthetic construct
AFLSTVKNTLTNVAGTMIDTFKCKITGVC

SEQ ID NO: 443 - protein, 7 aa, synthetic construct
VPLLPKR

SEQ ID NO: 444 - protein, 20 aa, synthetic construct
RRKLLLSAKNKMACFEFAKR

SEQ ID NO: 445 - protein, 15 aa, synthetic construct
FISSIASFLGKFLGK

SEQ ID NO: 446 - protein, 19 aa, synthetic construct
SLSGCWTKSFPRKPCLRNR

SEQ ID NO: 447 - protein, 14 aa, synthetic construct
SAWRRPSRKRRPRR

SEQ ID NO: 448 - protein, 16 aa, synthetic construct
SWLRSVPRRQFLRLRR

SEQ ID NO: 449 - protein, 11 aa, synthetic construct
FPAIICKVSKN

SEQ ID NO: 450 - protein, 19 aa, synthetic construct
KANRKLCGRKRCTSSRDER

SEQ ID NO: 451 - protein, 12 aa, synthetic construct
FPAIICKVPKNC

SEQ ID NO: 452 - protein, 16 aa, synthetic construct
GLKTHSVSKHFRIHHN

SEQ ID NO: 453 - protein, 26 aa, synthetic construct
FFPRVLPLANKFLPTIYCALPKSVGN

SEQ ID NO: 454 - protein, 22 aa, synthetic construct
CRMLYVGRTIRALRARFGEHRR

SEQ ID NO: 455 - protein, 12 aa, synthetic construct
FPAIICKVSKNC

SEQ ID NO: 456 - protein, 12 aa, synthetic construct
CVMCKTSRRRKR

SEQ ID NO: 457 - protein, 29 aa, synthetic construct
RDFIGCHTEGVVYILQCSFNLQYVGRTKR

SEQ ID NO: 458 - protein, 17 aa, synthetic construct
KIKLFGINSTHRVWRRR

SEQ ID NO: 459 - protein, 18 aa, synthetic construct
IRRGKIGPHGCAMGLQKK

SEQ ID NO: 460 - protein, 24 aa, synthetic construct
CSGYVPKDILKKKTLKTGKPPWHP

SEQ ID NO: 461 - protein, 19 aa, synthetic construct
SLSGCWTKSFPPKPCLRNR

SEQ ID NO: 462 - protein, 30 aa, synthetic construct
FLPIIGKILSTVFGKKPRNVETLKMELEII

SEQ ID NO: 463 - protein, 22 aa, synthetic construct
GIWDTIKSMGRAFAGRRLENVG

SEQ ID NO: 464 - protein, 26 aa, synthetic construct
AGYSRMIRRPPGCTPFRIAPASTLKR

SEQ ID NO: 465 - protein, 26 aa, synthetic construct
AISWGRIRRPLWCNPFRIAPASSLKR

SEQ ID NO: 466 - protein, 20 aa, synthetic construct
FLGGLIKIAPAMICAVTKKC

SEQ ID NO: 467 - protein, 16 aa, synthetic construct
GLKKLESLRPGLKIHT

SEQ ID NO: 468 - protein, 15 aa, synthetic construct
GIPFLGKILSPAFGK

SEQ ID NO: 469 - protein, 23 aa, synthetic construct
ALLKKLRVKVMEWLCDVMEEWKR

SEQ ID NO: 470 - protein, 19 aa, synthetic construct
SLRGGWTKSYPPQPCLGKR

SEQ ID NO: 471 - protein, 11 aa, synthetic construct
SLRGCWTKSYP

SEQ ID NO: 472 - protein, 24 aa, synthetic construct
FLPFIARLAAKVFPSITCSVTKKC

SEQ ID NO: 473 - protein, 26 aa, synthetic construct
ATAWRIPPPGMQPIIPIRIRPLCGKQ

SEQ ID NO: 474 - protein, 29 aa, synthetic construct
FLGGLIKIVPAMICAVTKKCTKTNKRPLM

SEQ ID NO: 475 - protein, 25 aa, synthetic construct
SILSVLKNLGKVGLGFVACEINKQC

SEQ ID NO: 476 - protein, 18 aa, synthetic construct
VESLCRTPSPATISRRKR

SEQ ID NO: 477 - protein, 17 aa, synthetic construct
YCRVMQGIAGYCRALQG

SEQ ID NO: 478 - protein, 10 aa, synthetic construct
VLLLKILSKR

SEQ ID NO: 479 - protein, 17 aa, synthetic construct
CSAQRAGVMGIQSRECR

SEQ ID NO: 480 - protein, 30 aa, synthetic construct
GIQGLFDNLKGYYKCGRCRVCRLNSVTSRR

SEQ ID NO: 481 - protein, 27 aa, synthetic construct
SFLQNLTGFYRCGRCQVCSLNTNRSRR

SEQ ID NO: 482 - protein, 24 aa, synthetic construct
IKDHIAPLYKHLTTTALKACSHRA

SEQ ID NO: 483 - protein, 8 aa, synthetic construct
RRLCRFSR

SEQ ID NO: 484 - protein, 8 aa, synthetic construct
LRPKSSKR

SEQ ID NO: 485 - protein, 25 aa, synthetic construct
GPGTSPPDAGKSARCRLSRKRRPRR

SEQ ID NO: 486 - protein, 29 aa, synthetic construct
KTISTFLDRGKVFITALGAKQVKRLRTKR

SEQ ID NO: 487 - protein, 24 aa, synthetic construct
FLPFIARLAAEVFPSIICSVTKKC

SEQ ID NO: 488 - protein, 14 aa, synthetic construct
GVKTHSVSRHFRLC

SEQ ID NO: 489 - protein, 29 aa, synthetic construct
ATIGCALHKSGLYGSMAKRKPLLKESHKK

SEQ ID NO: 490 - protein, 24 aa, synthetic construct
HQRLCKRKAYWIFTLGSMTPGGVE

SEQ ID NO: 491 - protein, 20 aa, synthetic construct
KLKLKIVDAHKAGYKKIAKR

SEQ ID NO: 492 - protein, 29 aa, synthetic construct
GQKWITHKSVYRVQPKITPSSQFKSFLQS

SEQ ID NO: 493 - protein, 27 aa, synthetic construct
FSLHPKKWLGQTIKHLQHHRTSPAECD

SEQ ID NO: 494 - protein, 19 aa, synthetic construct
ALKRCFTKDNRPIPCFGKR

SEQ ID NO: 495 - protein, 20 aa, synthetic construct
GVLQSMGGIAEYGGYCRVRR

SEQ ID NO: 496 - protein, 8 aa, synthetic construct
LMVPRKKR

SEQ ID NO: 497 - protein, 29 aa, synthetic construct
GLFLDTLKGAAKDVAGKLQEGLKCKITGC

SEQ ID NO: 498 - protein, 26 aa, synthetic construct
ATAWRIPPPGMQPIIPIRIRPLCGKR

SEQ ID NO: 499 - protein, 20 aa, synthetic construct
GFLDIIKKLGKTFAGHMLDK

SEQ ID NO: 500 - protein, 18 aa, synthetic construct
GLKTHYVSKHFRICHNKD

SEQ ID NO: 501 - protein, 15 aa, synthetic construct
GIPSLGKILSPAFGK

SEQ ID NO: 502 - protein, 23 aa, synthetic construct
GLFLDTLKGAAKDVARKLLEGLK

SEQ ID NO: 503 - protein, 19 aa, synthetic construct
LASHLLHIIPVGYNAVFKR

SEQ ID NO: 504 - protein, 7 aa, synthetic construct
RLSLLLR

SEQ ID NO: 505 - protein, 25 aa, synthetic construct
GSNIVIHCDLVLRASSELNSKRCKR

SEQ ID NO: 506 - protein, 6 aa, synthetic construct
SWVWKR

SEQ ID NO: 507 - protein, 18 aa, synthetic construct
SLRGCWTKSFPPQPLGKR

SEQ ID NO: 508 - protein, 29 aa, synthetic construct
EQERSMLSVLKNLGKVGLGFVACKINKQC

SEQ ID NO: 509 - protein, 28 aa, synthetic construct
EEERFLPFIARLAAKVFPSIICSVTKKC

SEQ ID NO: 510 - protein, 21 aa, synthetic construct
AMGRVSRAEDRVSRAEKRVKK

SEQ ID NO: 511 - protein, 14 aa, synthetic construct
AMDRVSRAEKRVKK

SEQ ID NO: 512 - protein, 29 aa, synthetic construct
AGYSRMIRRPPGCTPFRIAPASTLKRAGN

SEQ ID NO: 513 - protein, 29 aa, synthetic construct
AISWGRIRRPLWCNPFRIAPASSLKRAGN

SEQ ID NO: 514 - protein, 25 aa, synthetic construct
DTSLSMFLSRGIPFLGKILSPAFGK

SEQ ID NO: 515 - protein, 23 aa, synthetic construct
DQERSLSGCWTKSFPRKPCLRNR

SEQ ID NO: 516 - protein, 28 aa, synthetic construct
EEKRFLPFIARLAAKVFPSIICSVTKKC

SEQ ID NO: 517 - protein, 16 aa, synthetic construct
FLFPLITSFLSKFLGK

SEQ ID NO: 518 - protein, 25 aa, synthetic construct
SMLSVLKNLGKVGLGFVACKVNKQC

SEQ ID NO: 519 - protein, 5 aa, synthetic construct
QPKPR

SEQ ID NO: 520 - protein, 26 aa, synthetic construct
ATAWRIPPPGLQPIIPIRIRPLCGKQ

SEQ ID NO: 521 - protein, 19 aa, synthetic construct
SLRGCWTKSYPPQKKKKKR

SEQ ID NO: 522 - protein, 17 aa, synthetic construct
KKNVWKKKKNVPNPPKR

SEQ ID NO: 523 - protein, 26 aa, synthetic construct
FFPRVLPLANKFLPTIYCALPKGVGN

SEQ ID NO: 524 - protein, 22 aa, synthetic construct
IRLPHQGHHGKAKAKAVSRSQR

SEQ ID NO: 525 - protein, 17 aa, synthetic construct
AGLQFPVGRIHGHLKTR

SEQ ID NO: 526 - protein, 27 aa, synthetic construct
GVFLDTLKGLAGKMLGSLKCKIAGCKP

SEQ ID NO: 527 - protein, 21 aa, synthetic construct
SSPGMRRRQRWPSPARAQSKR

SEQ ID NO: 528 - protein, 26 aa, synthetic construct
AISWGRIRRPPGYTPFRIAPASTLKR

SEQ ID NO: 529 - protein, 20 aa, synthetic construct
GVCLDTLKGLAGKMLESLKR

SEQ ID NO: 530 - protein, 26 aa, synthetic construct
RLQCMPTVKGALFGGLVKPGSCPIPR

SEQ ID NO: 531 - protein, 20 aa, synthetic construct
FLGGLIRIVPAMICAVTKKC

SEQ ID NO: 532 - protein, 24 aa, synthetic construct
GLFLDTLKGAAKKRPLFTSSSVIS

SEQ ID NO: 533 - protein, 21 aa, synthetic construct
VGMHWSLPTVKVKPGSCPIPR

SEQ ID NO: 534 - protein, 26 aa, synthetic construct
GFLDIIKNLGKTFAGHMLDKIRCTIG

SEQ ID NO: 535 - protein, 9 aa, synthetic construct
FNRRGWRRR

SEQ ID NO: 536 - protein, 26 aa, synthetic construct
ARSWGRIRRPPGCNPFPITPASYLKR

SEQ ID NO: 537 - protein, 27 aa, synthetic construct
FLPIIGKILSTVFGKKPRNVETLKMEL

SEQ ID NO: 538 - protein, 21 aa, synthetic construct
GIWDTIKSMGRAFAGRRLENV

SEQ ID NO: 539 - protein, 27 aa, synthetic construct
AGKPPRRPPVRPPRRPPVRPPRRPRFP

SEQ ID NO: 540 - protein, 19 aa, synthetic construct
SLRECWTKSYPPQKKKKKR

SEQ ID NO: 541 - protein, 27 aa, synthetic construct
GVFLDTRKGLAGKMLESLKCKIAGCKP

SEQ ID NO: 542 - protein, 30 aa, synthetic construct
GKCSSQTSGVPRFATTTIETLWGIVKRKMR

SEQ ID NO: 543 - protein, 26 aa, synthetic construct
FFSELTGYFQFRNCPVCSLNGCRSRR

SEQ ID NO: 544 - protein, 16 aa, synthetic construct
ILFSAIGNALSRLFGK

SEQ ID NO: 545 - protein, 15 aa, synthetic construct
KVSLLKMRAKWRKKR

SEQ ID NO: 546 - protein, 29 aa, synthetic construct
AFLSTVKNTLTNVAGTMIDTFKCKVTGVC

SEQ ID NO: 547 - protein, 29 aa, synthetic construct
GTPHGQVRPPSRVKPGVCQPPSPECRFIR

SEQ ID NO: 548 - protein, 21 aa, synthetic construct
GQCKFYETFAPCQSITRKCHG

SEQ ID NO: 549 - protein, 28 aa, synthetic construct
ALPILGGVAAEVLPQVYCRIIRNSKKMN

SEQ ID NO: 550 - protein, 11 aa, synthetic construct
TLFRSVCLRKR

SEQ ID NO: 551 - protein, 13 aa, synthetic construct
SLRGCWTKSFPPQ

SEQ ID NO: 552 - protein, 19 aa, synthetic construct
FLPIIGKILSTVFGKKPRN

SEQ ID NO: 553 - protein, 9 aa, synthetic construct
RRWMGRIRR

SEQ ID NO: 554 - protein, 17 aa, synthetic construct
GGLQFPVGRIHRHLKTR

SEQ ID NO: 555 - protein, 28 aa, synthetic construct
GLFLDTLKGAAKDVALSVSKKRPLFTSS

SEQ ID NO: 556 - protein, 24 aa, synthetic construct
FLPFIARLAAKAFPSIICSVTKKC

SEQ ID NO: 557 - protein, 19 aa, synthetic construct
AVFSKCSGKAIKNLLIKQL

SEQ ID NO: 558 - protein, 24 aa, synthetic construct
SLRGCWTKSYLPQPCLGSCKKITQ

SEQ ID NO: 559 - protein, 27 aa, synthetic construct
FKEKKKSKKKWKMEKAWLKPTRKRKRR

SEQ ID NO: 560 - protein, 7 aa, synthetic construct
RNAILKR

SEQ ID NO: 561 - protein, 22 aa, synthetic construct
FFGGLIKIVPAMICAVTKMLKL

SEQ ID NO: 562 - protein, 24 aa, synthetic construct
FLPFIARLAAKVFPSIICSVTKRC

SEQ ID NO: 563 - protein, 20 aa, synthetic construct
FLGGLIKIVPAMICAVTKRC

SEQ ID NO: 564 - protein, 13 aa, synthetic construct
RRSNIFTGVLLKR

SEQ ID NO: 565 - protein, 30 aa, synthetic construct
FLGGLIKIVPAMICAVTKMLKLWKLELEII

SEQ ID NO: 566 - protein, 15 aa, synthetic construct
GIPLLGKILSPAFGK

SEQ ID NO: 567 - protein, 29 aa, synthetic construct
EQERSMLSVLKNLGKVGLGFVACKVNKQC

SEQ ID NO: 568 - protein, 29 aa, synthetic construct
AISWGRIRRPPGYTPFRIAPASTLKRAGN

SEQ ID NO: 569 - protein, 29 aa, synthetic construct
ARSWGRIRRPPGCNPFPITPASYLKRAGN

SEQ ID NO: 570 - protein, 26 aa, synthetic construct
VARKKRFLGGLIKIVPAMICAVTKKC

SEQ ID NO: 571 - protein, 28 aa, synthetic construct
EEERFLPFIARLAAKAFPSIICSVTKKC

SEQ ID NO: 572 - protein, 28 aa, synthetic construct
EEERFLPFIARLAAKVFPSIICSVTKRC

SEQ ID NO: 573 - protein, 10 aa, synthetic construct
SMMTGGWRKR

SEQ ID NO: 574 - protein, 15 aa, synthetic construct
GILTLKYPSLLQCRV

SEQ ID NO: 575 - protein, 19 aa, synthetic construct
HPQHLRKGIPKGQFYRIKR

SEQ ID NO: 576 - protein, 7 aa, synthetic construct
TKAWFKR

SEQ ID NO: 577 - protein, 11 aa, synthetic construct
ILQKELRWIHR

SEQ ID NO: 578 - protein, 19 aa, synthetic construct
GILQRNVLHDVRKLGLSRR

SEQ ID NO: 579 - protein, 7 aa, synthetic construct
RKWRRGR

SEQ ID NO: 580 - protein, 30 aa, synthetic construct
CVYSVINKFKAHGTVANLPRCGRKRKIDKR

SEQ ID NO: 581 - protein, 19 aa, synthetic construct
GILEKNVLPCVRKLGLSRR

SEQ ID NO: 582 - protein, 16 aa, synthetic construct
GVEAIKGVRKYGKREK

SEQ ID NO: 583 - protein, 16 aa, synthetic construct
WGLPKLTLKSLKRFGR

SEQ ID NO: 584 - protein, 17 aa, synthetic construct
TGLHFPVGRIHRHFKTR

SEQ ID NO: 585 - protein, 26 aa, synthetic construct
SRLWWNRQFLEKYKSNGMIPRGLRVR

SEQ ID NO: 586 - protein, 24 aa, synthetic construct
NKFKAHGTVANLPRCGRRRKIDKR

SEQ ID NO: 587 - protein, 11 aa, synthetic construct
RGRIQKKLSRR

SEQ ID NO: 588 - protein, 21 aa, synthetic construct
HLFHGVLHVKRSVHNKKTQVQ

SEQ ID NO: 589 - protein, 9 aa, synthetic construct
KNLKKKLKR

SEQ ID NO: 590 - protein, 29 aa, synthetic construct
GTEAIKGVRKYERRVKELTYQVKSSSRHR

SEQ ID NO: 591 - protein, 22 aa, synthetic construct
LIASYLKCLIAVVAAKGGPTSY

SEQ ID NO: 592 - protein, 23 aa, synthetic construct
GRGKSSPGPLDTRAPPPPKRSPR

SEQ ID NO: 593 - protein, 19 aa, synthetic construct
GLLGKVLGVRKKEEMSLMK

SEQ ID NO: 594 - protein, 15 aa, synthetic construct
VIPIVSGRLSSLLGK

SEQ ID NO: 595 - protein, 27 aa, synthetic construct
GIFSLIKGAAKVVAKGLGKEVGSSGGR

SEQ ID NO: 596 - protein, 21 aa, synthetic construct
GLLGKILGVGKKVLCGVSVLC

SEQ ID NO: 597 - protein, 26 aa, synthetic construct
VIPFVASVAAEMMHHVYCASSKRCKN

SEQ ID NO: 598 - protein, 16 aa, synthetic construct
GLLGKILGVGKKVLCG

SEQ ID NO: 599 - protein, 11 aa, synthetic construct
GIFSKLAGKKI

SEQ ID NO: 600 - protein, 21 aa, synthetic construct
GLLGKILGVGKKVLCGVSGRC

SEQ ID NO: 601 - protein, 13 aa, synthetic construct
GLLGKILGVGKKR

SEQ ID NO: 602 - protein, 30 aa, synthetic construct
GILLNTLKGAAKNVAVVLLDKLKCKITGGC

SEQ ID NO: 603 - protein, 22 aa, synthetic construct
GLLGKILGVGKKALTPHSTFFP

SEQ ID NO: 604 - protein, 24 aa, synthetic construct
GLLGKILGVGKKVLCGGGPPILHP

SEQ ID NO: 605 - protein, 20 aa, synthetic construct
GAPKGCWTKSYPPKPCSGIR

SEQ ID NO: 606 - protein, 15 aa, synthetic construct
GLWTTIKEGGKLQKK

SEQ ID NO: 607 - protein, 23 aa, synthetic construct
GIFSLIKGAAKVVAKPLATTLAA

SEQ ID NO: 608 - protein, 24 aa, synthetic construct
GIFSKLAGKKMFTLKKPLLLIVLL

SEQ ID NO: 609 - protein, 18 aa, synthetic construct
GLLGKVLGVGKKELCGVS

SEQ ID NO: 610 - protein, 28 aa, synthetic construct
GFLSLFSNAAKFLGKTLLKNVGKAGLET

SEQ ID NO: 611 - protein, 27 aa, synthetic construct
GIFSKLAGKKIKNLLISETKRGIFSKL

SEQ ID NO: 612 - protein, 15 aa, synthetic construct
GLLSGILGAGKKIVC

SEQ ID NO: 613 - protein, 16 aa, synthetic construct
GIHLNTLKGAAKNVAG

SEQ ID NO: 614 - protein, 18 aa, synthetic construct
HPPGFSPFRVAPASSLKR

SEQ ID NO: 615 - protein, 27 aa, synthetic construct
GIFSKLAGKMIKNLLISGLKNIGKEAG

SEQ ID NO: 616 - protein, 29 aa, synthetic construct
GIFSKLARKKIKNLLISGLKNIGKEVGMD

SEQ ID NO: 617 - protein, 20 aa, synthetic construct
GLLGKVLGVGKKEEMSLMKG

SEQ ID NO: 618 - protein, 26 aa, synthetic construct
AIPFVASVAAEMMHHVYCAASKRCKN

SEQ ID NO: 619 - protein, 19 aa, synthetic construct
FRFPLLHHHPSLSLCEQKR

SEQ ID NO: 620 - protein, 19 aa, synthetic construct
GLLGKILGVGKKVLCGVSG

SEQ ID NO: 621 - protein, 25 aa, synthetic construct
GLWTTIKEGVKNFSVGVLDKIRCKI

SEQ ID NO: 622 - protein, 17 aa, synthetic construct
DLLGKILGVGKKVLCGV

SEQ ID NO: 623 - protein, 26 aa, synthetic construct
VIPFVASVAAEMMHHVYRAASKRCKN

SEQ ID NO: 624 - protein, 23 aa, synthetic construct
RLFLFRFPLLHHHPSFAHREIRR

SEQ ID NO: 625 - protein, 27 aa, synthetic construct
FISISLKRRRPPGFSPFRVAPASSLKR

SEQ ID NO: 626 - protein, 30 aa, synthetic construct
GILLNTLKGAAKNVAGVLIDKLKCKITGGC

SEQ ID NO: 627 - protein, 26 aa, synthetic construct
GIPFVASVAAEMMHHVYCAASKRCKN

SEQ ID NO: 628 - protein, 21 aa, synthetic construct
GLLGKVLGVGKKVLCGVSGRC

SEQ ID NO: 629 - protein, 21 aa, synthetic construct
GILSGLLGAGKKIVCGLSGMC

SEQ ID NO: 630 - protein, 28 aa, synthetic construct
GLLDSFKNLALNAAKSAGVSVLNYPSPK

SEQ ID NO: 631 - protein, 20 aa, synthetic construct
FFPLLAGLAANFLPKIFCKI

SEQ ID NO: 632 - protein, 12 aa, synthetic construct
GLLSGILGAGKK

SEQ ID NO: 633 - protein, 21 aa, synthetic construct
KKRSKRVIPIVSGLLSSLLGK

SEQ ID NO: 634 - protein, 20 aa, synthetic construct
EQERGLLGKILGVGKKVLCG

SEQ ID NO: 635 - protein, 27 aa, synthetic construct
VPPGFTPFRIAPASSLKRVPPGFTPFR

SEQ ID NO: 636 - protein, 29 aa, synthetic construct
GILTASHLPISPAPGTPSHHQKNGRRNCR

SEQ ID NO: 637 - protein, 12 aa, synthetic construct
GRPRWSHRSRRR

SEQ ID NO: 638 - protein, 10 aa, synthetic construct
KEVVAKILKK

SEQ ID NO: 639 - protein, 28 aa, synthetic construct
SSESGMTPLAYAAASGHLTIVMLLCKRR

SEQ ID NO: 640 - protein, 25 aa, synthetic construct
QSGLSICTYCCKCCQNKQCGYCCRT

SEQ ID NO: 641 - protein, 12 aa, synthetic construct
GIPRGQFFRVKR

SEQ ID NO: 642 - protein, 22 aa, synthetic construct
GLGKILSAAVKVIGVVYRVLNQ

SEQ ID NO: 643 - protein, 21 aa, synthetic construct
GGGSRRGGGGRGGGSRRGGGG

SEQ ID NO: 644 - protein, 9 aa, synthetic construct
IKFTCKLSA

SEQ ID NO: 645 - protein, 21 aa, synthetic construct
GGGSRRGGGGRGGRASSRSTA

SEQ ID NO: 646 - protein, 25 aa, synthetic construct
HSHLSICSFCCNCCKYKGCGWCCLT

SEQ ID NO: 647 - protein, 14 aa, synthetic construct
RLALGFRGASVSKR

SEQ ID NO: 648 - protein, 28 aa, synthetic construct
GGGSRRGGGGRGGRGGRPGHRCGELPEP

SEQ ID NO: 649 - protein, 16 aa, synthetic construct
GGGSRRGGGGRGGRGG

SEQ ID NO: 650 - protein, 24 aa, synthetic construct
LIYLMKCRCGAFYVGKTIRPLKKR

SEQ ID NO: 651 - protein, 30 aa, synthetic construct
VRKWKTTGTVLVKARSGRPHKILERRRMVR

SEQ ID NO: 652 - protein, 16 aa, synthetic construct
FLPGILPAICAATKKC

SEQ ID NO: 653 - protein, 15 aa, synthetic construct
WISNIPKSQFCRLRR

SEQ ID NO: 654 - protein, 17 aa, synthetic construct
HTKWLTCKPTGPWRPTD

SEQ ID NO: 655 - protein, 27 aa, synthetic construct
HTKWRTSRCTGHWRPTETIGALRKMLG

SEQ ID NO: 656 - protein, 26 aa, synthetic construct
FIPKLPPLFNKFLPKIYCALPNAVEN

SEQ ID NO: 657 - protein, 16 aa, synthetic construct
FLPIFLPLICSVTKKC

SEQ ID NO: 658 - protein, 19 aa, synthetic construct
GILTLTNTGKKTQHLNVKL

SEQ ID NO: 659 - protein, 30 aa, synthetic construct
ALPILGGVAAEVLPQVYCRIPLKSSGRLHS

SEQ ID NO: 660 - protein, 24 aa, synthetic construct
GLGSILKVGAKLLGKTLLKTAGKA

SEQ ID NO: 661 - protein, 23 aa, synthetic construct
GGRTPRNSRRGPSREPVSPQLRR

SEQ ID NO: 662 - protein, 20 aa, synthetic construct
FLPVVASLATKIRCAVTKKC

SEQ ID NO: 663 - protein, 16 aa, synthetic construct
RTLIRCWLNGHRSMRR

SEQ ID NO: 664 - protein, 16 aa, synthetic construct
HTIKGIPVGQFLRLKR

SEQ ID NO: 665 - protein, 28 aa, synthetic construct
SSKKKKCKFFCKLKKKINSIPIISIPFK

SEQ ID NO: 666 - protein, 13 aa, synthetic construct
AVTLLGILKKRLK

SEQ ID NO: 667 - protein, 24 aa, synthetic construct
FLPVVASLAAKVLPSIICAVTKKC

SEQ ID NO: 668 - protein, 13 aa, synthetic construct
FIPKLPPLFNKFS

SEQ ID NO: 669 - protein, 9 aa, synthetic construct
RLNLKLLKR

SEQ ID NO: 670 - protein, 29 aa, synthetic construct
GILTLKYPIEHGIITNRGVHCRRCGARSS

SEQ ID NO: 671 - protein, 20 aa, synthetic construct
GALRGCWTKSYPPQPCSGKR

SEQ ID NO: 672 - protein, 17 aa, synthetic construct
RTLKNKIQKETKKRSAR

SEQ ID NO: 673 - protein, 28 aa, synthetic construct
EEQRFLPVVAGLAAKVLPSIICAVTKKC

SEQ ID NO: 674 - protein, 28 aa, synthetic construct
EQERGLGSILKVGAKLLGKTLLKTAGKA

SEQ ID NO: 675 - protein, 24 aa, synthetic construct
EEERFLPVVASLATKIRCAVTKKC

SEQ ID NO: 676 - protein, 27 aa, synthetic construct
DAEEERRFLPVVASLATKIRCAVTKKC

SEQ ID NO: 677 - protein, 22 aa, synthetic construct
ALSSALGRPRRIAATCPAILSR

SEQ ID NO: 678 - protein, 15 aa, synthetic construct
FIGALVQALSRVLGK

SEQ ID NO: 679 - protein, 8 aa, synthetic construct
FWPPFGRR

SEQ ID NO: 680 - protein, 9 aa, synthetic construct
FWPFPFGRR

SEQ ID NO: 681 - protein, 25 aa, synthetic construct
QSHLSICVHCCNCCKFKGCGKCCLT

SEQ ID NO: 682 - protein, 25 aa, synthetic construct
GVGKFLHAAGKFGKALMGEMMKSKR

SEQ ID NO: 683 - protein, 28 aa, synthetic construct
AIRGVGKFLHAAGKFGKALMGEMLKSKR

SEQ ID NO: 684 - protein, 29 aa, synthetic construct
FVRGMASKAGTIVGKLAKVALNALGRRDS

SEQ ID NO: 685 - protein, 28 aa, synthetic construct
GVGKFLHAAGKFGKALMGEMMKSKRWVE

SEQ ID NO: 686 - protein, 8 aa, synthetic construct
VIRKSCRR

SEQ ID NO: 687 - protein, 25 aa, synthetic construct
HSHLSICVHCCNCCKYKGCGKCCLT

SEQ ID NO: 688 - protein, 21 aa, synthetic construct
SSSTGSHGKVVRRILHCCRIS

SEQ ID NO: 689 - protein, 17 aa, synthetic construct
HRPGGKETLNDAQPCRR

SEQ ID NO: 690 - protein, 14 aa, synthetic construct
GAVPGLPSKGRLPH

SEQ ID NO: 691 - protein, 26 aa, synthetic construct
EPEPGNNRPVYIPQPRPPHPRLRREP

SEQ ID NO: 692 - protein, 7 aa, synthetic construct
RLIRHKR

SEQ ID NO: 693 - protein, 25 aa, synthetic construct
TRPGCGRGRCRWTVCRSDRSRTSCA

SEQ ID NO: 694 - protein, 21 aa, synthetic construct
AAGRRWKFGRRRLLGHGVRHT

SEQ ID NO: 695 - protein, 24 aa, synthetic construct
GIYTGRLLPVYIPQPRPPHPRLRR

SEQ ID NO: 696 - protein, 9 aa, synthetic construct
KNILLKGKR

SEQ ID NO: 697 - protein, 17 aa, synthetic construct
EIPHIYKKVKKGKKVKN

SEQ ID NO: 698 - protein, 26 aa, synthetic construct
FFFFFFQIIYVPSCNRNLMRIGAIFH

SEQ ID NO: 699 - protein, 15 aa, synthetic construct
YARPCAARRPSSRWS

SEQ ID NO: 700 - protein, 14 aa, synthetic construct
SSRSVSRTRPGSKR

SEQ ID NO: 701 - protein, 14 aa, synthetic construct
ITVTSTFSIFRKCT

SEQ ID NO: 702 - protein, 20 aa, synthetic construct
GRQIQRLKLHQSLERARGQR

SEQ ID NO: 703 - protein, 8 aa, synthetic construct
YAPWMRKR

SEQ ID NO: 704 - protein, 16 aa, synthetic construct
AKIMEKMPRRIKRRRR

SEQ ID NO: 705 - protein, 11 aa, synthetic construct
GVVWENLRKIR

SEQ ID NO: 706 - protein, 25 aa, synthetic construct
DNKWQNVHFHRSAVTGPSPFSFNHK

SEQ ID NO: 707 - protein, 24 aa, synthetic construct
GISTYFVFTRPHGVTLTFHEILKR

SEQ ID NO: 708 - protein, 17 aa, synthetic construct
AFVRILCYCCPRKMKRR

SEQ ID NO: 709 - protein, 22 aa, synthetic construct
SRSPSKSCKASRKLKSKSPRPR

SEQ ID NO: 710 - protein, 20 aa, synthetic construct
KKFPNTPLELPLIRNKLTKR

SEQ ID NO: 711 - protein, 26 aa, synthetic construct
IPTTICVCIFIYLRMIYIGTSRSIKR

SEQ ID NO: 712 - protein, 10 aa, synthetic construct
GMIHSGGRGR

SEQ ID NO: 713 - protein, 24 aa, synthetic construct
GISTYFVFTRPHGIALTFQEILKR

SEQ ID NO: 714 - protein, 16 aa, synthetic construct
GTSLVKLISLFLNKSI

SEQ ID NO: 715 - protein, 12 aa, synthetic construct
FLSTPKKARMKL

SEQ ID NO: 716 - protein, 25 aa, synthetic construct
GFKSQQRGEGREAFKYCVKQEMKAR

SEQ ID NO: 717 - protein, 18 aa, synthetic construct
ITFLECVTRVTQAVRRKR

SEQ ID NO: 718 - protein, 25 aa, synthetic construct
DNKWQNVHFHRSAVTGPTSFSFSHK

SEQ ID NO: 719 - protein, 13 aa, synthetic construct
AVKLRSNTITKKL

SEQ ID NO: 720 - protein, 24 aa, synthetic construct
FFGFWKYGEKTAASITLNPPQWRS

SEQ ID NO: 721 - protein, 19 aa, synthetic construct
GILRSLGWIQMPRSRRRHR

SEQ ID NO: 722 - protein, 23 aa, synthetic construct
DAEPSRPRPQQVPPRPPHPRLRR

SEQ ID NO: 723 - protein, 18 aa, synthetic construct
AAGAGKVTKSAQKAQKAK

SEQ ID NO: 724 - protein, 16 aa, synthetic construct
RSKRAKKFSGKTLLTG

SEQ ID NO: 725 - protein, 16 aa, synthetic construct
RVLTIASRANSRRKKR

SEQ ID NO: 726 - protein, 14 aa, synthetic construct
KHHHIKLRHERHRR

SEQ ID NO: 727 - protein, 21 aa, synthetic construct
GGGAGGARRSSPPSRRPSSRR

SEQ ID NO: 728 - protein, 12 aa, synthetic construct
IQILAPVIRSKR

SEQ ID NO: 729 - protein, 7 aa, synthetic construct
VKCRVRR

SEQ ID NO: 730 - protein, 30 aa, synthetic construct
ALLKLEKPLKLGGAVQPINLPCIPSTPSGR

SEQ ID NO: 731 - protein, 24 aa, synthetic construct
IAFLAVVASAKPYRGFRISLFGTR

SEQ ID NO: 732 - protein, 27 aa, synthetic construct
VYLYCLSRGGPSAKPYRGFRISLFDTR

SEQ ID NO: 733 - protein, 22 aa, synthetic construct
FLSVVASSLPYRGFRISLFGTR

SEQ ID NO: 734 - protein, 24 aa, synthetic construct
IAFLAVVASAKPYRGFRISLFATR

SEQ ID NO: 735 - protein, 19 aa, synthetic construct
NIKIPPVLHKVSVPLVSKR

SEQ ID NO: 736 - protein, 9 aa, synthetic construct
ISLSSLFKR

SEQ ID NO: 737 - protein, 17 aa, synthetic construct
PMAPDISVNIHGSLKKR

SEQ ID NO: 738 - protein, 22 aa, synthetic construct
FLAVVASSKPYRGFRISLFATR

SEQ ID NO: 739 - protein, 14 aa, synthetic construct
VLSYYKLKSKNIFV

SEQ ID NO: 740 - protein, 25 aa, synthetic construct
RAIFTINSLHAIPFAGCTVTGRLTR

SEQ ID NO: 741 - protein, 28 aa, synthetic construct
LKLEKPLKLGGAVQPINLPSISSTPSGR

SEQ ID NO: 742 - protein, 12 aa, synthetic construct
IQLLAPVIRSKR

SEQ ID NO: 743 - protein, 20 aa, synthetic construct
VMLPDKFGNTALHVATRKKR

SEQ ID NO: 744 - protein, 27 aa, synthetic construct
KLEKPLKLGGAVQPINLPSIPSTPSGR

SEQ ID NO: 745 - protein, 10 aa, synthetic construct
SILSTLSHKR

SEQ ID NO: 746 - protein, 24 aa, synthetic construct
GWGLINIKIPPVLHKVSVPLVSKR

SEQ ID NO: 747 - protein, 22 aa, synthetic construct
NVKKLPLQAWKRWCTQILSALR

SEQ ID NO: 748 - protein, 23 aa, synthetic construct
YPCPVRRIDDRRGHLPHLRPPIL

SEQ ID NO: 749 - protein, 17 aa, synthetic construct
VIFYFVTIKFRKKFNLI

SEQ ID NO: 750 - protein, 25 aa, synthetic construct
TGWGLTNIKIPPVLHKVSVPLVSKR

SEQ ID NO: 751 - protein, 22 aa, synthetic construct
FLAVVASAKPYRGFRIPLFDTR

SEQ ID NO: 752 - protein, 9 aa, synthetic construct
FRISLFATR

SEQ ID NO: 753 - protein, 29 aa, synthetic construct
FIRTITKCTTTRTSSVSNVSGSGTIQPEH

SEQ ID NO: 754 - protein, 16 aa, synthetic construct
RRRTSPGPWTVSLFRR

SEQ ID NO: 755 - protein, 25 aa, synthetic construct
IKLTGELPSPMNPPPGCAFNARCRR

SEQ ID NO: 756 - protein, 23 aa, synthetic construct
RKRRWNTTFNTCQKMTRFTRRRR

SEQ ID NO: 757 - protein, 8 aa, synthetic construct
YRKFIFKR

SEQ ID NO: 758 - protein, 8 aa, synthetic construct
KVEHLSKR

SEQ ID NO: 759 - protein, 22 aa, synthetic construct
KQKLVKNIIFKYCIKIFIYFLK

SEQ ID NO: 760 - protein, 17 aa, synthetic construct
SVSSLAKNSAWPVSLKR

SEQ ID NO: 761 - protein, 19 aa, synthetic construct
IPPLIYNLFPNLREMRGRR

SEQ ID NO: 762 - protein, 17 aa, synthetic construct
AFVRILCACCPGRVRRR

SEQ ID NO: 763 - protein, 28 aa, synthetic construct
IALPTEKLKKFIVRRKWQWPARLRTRGY

SEQ ID NO: 764 - protein, 27 aa, synthetic construct
TLVLWRRGFFFCGSSGPASFSRAFYKR

SEQ ID NO: 765 - protein, 24 aa, synthetic construct
GGGLTNIKIPPLLHKVSVPLVSKR

SEQ ID NO: 766 - protein, 22 aa, synthetic construct
FLSVVASSLHYRGFRISLFDTR

SEQ ID NO: 767 - protein, 28 aa, synthetic construct
LKLAKPLKLGGAVQPINLPSIPSTPSGR

SEQ ID NO: 768 - protein, 19 aa, synthetic construct
NVKLFLFQLLRGLAYCHRR

SEQ ID NO: 769 - protein, 12 aa, synthetic construct
GTKGIINFRIKR

SEQ ID NO: 770 - protein, 25 aa, synthetic construct
RARKIRRRRGSLRHCVTIPSTPSGR

SEQ ID NO: 771 - protein, 9 aa, synthetic construct
FRISLFGTR

SEQ ID NO: 772 - protein, 16 aa, synthetic construct
VRTWAQLVNVEKLLKR

SEQ ID NO: 773 - protein, 7 aa, synthetic construct
WMAISKR

SEQ ID NO: 774 - protein, 22 aa, synthetic construct
FLSVVASAIHYRGFRISLFDTR

SEQ ID NO: 775 - protein, 28 aa, synthetic construct
LKLEKPLKLGGAVQPINLPSIASTPSGR

SEQ ID NO: 776 - protein, 23 aa, synthetic construct
GKKLRTLRRHIGMIFQSFNLVKR

SEQ ID NO: 777 - protein, 15 aa, synthetic construct
KIRDFRKEVKKVLKR

SEQ ID NO: 778 - protein, 23 aa, synthetic construct
KTSSFLTWIGNILPSIPSTPSGR

SEQ ID NO: 779 - protein, 23 aa, synthetic construct
GAHKEVFKRDTALTKKASEKPGK

SEQ ID NO: 780 - protein, 10 aa, synthetic construct
SRRVKCRVRR

SEQ ID NO: 781 - protein, 21 aa, synthetic construct
KHHHIKLRHERHRRYILKSLI

SEQ ID NO: 782 - protein, 23 aa, synthetic construct
GAHKEVFKRDTALTKEAAKKAKK

SEQ ID NO: 783 - protein, 26 aa, synthetic construct
GAHKEVFKRALAQKLNRNQTKPGKYR

SEQ ID NO: 784 - protein, 23 aa, synthetic construct
GAHKEVFKRDTALTKKASQKTGK

SEQ ID NO: 785 - protein, 23 aa, synthetic construct
GAHKEVFKRDTALTKQTVKKDNK

SEQ ID NO: 786 - protein, 14 aa, synthetic construct
VTISIARRVSSHKR

SEQ ID NO: 787 - protein, 8 aa, synthetic construct
PVKLAKCR

SEQ ID NO: 788 - protein, 25 aa, synthetic construct
GFKSQQRHKHLQAFTSCVKQEMKSR

SEQ ID NO: 789 - protein, 17 aa, synthetic construct
SVASLAKNSAWPVSLKR

SEQ ID NO: 790 - protein, 30 aa, synthetic construct
GILRLVTRRFRFSPTNLNRYTVARLVSGVP

SEQ ID NO: 791 - protein, 17 aa, synthetic construct
LMWVLSEMILFGKNCKR

SEQ ID NO: 792 - protein, 15 aa, synthetic construct
PPHALNTLNSQHHKR

SEQ ID NO: 793 - protein, 16 aa, synthetic construct
ATAAECLKHPWLKIKK

SEQ ID NO: 794 - protein, 26 aa, synthetic construct
PGSGSGSASRPVYIPPPRPPHPRLRR

SEQ ID NO: 795 - protein, 17 aa, synthetic construct
KEVVRVWFCNRRQKEKR

SEQ ID NO: 796 - protein, 15 aa, synthetic construct
VTISIARRVSSHKRG

SEQ ID NO: 797 - protein, 19 aa, synthetic construct
IIRATAAECLKHPWLKIKK

SEQ ID NO: 798 - protein, 16 aa, synthetic construct
RTNIKSFFLSQLRRRR

SEQ ID NO: 799 - protein, 8 aa, synthetic construct
GRDKISKR

SEQ ID NO: 800 - protein, 22 aa, synthetic construct
NKIKFINKYVKKVQLKKILVKS

SEQ ID NO: 801 - protein, 7 aa, synthetic construct
ILLKGKR

SEQ ID NO: 802 - protein, 22 aa, synthetic construct
IIGEWRKHKQRKNKLPSKVRVM

SEQ ID NO: 803 - protein, 24 aa, synthetic construct
AKMPISVLSKVSDHVKFIKKAAKM

SEQ ID NO: 804 - protein, 23 aa, synthetic construct
APEPEPGRPRPQQVPPRPPHPRL

SEQ ID NO: 805 - protein, 24 aa, synthetic construct
AKMPISVLSKVSEHVKFIKKAAKM

SEQ ID NO: 806 - protein, 18 aa, synthetic construct
GAVHRPYLGVNLRNLSPR

SEQ ID NO: 807 - protein, 21 aa, synthetic construct
FAVRDMRQTVAVGVIKSVTFK

SEQ ID NO: 808 - protein, 18 aa, synthetic construct
GIFLPGSVILRALSRHGR

SEQ ID NO: 809 - protein, 14 aa, synthetic construct
VSSRTICLRVRKKR

SEQ ID NO: 810 - protein, 15 aa, synthetic construct
VYIPPPRPPHPRLRR

SEQ ID NO: 811 - protein, 22 aa, synthetic construct
SRWLITKKRTRHYSLTRLRNRR

SEQ ID NO: 812 - protein, 25 aa, synthetic construct
GFKSQRRHKHLQAFSSCVKQEVKTR

SEQ ID NO: 813 - protein, 21 aa, synthetic construct
ITSLSLIRYPADKFVILRKGR

SEQ ID NO: 814 - protein, 17 aa, synthetic construct
KLIEWFKVVLQWDPKKR

SEQ ID NO: 815 - protein, 16 aa, synthetic construct
RKIIAVSVHKLCRVKR

SEQ ID NO: 816 - protein, 24 aa, synthetic construct
RRFFFATAPCGYSRKFCKITRRKR

SEQ ID NO: 817 - protein, 9 aa, synthetic construct
FRPPSPKRR

SEQ ID NO: 818 - protein, 13 aa, synthetic construct
FFRKPGHGHHKKR

SEQ ID NO: 819 - protein, 25 aa, synthetic construct
FIKTQVLKHLVAGVRVARGLDWKWR

SEQ ID NO: 820 - protein, 14 aa, synthetic construct
KHHHIKLRHGRHRR

SEQ ID NO: 821 - protein, 18 aa, synthetic construct
GRMASGRIIARVLNRHGR

SEQ ID NO: 822 - protein, 24 aa, synthetic construct
HIGALARLGWLPSFRAPRFSRSPR

SEQ ID NO: 823 - protein, 18 aa, synthetic construct
VGKIPGSFLSGPARRGRR

SEQ ID NO: 824 - protein, 25 aa, synthetic construct
DSKWQNVHFHRSAVTGPSPFSFNHK

SEQ ID NO: 825 - protein, 22 aa, synthetic construct
IAWSFRFPSMNTLQNSSRRRKR

SEQ ID NO: 826 - protein, 12 aa, synthetic construct
FACPIGFFRLKR

SEQ ID NO: 827 - protein, 13 aa, synthetic construct
FCKFLRRLGNKKR

SEQ ID NO: 828 - protein, 25 aa, synthetic construct
GEFRTVRRQRPIDPRPPRHGPPAKR

SEQ ID NO: 829 - protein, 11 aa, synthetic construct
FRPPSPKRRSR

SEQ ID NO: 830 - protein, 17 aa, synthetic construct
GVDFTHIRKRMTSWFKR

SEQ ID NO: 831 - protein, 21 aa, synthetic construct
KHHHIKLRHGRHRRSVLRTLV

SEQ ID NO: 832 - protein, 28 aa, synthetic construct
TVSSLFRRAVNFVGARSRGSRHPSPIRR

SEQ ID NO: 833 - protein, 8 aa, synthetic construct
VPFGLKPR

SEQ ID NO: 834 - protein, 16 aa, synthetic construct
GLLPLIDPRVCTRSKA

SEQ ID NO: 835 - protein, 26 aa, synthetic construct
VALPTDKLKKFIVRRKWQTTKKKSHG

SEQ ID NO: 836 - protein, 13 aa, synthetic construct
SPPGKLPRSTRKR

SEQ ID NO: 837 - protein, 22 aa, synthetic construct
TPALCAAAKGQLETLKILALHR

SEQ ID NO: 838 - protein, 25 aa, synthetic construct
WVPPINELWKKFIKQGTAIWRSGKQ

SEQ ID NO: 839 - protein, 16 aa, synthetic construct
RRHAHSYFGEKLKVKR

SEQ ID NO: 840 - protein, 23 aa, synthetic construct
YVIKKRWVKAVNTILALQRMGAH

SEQ ID NO: 841 - protein, 15 aa, synthetic construct
IWVFLRQKSRRVQFA

SEQ ID NO: 842 - protein, 22 aa, synthetic construct
YVIKKRWVKAVNTILALQRMGA

SEQ ID NO: 843 - protein, 8 aa, synthetic construct
RSHSLVKR

SEQ ID NO: 844 - protein, 25 aa, synthetic construct
IIPIQLGKCFKWGTHDKTGNPRSSR

SEQ ID NO: 845 - protein, 15 aa, synthetic construct
GIVFFTLLSKTRRKR

SEQ ID NO: 846 - protein, 21 aa, synthetic construct
IVVCYARIFYIVRKTAKRSRR

SEQ ID NO: 847 - protein, 15 aa, synthetic construct
LKRHKKQSKKKPSKK

SEQ ID NO: 848 - protein, 21 aa, synthetic construct
VCDYPRRAKCEGIGGSGHAKA

SEQ ID NO: 849 - protein, 18 aa, synthetic construct
ITICDWAAAVIHKVRSKK

SEQ ID NO: 850 - protein, 23 aa, synthetic construct
VALPTDKLKKFIVRRKWQTRKTR

SEQ ID NO: 851 - protein, 9 aa, synthetic construct
FPLEQRLKK

SEQ ID NO: 852 - protein, 11 aa, synthetic construct
GMPGSNSRTKR

SEQ ID NO: 853 - protein, 8 aa, synthetic construct
IAAIFHKR

SEQ ID NO: 854 - protein, 9 aa, synthetic construct
VPMRLLSRR

SEQ ID NO: 855 - protein, 28 aa, synthetic construct
GIFFFGMSNSLVNPLIYGAFHLWPRRRR

SEQ ID NO: 856 - protein, 10 aa, synthetic construct
SMVKLLLSRR

SEQ ID NO: 857 - protein, 8 aa, synthetic construct
RSHALVKR

SEQ ID NO: 858 - protein, 12 aa, synthetic construct
FFRFPLEQRLKK

SEQ ID NO: 859 - protein, 10 aa, synthetic construct
TTRGRRSGFS

SEQ ID NO: 860 - protein, 20 aa, synthetic construct
SRSPRKSRSPKLLKSLRKHR

SEQ ID NO: 861 - protein, 15 aa, synthetic construct
KASLSLMLGQKSSKK

SEQ ID NO: 862 - protein, 18 aa, synthetic construct
PPPKTHQSRIDRPESPRR

SEQ ID NO: 863 - protein, 12 aa, synthetic construct
LSALIWLLKRKR

SEQ ID NO: 864 - protein, 15 aa, synthetic construct
GAFVLWGPTPRPRRR

SEQ ID NO: 865 - protein, 29 aa, synthetic construct
EMMLNNHMKIQRIILEEAAESRLKCRNTR

SEQ ID NO: 866 - protein, 23 aa, synthetic construct
STRSRSPRKSRSPKLLKSLRKHR

SEQ ID NO: 867 - protein, 8 aa, synthetic construct
VMLPKFKR

SEQ ID NO: 868 - protein, 23 aa, synthetic construct
GSKPTTPTGPSPSLPASPAPSRR

SEQ ID NO: 869 - protein, 18 aa, synthetic construct
QLWIYWKYGTPRNHLLPR

SEQ ID NO: 870 - protein, 18 aa, synthetic construct
GEKRPRAVPAGAGFHLKF

SEQ ID NO: 871 - protein, 18 aa, synthetic construct
HKRSKRAVPAGAGFHLKF

SEQ ID NO: 872 - protein, 21 aa, synthetic construct
HKRGEKRPRAVPAGAGFHLKF

SEQ ID NO: 873 - protein, 16 aa, synthetic construct
HKRAVPAGAGFHLKFW

SEQ ID NO: 874 - protein, 19 aa, synthetic construct
GEKRPRAVPAGAGFHLKFW

SEQ ID NO: 875 - protein, 19 aa, synthetic construct
HKRSKRAVPAGAGFHLKFW

SEQ ID NO: 876 - protein, 22 aa, synthetic construct
HKRGEKRPRAVPAGAGFHLKFW

SEQ ID NO: 877 - protein, 18 aa, synthetic construct
GRPNPVNTKPTPYPRLRR

SEQ ID NO: 878 - protein, 24 aa, synthetic construct
VIAEIQHITYKEWLPNFLGRRYTR

SEQ ID NO: 879 - protein, 23 aa, synthetic construct
AFRHRRTLRTWAASRCGLRACYK

SEQ ID NO: 880 - protein, 17 aa, synthetic construct
NKSTSKKKPKKPKKLKK

SEQ ID NO: 881 - protein, 17 aa, synthetic construct
RRAIFASIRGYLGLRKR

SEQ ID NO: 882 - protein, 5 aa, synthetic construct
KIVKR

SEQ ID NO: 883 - protein, 21 aa, synthetic construct
TILTPKRHFSQINVFAKLLKL

SEQ ID NO: 884 - protein, 25 aa, synthetic construct
FIKTQVLRHLVAGVRVARGLDWKWR

SEQ ID NO: 885 - protein, 30 aa, synthetic construct
SLVGCPRPNFLRSWIRCKFFCKNNKPMCRK

SEQ ID NO: 886 - protein, 25 aa, synthetic construct
GFGSGGRCNFHRKRKLIVRPGISKR

SEQ ID NO: 887 - protein, 27 aa, synthetic construct
WPAWVYCTRYSDRPSSGRSPRTRRPKR

SEQ ID NO: 888 - protein, 6 aa, synthetic construct
RPRKHR

SEQ ID NO: 889 - protein, 25 aa, synthetic construct
SSLSPLSSSSGLGKKKKRKSKRASR

SEQ ID NO: 890 - protein, 25 aa, synthetic construct
RVSRLRNTLCSWVYRHFVDLRKKRR

SEQ ID NO: 891 - protein, 16 aa, synthetic construct
NIGLCITCHNRNFLRR

SEQ ID NO: 892 - protein, 17 aa, synthetic construct
TPLSDIFRGQLRSRVSR

SEQ ID NO: 893 - protein, 15 aa, synthetic construct
SSKTLPFGKRKSKQR

SEQ ID NO: 894 - protein, 20 aa, synthetic construct
DMSSSLIAPPAPPKRLSHKR

SEQ ID NO: 895 - protein, 11 aa, synthetic construct
KLFLTLWKLKR

SEQ ID NO: 896 - protein, 24 aa, synthetic construct
TIKIIVGTLISKKHVLTAAHCTHD

SEQ ID NO: 897 - protein, 21 aa, synthetic construct
IYMYSYCKKGFTGKILIGCAC

SEQ ID NO: 898 - protein, 28 aa, synthetic construct
GGTLISKKHVLTAAHCTHDWIMQRRDKR

SEQ ID NO: 899 - protein, 12 aa, synthetic construct
GVGRRMGVRHYM

SEQ ID NO: 900 - protein, 7 aa, synthetic construct
SKPILKR

SEQ ID NO: 901 - protein, 6 aa, synthetic construct
RRLKPR

SEQ ID NO: 902 - protein, 19 aa, synthetic construct
VKGFRRFFVKHVFLTSSFA

SEQ ID NO: 903 - protein, 26 aa, synthetic construct
NIRPRPTARPPYINRPPNPFKPRWKR

SEQ ID NO: 904 - protein, 20 aa, synthetic construct
HIFQVLRGLNFCHNNHIMHR

SEQ ID NO: 905 - protein, 10 aa, synthetic construct
LLCGGLRHRR

SEQ ID NO: 906 - protein, 20 aa, synthetic construct
GSSSRSCRCIRLSRLSSKRT

SEQ ID NO: 907 - protein, 21 aa, synthetic construct
GGGGARHGRQTIWGNIRRNKR

SEQ ID NO: 908 - protein, 15 aa, synthetic construct
ERKGVGRRMGVRHYM

SEQ ID NO: 909 - protein, 20 aa, synthetic construct
RYHHVKLRHDRHRRSLFSFL

SEQ ID NO: 910 - protein, 21 aa, synthetic construct
TRVKGFRRFFVKHVFLTSSFA

SEQ ID NO: 911 - protein, 15 aa, synthetic construct
GGGGARHGRGLLAYC

SEQ ID NO: 912 - protein, 29 aa, synthetic construct
FLSFLKNKPVTPPPSPCYCSCGLRNEESR

SEQ ID NO: 913 - protein, 19 aa, synthetic construct
CRQQCLQPGRSRESCSLPR

SEQ ID NO: 914 - protein, 25 aa, synthetic construct
RVSRLRNTLCSWAYRHFVDLRKKRR

SEQ ID NO: 915 - protein, 25 aa, synthetic construct
CRQQCLQPGRSRESCSLPRSLFSFL

SEQ ID NO: 916 - protein, 13 aa, synthetic construct
AAKLRSNAITKKL

SEQ ID NO: 917 - protein, 25 aa, synthetic construct
SKRHLLIAKRRKDLLKSLVEKKLKR

SEQ ID NO: 918 - protein, 21 aa, synthetic construct
VRDGTLLKVVHVVRCAESSPR

SEQ ID NO: 919 - protein, 29 aa, synthetic construct
GSRRCREAIPRRSASRRAAPLRHFVSDPR

SEQ ID NO: 920 - protein, 15 aa, synthetic construct
RTLRFLSVRENLLRR

SEQ ID NO: 921 - protein, 30 aa, synthetic construct
AMPYPGTPTNRILQLLKSGYRMERPPNCGR

SEQ ID NO: 922 - protein, 19 aa, synthetic construct
ALPLMVGLYRTKQLKKRRR

SEQ ID NO: 923 - protein, 12 aa, synthetic construct
SWLARKLLFGAC

SEQ ID NO: 924 - protein, 16 aa, synthetic construct
STQSTWGITGGFRFKR

SEQ ID NO: 925 - protein, 25 aa, synthetic construct
GRPQFPGYGTFNPKGKLPVPFPKPF

SEQ ID NO: 926 - protein, 29 aa, synthetic construct
NGHFASFPHYARARVDTRIFPRRRCCTTT

SEQ ID NO: 927 - protein, 17 aa, synthetic construct
VAAFAIIGCLCCRRPRR

SEQ ID NO: 928 - protein, 18 aa, synthetic construct
RRPLVSYRCVRRRRRRRR

SEQ ID NO: 929 - protein, 8 aa, synthetic construct
TVCKKFTT

SEQ ID NO: 930 - protein, 20 aa, synthetic construct
ILLHLLLPSVLPLLLTAIRR

SEQ ID NO: 931 - protein, 15 aa, synthetic construct
GVIRYSSYTLKHFLP

SEQ ID NO: 932 - protein, 22 aa, synthetic construct
FNRAKRPASRALYAAKIMRKRR

SEQ ID NO: 933 - protein, 24 aa, synthetic construct
DVPKLRNHRRAVVRKVVKNHRSKR

SEQ ID NO: 934 - protein, 15 aa, synthetic construct
AVLPPFRIPRLRHHR

SEQ ID NO: 935 - protein, 19 aa, synthetic construct
FTEPIPHLHQSYSKRRGKR

SEQ ID NO: 936 - protein, 23 aa, synthetic construct
ISIKEALEHSFFHTVPRKWCKKH

SEQ ID NO: 937 - protein, 21 aa, synthetic construct
HIGSLARQGLLPSIRAARFSR

SEQ ID NO: 938 - protein, 20 aa, synthetic construct
GRLWDFIMKNYKLCDHSFKR

SEQ ID NO: 939 - protein, 17 aa, synthetic construct
KKKKKKKKKKKKKKQKR

SEQ ID NO: 940 - protein, 19 aa, synthetic construct
IKVWFQNRRTKIKKQLTQQ

SEQ ID NO: 941 - protein, 22 aa, synthetic construct
FITIIPLFGLLVMNRLSNAFNK

SEQ ID NO: 942 - protein, 17 aa, synthetic construct
TALKSLSILKKLAKLNM

SEQ ID NO: 943 - protein, 17 aa, synthetic construct
WMLYSRYQNRKALLRRR

SEQ ID NO: 944 - protein, 23 aa, synthetic construct
KFYQHLHMLYPNVTRVHVLLLKK

SEQ ID NO: 945 - protein, 9 aa, synthetic construct
KTISTRLKR

SEQ ID NO: 946 - protein, 22 aa, synthetic construct
GIMFAGRVVQPGVPSVAPGRSF

SEQ ID NO: 947 - protein, 17 aa, synthetic construct
FLCTRRENEGRRRRFRR

SEQ ID NO: 948 - protein, 10 aa, synthetic construct
GFPKFRGIWN

SEQ ID NO: 949 - protein, 12 aa, synthetic construct
VIVVSPRKRAKR

SEQ ID NO: 950 - protein, 14 aa, synthetic construct
KYHHIKLRHGRHRR

SEQ ID NO: 951 - protein, 10 aa, synthetic construct
IPHPHHLVKR

SEQ ID NO: 952 - protein, 19 aa, synthetic construct
FTEPIPHLHQSSSKRRGKR

SEQ ID NO: 953 - protein, 26 aa, synthetic construct
LEGSGSASRRRPQQVPPRPPHPRLRR

SEQ ID NO: 954 - protein, 16 aa, synthetic construct
SLPKPPQASPRPRIRR

SEQ ID NO: 955 - protein, 24 aa, synthetic construct
ITEPVGTKAPTFTSELRGGWLKKR

SEQ ID NO: 956 - protein, 18 aa, synthetic construct
RPRPQQVPPRPPHPRLRR

SEQ ID NO: 957 - protein, 21 aa, synthetic construct
RNRDHGLASYNDFRKYCHLKR

SEQ ID NO: 958 - protein, 30 aa, synthetic construct
ICQIVCIMNIMLILFFFKSFSHSQLKNYLH

SEQ ID NO: 959 - protein, 21 aa, synthetic construct
VALPTDKLKKFIMPMKIRMQG

SEQ ID NO: 960 - protein, 9 aa, synthetic construct
RPPHPRLRR

SEQ ID NO: 961 - protein, 17 aa, synthetic construct
KYHHIKLRHGRHRRTIH

SEQ ID NO: 962 - protein, 24 aa, synthetic construct
KYHHIKLRHGRHRRDACFLPKTIH

SEQ ID NO: 963 - protein, 21 aa, synthetic construct
HPKKKFDPYVPVRNKTSIRFS

SEQ ID NO: 964 - protein, 13 aa, synthetic construct
ATKLVNSAPAKKI

SEQ ID NO: 965 - protein, 5 aa, synthetic construct
RRIKR

SEQ ID NO: 966 - protein, 22 aa, synthetic construct
SPKKKGGHVSVNVQQEVCLPSC

SEQ ID NO: 967 - protein, 8 aa, synthetic construct
FWGARIRR

SEQ ID NO: 968 - protein, 8 aa, synthetic construct
WALRWKTR

SEQ ID NO: 969 - protein, 22 aa, synthetic construct
SPKKKGGHVSVNVQQDGRHTSW

SEQ ID NO: 970 - protein, 20 aa, synthetic construct
LIFSMLPCIVQRTSHHSTRR

SEQ ID NO: 971 - protein, 21 aa, synthetic construct
SPKKKGGHVSVNKISHEILRQ

SEQ ID NO: 972 - protein, 24 aa, synthetic construct
RAPSLFRGASGVCLISRCSCSHPR

SEQ ID NO: 973 - protein, 11 aa, synthetic construct
FSVWALRWKTR

SEQ ID NO: 974 - protein, 23 aa, synthetic construct
IKLHEVEKGVVVLKLDLRLFSSR

SEQ ID NO: 975 - protein, 7 aa, synthetic construct
TSLLVKR

SEQ ID NO: 976 - protein, 13 aa, synthetic construct
AVKLRTSAATKKL

SEQ ID NO: 977 - protein, 14 aa, synthetic construct
AALLWSLKKRNRKR

SEQ ID NO: 978 - protein, 20 aa, synthetic construct
LSVSHRRPASLPGIALRASR

SEQ ID NO: 979 - protein, 18 aa, synthetic construct
FTLQQVKRHPWTICRHPR

SEQ ID NO: 980 - protein, 25 aa, synthetic construct
GFKSQQRREGWPAFTFCVKQEMKAP

SEQ ID NO: 981 - protein, 6 aa, synthetic construct
AAFGKR

SEQ ID NO: 982 - protein, 22 aa, synthetic construct
GTSTVAGSTSSSTPSGRASSKR

SEQ ID NO: 983 - protein, 28 aa, synthetic construct
WRASRATIGLQYSLYFFRTLIALTNRRY

SEQ ID NO: 984 - protein, 24 aa, synthetic construct
QNMGLRRRSSILSSVTGVRISFWW

SEQ ID NO: 985 - protein, 6 aa, synthetic construct
TVLQKR

SEQ ID NO: 986 - protein, 28 aa, synthetic construct
FHAVAKIKIPWGKVKDFLVGGMKAVGKK

SEQ ID NO: 987 - protein, 28 aa, synthetic construct
FDAVAKIKIPWGKVKDFLVGGMKAVGKK

SEQ ID NO: 988 - protein, 23 aa, synthetic construct
HSIGHSIRHSFGHSFGHSFSFRR

SEQ ID NO: 989 - protein, 20 aa, synthetic construct
GCRLPRYFWYHRWRLLRDTR

SEQ ID NO: 990 - protein, 21 aa, synthetic construct
AAIGRAGRRGTRGGAASRIPR

SEQ ID NO: 991 - protein, 27 aa, synthetic construct
FFFFCNFYKDNFKADAIISATRRRSPR

SEQ ID NO: 992 - protein, 20 aa, synthetic construct
TLPLMVGRYRAKRRLREKRR

SEQ ID NO: 993 - protein, 28 aa, synthetic construct
AVLSFVHKLFLNFLHVDTSKGKCRATLQ

SEQ ID NO: 994 - protein, 17 aa, synthetic construct
AFVRILCYCCPRRIKRR

SEQ ID NO: 995 - protein, 10 aa, synthetic construct
VPEHARVRGR

SEQ ID NO: 996 - protein, 30 aa, synthetic construct
SWLSKSVKKLVNKKNYTRLEKLAKKKLFNE

SEQ ID NO: 997 - protein, 15 aa, synthetic construct
VGTLIWILRNRSRRR

SEQ ID NO: 998 - protein, 7 aa, synthetic construct
VWSLRKR

SEQ ID NO: 999 - protein, 16 aa, synthetic construct
NIGLCIICHNRNFLRR

SEQ ID NO: 1000 - protein, 8 aa, synthetic construct
SIKLLPKP

SEQ ID NO: 1001 - protein, 23 aa, synthetic construct
LFYKTVLNSSKNIHNRMFSRLVR

SEQ ID NO: 1002 - protein, 22 aa, synthetic construct
IFPHTTRFFTFRPLASRSLYSS

SEQ ID NO: 1003 - protein, 28 aa, synthetic construct
RHRSLLCVRARTYIDCVRSCEWGHWHRR

SEQ ID NO: 1004 - protein, 13 aa, synthetic construct
RNMALAVSRLSRR

SEQ ID NO: 1005 - protein, 26 aa, synthetic construct
VIPLFLRVHILPKMKRHFHPALYLYP

SEQ ID NO: 1006 - protein, 6 aa, synthetic construct
RRLHKR

SEQ ID NO: 1007 - protein, 19 aa, synthetic construct
DIFRSLIWMQMPRSRRRHR

SEQ ID NO: 1008 - protein, 9 aa, synthetic construct
VLKLASSKK

SEQ ID NO: 1009 - protein, 25 aa, synthetic construct
EADPGSQKPRPPQVPPRPPHPRLRR

SEQ ID NO: 1010 - protein, 8 aa, synthetic construct
VTLKPVKR

SEQ ID NO: 1011 - protein, 8 aa, synthetic construct
PPHPRLRR

SEQ ID NO: 1012 - protein, 20 aa, synthetic construct
RNMALAVSRLSRRGFKVSSN

SEQ ID NO: 1013 - protein, 29 aa, synthetic construct
VPSHQSHPRLRRSQIPIPTPVPIRPRPRR

SEQ ID NO: 1014 - protein, 15 aa, synthetic construct
RPPHPRLRREAVTES

SEQ ID NO: 1015 - protein, 20 aa, synthetic construct
VPSHQSHPRLRRSLGCGGRG

SEQ ID NO: 1016 - protein, 30 aa, synthetic construct
GNPGPQPPAKSMNTLVIKKRKKKKRKCFIR

SEQ ID NO: 1017 - protein, 15 aa, synthetic construct
VVGQLLGQPKLRHSF

SEQ ID NO: 1018 - protein, 24 aa, synthetic construct
GISTYFVFTRPHGITLTFQEILKR

SEQ ID NO: 1019 - protein, 26 aa, synthetic construct
VIPLCLRVHILPKMKRHFLPALYLHA

SEQ ID NO: 1020 - protein, 9 aa, synthetic construct
STPGKIFKF

SEQ ID NO: 1021 - protein, 18 aa, synthetic construct
NKKKIRRRRRRNRRRSRR

SEQ ID NO: 1022 - protein, 12 aa, synthetic construct
FILHAKKTRSAK

SEQ ID NO: 1023 - protein, 27 aa, synthetic construct
SIRKSRKKKKNYISRKFNLFNSIEYKW

SEQ ID NO: 1024 - protein, 19 aa, synthetic construct
DIFRSLIWIQMPRSRRRHR

SEQ ID NO: 1025 - protein, 41 aa, synthetic construct
MFTMKKSLLVLFFLGIVSLSLCEEERNADEDDGEMTEEVKR

SEQ ID NO: 1026 - protein, 29 aa, synthetic construct
LGIVSLSLCQEERSADDEEGEVIEEEVKR

SEQ ID NO: 1027 - protein, 43 aa, synthetic construct
MFTMKKSLLFFFLGTIALSLCEEERGADEEENGGEITDEEVKR

SEQ ID NO: 1028 - protein, 43 aa, synthetic construct
MFTMKKSLLLVFFLGTIALSLCEEERGADDDNGGEITDEEIKR

SEQ ID NO: 1029 - protein, 41 aa, synthetic construct
MFTLRKSLLLLFFLGMVSLSLCEQERDADEDEGEVTEEVKR

SEQ ID NO: 1030 - protein, 203 aa, synthetic construct
MKLLALVLVLSCVVAYTTARKRGQYWPTNTKIFTTPYRFRREADQGSIVANLKNTPQLPFDDNENLRLVLFDNDPTVDLGEDDKEIPGPQSQPNALSNNLHLIDENDYFSSYTSQPGTYRSFPRNFGTSGRYRWRREAGGHVEPRLRFDAETQRGNSFFTDFADLQRRANGRGIEPTVSATAGIRFRQEADQINPLAVRRERR

SEQ ID NO: 1031 - protein, 28 aa, synthetic construct
IFLVGCKLFGNFILQRMQLLLALADAVA

SEQ ID NO: 1032 - protein, 70 aa, synthetic construct
MFTMKKSLLVLFFLGIVSLSLCEEERNADEDDGEMTEEVKRGILDTLKQLGKAAVQGLLSKAACKLAKTC

SEQ ID NO: 1033 - protein, 70 aa, synthetic construct
MFTMKKSLLVLFFLGIVSLSLCEEERNADEDDGEMTEEVKRGILDTLKQLGKAAAQSLLSKAACKLAKTC

SEQ ID NO: 1034 - protein, 58 aa, synthetic construct
LGIVSLSLCQEERSADDEEGEVIEEEVKRGFMDTAKNVAKNVAVTLLYNLKCKITKAC

SEQ ID NO: 1035 - protein, 71 aa, synthetic construct
MFTMKKSLLVLFFLGIVSLSLCQEERSADDEEGEVIEEEVKRGFMDTAKNVAKNVAVTLLDNLKCKITKAC

SEQ ID NO: 1036 - protein, 73 aa, synthetic construct
MFTMKKSLLLVFFLGTIALSLCEEERGADDDNGGEITDEEIKRGILTDTLKGAAKNVAGVLLDKLKCKITGGC

SEQ ID NO: 1037 - protein, 74 aa, synthetic construct
MFTMKKSLLFFFFLGTIALSLCEEERGADEEENGGEITDEEVKRGLLLDTVKGAAKNVAGILLNKLKCKVTGDC

SEQ ID NO: 1038 - protein, 60 aa, synthetic construct
GTISLSLCEEERGADEEENGGEITDEEVKRGLLLDTVKGAAKNVAGILLNKLKCKVTGDC

SEQ ID NO: 1039 - protein, 73 aa, synthetic construct; identical to SEQ ID NO: 1036

SEQ ID NO: 1040 - protein, 73 aa, synthetic construct; identical to SEQ ID NO: 1036

SEQ ID NO: 1041 - protein, 70 aa, synthetic construct
MFTLRKSLLLLFFLGMVSLSLCEQERDADEDEGEVTEEVKRGLWTTIKEGVKNFSVGVLDKIRCKITGGC

SEQ ID NO: 1042 - protein, 68 aa, synthetic construct
MFTLRKSLLLLFFLGMVSLSLCEQERGADEDEVTEEVKRGLWTTIKEGLKKFSLGVLDKIRCKIAGGC

SEQ ID NO: 1043 - protein, 120 aa, synthetic construct
MKLLALVLVLSCVVAYTTARKKGQHWPTNTKIFTTPYRFRREADPQGSIVANLKNTPQLPFDDNENLRLVLFDNDPTVDLGEDGKEIPGPQSQPNTLLNNLHLIDENDYLDDYFSSYTFQ

SEQ ID NO: 1044 - protein, 116 aa, synthetic construct
MKLLALVLVLSCVVAYTTARKRGQYWPTNTKIFTTPYRFRREADPQGSIVANLKNTPQLPFDDNENLRLVLFDNDPTVDLGEDDKEIPGPQSQPNALSNNLHLIDENDYFSSYTSQ

SEQ ID NO: 1045 - protein, 116 aa, synthetic construct
MKLLALVLVLSCVVAYTTARKRGQHWPTNTKIFTTPYRFRREADPQGSIVANLKNTPQLPFDDNENLRLVLFDNDSTVDLGEDGKEIPGPQSQPNTLSNNLHLIDENDYFSSYTFQ

SEQ ID NO: 1046 - protein, 115 aa, synthetic construct
PGTYRSFPRNYGTGGRREAGGHVEPRLRFDAETQRGNSFFTDFADLQRRANGRGIEPTAVGATAGIRFRREADQINPLAVRREKRSWLSKKVKKLVNKKNYTRLEKLAKKKLFNE

SEQ ID NO: 1047 - protein, 118 aa, synthetic construct
PGTYRSFPRNFGTSGRYRWRREAGGHVEPRLRFDAETQRGNSFFTDFADLQRRANGRGIEPTVSATAGIRFRQEADQINPLAVRRERRSWLSKSVKKLVNKKNYTRLEKLAKKKLFNE

SEQ ID NO: 1048 - protein, 118 aa, synthetic construct
PGTYRSFPRNFETGGRYRWRREAGGHVEPRLRFDAETQRGNSFFTDFADLQRRANGHGVEPTIGATAGIRFRREADQINPLAVRREKRSWLSKKVKKLVNKKNYTRLEKLAKKKLFNE

SEQ ID NO: 1049 - protein, 51 aa, synthetic construct
IFLVGCKLFGNFILQRMQLLLALADAVAKIKIPWGKVKDFLVGGMKAVGKK

SEQ ID NO: 1050 - protein, 79 aa, synthetic construct
MKLSFLSLVLAIILVMALMYTPHAEAKAWADADADATAAADADADAVADALADAVAKIKIPWGKVKDFLVGGMKAVGKK

SEQ ID NO: 1051 - protein, 7 aa, synthetic construct
VFLFIAL

SEQ ID NO: 1052 - protein, 60 aa, synthetic construct
PSLKSSSCLHSQYQLNYLGRHSLKSSSCLHSQHQLNYPSPKMFTMKKPLLFLFFLGTITL

SEQ ID NO: 1053 - protein, 19 aa, synthetic construct
MFTMKKSLLFFFFLGTIAL

SEQ ID NO: 1054 - protein, 37 aa, synthetic construct
EVFQLSIFSALNRTTRPKMFTMKKSLLFFFFLGTIAL

SEQ ID NO: 1055 - protein, 19 aa, synthetic construct
MFTMKKSLLFFFFLGTIAL

SEQ ID NO: 1056 - protein, 55 aa, synthetic construct
SLCEEERGADEEENGGEITDEEVKRGLLLDTVKGAAKNVAGILLNKLKCKVTGDC

SEQ ID NO: 1057 - protein, 55 aa, synthetic construct; identical to SEQ ID NO: 1056

SEQ ID NO: 1058 - protein, 55 aa, synthetic construct; identical to SEQ ID NO: 1056

SEQ ID NO: 1059 - protein, 55 aa, synthetic construct; identical to SEQ ID NO: 1056

SEQ ID NO: 1060 - protein, 55 aa, synthetic construct; identical to SEQ ID NO: 1056

SEQ ID NO: 1061 - protein, 55 aa, synthetic construct; identical to SEQ ID NO: 1056

SEQ ID NO: 1062 - protein, 21 aa, synthetic construct
LLFVFFLGTITLSLCEEERGA

SEQ ID NO: 1063 - protein, 54 aa, synthetic construct
IPLFISSLKSSSCLHSQHQLNYPSPKMFTMKKSLLLVFFLGTIALSLCEEERGA

SEQ ID NO: 1064 - protein, 46 aa, synthetic construct
KSSSCLHSQHQLNYPSPKMFTMKKSLLLVFFLGTIALSLCEEERGA

SEQ ID NO: 1065 - protein, 46 aa, synthetic construct; identical to SEQ ID NO: 1064

SEQ ID NO: 1066 - protein, 60 aa, synthetic construct
MTITEGKLQMRHSLKSSSCLHSQHQLNYPSPKMFTMKKSLLLVFFLGTIALSLCEEERGA

SEQ ID NO: 1067 - protein, 28 aa, synthetic construct
MFTMKKSLLLVFFLGTIALSLCEEERGA

SEQ ID NO: 1068 - protein, 45 aa, synthetic construct
DEDNGGEITDEEIKRGILTDTLKGAAKNVAGVLLDKLKCKITGGC

SEQ ID NO: 1069 - protein, 45 aa, synthetic construct; identical to SEQ ID NO: 1068

SEQ ID NO: 1070 - protein, 45 aa, synthetic construct
DDDNGGEITDEEIKRGILTDTLKGAAKNVAGVLLDKLKCKITGGC

SEQ ID NO: 1071 - protein, 45 aa, synthetic construct; identical to SEQ ID NO: 1070

SEQ ID NO: 1072 - protein, 45 aa, synthetic construct; identical to SEQ ID NO: 1070

SEQ ID NO: 1073 - protein, 45 aa, synthetic construct; identical to SEQ ID NO: 1070

SEQ ID NO: 1074 - protein, 42 aa, synthetic construct
RLSLAHKETRRPSLLLLFFLGMVSLSLCEQERDADEDEGEVT

SEQ ID NO: 1075 - protein, 53 aa, synthetic construct
SRHQLNYLNSNYPQSPKMFTLRKSLLLLFFLGIVSLSLCEQERDADEDEGEVT

SEQ ID NO: 1076 - protein, 49 aa, synthetic construct
LHSRHQLNYLSPKMFTLRKSLLLLFFLGMVSLSLCEQERDADEDEGEVT

SEQ ID NO: 1077 - protein, 54 aa, synthetic construct
KFSSCLHSRHQLNYLSPKMFTLRKSLLLLFFLGMVSLSLCEQERDADEDEGEVT

SEQ ID NO: 1078 - protein, 36 aa, synthetic construct
MFTLRKSLLLLFFLGMVSLSLCEQERDADEDEGEVT

SEQ ID NO: 1079 - protein, 36 aa, synthetic construct; identical to SEQ ID NO: 1078

SEQ ID NO: 1080 - protein, 34 aa, synthetic construct
EEVKRGLWTTIKEGVKNFSVGVLDKIRCKITGGC

SEQ ID NO: 1081 - protein, 34 aa, synthetic construct; identical to SEQ ID NO: 1080

SEQ ID NO: 1082 - protein, 34 aa, synthetic construct; identical to SEQ ID NO: 1080

SEQ ID NO: 1083 - protein, 34 aa, synthetic construct; identical to SEQ ID NO: 1080

SEQ ID NO: 1084 - protein, 34 aa, synthetic construct; identical to SEQ ID NO: 1080

SEQ ID NO: 1085 - protein, 34 aa, synthetic construct; identical to SEQ ID NO: 1080

SEQ ID NO: 1086 - protein, 30 aa, synthetic construct
  REGION 1..30: note = Antimicrobial peptide
GLLLDTVKGAAKNVAGILLNKLKCKVTGDC

SEQ ID NO: 1087 - protein, 30 aa, synthetic construct
  REGION 1..30: note = Antimicrobial Peptide
GILTDTLKGAAKNVAGVLLDKLKCKITGGC

SEQ ID NO: 1088 - protein, 26 aa, synthetic construct
  REGION 1..26: note = Antimicrobial Peptide
VIPFVASVAAEMMHHVYCAASKRCKN

SEQ ID NO: 1089 - protein, 26 aa, synthetic construct
  REGION 1..26: note = Antimicrobial Peptide
AGYSRMIRRPPGFSPFRVAPASSLKR

SEQ ID NO: 1090 - protein, 23 aa, synthetic construct
  REGION 1..23: note = Antimicrobial Peptide
KIKIPWGKVKDFLVGGMKAVGKK

SEQ ID NO: 1091 - protein, 21 aa, synthetic construct
  REGION 1..21: note = Antimicrobial Peptide
GILSGLLGAGKKIVCGLSGLC

SEQ ID NO: 1092 - protein, 21 aa, synthetic construct
  REGION 1..21: note = Antimicrobial Peptide
GLLSGVLGVGKKIVCGLSGLC

SEQ ID NO: 1093 - protein, 21 aa, synthetic construct
  REGION 1..21: note = Antimicrobial Peptide
GLLSGVLGVGKKVLCGLSGLC

SEQ ID NO: 1094 - protein, 21 aa, synthetic construct
  REGION 1..21: note = Antimicrobial Peptide
GLISGILGAGKKVLCGLSGLC

SEQ ID NO: 1095 - protein, 29 aa, synthetic construct
  REGION 1..29: note = Antimicrobial Peptide
GFMDTAKNVAKNVAVTLLDNLKCKITKAC

SEQ ID NO: 1096 - protein, 18 aa, synthetic construct
  REGION 1..18: note = Antimicrobial Peptide
GIFSLIKGAAKVVAKGLG

SEQ ID NO: 1097 - protein, 30 aa, synthetic construct
  REGION 1..30: note = Antimicrobial Peptide
GILLNTLKGAAKNVAGVLLDKLKCKITGGC

SEQ ID NO: 1098 - protein, 24 aa, synthetic construct
  REGION 1..24: note = Antimicrobial Peptide
FLPVVAGLAAKVLPSIICAVTKKC

SEQ ID NO: 1099 - protein, 19 aa, synthetic construct
  REGION 1..19: note = Antimicrobial Peptide
GIFSLIKGAAKVVAKGLGK

SEQ ID NO: 1100 - protein, 11 aa, synthetic construct
  REGION 1..11: note = Antimicrobial Peptide
GIFSLIKGAAK

SEQ ID NO: 1101 - protein, 20 aa, synthetic construct
  REGION 1..20: note = Antimicrobial Peptide
GAPKGCWTKSYPPKPCSGKR

SEQ ID NO: 1102 - protein, 19 aa, synthetic construct
  REGION 1..19: note = Antimicrobial Peptide
FLPSSPWNEGTYVLKKLKS

SEQ ID NO: 1103 - protein, 22 aa, synthetic construct
  REGION 1..22: note = Artificial Sequence
RMIRRPPGFSPFRVAPASSLKR

(19) United States
(12) Patent Application Publication
(10) Pub. No.: US 2026/0242416 A1
(43) Pub. Date: Aug. 20, 2026

(54) ANTIMICROBIAL PEPTIDES

(71) Applicants: PROVINCIAL HEALTH SERVICES AUTHORITY, Vancouver (CA); UVIC INDUSTRY PARTNERSHIPS INC., Victoria (CA)

(72) Inventors: Inanc BIROL, Vancouver (CA); Diana LIN, Vancouver (CA); René Louis WARREN, Vancouver (CA); Darcy SUTHERLAND, Vancouver (CA); Chenkai LI, Vancouver (CA); Linda HOANG, West Vancouver (CA); Anat YANAI, Vancouver (CA); Caren C. HELBING, Victoria (CA); Fraser HOF, Victoria (CA); Erin FRASER, Victoria (CA)

(21) Appl. No.: 19/160,691

(22) PCT Filed: Jul. 13, 2023
(86) PCT No.: PCT/CA2023/050941

§ 371 Date: Aug. 28, 2025

Related U.S. Application Data
(60) Provisional application No. 63/487,920, filed on Mar. 2, 2023.

(51) Int. Cl. C07K 7/08; A61K 38/00; A61P 31/04; A61P 31/12; A61P 35/00; C07K 14/00; G01N 33/569

(52) U.S. Cl. CPC C07K 7/08; A61P 31/04; A61P 31/12; A61P 35/00; C07K 14/001; G01N 33/56911; A61K 38/00; G01N 2333/245; G01N 2333/255; G01N 2333/31

(57) ABSTRACT

Antimicrobial peptides (AMPs) exhibiting broad spectrum antimicrobial activity are described. Such peptides are useful in treating or preventing infections and other conditions, and are of special interest for treating antibiotic-resistant bacterial pathogens. Development of new AMPs is arduous due to the practical limitations of classical protein-based discovery approaches. A high throughput bioinformatics approach leading to identification of numerous antimicrobial peptides from known genomic sequences is described.

### CROSS-REFERENCE TO RELATED APPLICATIONS

The present application claims the benefit of priority to U.S. Provisional Patent Application No. 63/487,920 filed Mar. 2, 2023 entitled ANTIMICROBIAL PEPTIDES, the entirety of which is hereby incorporated by reference.

### BACKGROUND OF THE INVENTION

The present disclosure relates generally to antimicrobial peptides for the treatment or mitigation of disease.

There is a need for peptides and pharmaceutical compositions thereof which are useful as therapies for microbial infections or as chemopreventative agents to slow or arrest the progression of microbial infections.

Use of antibiotics in livestock may have direct and indirect impact on medical use in addressing human disease. The ubiquitous use of antibiotics in all industries has contributed to the emergence of superbugs which have become resistant to the most common antibiotics. Some strains illustrate multi-drug resistance, which is a global concern. Although the search for new antibiotic approaches continues in earnest to address challenges in both human and animal health.

Consumers have concerns about the use of prophylactic antibiotics due to the potential environmental impact, increasing drug resistance, and the possible consumption of antibiotic lace meat or dairy products. Restrictions on prophylactic antibiotic use in livestock that have been implemented to address these concerns, but have downstream consequences such as increased rates of animal infections, leading to productivity loss due to the increase disease burden. Sick animals that are then treated with antibiotics will continue to contribute to potential drug resistance. Poultry and swine raised in close quarters are particularly susceptible to the rapid spread of disease. Different approaches to reducing infections disease in livestock animals are under development, including investigation of new antibiotic approaches, and development of vaccines. While small molecule drugs have conventionally been used, antimicrobial peptide and polypeptide therapeutic approaches are also under consideration.

International Patent Publication No. WO 2020/118427 (Birol et al.) describes antimicrobial peptides.

It is desirable to find new antimicrobial approaches to reduce the onset and spread of disease in humans and animals.

### SUMMARY

Peptides and/or amino acid sequences with antimicrobial properties are described herein. A bioinformatics approach, starting with sequences exhibiting effect, and making strategic modifications thereto, has led to the discovery of antimicrobial peptides. In a bioinformatics approach, sufficient similarity among sequences can be maintained so as to permit functional equivalency. Sequences similar to isolated sequences from which a consensus is derived are also described. Such similar sequences contain conserved amino acid substitutions and a limited number of non-conserved modifications.

It is an object of the present disclosure to provide antimicrobial peptides, which may obviate or mitigate at least one disadvantage of previous antimicrobial approaches.

There is described herein an antimicrobial peptide comprising: an amino acid sequence according to any one of SEQ ID NO:1-SEQ ID NO:1024 and SEQ ID NO:1086-SEQ ID NO: 1103, or a fragment or variant thereof, having at least 85% amino acid sequence identity thereto.

Further, there is described herein a composition comprising the described antimicrobial peptide together with a suitable excipient.

The composition comprising the described antimicrobial peptide may be a composition for use in in treatment or prevention of a disease or condition, such as infectious disease.

A use for the antimicrobial peptide is provided, for treatment or prevention of a disease or condition in a subject in need thereof. Further, the use of the antimicrobial peptide for preparation of a medicament for treatment or prevention of a disease or condition in a subject in need thereof is also described herein. Additionally, a method of treating or preventing a disease or condition is described, comprising administering to a subject in need thereof an effective amount of the antimicrobial peptide or composition thereof. The disease may be, for example, an infectious disease. The subject may be a human or an animal, such as a livestock animal or a companion animal, such as a domestic animal or pet.

A lipid vesicle comprising the antimicrobial peptide is described. A nucleic acid molecule encoding the antimicrobial peptide is also provided, as is a vector comprising such a nucleic acid molecule.

A method of identifying a target molecule associated with an infectious agent is described, in which the target molecule binds to the antimicrobial peptide. The method comprises the step of screening a library of candidate target molecules associated with the infectious agent, for a molecule that binds to the antimicrobial peptide. A kit for conducting such a method for identifying a target molecule associated with an infectious agent is also described, in which the kit comprises the antimicrobial peptide described herein together with instructions.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the attached Figures.

FIG. 1 shows statistics and attrition as the sequencing data are processed by the antimicrobial peptide discovery pipeline.

FIG. 2 shows antimicrobial susceptibility and hemolysis test results of seven moderately and highly active putative AMPs.

FIG. 3 illustrates rAMPage workflow, pipeline and downstream selection of putative AMPs for validation.

FIG. 4 shows runtime and memory of each dataset through rAMPage Datasets with >1 billion reads (not depicted) finished within one week. Benchmarking statistics time (hours) and peak memory (GB) of each dataset through rAMPage, ordered from least to most reads. For these datasets, rAMPage was run using 48 threads on a CentOS 7.6 machine equipped with an Intel Xeon E5-2650 @2.20 GHz CPU with 380 GB of available RAM and 48 cores. Two large datasets failed to run on that machine and were run on a CentOS 7.6 machine equipped with an Intel Xeon E7-8860 v3 @2.20 GHz CPU with 1500 GB of available RAM and 128 cores.

FIG. 5 shows putative AMP filters used for amphibians and insects Amphibians have a higher threshold for AMPlify score as a filter than the insects do. Other filters remain the same.

FIG. 6 shows score, length, and charge distribution of filtered putative AMPs AMPs tend to be positively charged, they are most commonly between +2 and +9. Histogram is coloured by taxonomic class.

FIG. 7 shows amino acid composition of filtered putative AMPs In the filtered set of putative AMPs, there are more positively charged amino acids than there are negatively charged ones. The three positively charged amino acids give the AMPs their overall positive charge. There also seems to be a higher composition of non-polar residues as well, allowing for easier insertion into the hydrophobic layer of target cell membranes. Histograms are organized by amino acid characteristic and coloured by taxonomic class.

FIG. 8 shows antimicrobial susceptibility and hemolysis testing of 21 putative AMPs Twenty-one putative AMPs were tested (n=3) for bioactivity against E. coli and S. aureus to determine MIC and MBC values, and then tested (n=3) for their hemolytic activity against pig red blood cells to determine HC50 values.

FIG. 9 and FIG. 10 show multiple sequence alignments of moderately to highly active AMPs Multiple sequence alignments of moderate- to highly active putative AMPs and their top blastp hit(s).

FIG. 11 shows multiple sequence alignments of moderately to highly active AMP precursors Multiple sequence alignments of moderate- to highly active putative AMP complete and partial precursors found in multiple species.

FIG. 12 shows distribution of alignment of filtered putative AMPs to mature reference AMPs.

FIG. 13 shows distribution of reference mature AMPs (A) The score, length, and charge distribution of the reference AMPs. The percentages above within the green rectangles indicate the sensitivity of the single filter. (B) The distribution of length, and charge, with respect to score. The percentages above within the green rectangles indicate the sensitivity of two filters.

FIG. 14 shows approach for peptides with multiple cleavage sites Cleaved peptides are combined together in a way that preserves order and orientation. Dashed lines represent excised (cleaved) pieces, and “re-stitching” of remaining pieces, in a manner similar to alternative splicing. Recombined sequences are stored in a separate file and can be filtered out if desired. Implemented in Python v3.7.7.

FIG. 15 shows agglomerative hierarchical clustering of peptides based on their predicted 3D structures.

FIG. 16 shows antimicrobial activity and mammalian cell toxicity of peptides discovered. (Part A) Minimum inhibitory and hemolytic 50 concentration (MIC and HC50, respectively) of peptides with antimicrobial activity against at least one bacterial strain within a panel of 3. (Part B) Mean relative cellular viability of HEK293 (cultured human kidney cells) after 24 h incubation with the six most active insect and amphibian peptides at concentrations up to 128 μg/mL.

FIG. 17 illustrates phylogenetic analysis of validated AMPs. (Part A) Circular phylogenetic tree based on a multiple sequence alignment of active peptides with family classifications highlighted (colour-coded AMP names). Multiple sequence alignments are shown for the Nigrocin-2 (part B), Brevinin-1 (part C) and Brevinin-2 families (part D), with the outlines indicating the identified family signature.

FIG. 18 shows predicted structural content and antimicrobial activity of various bioactive peptides against E. coli, Salmonella Enteritidis, and S. aureus. Secondary structure content indicated by the percentage of residues assigned to participate in helical vs. linear extended regions.

FIG. 19 depicts superimposed predicted 3D structures of AMP family members for peptides identified as belonging to the Apidaecin, Brevinin-1, Brevinin-2 and Nigrocin-2 groups.

### DETAILED DESCRIPTION

Peptides and/or amino acid sequences with antimicrobial properties are described herein. A bioinformatics approach, starting with sequences exhibiting effect, and making strategic modifications thereto, has led to the discovery of antimicrobial peptides. In a bioinformatics approach, sufficient similarity among sequences can be maintained so as to permit functional equivalency. Sequences similar to isolated sequences from which a consensus is derived are also described. Such similar sequences may contain conserved amino acid substitutions together with a limited number of non-conserved substitutions, such as modifications or deletions, but while still maintaining functionality.

These peptides and their pharmaceutical compositions and modifications thereof are also useful as therapies for microbial infections or as chemopreventative agents to slow or arrest the progression of microbial infections. Modifications of peptides described herein may include but are not limited to incorporation of the peptides or their modifications in lipid vesicles for enhanced therapeutic delivery and the modulation of other ADMET properties (absorption, distribution, metabolism, excretion, toxicity) as well.

Chemical modifications of the peptides are described, which are known to individuals skilled in the art of peptide chemistry to be useful to enhance stability and otherwise make the peptides more drug-like and useful for the desired applications. Such modifications include peptide cyclization and the use of amino acids of opposite chirality—so-called D-amino acids. Such modifications also include alternative backbone chemistries and novel side chains that retain the binding specificity.

Also described is the application of the peptides, and modifications of the peptides obvious to those skilled in the art, to other microbial targets. Antimicrobial therapies useful and effective in one type of infection may be useful and effective in other diseases.

Also described are vector constructs incorporating the disclosed peptides and/or their amino acid sequences and coding nucleic acid sequences for the purposes of the production of antimicrobial peptides.

The peptides described herein, and the modifications thereof are also useful in combination with other antimicrobial agents for the treatment or prevention of disease, such as an infectious disease or a cancer.

Uses of the AMPs either alone or as part of a kit in a procedure to isolate and identify their binding partners (target molecules) associated with the infectious agent are also described.

The peptides and/or amino acid sequences described herein have selective antimicrobial properties. Further aspects and advantages will become apparent from consideration of the ensuing description of various embodiments. A person skilled in the art will realize that other embodiments, combinations and variations are possible, and that the details described herein can be modified in a number of respects, all without departing from the overall concept. Thus, the following drawings, descriptions and examples are to be regarded as illustrative in nature and not restrictive.

Treatment or prevention of a disease or condition encompasses treatment before and after outward signs or symptoms of the disease or condition are present in the subject. For example, a subject exposed an infectious agent may or may not exhibit symptoms. Further, the prevention or prophylaxis of a disease or condition may encompass partial prevention, lessening of severity when onset occurs, decreasing likelihood of outward signs or symptoms, or preventing the spread of infection by keeping severity so low as to be undetectable or negligible. Treatment and prevention may involve modulating the immune system of the subject to the extent that the subject's own defenses ward off the disease or condition, such as infection. An inflammatory or anti-inflammatory effect of the peptides described herein may modulate the outward signs or symptoms of a disease or condition.

Anti-cancer activity, such as against solid tumours or liquid tumours, may be modulated by peptides as described herein. Indirect attack on cancer cells by the peptides described herein through effects on the immune system by the peptides may alleviate cancerous cell growth.

An antimicrobial peptide comprising: an amino acid sequence according to any one of SEQ ID NO:1 to SEQ ID NO:1024 and SEQ ID NO:1086 to SEQ ID NO:1103, or a fragment or variant thereof, having at least 85% amino acid sequence identity thereto. The threshold of amino acid sequence identity for the variant or fragment may optionally be at least 90%, at least 95%, at least 96%, at least 97%, at least 98%, at least 99%, or may be 100% amino acid sequence identity to any one of SEQ ID NO:1 to SEQ ID NO:1024 and SEQ ID NO:1086 to SEQ ID NO:1103.

The antimicrobial peptide may be modified, or may be a variant which comprises a modification that is a conservative amino acid substitution. Such an amino acid sequences as are known in the art may include the following candidates, with the substitutable options shown in parentheses: Ala (Gly, Ser); Arg (Gly, Gln); Asn (Gln, His); Asp (Glu); Cys (Ser); Gln (Asn, Lys); Glu (Asp); Gly (Ala, Pro); His (Asn, Gln); Ile (Leu, Val); Leu (lie, Val); Lys (Arg, Gln); Met (Leu, lie); Phe (Met, Leu, Tyr); Ser (Thr, Gly); Thr (Ser; Val); Trp (Tyr); Tyr (Trp, Phe); and Val (lie, Leu). Furthermore, ‘functional’ variants, mutations, insertions, or deletions encompass sequences in which the activity or function is substantially the same as that of the reference sequence from which the altered sequence is derived. Activity or function may be tested according to such parameters as described herein, such as MIC or MBC. Further, it may be desirable to reduce the antigenicity of a peptide, for example by PEGylated, or the peptide may comprise a D-amino acid. The peptide may be cyclized.

An exemplary antimicrobial peptide may be one that comprises or consists of an amino acid sequence according to any one of SEQ ID NO:1 to SEQ ID NO:1024 and SEQ ID NO:1086 to SEQ ID NO:1103. Exemplary peptides include: PeNi10 (SEQ ID NO:1086), PeNi11 (SEQ ID NO:1087), PeNi7 (SEQ ID NO:1088), RaOm5 (SEQ ID NO: 1089), TeBi1 (SEQ ID NO: 1090), AmMa1 (SEQ ID NO:12), OdMa12 (SEQ ID NO:69), PeNi14 (SEQ ID NO:15), PeNi16 (SEQ ID NO: 200), TeRu4 (SEQ ID NO: 996), TeRu2 (SEQ ID NO: 994), PoSn1 (SEQ ID NO: 936), PoSn2 (SEQ ID NO: 942), BoAr6 (SEQ ID NO: 790), TeRu3 (SEQ ID NO: 993), TeRu1 (SEQ ID NO: 833), PaVa2 (SEQ ID NO: 961), PaVa3 (SEQ ID NO: 955), PaVi1 (SEQ ID NO: 968), PoRo1 (SEQ ID NO: 927), or VeSi1 (SEQ ID NO: 1022).

A composition is described herein which comprises the antimicrobial peptide as described herein, together with a suitable excipient, such as a pharmaceutically acceptable carrier. The composition may be one that is suitable for use in treatment or prevention of a disease or condition, such as an infectious disease, or a cancer, such as may be attributable to a solid tumour or a liquid tumour.

The composition may be formulated for oral, injectable, rectal, topical, transdermal, nasal, or ocular delivery. Such compositions can thus be administered to subjects in need thereof through any acceptable route, such as topically (as by powders, ointments, or drops); oral tablets, capsules, gels or liquids; or rectal suppositories. Further modes of delivery include mucosally, sublingually, parenterally, intravaginally, intraperitoneally, bucally, ocularly, or intranasally.

When formulated for oral use or administration in a liquid formulation, the excipients or ingredients may include but are not limited to those accepted in the art of pharmaceutical formulations, for example emulsions, microemulsions, solutions, suspensions, syrups and elixirs. Liquid dosage forms may contain inert diluents such as water or other solvents, solubilizing agents, emulsifiers, ethyl alcohol, isopropyl alcohol, ethyl carbonate, ethyl acetate, benzyl alcohol, benzyl benzoate, propylene glycol, 1,3-butylene glycol, or dimethylformamide. Further, a liquid formulation may comprise oils such as cottonseed, groundnut, corn, germ, olive, castor, and sesame oils; glycerol, tetrahydrofurfuryl alcohol, polyethylene glycols and fatty acid esters of sorbitan; and mixtures thereof. Besides inert diluents, such oral compositions can also include adjuvants such as wetting agents, emulsifying and suspending agents, sweetening, flavoring, and perfuming agents.

The composition may be one that is lyophilized. The composition may comprise a suitable preservative.

The composition may be one that is distributed evenly in a diet intended for livestock, such as swine or poultry. Such a composition may be sprayed or mixed into a ground or powdered ingredient, and then mixed evenly into a coarser animal feed to ensure even distribution.

A use of the antimicrobial peptide is provided herein, for treatment or prevention of a disease or condition in a subject in need thereof, such as an infectious disease. The disease or condition may be a cancer, such as a solid tumour or a liquid tumour.

Further, a use is provided for preparation of a medicament for treatment or prevention of such a disease or condition in a subject in need thereof. A method of treating or preventing such a disease or condition is also described herein, which comprises administering to a subject in need thereof an effective amount of the peptide or the composition described herein.

The disease or condition may be one attributable to Gram-negative bacteria, or it may be a disease or condition attributable to Gram-positive bacteria. The disease or condition may be one that is attributable to acid fast bacteria, or one that is attributable to bacteria that has become resistant to other drugs. Such diseases or conditions may be ones attributable to E. coli, S. enterica, S. aureus, P. aeruginosa, S. pyogenes, M. smegmatis, MRSA, S. Enteritidis or S. Heidelberg bacteria, for example.

Further, the disease or condition may be a cancer, such as a solid tumour or a liquid tumour.

A lipid vesicle may be used to deliver the antimicrobial peptide described herein. A nucleic acid molecule encoding the antimicrobial peptide described is also envisioned. A vector comprising the nucleic acid molecule is also encompassed.

A method of identifying a target molecule associated with an infectious agent is described, wherein the target molecule binds to the antimicrobial peptide described herein. Such a method involves the step of screening a library of candidate target molecules associated with the infectious agent, for a molecule that binds to the antimicrobial peptide. The infectious agent may be Gram-negative bacteria, or may be Gram-positive bacteria. Further, the infections agent may be acid fast bacteria, or bacteria that has become resistant to other drugs. Exemplary infectious agents include but are not limited to E. coli, S. enterica, S. aureus, P. aeruginosa, S. pyogenes, M. smegmatis, MRSA, S. Enteritidis or S. Heidelberg bacteria. Further, a method of identifying a target molecule for modulating biological activity is described, wherein the target molecule binds to a peptide as described herein. The method comprising the step of screening a library of candidate target molecules for a molecule that binds to the peptide. Modulating of biological activity may comprise anti-tumour action, anti-inflammatory action, or inflammatory action. In such methods of target identification, the screening of a library of candidate target molecules may comprise in silico screening.

A kit is encompassed herein for identifying a target molecule associated with an infectious agent. Such a kit comprises an antimicrobial peptide as described herein together with instructions for conducting the method described herein for identifying a target molecule associated with the infectious agent. Optionally, additional reagents may be provided with the kit. A kit for identifying a target molecule for modulating biological activity, is also described. Such a kit comprises a peptide, as described herein, together with instructions for conducting a screening method for molecules that bind to the peptide.

There are instances herein where the term “putative” precedes the term “antimicrobial peptide”. The term makes no implication regarding antimicrobial action of the peptide but acknowledges difference between the establishment of antimicrobial effect versus use as an approved drug, given the years of downstream efforts after antimicrobial activity is established. Thus the term putative as used herein acknowledges the long term efforts required in establishing commercial viability and bringing such a product to market.

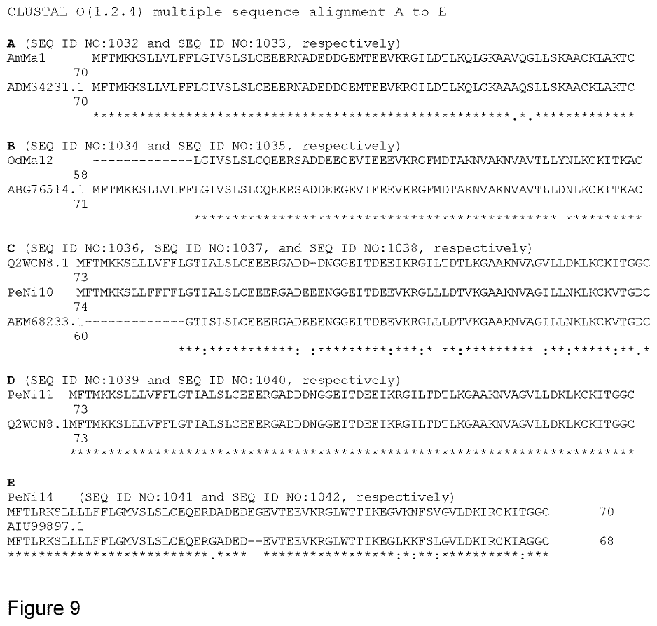

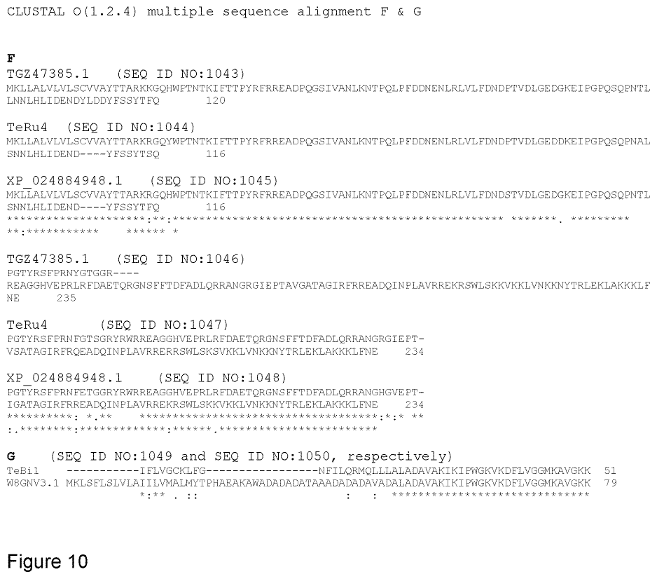

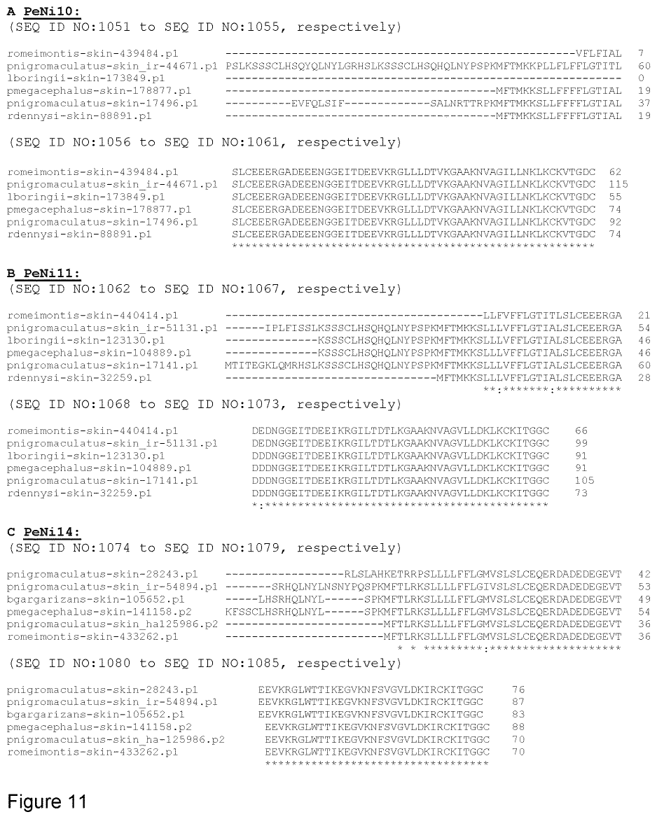

Overview of Sequence ID number Assignments. SEQ ID NO: 1-1024 are antimicrobial peptides as described in Example 2 and elsewhere. SEQ ID NO: 1025-1031 are prepro sequences of from Table 1 of Example 1. The corresponding 7 AMP sequences in Table 1 have a match within SEQ ID NO: 1 to 1024. The AMP sequences in Table 2, Table 14, and Table 15 either have a match in the first 1024 sequences, or in SEQ ID NO:1086-1103. SEQ ID NO: 1032-1085 represent sequences in FIG. 9 (SEQ ID NO: 1032-SEQ ID NO:1042) and FIG. 10 (SEQ ID NO: 1043 to 1050) and FIG. 11 (35 sequences, in order SEQ ID NO: 1051 to 1085).

### EXAMPLES

The following Examples outline exemplary embodiments and/or studies conducted pertaining thereto. While the Examples are illustrative, they should not be viewed as limiting.

### Example 1

### Mining Amphibian and Insect Transcriptomes for Antimicrobial Peptide Sequences with rAMPage

Summary Antibiotic resistance is a global health crisis increasing in prevalence every day. To combat this crisis, alternative antimicrobial therapeutics are urgently needed. Antimicrobial peptides (AMPs), a family of short defense proteins, are produced naturally by all organisms and hold great potential as effective alternatives to small molecule antibiotics. Here, “rAMPage” is described as a scalable bioinformatics discovery platform for identifying AMP sequences from RNA sequencing (RNA-seq) datasets. In the present Example, the utility and scalability of rAMPage is demonstrated, running it on 84 publicly available RNA-seq datasets from 75 amphibian and insect species-species known to have rich AMP repertoires. Across these datasets, 1137 putative AMPs were identified, 1024 of which were heretofore unknown, and deemed novel by a homology/identity search in cataloged AMPs in public databases. Twenty-one peptide sequences were selected from this set for antimicrobial susceptibility testing against Escherichia coli and Staphylococcus aureus and it was observed that seven of them have high antimicrobial activity. The present Example illustrates how in silico methods such as rAMPage can enable the fast and efficient discovery of novel antimicrobial peptides as an effective first step in the strenuous process of antimicrobial drug development.

### 1.1. Introduction

Due in large part to the overuse and misuse of antibiotics, the prevalence of multidrug-resistant bacteria is rapidly growing at a rate that cannot be matched by antibiotic discovery efforts [1]. As a consequence, the world is currently in an arms race and is at the cusp of a post-antibiotic era [1]. The slow pace of new antibiotic drug discovery, development, and regulation, combined with the accelerated emergence of resistance to existing antibiotics creates what is referred to as the “discovery void” [2]. This gap between discovery and emergence of resistance highlights an urgency to develop new antimicrobial therapeutics. One such alternative is formulations based on the antimicrobial peptides (AMPs) [3].

AMPs are short amphipathic host defense peptides that are produced in all multicellular organisms as part of the innate immune system [3]. Many AMPs operate through nonspecific mechanisms [4], such as direct electrostatic interactions with the cell membrane and immunomodulation [3], allowing for a broad spectrum of efficacy against bacteria [5], viruses [6], and fungi [7]. Furthermore, pathogens develop a slower rate of resistance to AMPs compared to conventional antibiotics [8]. It is these qualities that position AMPs as attractive alternatives to conventional antibiotics [9].

AMPs are often produced as precursor peptides within cells that consist of an N-terminal signal peptide, followed by an acidic pro-sequence, and a C-terminal basic bioactive mature peptide sequence [3]. The acidic pro-sequence neutralizes the basic mature peptide to keep the AMP in its inactive form and the signal peptide and acidic pro-sequence together are referred to as the prepro domain [3]. AMPs are then activated by proteolytic cleavage of the prepro sequence and the release of the mature peptide [3]. While the signal peptide is often highly conserved, the acidic pro-sequence and mature AMP can be quite variable [10]. However, there is evidence that the prepro sequence can vary across different organisms [3] and even within organisms [11].

Past research has shown that amphibians, such as the American bullfrog Rana [Lithobates]catesbeiana, possess a rich diversity of AMPs due to their aquatic and terrestrial life cycle, where the species encounter a wide spectrum of pathogens in these two environments [11]. In amphibians, AMPs such as ranatuerin are secreted at the skin surface upon pathogen exposure and can also stimulate an adaptive immune response [12]. In contrast, insects lack a sophisticated adaptive immune system and yet are highly tolerant to bacterial infection [13,14]. This may be due to the production of AMPs by the innate immune system [14]. In insects, AMPs are found in venom or salivary gland secretions. For example, melittin, a 26 amino acid (AA) peptide is the main component of honeybee venom [13]. While there are many known amphibian AMPs, there are far fewer known insect AMPs. Amphibian AMPs have their own designated database of 1923 peptides in the Database of Anuran Defense Peptides (DADP) [15]. Additionally, they also comprise 34% (1128 sequences) of the curated Antimicrobial Peptide Database 3 (APD3) [16]. Insect AMPs, however, only contribute 10% (325 sequences) in APD3, despite being the next largest non-mammalian classification. Better characterization of these AMP arsenals holds great potential in aiding the discovery of novel AMPs.

Because most AMPs under therapeutic investigation are derived from naturally occurring AMPs in various organisms [2], effective methods to discover natural AMPs would expand the number of potential candidates. Current wet lab screening protocols consist of extraction, isolation, and purification of AMPs through laborious methods such as the collection of skin secretions followed by liquid chromatography and sequence identification using mass spectrometry [17,18,19,20,21]. However, these protocols are costly, time-consuming, and expertise intensive. To resolve this, a scalable, rapid, high throughput in silico methodology built on genomics technologies and able to mine RNA sequencing (RNA-seq) datasets, would greatly aid in the discovery of AMPs funneling into drug development and enhancement processes. There are in silico AMP discovery methodologies presented in earlier studies [22,23,24,25,26], most of which start with processed data such as assembled genomic or protein sequences. Additionally, there are several state-of-the-art tools that perform AMP prediction [27]. Because AMP precursor genes have conserved sequence characteristics, these properties can be leveraged for filtering, and their inferred mature products can be classified as an AMP or not using machine learning methods. With the current unprecedented expansion of data generation and large amounts of sequencing data available in public repositories [28], there exists a rich untapped resource for AMP discovery.

To help fill the antibiotic discovery void, the rAMPage (Rapid Antimicrobial Peptide Annotation and Gene Estimation) technology described herein is useful. rAMPage is a homology-based AMP discovery pipeline to mine for putative AMP sequences in publicly available genomic resources. To classify AMP sequences, rAMPage employs AMPlify [27], an attentive deep learning model. Currently, existing AMP databases, (e.g., APD3, DADP) contain less than 4000 validated nonredundant AMP sequences in total. In comparison, over 1000 putative mature AMPs were found in the present Example, with the potential to discover thousands more. Realizing the full potential of such pipelines would require the synthesis and validation of AMP candidates. Herein, results are provided on a select list of 21 peptides detected using rAMPage.

Reference is made herein to Applicant's publication: Lin et al., entitled: Mining Amphibian and Insect Transcriptomes for Antimicrobial Peptide Sequences with rAMPage, Antibiotics (Basel), 2022 Jul 15; 11(7):952 (119).

### 1.2. Results

### 1.2.1. Identification of Putative AMPs

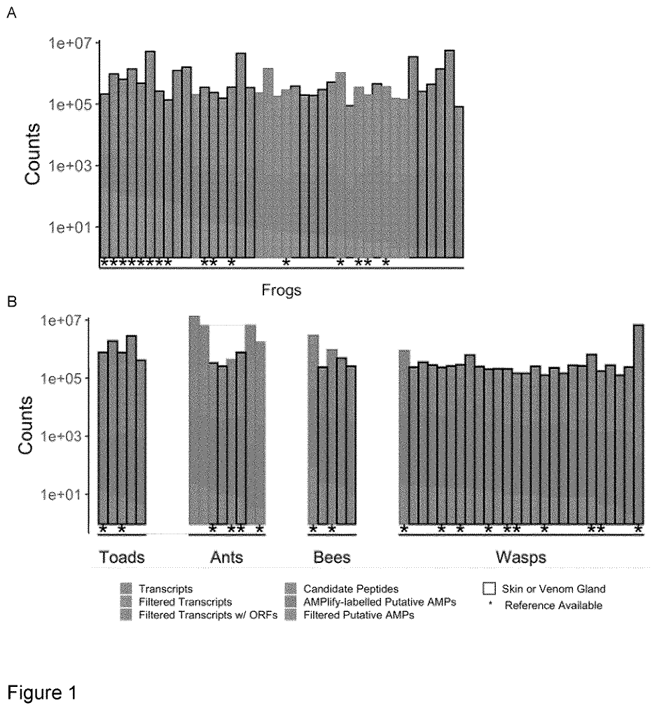

FIG. 1 shows statistics and attrition as the sequencing data are processed by the rAMPage AMP discovery pipeline. rAMPage processes RNA-seq datasets from raw reads to transcripts to putative AMPs. In this case, a putative AMP is defined as a sequence with an AMPlify score 10 for amphibians or ≥7 for insects, a length s 30 AA, and a charge 2. Datasets with a reference transcriptome used during assembly are indicated with an asterisk. The total number of putative AMPs (n=1478, including 341 duplicates) are shown in purple, discovered from a total of ~53 million assembled transcripts.

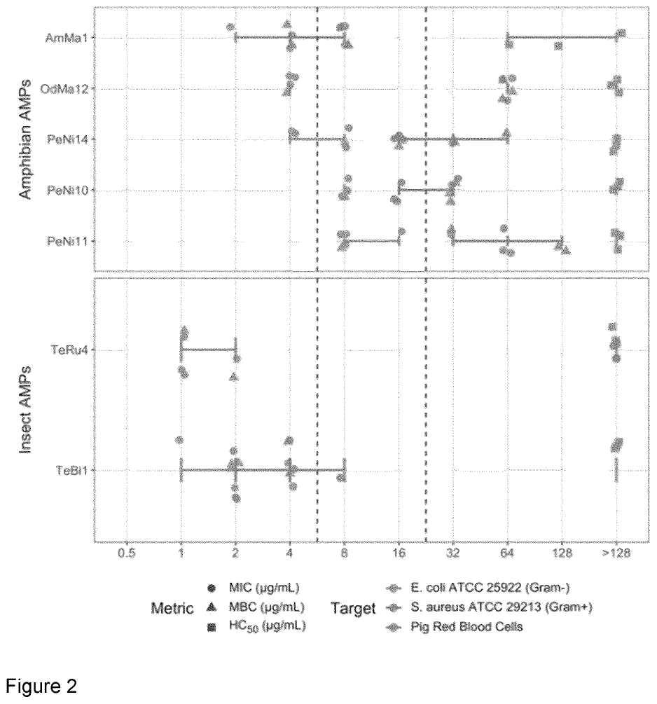

FIG. 2 shows antimicrobial susceptibility and hemolysis test results of seven moderately and highly active putative AMPs. AMPs were tested for their bioactivity against E. coli and S. aureus to determine minimum inhibitory and bactericidal concentrations (MIC and MBC, respectively). AMPs were also tested for their hemolytic activity using pig red blood cells to determine hemolytic concentration (HC₅₀) values. Moderate activity (MIC and MBC in the range of 8-16 μg/mL) and high activity (≤4 μg/mL) thresholds indicated by the dashed lines. AMPs are ordered by increasing MIC values against E. coli ATCC 25922.

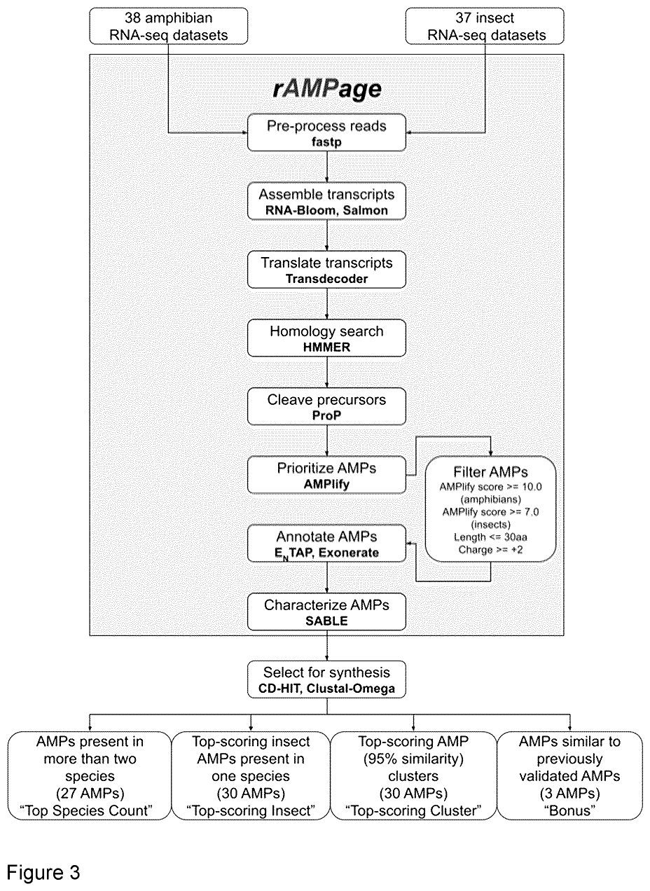

FIG. 3 illustrates rAMPage workflow. The rAMPage pipeline and downstream selection of putative AMPs for validation.

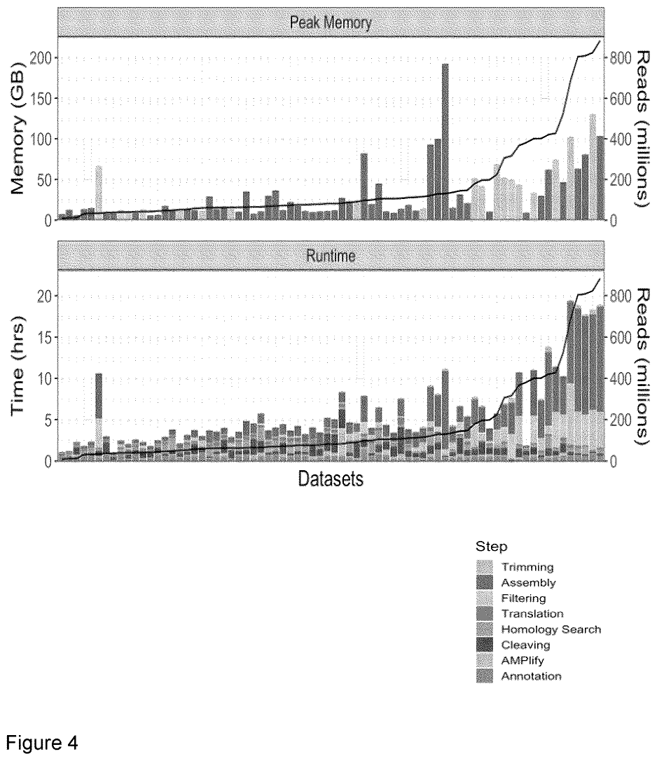

Using rAMPage, ~53 million transcripts were assembled from 84 RNA-seq datasets derived from the transcriptomes of 38 amphibian (33 frogs, five toads; anurans) and 37 hymenopteran insect (eight ants, five bees, 24 wasps) species and flagged 203,758 candidate peptide sequences to be classified (FIG. 1). To select a list of high-confidence putative AMPs, duplicates were collapsed from multiple samples and applied three filters: AMPlify prediction score, peptide charge, and peptide length to obtain 1137 peptide sequences. Of these, 795 originate from amphibians, and 342 from insects. Running rAMPage on all 84 datasets took one week, with all datasets (comprising <1 billion reads) taking less than 24 h (see FIG. 4 for details on the computational platform and resource usage statistics).

For each sequence, AMPlify [27] reports a prediction score s from 0 to 80, where s is a log-transformation of the AMPlify probability score p and 80 represents the highest confidence.

$$s = - 10 {log}_{10} ( 1 - p ) , \qquad ( 1 )$$

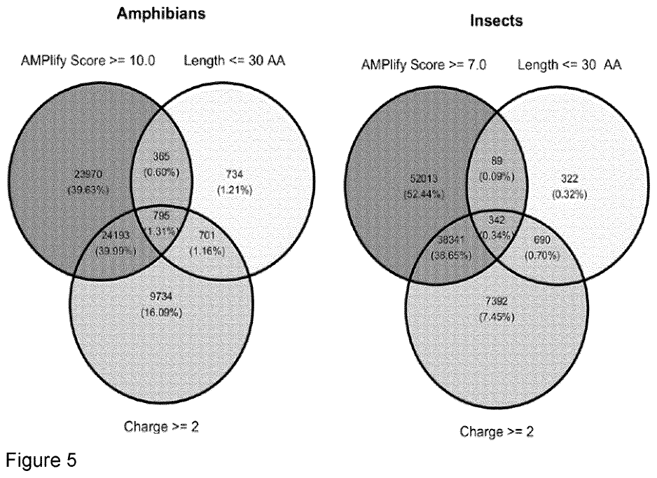

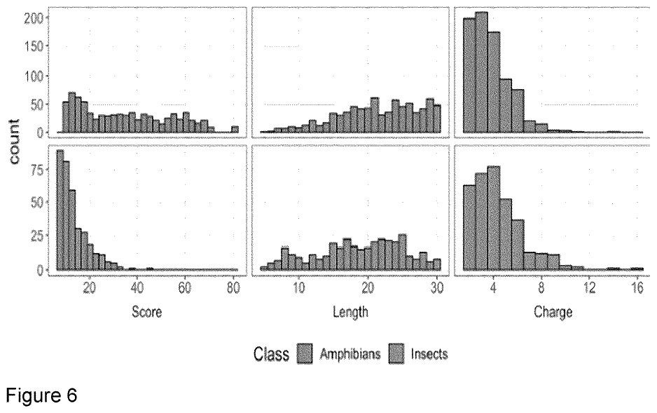

The training data set for the AMPlify model had an over-representation of AMPs from amphibian species [27]; hence, it is biased towards assigning higher scores for amphibian AMPs. To compensate, separate score cut-offs were applied for the two groups: 10 for amphibians and 7 for insects. Since the majority of AMPs are positively charged, a net charge threshold of >+2 was applied. As for length, sequences were filtered for those that are 530 AA, because shorter peptides are more cost-effective to synthesize for downstream validation studies. FIG. 5 shows that the length filter used is the most restrictive filter of the three, with only 4.28% and 1.45% of the sequences for amphibians and insects, respectively, meeting this criterion.

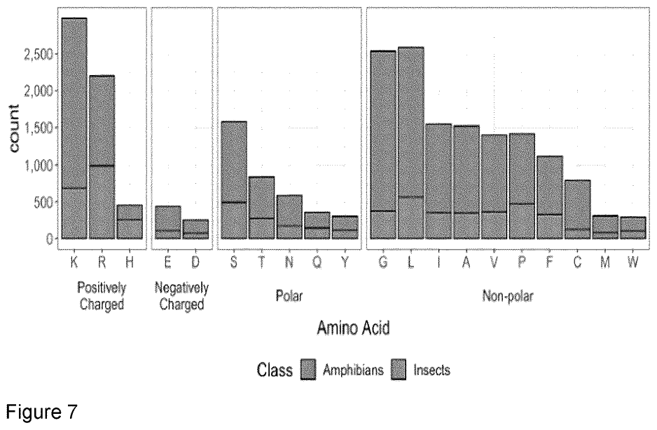

Score, charge, length distributions, and AA compositions of the 1137 putative AMPs are characterized in FIG. 4 and FIG. 7. From this set, 21 AMPs were selected for synthesis and validation, using three prioritization strategies: “Species Count”, “Insect Peptide”, and “AMPlify Score” (see Section 4). The peptides have been named after the species they were discovered from (Table 1), then numbered in order using their AMPlify scores.

Table 1 shows peptide naming convention. Legend for the peptide naming convention. The first two letters of the binomial species name are taken to form the abbreviated peptide prefix. Binomial and common names taken from the NCBI [29] Taxonomy Browser.

TABLE 1
Peptide Naming Convention

| Name | Binomial Name | Common Name or Description |
|---|---|---|
| AmMa | Amolops mantzorum | Mouping sucker frog |
| BoAr | Bombus ardens | Bumblebee |
| OdMa | Odorrana margaretae | Green odorous frog |
| PaVa | Parapolybia varia | Lesser paper wasp |
| PaVi | Pachycrepoideus vindemmiae | Parasitic wasp |
| PeNi | Pelophylax nigromaculatus | Dark-spotted frog |
| PoRo | Polistes rothneyi | Polistine wasp |
| PoSn | Polistes snelleni | Japanese paper wasp |
| RaOm | Rhacophorus omeimontis | Omei tree frog |
| RaSy | Rana sylvatica | Wood frog |
| TeBi | Tetramorium bicarinatum | Tramp ant |
| TeRu | Temnothorax rugatulus | Small myrmicine ant |
| VeSi | Vespa simillima | Japanese hornet |

### 1.2.2. Antimicrobial Susceptibility Testing (AST) Results

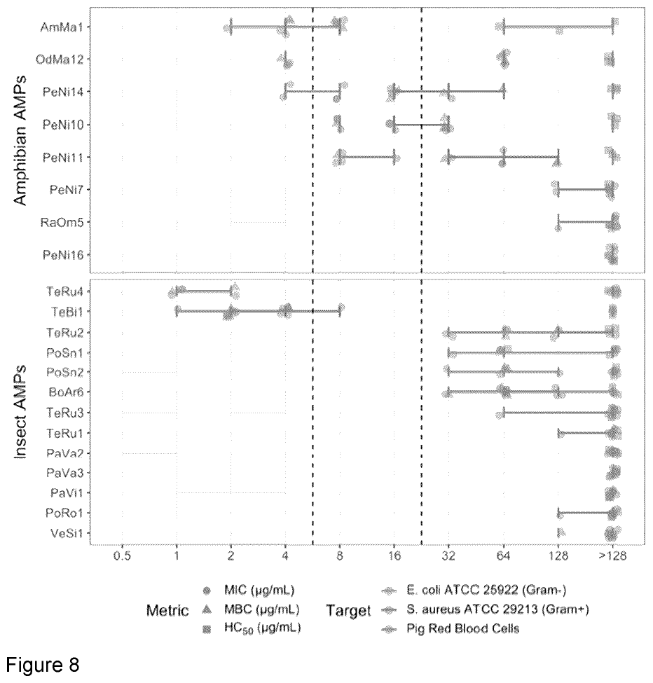

A total of 21 of the 1137 putative AMPs (Table 2) were synthesized (Genscript Biotech, Piscataway, NJ, USA) and tested for their antimicrobial activity against Escherichia coiATeC 25922 and Staphylococcus aureus ATSC 29213 in a minimum of three independent experiments (see FIG. 8 for a full set of experimental results). In these antimicrobial susceptibility tests, AMP activity was assessed using two metrics: minimum inhibitory and bactericidal concentrations (MIC and MBC, respectively). Lower MIC and MBC values are desirable as they indicate that lower AMP concentrations are sufficient for inhibitory or bactericidal activity, respectively. AMP toxicity was measured by HC₅₀ hemolytic concentration values—the concentration required to lyse ≥50% of porcine red blood cells. In contrast to MIC/MBC assays, it is desirable to have higher HC₅₀ values. All 21 putative AMPs exhibited minimal to no hemolytic activity with HC₅₀ values of 64 μg/mL or higher.

Table 2 shows a subset of 21 AMPs synthesized and validated against E. coli and S. aureus. The prioritization methodology and AMPlify score for each validated AMP are indicated along with the derived mature peptide sequences and their characteristics. Refer to Table 1 for the naming convention.

TABLE 2

| | | | | | | Selection |
|---|---|---|---|---|---|---|
| Name | Sequence | Class | Score | Length | Charge | Method |
| AmMa1 | GILDTLKQLGKAA | Amphibia | 80.0 | 29 | 4 | TopCluster |
| SEQ ID NO: 12 | VQGLLSKAACKL | | | | | |
| | AKTC | | | | | |
| OdMa12 | GFMDTAKNVAKN | Amphibia | 69.2 | 29 | 4 | TopCluster |
| SEQ ID NO: 69 | VAVTLLYNLKCKI | | | | | |
| | TKAC | | | | | |
| PeNi14 | GLWTTIKEGVKN | Amphibia | 67.5 | 29 | 3 | SpeciesCount |
| SEQ ID NO: 15 | FSVGVLDKIRCKI | | | | | |
| | TGGC | | | | | |
| PeNi10 | GLLLDTVKGAAK | Amphibia | 61.8 | 30 | 3 | SpeciesCount |
| SEQ ID NO: 1086 | NVAGILLNKLKCK | | | | | |
| | VTGDC | | | | | |
| PeNi11 | GILTDTLKGAAKN | Amphibia | 61.8 | 30 | 3 | SpeciesCount |
| SEQ ID NO: 1087 | VAGVLLDKLKCKI | | | | | |
| | TGGC | | | | | |
| PeNi7 | VIPFVASVAAEM | Amphibia | 43.2 | 26 | 2 | SpeciesCount |
| SEQ ID NO: 1088 | MHHVYCAASKR | | | | | |
| | CKN | | | | | |
| RaOm5 | AGYSRMIRRPPG | Amphibia | 12.5 | 26 | 6 | SpeciesCount |
| SEQ ID NO: 1089 | FSPFRVAPASSL | | | | | |
| | KR | | | | | |
| PeNi16 | ATAWKVPPGLQP | Amphibia | 26.6 | 27 | 4 | SpeciesCount |
| SEQ ID NO: 200 | IRPIRIRPLCGND | | | | | |
| | KS | | | | | |
| TeRu4 | SWLSKSVKKLVN | Insecta | 25.5 | 30 | 8 | TopInsect |
| SEQ ID NO: 996 | KKNYTRLEKLAK | | | | | |
| | KKLFNE | | | | | |
| TeBi1 | KIKIPWGKVKDFL | Insecta | 45.0 | 23 | 6 | TopInsect |
| SEQ ID NO: 1090 | VGGMKAVGKK | | | | | |
| TeRu2 | AFVRILCYCCPR | Insecta | 25.5 | 17 | 6 | TopInsect |
| SEQ ID NO: 994 | RIKRR | | | | | |
| PoSn1 | ISIKEALEHSFFH | Insecta | 30.4 | 23 | 3 | TopInsect |
| SEQ ID NO: 936 | TVPRKWCKKH | | | | | |
| PoSn2 | TALKSLSILKKLA | Insecta | 23.7 | 17 | 4 | TopInsect |
| SEQ ID NO: 942 | KLNM | | | | | |
| BoAr6 | GILRLVTRRFRFS | Insecta | 22.1 | 30 | 6 | TopInsect |
| SEQ ID NO: 790 | PTNLNRYTVARL | | | | | |
| | VSGVP | | | | | |
| TeRu3 | AVLSFVHKLFLNF | Insecta | 26.3 | 28 | 3 | TopInsect |
| SEQ ID NO: 993 | LHVDTSKGKCRA | | | | | |
| | TLQ | | | | | |
| TeRu1 | VPFGLKPR | Insecta | 25.8 | 8 | 2 | SpeciesCount |
| SEQ ID NO: 833 | | | | | | |
| PaVa2 | KYHHIKLRHGRH | Insecta | 26.0 | 17 | 6 | TopInsect |
| SEQ ID NO: 961 | RRTIH | | | | | |
| PaVa3 | ITEPVGTKAPTFT | Insecta | 24.0 | 24 | 3 | TopInsect |
| SEQ ID NO: 955 | SELRGGWLKKR | | | | | |
| PaVi1 | WALRWKTR | Insecta | 25.0 | 8 | 3 | TopInsect |
| SEQ ID NO: 968 | | | | | | |
| PoRo1 | VAAFAIIGCLCCR | Insecta | 23.0 | 17 | 4 | TopInsect |
| SEQ ID NO: 927 | RPRR | | | | | |
| VeSi1 | FILHAKKTRSAK | Insecta | 22.5 | 12 | 4 | TopInsect |
| SEQ ID NO: 1022 | | | | | | |

Of these 21 putative AMPs, three displayed moderate activity (MIC and MBC in the range 8-16 μg/mL) and four displayed high activity (54 μg/mL) against E. coli and/or S. aureus, all with minimal hemolytic activity, as shown in FIG. 2. The characteristics of these seven sequences are described in Table 3. All seven AMPs with moderate to high antimicrobial activity have AMPlify scores greater than 25.

Table 3 shows characteristics of putative AMP sequences with moderate to high in vitro bioactivity against E. coli or S. aureus. Each sequence is separated into the prepro sequence and the predicted mature peptide sequence. Conserved proteolytic cleavage sites are underlined in the prepro sequences.

TABLE 3
Prepro Sequences and Characteristics of Mature AMP Peptides
With in vitro bioactivity against E. coli or S. aureus

| | | | | | MIC* | |
|---|---|---|---|---|---|---|
| | | | | | (μg/mL) | |
| | | | | | E. coli^{†}/ | |
| | | | | AMPlify | S. | Peptide |
| Prepro sequence | Peptide Sequence | Length | Charge | Score | aureus^{†} | ID |
| MFTMKKSLLVLFFLGIVS | GILDTLKQLGKAAV | 29 | 4 | 80.0 | 2-4/ | AmMa1 |
| LSLCEEERNADEDDGE | QGLLSKAACKLAKT | | | | 4-8 | |
| MTEEVKR | C | | | | | |
| SEQ ID NO: 1025 | SEQ ID NO: 12 | | | | | |
| LGIVSLSLCQEERSADD | GFMDTAKNVAKNV | 29 | 4 | 69.2 | 4/ | OdMa12 |
| EEGEVIEEEVKR | AVTLLYNLKCKITK | | | | 64 | |
| SEQ ID NO: 1026 | AC | | | | | |
| | SEQ ID NO: 69 | | | | | |
| MFTMKKSLLFFFLGTIAL | GLLLDTVKGAAKN | 30 | 3 | 61.8 | 8/ | PeNi10 |
| SLCEEERGADEEENGG | VAGILLNKLKCKVT | | | | 16-32 | |
| EITDEEVKR | GDC | | | | | |
| SEQ ID NO: 1027 | SEQ ID NO: 1086 | | | | | |
| MFTMKKSLLLVFFLGTIA | GILTDTLKGAAKNV | 30 | 3 | 61.8 | 8-16/ | PeNi11 |
| LSLCEEERGADDDNGG | AGVLLDKLKCKITG | | | | 32-128 | |
| EITDEEIKR | GC | | | | | |
| SEQ ID NO: 1028 | SEQ ID NO: 1087 | | | | | |
| MFTLRKSLLLLFFLGMV | GLWTTIKEGVKNFS | 29 | 3 | 67.5 | 4-8/ | PeNi14 |
| SLSLCEQERDADEDEG | VGVLDKIRCKITGG | | | | 16-64 | |
| EVTEEVKR | C | | | | | |
| SEQ ID NO: 1029 | SEQ ID NO: 15 | | | | | |
| MKLLALVLVLSCVVAYT | SWLSKSVKKLVNK | 30 | 8 | 25.5 | 1-2/ | TeRu4 |
| TARKRGQYWPTNTKIFT | KNYTRLEKLAKKKL | | | | >128 | |
| TPYRFRREA | FNE | | | | | |
| DQGSIVANLKNTPQLPF | SEQ ID NO: 996 | | | | | |
| DDNENLRLVLFDNDPTV | | | | | | |
| DLGEDDKEI | | | | | | |
| PGPQSQPNALSNNLHLI | | | | | | |
| DENDYFSSYTSQPGTY | | | | | | |
| RSFPRNFGTS | | | | | | |
| GRYRWRREAGGHVEP | | | | | | |
| RLRFDAETQRGNSFFTD | | | | | | |
| FADLQRRANGR | | | | | | |
| GIEPTVSATAGIRFRQEA | | | | | | |
| DQINPLAVRRERR | | | | | | |
| SEQ ID NO: 1030 | | | | | | |
| IFLVGCKLFGNFILQRMQ | KIKIPWGKVKDFLV | 23 | 6 | 45.0 | 1-4/ | TeBi1 |
| LLLALADAVA | GGMKAVGKK | | | | 2-8 | |
| SEQ ID NO: 1031 | SEQ ID NO: 1090 | | | | | |

*MIC: Minimum inhibitory concentration
^{†}E. coli: wild-type Escherichia coli ATCC 29522; S. aureus: wild-type Staphylococcus aureus ATCC 29213

### 1.2.3. Novelty of Discovered AMPs

To assess if the putative AMPs discovered using rAMPage are novel, a BLASTp [29](basic local alignment search tool) protein search was performed using the 1137 sequences that met the selection criteria. Of these, 1024 sequences are reported as novel, providing no antimicrobial characterization or exact match (sequence identity=100%; query coverage=100%) within the NCBI non-redundant protein database [29]. The novelty analysis results for the seven moderately to highly active AMPs are presented in Table 4. Four of the queried putative AMPs (AmMa1, OdMa12, PeNi10, and PeNi14) are novel in sequence, aligning with high sequence identity (≥90%) to existing NCBI annotations [29]. Two putative AMPs (PeNi11 and TeBi1) are known and published AMPs, aligning with 100% sequence identity of the precursor protein and across the prepro and mature regions. One putative AMP (TeRu4) aligns with high sequence identity to an uncharacterized protein in the NCBI non-redundant protein database.

Table 4 shows comparison of sequence identities (%) of the discovered AMPs with their best-known AMP blastp matches to the NCBI non-redundant (nr) protein database over the entire sequence (precursor), prepro or mature sequences.

TABLE 4
Comparison of sequence identities (%) of the putative AMPs with their best-known AMP
blastp matches over the entire sequence (precursor) or by prepro or mature sequences

| Peptide | | Highest scoring blastp | Sequence Identity (%) | | |
|---|---|---|---|---|---|
| ID | Source Organism | match | Precursor | Prepro | Mature |
| AmMa1 | Amolops mantzorum | Palustrin-2GN3 | 97 | 100 | 93 |
| | | (ADM34231.1) | | | (SEQ ID |
| | | [Amolops granulosus] | | | NO: 12) |
| OdMa12 | Odorrana margaretae | Odorranain-F2 | 98 | 100 | 97 |
| | | (ABG76517.1) | | | (SEQ ID |
| | | [Odorrana grahami] | | | NO: 69) |
| PeNi10 | Leptobrachium boringii | Pelophylaxin-1 | 82 | 86 | 77 |
| | Polypedates | (Q2WCN8.1) | 98 | 97 | 100 |
| | megacephalus | [Pelophylax fukienensis] | | | (SEQ ID |
| | Pelophylax | Ranatuerin-2N | | | NO: 1086) |
| | nigromaculatus | (AEM68233.1)* | | | |
| | Rhacophorus dennysi | [Pelophylax nigromaculatus] | | | |
| | Rhacophorus | | | | |
| | omeimontis | | | | |
| PeNi11 | Leptobrachium boringii | Pelophylaxin-1 | 100 | 100 | 100 |
| | Polypedates | (Q2WCN8.1) | | | (SEQ ID |
| | megacephalus | [Pelophylax fukienensis] | | | NO: 1087) |
| | Pelophylax | | | | |
| | nigromaculatus | | | | |
| | Rhacophorus dennysi | | | | |
| | Rhacophorus | | | | |
| | omeimontis | | | | |
| PeNi14 | Bufo gargarizans | Palustrin-2HB1 | 90 | 93 | 86 |
| | Polypedates | (AIU998997.1) | | | (SEQ ID |
| | megacephalus | [Pelophylax hubeiensis] | | | NO: 15) |
| | Pelophylax | | | | |
| | nigromaculatus | | | | |
| | Rhacophorus | | | | |
| | omeimontis | | | | |
| TeRu4 | Temnothorax rugatulus | Uncharacterized protein | 94 | 93 | 97 |
| | | (XP_024884948.1) | 91 | 90 | 97 |
| | | [Temnothorax curvispinosus] | | | (SEQ ID |
| | | Uncharacterized protein | | | NO: 996) |
| | | (TGZ47385.1)* | | | |
| | | [Temnothorax longispinosus] | | | |
| TeBi1 | Tetramorium bicarinatum | M-myrmicitoxin(01)-Tb1a | 100 | — | 100 |
| | | (W8GNV3.1) | | | (SEQ ID |
| | | [Tetramorium bicarinatum] | | | NO: 1090) |

*Highest scoring blastp match if query sequence consists of only the mature sequence

AmMa1, derived from the Mouping sucker frog, A. mantzorum, aligned with 97% sequence identity to Palustrin-2GN3 [30] from a species of the same genus, A. granulosus, differing only by two AA in the mature region (FIG. 9, part A). Similarly, OdMa12, found in the green odorous frog, O. margaretae, aligned with 98% sequence identity to odorranain-F2 [31] from a species of the same genus, O. grahami, differing only by one AA in the mature region (FIG. 9, part B). While these two sequences (AmMa1 and OdMa2) are very similar to known sequences, it was additionally found that each of them in a different species of the same genus.

PeNi10 was detected in the dark-spotted frog P. nigromaculatus, and aligned with 82% identity to pelophylaxin-1 [32] from a species of the same genus, P. fukienensis (FIG. 9, part C). PeNi10 was also identified in four other species of frogs: L. boringii, P. megacephalus, R. dennysi, R. omeimontis (FIG. 11, part A). Although the PeNi10 precursor aligns best to pelophylaxin-1, the mature region aligns with complete sequence identity to ranatuerin-2N.

PeNi14, also derived from the dark-spotted frog, P. nigromaculatus, aligned with 90% sequence identity to palustrin-2HB1 [33] from a species of the same genus, P. hubeiensis (FIG. 9, part D). PeNi14 was also detected in three other species of frogs: B. gargarizans, P. megacephalus, R. omeimontis (FIG. 11, part B).

Originating from the dark-spotted frog, P. nigromaculatus, PeNi11 aligned with 100% sequence identity to pelophylaxin-1 [32] from a species of the same genus, P. fukienensis, meaning it is identical to a known AMP precursor (FIG. 8, part E). However, in addition to P. nigromaculatus, PeNi11 was also detected in four other species of frogs: L. boringii, P. megacephalus, R. dennysi, R. omeimontis (FIG. 11, part C).

Found in the venom of tramp ant, T. bicarinatum, TeBi1 aligned with 100% sequence identity with bicarinalin [34,35] of the same species (FIG. 10, part F). In the case of TeBi1, its precursor was partial on the 5′ end, accounting for no alignments in the prepro sequence.

TeRu4, discovered in the brain of the small myrmicine ant, T. rugatulus, aligned with 100% sequence identity to an uncharacterized protein [36] from a species of the same genus, T. longispinosus (FIG. 10, part G). While TeRu4 is not a novel protein, it is a novel mature AMP as it has not been previously characterized to have antimicrobial properties.

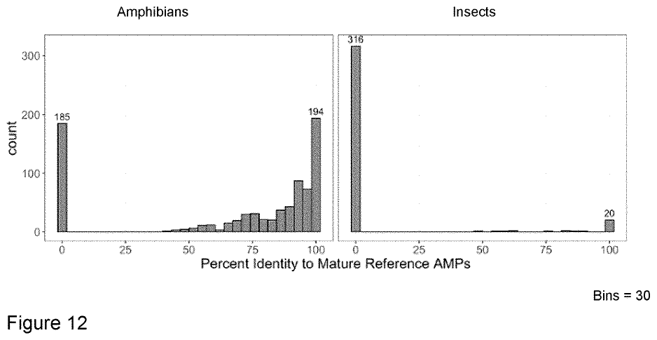

Additional annotation of the seven bioactive peptides (five amphibians, two insects) can be found in Table 5. The underrepresentation of insect AMPs in the literature, compared to amphibians, is further demonstrated here; while the amphibian peptides have been annotated with “frog antimicrobial peptide” domains in both InterProScan [37] and Pfam [38], the insect sequences have no protein family annotations. FIG. 12 illustrates the sequence identity between AMPs identified by rAMPage and known AMPs for amphibian and insect AMPs. Although the majority of putative AMPs from rAMPage were novel sequences, previously reported AMP sequences were also identified and are a good demonstration and internal validation of the robustness of this methodology.

Table 5 shows annotation of moderately to highly active putative mature AMPs Annotation executed with ENTAP, Exonerate, and BLAST web server [118] when there are no significant alignments to reference AMPs.

TABLE 5
Annotation of Moderately to Highly Active Mature AMPs

| Name | Species | Annotation |
|---|---|---|
| AmMa1 | Amolops mantzorum | ADM34231.1: palustrin-2GN3 antimicrobial peptide |
| | | precursor [Amolops granulosus] (93.10%) |
| | | AP02474: Palustrin-2GN3 [Hylarana taipehensis] (93.10%); |
| | | SP_E1AXE7: Granulosusin-E1 [Amolops granulosus] (93.10%) |
| | | IPR012521: Frog antimicrobial peptide, brevinin- |
| | | 2/esculentin type |
| | | PF08023: Frog antimicrobial peptide |
| OdMa12 | Odorrana margaretae | ABG76517.1: Odorranain-F2 antimicrobial peptide |
| | | precursor [Odorrana grahami] (96.55%) |
| | | SP_A6MBL6: Odorranain-F2 [Odorrana grahami] (96.55%) |
| | | IPR012521: Frog antimicrobial peptide, brevinin- |
| | | 2/esculentin type |
| | | PF08023: Frog antimicrobial peptide |
| PeNi10 | Leptobrachium boringii | AEM68233.1: rantauerin-2N protein precursor, partial |
| | Polypedates megacephalus | [Pelophylax nigromaculatus] (100%) |
| | Pelophylax nigromaculatus | AP02224: Pelophylaxin-2GY [Pelophylax nigromaculatus] (96.67%) |
| | Rhacophorus dennysi | IPR012521: Frog antimicrobial peptide, brevinin- |
| | Rhacophorus omeimontis | 2/esculentin type |
| | | PF08023: Frog antimicrobial peptide |
| PeNi11 | Leptobrachium boringii | AEM68231.1: rantauerin-2N protein precursor, partial |
| | Polypedates megacephalus | [Pelophylax nigromaculatus] (100%) |
| | Pelophylax nigromaculatus | AP01394: Pelophylaxin-1 [Pelophylax plancyi fukienensis] (100%) |
| | Rhacophorus dennysi | SP_Q2WCN8: Pelophylaxin-1 [Pelophylax fukienensis] (100%) |
| | Rhacophorus omeimontis | IPR012521: Frog antimicrobial peptide, brevinin- |
| | | 2/esculentin type |
| | | PF08023: Frog antimicrobial peptide |
| PeNi14 | Bufo gargarizans | AIU99897.1: palustrin-2HB1 [Pelophylax hubeiensis] (86.21%) |
| | Polypedates megacephalus | AP02834: Palustrin-2HB1 [Pelophylax hubeiensis] (86.21%) |
| | Pelophylax nigromaculatus | IPR012521: Frog antimicrobial peptide, brevinin- |
| | Rhacophorus omeimontis | 2/esculentin type |
| | | PF08023: Frog antimicrobial peptide |
| TeRu4 | Temnothorax rugatulus | TGZ47385.1: Uncharacterized protein DBV15_00074 |
| | | [Temnothorax longispinosus] (96.67%) |
| | | P48607.3: RecName: Full = Protein spaetzle; Contains: |
| | | RecName: Full = Protein spaetzle C-106; Flags: Precursor |
| | | [Drosophila melanogaster] (36.84%) |
| TeBi1 | Tetramorium bicarinatum | W8GNV3.1: RecName: Full = M-myrmicitoxin(01)-Tb1a; |
| | | Short = M-MYRTX(01)-Tb1a; AltName: Full = Ant peptide P16; |
| | | AltName: Full = Bicarinalin; AltName: Full = Bicarinaline; AltName: |
| | | Full = M-myrmicitoxin-Tb1a; Short = M-MYRTX-Tb1a; Flags: |
| | | Precursor [Tetramorium bicarinatum] (100%) |

### 1.3. Discussion

Using rAMPage, 84 RNA-seq datasets of 38 amphibian and 37 insect species were analyzed to discover 1137 putative AMPs, 1024 of which are previously unknown, and particularly: unknown for antimicrobial effect. In the present Example, validation results are provided on 21 putative AMPs, with over 1000 additional peptide sequences left to investigate. This list is by no means exhaustive; adjusting the described filtering parameters may yield thousands more discoveries (Table 6). Further, the rAMPage pipeline can be readily used on other transcriptome sequencing datasets, though this might call for modifications in experimental designs. For instance, in the case of bacterial RNA-seq datasets with reduced post-transcriptional polyadenylation, RNA-seq data from rRNA depleted libraries would be recommended as input for the pipeline, as opposed to data from poly(A) enriched libraries [39,40].

Table 6 shows major options for rAMPage.

TABLE 6
Major options for rAMPage

| Option | Description |
|---|---|
| -r <file> | Reference transcriptome |
| -s | Strand-specific library construction |
| -E <e-value> | E-value cut-off for HMMs (default: 1e−5) |
| -C <int> | Charge cut-off (default: 2) |
| -S <float> | AMPlify score cut-off (default: 10) |
| -L <int> | Length threshold (default: 30) |

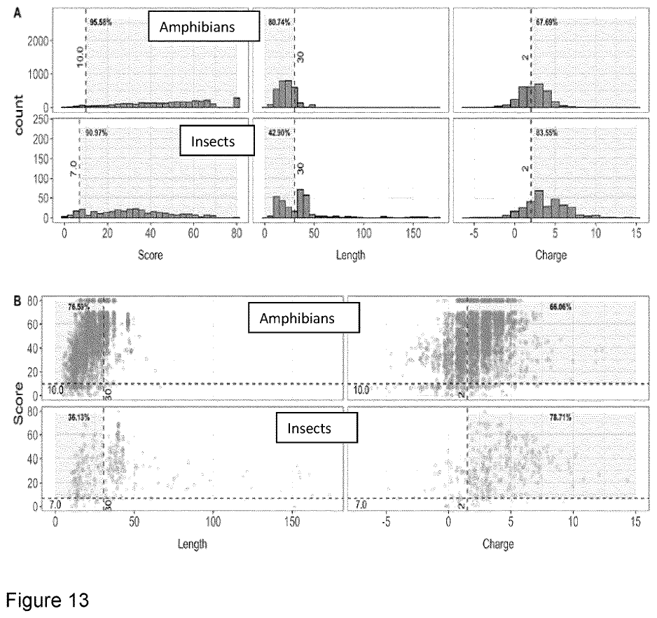

While the sensitivity (proportion of reference AMPs captured by the three putative AMP filters) of rAMPage is <50% (Table 7 and FIG. 13) with the default filtering thresholds, the filters are implemented to select for high confidence predictions that are also easier and more cost-effective to synthesize for validation. However, as more putative AMPs are discovered and the number of reference AMPs increase in public databases, the rAMPage filters can be adjusted accordingly to report more novel AMPs.

Table 7 shows sensitivity of all putative AMP filter combinations.

TABLE 7
Sensitivity of all putative AMP filter combinations

| # | | Sensitivity (%) | | |
|---|---|---|---|---|
| Filters | Filter Combinations | Amphibians | Insects | Overall |
| 1 | Score | 95.58 | 90.97 | 95.15 |
| | Length | 80.74 | 42.90 | 77.21 |
| | Charge | 67.69 | 83.55 | 69.17 |
| 2 | Score & Length | 76.59 | 36.13 | 72.81 |
| | Score & Charge | 66.06 | 78.71 | 67.24 |
| | Length & Charge | 49.19 | 35.16 | 47.88 |
| 3 | Score, Length, & Charge | 47.79 | 31.94 | 46.31 |

Although rAMPage captures most putative AMPs in their complete mature form, their associated precursor sequences may be incomplete, as shown using multiple sequence alignments with Clustal Omega v1.2.4 [41](FIG. 11). However, most partial transcripts are missing sequence on the 5′ end. Therefore, while the AMP precursors may be partial, the mature AMPs at the C-termini are more likely to be complete, thereby still detectable by rAMPage.

Because progress is rapid in bioinformatics, rAMPage is designed to be flexible as new technologies are developed. The pipeline is implemented as a Makefile with each step as a separate target, making the pipeline modular and providing analysis checkpoints. The tools for each step can be substituted with newer/improved tools if needed. Similarly, the pipeline is versatile and can be adapted for other sequencing technologies, for instance by assembling RNA/cDNA long reads from Pacific Biosciences of California (Menlo Park, CA, USA) or Oxford Nanopore Technologies Ltd. (Oxford, UK) instruments.

Recently, the inventors released AMPlify and compared its performance to other state-of-the-art tools for AMP prediction [27]. Other machine learning methods included iAMPpred [42], iAMP-2L [43], AMP Scanner Vr. 2 [44], with AMPlify outperforming all previously described AMP prediction tools in metrics of accuracy, sensitivity, and specificity [27]. For this reason, rAMPage employs AMPlify as its AMP prediction step, and will continue to until it is surpassed in performance. Machine learning in AMP discovery is a dynamic study, ranging from AMP sequence prediction and structure classification to de novo AMP sequence generation and design [45,46,47]. While there are existing methods to mine protein databases [48,49], rAMPage is an all-in-one tool to mine next-generation sequencing data directly from reads to AMP prediction.

While rAMPage can find a substantial number of putative AMPs, its main limitation lies in the fact that it uses homology-based sequence selection and machine learning-based sequence classification steps. These two steps are limited by the quantity and quality of data currently available for training the tools. The homology-based step of rAMPage would be less sensitive when there are more divergent signal sequences in the precursor genes. Similarly, the sequence classification engine in the pipeline, AMPlify, may be biased by known (and limited) classes of AMPs in the databases. However, this limitation is not restricted to only AMPlify, but all approaches dependent on AMP databases for training data sets [48,49,50].

Despite these limitations, which are expected to resolve over time as curated AMP sequence databases grow, a sizeable number (>1000 from 84 RNA-seq datasets) of AMPs were reported by the pipeline with the filters described herein. In the tested set of 21 peptides, seven demonstrated antimicrobial activity against a defined set of bacteria in vitro and 15 did not. AST experiments can assess activity against the tested pathogens but cannot rule in or out an activity against other targets. Further, AMPs have multiple modes of action, and the AST protocol used in this Example only validates direct action and does not test the putative immunomodulatory effects of these peptides, for instance. Of the seven active putative AMPs, three were moderately active, and all three are expressed in multiple amphibian species, potentially signaling the evolutionary significance of these AMPs.

An AMP of particular interest in the present Example is TeRu4, due to its novelty and specificity in bioactivity. The precursor sequence of TeRu4 is 234 AA long, indicating that TeRu4 may be a multi-functional protein, such as a histone whose subsequence includes antimicrobial properties [51]. Additionally, TeRu4 showed a 36.84% sequence similarity to the spaetzle protein from the fruit fly Drosophila melanogaster, a protein in the insect Toll pathway, which triggers AMP production [52]. TeRu4 is also the most specific of the active putative AMPs tested. While all the other active peptides tested are active against both E. coli and S. aureus, TeRu4 is active only against E. coli, a Gram-negative bacterium. This specificity may indicate a unique mechanism of action.

Despite the great promise of discovering putative AMPs with rAMPage, AMP-based drug development still faces some biological challenges, such as peptide stability and bacterial resistance. AMPs in their mature form are considered more unstable and more easily degraded by proteases. While synthesizing precursors for testing would increase stability, doing so would drive up the cost of synthesis using conventional synthetic chemistry methods. Although resistance to AMPs emerges at a slower rate compared to resistance to antibiotics, bacteria may develop resistance to AMPs through surface remodeling, modulation of AMP gene expression, proteolytic degradation, trapping, efflux pumps, and biofilms [4,53,54,55]. To combat specific mechanisms of resistance, targeted AMP discovery methods are being developed. A method to discover AMPs with anti-biofilm activity is described in a preprint [26], and a curated 3D structural and functional repository of AMPs relevant to biofilm studies called B-AMP was recently published [26]. Finding solutions to these and other challenges in developing AMPs as replacements for conventional small molecule antibiotics is an active field of research [56,57,58].

### 1.4. Materials and Methods

AMPage is an AMP discovery pipeline that takes short RNA-seq reads as input, and outputs candidate putative AMPs for wet lab validation. Since it is a homology-based method to select a list of candidates for classification, a set of reference AMPs is required. Here, it is described how input datasets and reference AMPs are collated, as well as each step of rAMPage.

### 1.4.1. Collating Input RNA-Seq Datasets

The RNA-seq reads from 38 amphibian and 37 insect species were downloaded from the Sequence Read Archive (SRA) [59] using fasterq-dump v2.10.5 (http://ncbi.github.io/sra-tools) from the NCBI SRA Tool Kit. Analyzing RNA-seq (transcriptomic) reads enables the discovery of expressed putative AMPs. Because some RNA-seq experiments were conducted with multiple tissues or treatments, there are 75 species in total, but 84 datasets are shown in Table 8 and Table 9.

Table 8 shows amphibian RNA-seq datasets SRA [59] accessions for amphibian RNA-seq reads, metadata taken from the SRA Run Selector, and strand-specificity determined by the methods of each respective published paper.

TABLE 8
Amphibian RNA-seq datasets

| Accession(s) | Species | Experimental Note(s) |
|---|---|---|
| SRX6761091-92; | Allobates | Strand-specific (77), skin tissue, males and females, alkaloid |
| SRX5102733-40 | femoralis | consumption and control; Not strand-specific (78), skin and liver |
| | | tissue, males and females |
| SRX2554903 | Amolops | Not strand-specific (79), mixed cerebrum, skeletal muscle, heart, |
| | mantzorum | liver, testicle, and skin, males |
| SRX5102747-50, | Amazophrynella | Not strand-specific (78), skin and liver tissue, males |
| SRX5102773-74, | minuta | |
| SRX5102777-78 | | |
| SRX5102731-32, | Ameerega | Not strand-specific (78), skin and liver tissue, males and females |
| SRX5102769-72, | petersi | |
| SRX5102775-76 | | |
| SRX2640691 | Bufo | Strand-specific (80), skin tissue, adult, pooled male and female |
| | gargarizans | |
| SRX205680-84, | Cyclorana | Not strand-specific (81), adult skeletal muscle, aestivation |
| SRX206002-05 | alboguttata | |
| SRX6761100-02; | Dendrobates | Strand-specific, skin tissue, alkaloid consumption and control, |
| ERP107602 | auratus | adults, males and females; Strand-specific |
| | | [81], skin tissue, colour morphology, metamorphosis |
| SRX6761103-06 | Dendrobates | Strand-specific (77), skin tissue, alkaloid consumption and |
| | leucomelas | control, adult, males and females |
| SRX6761093-99, | Dendrobates | Strand-specific (77), skin tissue, alkaloid consumption and |
| SRX6761107 | tinctorius | control, adult, males and females |
| SRP151854 | Hypsiboas | Strand-specific (82), skin tissue, adult |
| | pugnax | |
| SRX1720190 | Leptobrachium | Strand-specific (80), skin tissue, pooled male and female, |
| | boringii | terrestrial ecotype |
| SRP096145 | Litoria | Not strand-specific (83), skin tissue, chytridiomycosis exposure, |
| | verreauxii | adult |
| SRX1720191 | Megophrys | Strand-specific (80), skin tissue, pooled male and female, |
| | sangzhiensis | terrestrial ecotype |
| SRX317137; | Odorrana | Not strand-specific (84), mixed cerebrum, eye, skeletal muscle, |
| SRX1720189 | margaretae | heart, liver, testicle, ootheca, and tadpole tissue; Strand-specific, |
| | | skin tissue, pooled male and female, semi-aquatic ecotype |
| SRP124485 | Oreolalax | Not strand-specific (85), skin, eyeball, and liver tissue, light |
| | rhodostigmatus | exposure and high altitudes |
| SRP199453 | Oophaga | Strand-specific (86), skin, liver, and gut tissue, alkaloid |
| | sylvatica | consumption and control, adult, wild type and lab reared ecotype |
| SRX3845976-77 | Odorrana | Not strand-specific (87), skin tissue, adult female and males |
| | tormota | |
| DRX154877 | Pyxicephalus | Strand-specific (88), mixed skin, muscle, intestine, brain and |
| | adspersus | internal organ tissue, aestivation |
| SRX5734372 | Pseudophilautus | Not strand-specific (89), ventral skin tissue, adult, males |
| | amboli | |
| SRX1720193 | Polypedates | Strand-specific (80), skin tissue, pooled male and female, |
| | megacephalus | arboreal ecotype |
| SRX5734384 | Phrynomantis | Not strand-specific (89), dorsal skin tissue, adult, males |
| | microps | |
| SRX532382; | Pelophylax | Not strand-specific (90), mixed brain and gonad tissue; Strand- |
| SRX1720192; | nigromaculatus | specific (80), semi-aquatic ecotype, skin tissue, pooled male and |
| SRX2640690; | | female; Strand-specific (80), skin tissue, pooled male and |
| SRP144311 | | female, Hallowell cultivar; Not strand-specific (GEO |
| | | GSE113944), skin tissue, 30Gy radiation exposure and control |
| SRX5102741-46, | Pristimantis | Not strand-specific (78), male, skin and liver tissue, adult |
| SRX5102761-62 | toftae | |
| SRX2554902 | Quasipaa | Not strand-specific (79), mixed cerebrum, skeletal muscle, heart, |
| | boulengeri | liver, testicle, and skin tissue |
| SRX1080472-77, | Rana | Strand-specific (11), T3 exposure and control, back skin tissue, |
| SRX2034988, | catesbeiana | tadpoles, male; Not strand-specific (91), ventral skin tissue, |
| SRX2034994, | | fungal exposures and control, juveniles |
| SRX2036208; | | |
| SRX2989033-59 | | |
| SRX1720195 | Rhacophorus | Strand-specific (80), pooled male and female, skin tissue, |
| | dennysi | arboreal ecotype |
| ERP110499 | Ranitonmeya | Strand-specific (92), skin tissue, Huallaga, Sauce, Tarapoto, |
| | imitator | Varadero ecotypes |
| SRX1368943 | Rhacophorus | Strand-specific (80), pooled male and female, skin tissue, |
| | omeimontis | arboreal ecotype |
| SRX482652-57 | Rana | Not strand-specific (93), males and females, organ tissue |
| | pipiens | |
| SRX5102759-60, | Ranitomeya | Not strand-specific (78), male, skin and liver tissue, adult |
| SRX5102763-68 | sirensis | |
| SRX2989010-32 | Rana | Not strand-specific (91), fungal exposures and control, ventral |
| | sylvatica | skin tissue, juveniles |
| ERX993648-56 | Rana | Not strand-specific (94), liver tissue, metamorph stage, viral and |
| | temporaria | fungal exposures and control |
| SRX5102751-58 | Scinax | Not strand-specific (78), males and females, skin and liver |
| | ruber | tissue, adult |
| SRX1704778 | Xenopus | Not strand-specific (95), liver tissue, sub-adult |
| | allofraseri | |
| SRX1704840 | Xenopus | Not strand-specific (95), adult, females, liver tissue |
| | borealis | |
| SRX847156-57 | Xenopus | Not strand-specific (96), male, tadpole, liver tissue, T3 exposure |
| | laevis | and control |
| SRX1703541 | Xenopus | Not strand-specific (96), adult, liver tissue |
| | largeni | |
| SRX191164-68 | Xenopus | Not strand-specific (97), brain, liver, kidney, heart, and skeletal |
| | tropicalis | muscle tissue, males and females |

Table 9 shows insect RNA-seq datasets SRA [59] accessions for insect RNA-seq reads, metadata taken from the SRA Run Selector, and strand-specificity determined by the methods of each respective published paper.

TABLE 9
Insect RNA-seq Datasets

| Accession(s) | Species | Experimental Note(s) |
|---|---|---|
| SRP083113 | Apis cerana | Not strand-specific (30), gut tissue, fungal exposure, |
| | | subspecies cerana |
| SRX2414288 | Ampulex compressa | Not strand-specific (98), venom gland and venom sac |
| | | tissue, adult |
| ERP002002 | Acromyrmex echinatior | Not strand-specific (99), fungal exposures and control |
| SRX6999815 | Anterhynchium | Not strand-specific (100), venom gland tissue |
| | flavomarginatum | |
| SRP117554 | Apis mellifera | Strand-specific (101), gut tissue, parasitic exposure |
| SRX6999805 | Bombus ardens | Not strand-specific (100), venom gland tissue |
| SRX6999806 | Bombus consobrinus | Not strand-specific (100), venom gland tissue |
| SRX5818710 | Bracon nigricans | Strand-specific (102), venom gland tissue, females, adult |
| SRX6999807 | Bombus ussurensis | Not strand-specific (100), venom gland tissue |
| SRP057573 | Camponotus castaneus | Strand-specific (103), head tissue, fungal infection and |
| | | control |
| SRP069794 | Cardiocondyla | Not strand-specific (104), female, whole body tissue, |
| | obscurior | injured |
| SRX1973411, | Cotesia vestalis | Not strand-specific (105), adult, venom gland tissue |
| SRX377438 | | |
| SRX2190163, | Diadromus collaris | Not strand-specific (105), adult, female, venom gland |
| SRX371684 | | tissue |
| SRX7651835 | Diachasmimorpha | Strand-specific (106), female, venom gland tissue, |
| | longicaudata | unemerged adult |
| SRX337940 | Microplitis demolitor | Not strand-specific (107), venom gland tissue |
| SRX3556750 | Myrmecia gulosa | Strand-specific (108), venom apparatus tissue |
| SRX2241561 | Nasonia giraulti | Not strand-specific (109), venom gland and venom |
| | | reservoir tissue, adult female |
| SRX2263332 | Nasonia vitripennis × | Not strand-specific (109), venom gland and venom |
| | Nasonia giraulti F1 | reservoir tissue |
| SRP120985; | Nasonia vitripennis | Not strand-specific (110), females, head tissue; Not |
| SRX2241560; | | strand-specific (109), venom gland tissue, adult female; |
| SRP067692 | | Strand-specific (111), venom gland and ovary tissue |
| SRX6999813 | Oreumenes decoratus | Not strand-specific (100), venom gland tissue |
| DRR093871 | Odontomachus | Strand-specific (112), venom gland and venom sac tissue |
| | monticola | |
| DRP002507 | Pogonomyrmex | Not strand-specific (113), larva, pupae, and adult stages, |
| | barbatus | worker and gyne castes, males |
| SRX6999811 | Polistes rothneyi | Not strand-specific (100), venom gland tissue |
| SRX6999812 | Polistes snelleni | Not strand-specific (100), venom gland tissue |
| SRX6653167 | Pimpla turionellae | Not strand-specific (114), female, venom gland tissue |
| SRX6999810 | Parapolybia varia | Not strand-specific (100), venom gland tissue |
| SRX6897943-45 | Pachycrepoideus | Not strand-specific (115), female, venom apparatus tissue |
| | vindemmiae | |
| SRX6999804 | Sceliphron deforme | Not strand-specific (100), venom gland tissue |
| SRX6999814 | Sphecidae sp. KJ- | Not strand-specific (100), venom gland tissue |
| | 8906 | |
| SRX424883 | Tetramorium | Not strand-specific (116), venom gland tissue |
| | bicarinatum | |
| SRP133940 | Temnothorax | Not strand-specific (117), old and young age, brain and |
| | rugatulus | fatbody tissue, queens |
| SRX2241559 | Trichomalopsis | Not strand-specific (109), adult female, venom gland and |
| | sarcophagae | venom reservoir tissue |
| SRX2241558 | Urolepis rufipes | Not strand-specific (109), adult female, venom gland and |
| | | venom reservoir tissue |
| SRX6999802 | Vespa analis | Not strand-specific (100), venom gland tissue |
| SRX6999803 | Vespa crabro | Not strand-specific (100), venom gland tissue |
| SRX6999808 | Vespa dybowskii | Not strand-specific (100), venom gland tissue |
| SRX6999809 | Vespa simillima | Not strand-specific (100), venom gland tissue |

### 1.4.2. Collating Reference AMP Datasets

A set of 3306 AMP sequences were collated from two high-quality AMP databases: the Database of Anuran Defense Peptides (DADP; http://split4.pmfst.hr/dadp/) [15] and the Antimicrobial Peptide Database 3 (APD3; https://aps.unmc.edu) [16]. These databases are highly curated, where sequences have been validated for efficacy. To complement this list, 3835 precursor and mature AMP sequences of amphibian and insect origin were downloaded from the NCBI non-redundant (nr) protein database [29]. These sequences are less curated, including partial sequences and sequences with only in silico prediction, etc., accounting for the difference between numbers from DADP/APD3 and NCBI in Table 10.

Table 10 shows breakdown of AMP sequences in AMP databases AMP sequences across the APD3, DADP, and NCBI (nr) databases. The number of newly added sequences as of May 10, 2021 compared to date the shown below each database name are indicated in bold. The last column “All” includes all AMP sequences in the database, regardless of organism of origin. There is no “All” number for NCBI on Jul. 2020. NCBI search terms used: antimicrobial[All Fields] AND (amphibia[organism] OR insecta[organism]).

TABLE 10
Breakdown of AMP sequences in AMP databases

| | Amphibians | Insects | All |
|---|---|---|---|
| Database | (nr) | (nr) | (nr) |
| APD3 September 2020 (release) | 1,075 +34 | 310 +13 | 3,125 +97 |
| DADP September 2020 (downloaded) | 1,921 | 0 | 1,921 |
| NCBI July 2020 (downloaded) | 2,850 +41 | 985 +57 | +185,967 |
| Reference AMPs | 4,663 +73 | 1,204 +62 | N/A |

### 1.4.3. rAMPage Pipeline

rAMPage is implemented as a Makefile and written in bash, Python3, and R. It has been made publicly available by the inventors on GitHub (https:H/github.com/bcgsc/rAMPage, v1.0). The pipeline was tested for the dependencies listed in Table 11 and Table 12, and is highly customizable, with its major parameter options listed in Table 6. A flowchart of the rAMPage pipeline is shown in FIG. 3.

Table 11 shows shell scripting dependencies of rAMPage.

TABLE 11
Shell scripting dependencies of rAMPage

| Dependency | Tested Version |
|---|---|
| GNU bash | 5.0.11(1) |
| GNU make | 4.3 |
| GNU awk | 5.0.1 |
| GNU sed | 4.8 |
| GNU grep | 3.4 |
| GNU column | 2.36 |
| Miller mlr | 5.4.0 |
| bc | 1.07.1 |
| gzip | 1.10 |
| Python | 3.7.7 |
| Rscript* | 4.0.2 |

*requires tidyverse v1.3.0, glue v1.4.2, and docopt v0.7.1

Table 12 shows bioinformatic tool dependencies of rAMPage.

TABLE 12
Bioinformatic tool dependencies of rAMPage

| | Tested | |
|---|---|---|
| Tool | Version | Source |
| Fastp[60] | 0.20.0 | https://github.com/OpenGene/fastp/releases/tag/v0.20.0 |
| RNA-Bloom[61] | 1.3.1 | https://github.com/bcgsc/RNA-Bloom/releases/tag/v1.3.1 |
| Salmon [62] | 1.3.0 | https://github.com/COMBINE-lab/salmon/releases/tag/v1.3.0 |
| TransDecoder[63] | 5.5.0 | https://github.com/TransDecoder/TransDecoder/releases/ |
| | | tag/TransDecoder-v5.5.0 |
| HMMER[64] | 3.3.1 | https://github.com/EddyRivasLab/hmmer/releases/tag/ |
| | | hmmer-3.3.1 |
| CD-HIT[75] | 4.8.1 | https://github.com/weizhongli/cdhit/releases/tag/V4.8.1 |
| seqtk | 1.1-r91 | https://github.com/lh3/seqtk/releases/tag/v1.1 |
| SignalP | 3.0 | https://services.healthtech.dtu.dk/services/SignalP-5.0/ |
| | | 9-Downloads.php# |
| ProP [66] | 1.0c | https://services.healthtech.dtu.dk/services/ProP-1.0/ |
| | | 9-Downloads.php# |
| AMPlify [27] | 1.0.0 | https://github.com/bcgsc/AMPlify/releases/tag/v1.0.3 |
| E_{N}TAP [70] | 0.10.7- | https://github.com/harta55/EnTAP/tree/v0.10.7-beta |
| | beta | |
| Exonerate [73] | 2.4.0 | https://www.ebi.ac.uk/about/vertebrate- |
| | | genomics/software/exonerate |
| SABLE[74] | 4.0 | https://sourceforge.net/projects/meller-sable/ |
| Clustal Omega [41] | 1.2.4 | http://www.clustal.org/omega/ |

Because the datasets used for rAMPage originate from publicly available genomic resources and thus, the present inventors have no control over the experimental design or protocols used, rigorous quality control was performed. The RNA-seq reads were trimmed to remove adapter sequences using fastp v0.20.0 [60], which does not require the adapter sequences to be known, and instead infers adapter sequences from sequence overlaps between reads. This is particularly convenient when dealing with multiple datasets that possibly have different sequencing protocols.

To assemble the RNA-seq reads into transcripts RNA-Bloom v1.3.1 [61] was used, a de novo transcriptome assembler that works with single and paired-end reads. RNA-Bloom is able to assemble transcriptomes without a reference but also allows for reference-guided assembly if a reference is available. It also allows for multi-sample pooling, where, for instance, reads describing multiple tissues from the same individual or different treatments for the same species are assembled together while retaining the tissues or treatment specificity of assembled transcripts.

It was found that the transcripts with a smaller number of reads have less reconstruction evidence; thus, assembled sequences with lower measured expression levels may be enriched for misassemblies. To exclude such sequences from downstream analysis, Salmon v1.3.0 [62] was used to quantify assembled transcript expression levels, and filtered out transcripts with less than 1 TPM (transcripts per million) expression.

To obtain translated peptide sequences from the transcripts, TransDecoder v5.5.0 [63] was used to conduct an in silico six-frame open reading frame (ORF) translation, and ORFs that are at least 50 AA were selected for downstream analysis. In the case of nesting ORFs, the longest ORF was chosen.

To select putative AMP precursors from this vast pool of assembled and translated sequences, a homology search was conducted against a curated reference AMP dataset (Table 10) using HMMER v3.3.1 [64] and assigned an Expect (E) value to every sequence. The E-value describes the number of hits expected by chance when searching a database of a particular size [65]. Sequences that share a certain degree of identity, with E-values of less than 10⁻⁵, were selected as putative AMP precursors.

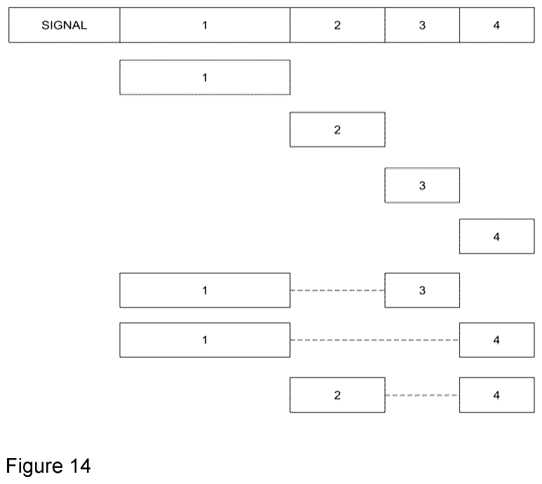

These putative precursor (or partial precursor) sequences were then cleaved in silico using ProP v1.0c [66] to obtain putative mature AMP sequences, to be further classified. However, cleavage prediction tools only predict where the cleavage occurs, not what each resulting cleaved peptide represents, and the AMP precursor organization shows inter- and intra-species variability [13,67,68]. While amphibian AMPs are typically cleaved at a lysine-arginine (KR) motif and their precursor structure follows a conserved structure (prepro sequence containing acidic AA residues and a mature bioactive AMP) [67], insect AMPs are typically cleaved at an RXXR motif (two arginine residues surrounding two optional AA) and the precursor structure is not always conserved [68]. Insect AMPs are more variable in structure [13], increasing the difficulty in identifying the putative mature peptide. This difficulty is especially present in precursor structures with multiple acidic regions (UniProtKB P54684.1) or multiple bioactive regions (UniProtKB P35581.1). In such multi-peptide precursors, it is unclear whether each bioactive region is its own isoform or part of a larger mature peptide. To account for this and to possibly discover novel but perhaps not naturally occurring putative AMPs, cleaved peptides were also recombined in a manner similar to alternative splicing (FIG. 14). In this procedure, the order and orientation of the cleaved peptides were maintained, and cleaved peptides that originally share cleavage sites were not recombined, with a maximum of three cleaved peptides within recombination. This recombination feature can be turned off in rAMPage's options.

The collected candidate peptide sequences were classified with AMPlify v1.0.3 [27] as AMP or non-AMP sequences. When given a sequence, AMPlify calculates a score between 0 to 80, with the score≈3.0103 corresponding to the classification probability cutoff of 50% through Equation (1).

To facilitate AMP synthesis for the validation experiments, the putative AMPs were filtered by length and charge, in addition to the AMPlify score. A maximum length of 30 AA was imposed to control the cost of peptide synthesis and to reduce the number of spurious hits from recombined sequences. A minimum charge of +2 was imposed as a proxy to assess the effectiveness of an AMP, as past evidence indicates that more positively charged AMPs show higher activity, especially when their mechanism of action is membrane disruption [69]. Because AMPlify was trained on mostly amphibian AMPs, different score thresholds were imposed for amphibian (>10) and insect (>7) datasets to compensate for the dearth of insect training AMPs.

To annotate the final set of filtered putative AMPs, E_{N}TAP v0.10.7, Eukaryotic Non-Model Transcriptome Annotation Pipeline [70], were used, along with UniProtKB (release 2020_06) [71], RefSeq (release 203, [72], and NCBI non-redundant (nr) (v5) [29] protein databases. For AMPs that E_{N}TAP failed to annotate, InterProScan 5 v5.30-69.0 [37] was run separately to annotate protein families, functions, and domains. Exonerate v2.4.0 [73] was used to align the filtered putative AMPs against the reference AMPs to assess how many of the labeled AMPs were already known AMPs. Finally, SABLE v4.0 [74] was optionally used to predict secondary structures of the filtered putative AMPs, for visualization.

### 1.4.4. Selecting Filtered Putative AMPs for Validation

To select peptides to validate from the filtered putative AMPs, their sequences were ranked using AMPlify and chose peptides based upon three selection criteria (FIG. 3): “Species Count” (n=7), “Insect Peptide” (n=12), or “AMPlify Score” (n=2), for a final total of 21 AMPs (Table 2). The sequences were first clustered using CD-HIT [75]v4.8.1 with a sequence similarity cutoff of 100%. The longest sequence was chosen for each of these clusters, removing duplicate and subsumed sequences to obtain a non-redundant sequence set.

In the first selection strategy of “Species Count”, sequences that were present in more than two species were chosen. In the “Insect Peptide” strategy, to balance the training bias of AMPlify towards AMPs of amphibian origin, insect-originating sequences were specifically selected using a reduced AMPlify score cutoff of >20. In the “AMPlify Score” strategy, the two highest-scoring peptides (AMPlify score=80.0, 69.9) with the highest charge (+4) were chosen for validation.

### 1.4.5. Antimicrobial Susceptibility Testing (AST)

Twenty-one putative AMP sequences identified using the rAMPage pipeline were validated through a minimum of three AST experiments performed independently on separate days. In these tests, the AMP activity was assessed using two metrics: minimum inhibitory concentration and minimum bactericidal concentration (MIC and MBC, respectively). MIC and MBC values were determined using procedures outlined by the Clinical and Laboratory Standards Institute (CLSI), with the recommended adaptations for the testing of cationic AMPs described previously [76]. “Wild-type” strains of Escherichia coli (E. coli 25922) and Staphylococcus aureus (S. aureus 29213) were purchased from the American Type Culture Collection (ATCC; Manassas, VA, USA) and were used for screening of antimicrobial activity. Briefly, putative AMPs were synthesized by Genscript (Piscataway, NJ, USA) and received in lyophilized format. These peptides were suspended using ultrapure water (Life Technologies, Grand Island, NY, USA; Invitrogen cat #10977-015), and an 11 μL two-fold serial dilution of 1280 to 2.5 μg/mL was prepared in duplicate rows in a 96-well microtiter plate, before being combined with 100 μL standardized bacterial inoculum yielding a final duplicate testing range of 128 to 0.25 μg/mL. The bacterial inoculum was prepared using colonies isolated on non-selective agar and combined with Mueller Hinton Broth. This suspension was measured and adjusted to achieve an optical density of 0.08-0.1, equivalent to a 0.5 McFarland standard (1-2×10⁸ cfu/mL). The inoculum was then diluted to a target concentration of 5±3×10⁵ cfu/mL; total viable counts from the final inoculum were routinely performed to confirm the target bacterial density was achieved. MIC values were reported at the concentrations in which no visible growth was detected following 20-24-h incubation at 37° C. The MIC and adjacent wells were plated onto non-selective agar; the concentration in which killed 99.9% of the inoculum following additional overnight incubation was determined to be the MBC.

### 1.4.6. Hemolysis Experiments

The twenty-one putative AMPs were evaluated for toxicity using three independent hemolysis experiments performed on separate days. Whole blood from healthy donor pigs was purchased from Lampire Biological Laboratories (Pipersville, PA, USA). Red blood cells (RBCs) were washed and isolated by centrifugation, using Roswell Park Memorial Institute medium (RPMI) (Life Technologies, Grand Island, NY, USA; Gibco cat #11835-030). Lyophilized AMPs were suspended and serially diluted from 128-1 μg/mL using RPMI in a 96-well plate, before being combined with 100 μL of a 1% RBC solution. Following a minimum 30 min incubation at 37° C., plates were centrifuged and % volume from each supernatant was transferred to a new 96-well plate. The absorbance of these wells was measured at 415 nm. To quantify hemolytic activity and determine the AMP concentration that kills 50% of the RBCs (HC₅₀), absorbance readings from wells containing RBCs treated with 11 μL of a 2% Triton-X100 solution or RPMI (AMP solvent-only) were used to define 100% and 0% hemolysis, respectively. All centrifugation steps were performed at 500× g for five minutes in an Allegra-6R centrifuge (Beckman Coulter, CA, USA).

### 1.5. Conclusions

rAMPage is a bioinformatics pipeline for high throughput identification of putative AMPs in RNA-seq datasets. It fills a current void in the AMP discovery process, bridging the gap between in silico and in vitro methods. The pipeline has the potential to accelerate the discovery of novel antibiotics, with the possibility to enrich existing AMP sequence repositories. The easy-to-run pipeline design with various checkpoints and the low computational resources required to run rAMPage increase its accessibility to users. By executing rAMPage on publicly available amphibian and insect transcriptome sequencing data, over 1000 putative AMPs were identified. Of those, functional tests were performed on twenty-one putative AMPs and demonstrated that seven have moderate to high activity against E. coli ATCC 25922 and/or S. aureus ATCC 29213. As the number of tested peptides increases, the wet lab validation results can feed back into rAMPage by augmenting the reference AMP datasets, helping refine the underlying homology and machine learning approaches. rAMPage has broad utility in the discovery of novel antimicrobials from a wide variety of transcriptome sequencing datasets.

### Example 2

### Associating Biological Activity and Predicted Structure of Antimicrobial Peptides from Amphibians and Insects

Summary Antimicrobial peptides (AMPs) are a diverse class of short, often cationic biological molecules that present promising opportunities in the development of new therapeutics to combat antimicrobial resistance. Newly developed in silico methods offer the ability to rapidly discover numerous novel AMPs with a variety of physiochemical properties. Herein, using the rAMPage AMP discovery pipeline, 51 AMP candidates were bioinformatically identified from amphibia and insect RNA-seq data and present their in-depth characterization. The studied AMPs demonstrate activity against a panel of bacterial pathogens and have undetected or low toxicity to red blood cells and human cultured cells. Amino acid sequence analysis revealed that 30 of these bioactive peptides belong to either the Brevinin-1, Brevinin-2, Nigrocin-2, or Apidaecin AMP families. Prediction of three-dimensional structures using ColabFold indicated an association between peptides predicted to adopt a helical structure and broad-spectrum antibacterial activity against the Gram-negative and Gram-positive species tested in the panel. These findings highlight the utility of associating the diverse sequences of novel AMPs with their estimated peptide structures in categorizing AMPs and predicting their antimicrobial activity.

2.1. Introduction. Antimicrobial resistance is an escalating global health concern, with multiple infectious diseases becoming increasingly difficult and expensive to treat. An estimated 1.27 million deaths occurred in 2019 due to bacterial resistance to antibiotics [120]. Antimicrobial resistance (AMR) develops through mutations in the bacterial genome as well as horizontal transfer of mobile genetic elements such as plasmids [121]. AMR is exacerbated by the routine administration of antibiotics both clinically and in agricultural settings [122]. Despite the steady increase in antibiotic resistant bacteria, there is a shortfall of novel antibiotics being developed, with no new classes approved for clinical use since the 1980s [123]. This gap between increasing antibiotic resistance and lagging discovery of new drug classes creates an urgent need for innovative therapeutic discovery methods to produce novel antimicrobials with different mechanisms of action to combat the emerging threat to public health [124].

Antimicrobial peptides (AMPs) are short, often cationic and amphipathic, biomolecules that are produced by the innate immune system of all living organisms [125]. They are a functionally and structurally diverse group of compounds that can defend against bacteria, viruses, fungi, and cancer [3]. AMPs can combat bacterial infections directly through interactions with the negatively charged membrane and with intracellular targets such as DNA and RNA [3, 126]. In addition, AMPs can function indirectly through inflammatory or immunomodulatory pathways, resulting in the recruitment of immune cells [127]. Because of these diverse mechanisms of action, it has been suggested that it is more difficult for bacteria to develop resistance to AMPs as compared to conventional antibiotics [8, 128]. However, bacteria intrinsically resistant to AMPs do exist [4], and resistance to colistin, an AMP-based therapeutic used as a last resort, has been observed [129]. Continuing the search for and characterization of AMPs with varying structure and associated mechanisms of action would increase the arsenal of available therapeutics against drug resistant bacteria.

The three-dimensional (3D) structures of AMPs can be classified into several types; α-helical, β-sheet containing, mixed, or linear extended structures [130]. It has been observed that many α-helical and linear extended peptides undergo conformational changes when interacting with the bacterial membrane [131]. The α-helix structure is reported to be the most effective conformation for AMPs to interact with the bacterial membrane, with size, sequence, charge and hydrophobicity all affecting the spectrum and level of the resulting antimicrobial activity [132]. However, the majority of discovered AMPs do not have a known structure, with approximately 25% of all entries in the Antimicrobial Peptide Database (APD3) reporting an experimentally determined structure [133]. Traditional methods for determining the structure of peptides, such as nuclear magnetic resonance (NMR), X-ray crystallography, and cryo-electron microscopy are laborious and expensive in comparison to in silico methods. While the latter only provide predictions, recent advances in artificial intelligence research bring the potential to make them enabling tools for characterizing AMPs. AlphaFold2 is a model that applies deep-learning to predict the 3D structure of proteins with high accuracy, including cases where there are no similar structures [134]. ColabFold has been reported to improve the speed of this prediction by coupling AlphaFold2 with an MMseqs2-based homology search [135].

The relationship between predicted AMP structure and observed antimicrobial activity was established. Specifically, ColabFold was used to predict the 3D structure of a list of 88 putative amphibian and insect AMPs discovered using rAMPage, a homology-based bioinformatic pipeline for in silico discovery of AMPs using RNA-seq reads [119]. The antimicrobial activity was examined for these peptides against two Gram-negative and one Gram-positive bacteria selected from among priority pathogens. Fifty-one AMPs in this list were observed, discovered from 16 amphibian and 16 insect species, with antimicrobial activity and low toxicity to porcine erythrocytes and human embryonic kidney cells. Amphibians possess a diverse repertoire of AMPs, which provide a first line of defense as they transition from an aquatic to a terrestrial habitat throughout their life cycle [11]. Further, unlike vertebrates, insects lack an adaptive immune system, and as such their innate immune system, including AMPs, is their only line of defense in combating bacterial infections [13]. Studying these peptides, an association between AMPs with a predicted helical structure and broader antibacterial activity against the bacterial isolates of the panel was established.

### 2.2. Results

### 2.2.1. 3D Structure Prediction and Clustering

A total of 1137 putative AMPs were identified by rAMPage, and 88 peptides were selected for synthesis using three selection criteria: “Species Count”, “Insect Peptide”, or “AMPlify Score” (see Methods). The shortlist of 88 putative AMPs include 21 peptides described previously [119]. Three independent experiments per peptide were used to investigate the antimicrobial activity of the 88 peptides against Escherichia coli ATCC 25922, a clinical strain of Salmonella enterica serovar Enteritidis, and Staphylococcus aureus ATCC 29213. Fifty-one peptides showed antimicrobial activity against at least one of these bacterial strains (Table 13). To investigate the relationship between estimated structure and bioactivity, the 3D structures of the 88 mature peptides was predicted using ColabFold [135]. These were clustered based on their structural similarities evaluated by TM-score, and STRIDE [136] was used to assign the secondary structure of these predictions. The bioactivity of the peptides was classified as high when the median minimum inhibitory concentration (MIC) was 54 μg/mL, moderate when 4<median MIC s 16 μg/mL, and low when 16<median MIC s 128 μg/mL. As the highest concentration of peptide tested was 128 μg/mL, a median MIC of >128 μg/mL was classified as ‘no inhibition observed’ (N/O). Approximately half (45/88) of the peptides were predicted to adopt a helical structure, three adopted a structure containing β-strands, 17 adopted a linear extended structure, and 23 peptides were predicted to contain both helical and linear extended regions. The latter 23 peptides were classified as either mainly extended (>50% residues fell in extended category; six peptides) or mainly helical (between 50-80% residues in helical structures; 17 peptides). All structural categories contained both amphibian- and insect-derived peptides of varying length, charge, and hydrophobicity profiles. There was an association between antimicrobial activity and structure, with 8% of active peptides being linear extended and 71% being helical, despite extended peptides accounting for 19% of total peptides and helical only accounting for 51%. This association was statistically significant at the alpha=0.05 level, with a p-value=6.6×10⁻⁵. Predicted helical/extended content did not correlate with observed in vitro antimicrobial activity when the percentage of helical residues was less than 80%. Approximately half of the mainly helical and mainly extended peptides were active.

Table 13 provides sequences and physiochemical properties of the 51 bioactive AMPs assessed in this example using rAMPage.

TABLE 13
Sequences and physiochemical properties of the 51 bioactive AMPs discovered
using rAMPage

| Peptide | Source | | | | AMPIify | MW |
|---|---|---|---|---|---|---|
| Name | Organism | Sequence | Length | Charge* | Score | (Da) |
| AmMa1 | Amolops | GILDTLKQLGKAAVQGLL | 29 | 4 | 80.0 | 2943.59 |
| SEQ ID | mantzorum | SKAACKLAKTC | | | | |
| NO: 12 | | | | | | |
| AnFI2 | Anterhynchium | GILRSLGWIQMPRSRRR | 19 | 6 | 31.8 | 2375.82 |
| SEQ ID | flavomarginatum | HR | | | | |
| NO: 721 | | | | | | |
| ApCe1 | Apis cerana | GIYTGRLLPVYIPQPRPP | 24 | 5 | 38.6 | 2853.39 |
| SEQ ID | | HPRLRR | | | | |
| NO: 695 | | | | | | |
| BoAr6 | Bombus ardens | GILRLVTRRFRFSPTNLN | 30 | 6 | 22.1 | 3460.06 |
| SEQ ID | | RYTVARLVSGVP | | | | |
| NO: 790 | | | | | | |
| BoUs1 | Bombus | RKIIAVSVHKLCRVKR | 16 | 6 | 29.9 | 1906.40 |
| SEQ ID | ussurensis | | | | | |
| NO: 815 | | | | | | |
| CaCa1 | Camponotus | FACPIGFFRLKR | 12 | 3 | 7.27 | 1454.79 |
| SEQ ID | castaneus, | | | | | |
| NO: 826 | Odontomachus | | | | | |
| | monticola, | | | | | |
| | Polistes | | | | | |
| | rothneyi, | | | | | |
| | Polistes | | | | | |
| | snelleni, | | | | | |
| | Sphecidae sp. | | | | | |
| | KJ- | | | | | |
| | 8906, Vespa | | | | | |
| | dybowskii | | | | | |
| CaCa2 | Camponotus | FIKTQVLKHLVAGVRVAR | 25 | 5 | 28.7 | 2977.57 |
| SEQ ID | castaneus, | GLDWKWR | | | | |
| NO: 819 | Odontomachus | | | | | |
| | monticola, | | | | | |
| | Temnothorax | | | | | |
| | rugatulus | | | | | |
| CaCa4 | Camponotus | RRFFFATAPCGYSRKFC | 24 | 9 | 23.6 | 2996.58 |
| SEQ ID | castaneus | KITRRKR | | | | |
| NO: 816 | | | | | | |
| DiLo | Diachasmimorpha | GAFVLWGPTPRPRRR | 15 | 4 | 26.0 | 1766.07 |
| SEQ ID | longicaudata | | | | | |
| NO: 864 | | | | | | |
| LiVe1 | Litoria | GWLDIAKKVASVVAGIVK | 19 | 3 | 80.0 | 2010.44 |
| SEQ ID | verreauxii | R | | | | |
| NO: 37 | | | | | | |
| LiVe2 | Litoria | GWLDIAKKVASVVAGLG | 19 | 3 | 70.0 | 1968.36 |
| SEQ ID | verreauxii | KR | | | | |
| NO: 40 | | | | | | |
| MyGu1 | Myrmecia | RRAIFASIRGYLGLRKR | 17 | 6 | 25.3 | 2033.44 |
| SEQ ID | gulosa | | | | | |
| NO: 881 | | | | | | |
| NaVi3 | Nasonia | KLFLTLWKLKR | 11 | 4 | 30.5 | 1445.84 |
| SEQ ID | vitripennis x | | | | | |
| NO: 895 | Nasonia | | | | | |
| | giraulti F1 | | | | | |
| OdMa1 | Odorrana | GLLSGILGAGKKIVCGFS | 21 | 2 | 80.0 | 1993.45 |
| SEQ ID | margaretae | GLC | | | | |
| NO: 71 | | | | | | |
| OdMa2 | Odorrana | GLLRGILGAGKKIVCGLS | 21 | 3 | 67.0 | 2028.54 |
| SEQ ID | margaretae | GLC | | | | |
| NO: 64 | | | | | | |
| OdMa3 | Odorrana | GLLSGLLGAGKKIVCGLS | 21 | 2 | 80.0 | 1977.47 |
| SEQ ID | margaretae | GMC | | | | |
| NO: 59 | | | | | | |
| OdMa4 | Odorrana | GILSGLLGAGKKIVC | 15 | 2 | 70.0 | 1428.79 |
| SEQ ID | margaretae | | | | | |
| NO: 62 | | | | | | |
| OdMa5 | Odorrana | GILSGLLGAGKKIVCGLS | 21 | 2 | 80.0 | 1959.43 |
| SEQ ID | margaretae | GLC | | | | |
| NO: 1091 | | | | | | |
| OdMa6 | Odorrana | GLLSGVLGVGKKIVCGLS | 21 | 2 | 80.0 | 1973.46 |
| SEQ ID | margaretae | GLC | | | | |
| NO: 1092 | | | | | | |
| OdMa7 | Odorrana | GLLSGVLGVGKKVLCGL | 21 | 2 | 80.0 | 1973.46 |
| SEQ ID | margaretae | SGLC | | | | |
| NO: 1093 | | | | | | |
| OdMa9 | Odorrana | GLISGILGAGKKVLC | 15 | 2 | 67.0 | 1428.79 |
| SEQ ID | margaretae | | | | | |
| NO: 68 | | | | | | |
| OdMa10 | Odorrana | GLISGILGAGKKVLCGLS | 21 | 2 | 70.0 | 1959.43 |
| SEQ ID | margaretae | GLC | | | | |
| NO: 1094 | | | | | | |
| OdMa12 | Odorrana | GFMDTAKNVAKNVAVTL | 29 | 4 | 70.0 | 3158.82 |
| SEQ ID | margaretae | LYNLKCKITKAC | | | | |
| NO: 69 | | | | | | |
| OdMa13 | Odorrana | GFMDTAKNVAKNVAVTL | 29 | 3 | 67.0 | 3110.73 |
| SEQ ID | margaretae | LDNLKCKITKAC | | | | |
| NO: 1095 | | | | | | |
| OdTo1 | Odorrana | GILSGLLGAGKKLACGLI | 21 | 2 | 80.0 | 1957.46 |
| SEQ ID | tormota | GLC | | | | |
| NO: 153 | | | | | | |
| OdTo2 | Odorrana | GIFGGHLKVGKKIACGLS | 21 | 3 | 67.0 | 2058.52 |
| SEQ ID | tormota | GLC | | | | |
| NO: 139 | | | | | | |
| OdTo3 | Odorrana | GIFGGLLKEGKKIACGLS | 21 | 2 | 48.5 | 2064.53 |
| SEQ ID | tormota | GLC | | | | |
| NO: 141 | | | | | | |
| OdTo4 | Odorrana | KLMIPRKKRGIFGGLLKV | 30 | 8 | 47.4 | 3186.06 |
| SEQ ID | tormota | GKKIACGLSGLC | | | | |
| NO: 190 | | | | | | |
| PaVa1 | Partula varia | RPRPQQVPPRPPHPRLR | 18 | 6 | 27.5 | 2240.63 |
| SEQ ID | | R | | | | |
| NO: 956 | | | | | | |
| PeNi1 | Pelophylax | GLLGKVLGVGKKVLCGV | 21 | 3 | 70.0 | 2014.55 |
| SEQ ID | nigromaculatus | TGLC | | | | |
| NO: 363 | | | | | | |
| PeNi2 | Pelophylax | GLLGKVLGVGKKVLCVV | 21 | 3 | 70.0 | 2042.61 |
| SEQ ID | nigromaculatus | SGLC | | | | |
| NO: 395 | | | | | | |
| PeNi3 | Pelophylax | GIFSLIKGAAKVVAKGLG | 18 | 3 | 65.2 | 1729.12 |
| SEQ ID | nigromaculatus | | | | | |
| NO: 1096 | | | | | | |
| PeNi4 | Pelophylax | GLLGKVLGVGKKVLC | 15 | 3 | 67.0 | 1483.91 |
| SEQ ID | nigromaculatus | | | | | |
| NO: 424 | | | | | | |
| PeNi5 | Pelophylax | GLLGKVLGVGKKVLCGV | 24 | 4 | 57.7 | 2471.01 |
| SEQ ID | nigromaculatus | TGRERCQ | | | | |
| NO: 359 | | | | | | |
| PeNi7 | Bufo | VIPFVASVAAEMMHHVY | 26 | 2 | 43.2 | 2863.42 |
| SEQ ID | gargarizans, | CAASKRCKN | | | | |
| NO: 1088 | Leptobrachium | | | | | |
| | boringii, | | | | | |
| | Megophrys | | | | | |
| | sangzhiensis, | | | | | |
| | Polypedates | | | | | |
| | megacephalus, | | | | | |
| | Pelophylax | | | | | |
| | nigromaculatus, | | | | | |
| | Rhacophorus | | | | | |
| | dennysi, | | | | | |
| | Rana | | | | | |
| | omeimontis | | | | | |
| PeNi8 | Bufo | GILLNTLKGAAKNVAGVL | 30 | 4 | 63.0 | 3012.69 |
| SEQ ID | gargarizans, | LDKLKCKITGGC | | | | |
| NO: 1097 | Megophrys | | | | | |
| | sangzhiensis, | | | | | |
| | Polypedates | | | | | |
| | megacephalus, | | | | | |
| | Pelophylax | | | | | |
| | nigromaculatus, | | | | | |
| | Rhacophorus | | | | | |
| | dennysi, | | | | | |
| | Rana | | | | | |
| | omeimontis | | | | | |
| PeNi9 | Leptobrachium | GLLGKILGVGKKVLCGVS | 21 | 3 | 62.2 | 2014.55 |
| SEQ ID | boringii, | GLC | | | | |
| NO: 32 | Megophrys | | | | | |
| | sangzhiensis, | | | | | |
| | Polypedates | | | | | |
| | megacephalus, | | | | | |
| | Pelophylax | | | | | |
| | nigromaculatus, | | | | | |
| | Rhacophorus | | | | | |
| | dennysi, | | | | | |
| | Rana | | | | | |
| | omeimontis | | | | | |
| PeNi10 | Leptobrachium | GLLLDTVKGAAKNVAGIL | 30 | 3 | 61.8 | 3056.70 |
| SEQ ID | boringii, | LNKLKCKVTGDC | | | | |
| NO: 1086 | Polypedates | | | | | |
| | megacephalus, | | | | | |
| | Pelophylax | | | | | |
| | nigromaculatus, | | | | | |
| | Rhacophorus | | | | | |
| | dennysi, | | | | | |
| | Rana | | | | | |
| | omeimontis | | | | | |
| PeNi11 | Leptobrachium | GILTDTLKGAAKNVAGVL | 30 | 3 | 61.8 | 3001.62 |
| SEQ ID | boringii, | LDKLKCKITGGC | | | | |
| NO: 1087 | Polypedates | | | | | |
| | megacephalus, | | | | | |
| | Pelophylax | | | | | |
| | nigromaculatus, | | | | | |
| | Rhacophorus | | | | | |
| | dennysi, | | | | | |
| | Rana | | | | | |
| | omeimontis | | | | | |
| PeNi14 | Bufo | GLWTTIKEGVKNFSVGVL | 29 | 3 | 67.00 | 3123.71 |
| SEQ ID | gargarizans, | DKIRCKITGGC | | | | |
| NO: 15 | Polypedates | | | | | |
| | megacephalus, | | | | | |
| | Pelophylax | | | | | |
| | nigromaculatus, | | | | | |
| | Rana | | | | | |
| | omeimontis | | | | | |
| PoSn1 | Polistes | ISIKEALEHSFFHTVPRK | 23 | 3 | 30.4 | 2822.31 |
| SEQ ID | snelleni | WCKKH | | | | |
| NO: 936 | | | | | | |
| PoSn2 | Polistes | TALKSLSILKKLAKLNM | 17 | 4 | 23.7 | 1872.37 |
| SEQ ID | snelleni | | | | | |
| NO: 942 | | | | | | |
| RaCa15 | Rana | FLPVVAGLAAKVLPSIICA | 24 | 3 | 67.0 | 2442.09 |
| SEQ ID | catesbeiana | VTKKC | | | | |
| NO: 1098 | | | | | | |
| RaOm2 | Rana | GILSGLLGAGKKIVCGLS | 21 | 2 | 80.0 | 1977.47 |
| SEQ ID | omeimontis | GMC | | | | |
| NO: 629 | | | | | | |
| RaOm3 | Rana | GIFSLIKGAAKVVAKGLG | 19 | 4 | 67.0 | 1857.30 |
| SEQ ID | omeimontis | K | | | | |
| NO: 1099 | | | | | | |
| RaOm4 | Rana | GLLGKVLGVGKKVLCGV | 21 | 4 | 67.0 | 2043.55 |
| SEQ ID | omeimontis | SGRC | | | | |
| NO: 628 | | | | | | |
| RaSi1 | Allobates | GLVGKLVKGGLKLIGHVA | 20 | 3 | 36.9 | 1930.35 |
| SEQ ID | femoralis, | NG | | | | |
| NO: 2 | Pristimantis | | | | | |
| | toftae, | | | | | |
| | Ranitomeya | | | | | |
| | sirensis | | | | | |
| RaSy2 | Rana sylvatica | EEQRFLPVVAGLAAKVLP | 28 | 2 | 21.9 | 2984.64 |
| SEQ ID | | SIICAVTKKC | | | | |
| NO: 673 | | | | | | |
| TeBi1 | Tetramorium | KIKIPWGKVKDFLVGGMK | 23 | 6 | 45.00 | 2528.17 |
| SEQ ID | bicarinatum | AVGKK | | | | |
| NO: 1090 | | | | | | |
| TeRu2 | Temnothorax | AFVRILCYCCPRRIKRR | 17 | 6 | 31.9 | 2153.70 |
| SEQ ID | rugatulus | | | | | |
| NO: 994 | | | | | | |
| TeRu4 | Temnothorax | SWLSKSVKKLVNKKNYT | 30 | 8 | 25.5 | 3622.33 |
| SEQ ID | rugatulus | RLEKLAKKKLFNE | | | | |
| NO: 996 | | | | | | |

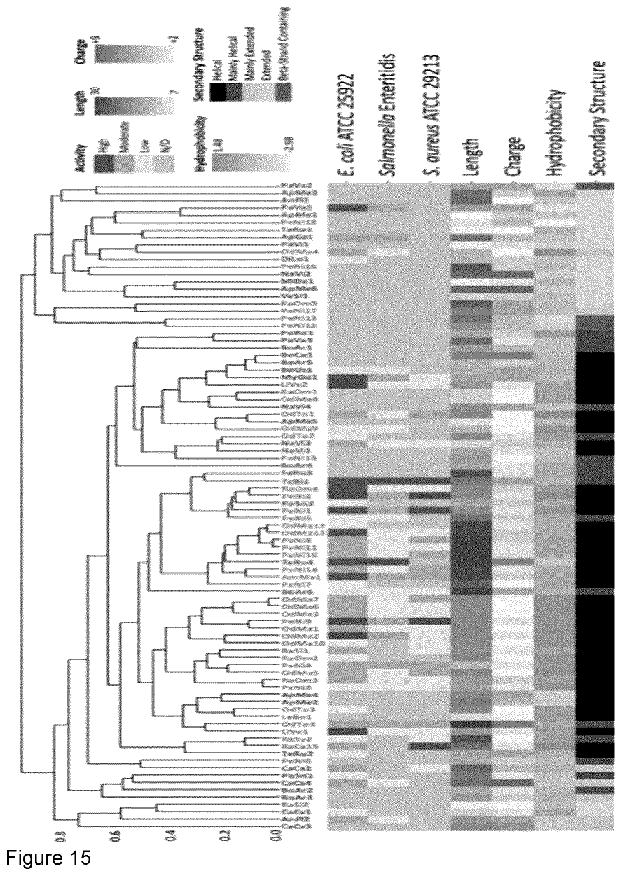

FIG. 15 shows agglomerative hierarchical clustering of peptides was conducted based on their predicted 3D structures. The bioactivity of each peptide against the three organisms tested (E. coli ATCC 25922, Salmonella Enteritidis, and S. aureus ATCC 29213). Bioactivity was assigned based on the median MIC and categorized as high for peptides with a MIC s 4 μg/mL, moderate for 4<MIC s 16 μg/mL and low for 16<MIC s 128 μg/mL. Peptides that did not inhibit bacterial growth at tested concentrations were classified as ‘no inhibition observed’ (N/O). Physiochemical attributes of the AMPs tested (length, charge, and hydrophobicity) are displayed in rows 4, 5 and 6 using purple, pink and green gradients, respectively. The colour-coded peptide secondary structures (helical, mainly helical, mainly extended, linear extended and p-strand containing) shown in row 7 were assigned based on 3D coordinates of predicted structures. AMP name labels below the dendogram are colour-coded to indicate their amphibian (green) or insect (brown) origin.

Sequences in FIG. 15 are listed in order of appearance in the figure, below:
PaVa2 (SEQ ID NO:961),
ApMe3 (SEQ ID NO:746),
AnFI1 (SEQ ID NO:718),
PaVa1 (SEQ ID NO:956),
ApMe1 (SEQ ID NO:729),
PeNi18 (SEQ ID NO:308),
TeRu1 (SEQ ID NO: 833)
ApCe1 (SEQ ID NO:695)
PaVi1 (SEQ ID NO: 968)
OdMa4 (SEQ ID NO:62)
DiLo (SEQ ID NO:864)
PeNi16 (SEQ ID NO:200)
NaVi2 (SEQ ID NO:889),
MiDe1 (SEQ ID NO:867),
Apme6 (SEQ ID NO:770),
VeSi1 (SEQ ID NO:1022),
RaOm5 (SEQ ID NO:1089)
PeNi17 (SEQ ID NO:1103)
PeNi13 (SEQ ID NO:16)
PeNi12 (SEQ ID NO:1101)
PoRo1 (SEQ ID NO:927)
PaVa3 (SEQ ID NO:955)
BoAr2 (SEQ ID NO:793),
BoCo1 (SEQ ID NO:800),
BoAr5 (SEQ ID NO:796),
BoUi1 (SEQ ID NO:815)
MyGu1 (SEQ ID NO:881)
LiVe2 (SEQ ID NO:40)
RaOm1 (SEQ ID NO:632),
OdMa3 (SEQ ID NO:59)
NaVi4 (SEQ ID NO:906),
OdTo1 (SEQ ID NO:153),
ApMe5 (SEQ ID NO:745),
OdMa9 (SEQ ID NO:68),
OdTe2 (SEQ ID NO:139),
NaVi3 (SEQ ID NO:895)
NaVi1 (SEQ ID NO:892),
PeNi15 (SEQ ID NO:1102),
BoAr4 (SEQ ID NO:789),
TeRu3 (SEQ ID NO:993)
TeRu1 (SEQ ID NO:833)
RaOm4 (SEQ ID NO:628)
PeNi2 (SEQ ID NO:395)
PoSn2 (SEQ ID NO:942)
PeNi5 (SEQ ID NO:359)
OdMa13 (SEQ ID NO:1095)
OdMa12 (SEQ ID NO:69)
PeNi2 (SEQ ID NO:395)
PeNi11 (SEQ ID NO:1087)
PeNi10 (SEQ ID NO:1086)
TeRu4 (SEQ ID NO:996)
PeNi14 (SEQ ID NO:15)
AmMa1 (SEQ ID NO:12)
PeNi7 (SEQ ID NO:1088)
BoAr6 (SEQ ID NO:790)
OdMa7 (SEQ ID NO:1093)
OdMa6 (SEQ ID NO:1092)
OdMa3 (SEQ ID NO:59)
PeNi9 ((SEQ ID NO:32)
OdMa1 (SEQ ID NO:71)
OdMa2 (SEQ ID NO:64)
OdMa10 (SEQ ID NO:1094)
RaSi1 (SEQ ID NO:2)
RaOm2 (SEQ ID NO:629)
PeNi4 (SEQ ID NO:424)
OdMa5 (SEQ ID NO:1091)
RaOm3 (SEQ ID NO:1099)
PeNi3 (SEQ ID NO:1096)
ApMe4 (SEQ ID NO:781),
ApMe2 (SEQ ID NO:782),
OdTo3 (SEQ ID NO:141),
LeBo1 (SEQ ID NO:1100),
OdTo4 (SEQ ID NO:141),
LiVe1 (SEQ ID NO:37)
RaSy2 (SEQ ID NO:673)
RaCa15 (SEQ ID NO:1098)
TeRu2 (SEQ ID NO:994)
PeNi6 (SEQ ID NO:1),
CaCa2 (SEQ ID NO:819)
PoSn1 (SEQ ID NO:936)
CaCa4 (SEQ ID NO:816)
BoAr2 (SEQ ID NO:793),
BoAr3 (SEQ ID NO:797),
RaSi2 (SEQ ID NO:4),
CaCa1 (SEQ ID NO:826)
AnFI2 (SEQ ID NO:721),
CaCa3 (SEQ ID NO:831).

Table 14 provides information on Minimum Inhibitory Concentration (MIC) of Peptides from three independent experiments, listing the peptides in alphabetical order according to the alphanumeric designation such as “AmMa1”, “AnFI1”, etc. The peptides are also presented in FIG. 15 and Table 13. Those peptides exhibiting inhibitory activity against at least one of E. coli ATCC 25922, Salmonella Enteritidis, or S. aureus ATCC 29213 at a concentration lower than 128 ug/mL in the particular tests conducted are indicated by an asterisk: SEQ ID NOS: 12, 721, 695, 709, 819, 816, 37, 40, 881, 895, 71, 1094, 69, 1095, 64, 59, 62, 1091, 1092, 1093, 68, 139, 141, 190, 956, 363, 1086, 1087, 15, 395, 1096, 424, 359, 1097, 32, 936, 942, 1098, 629, 1099, 628, 2, 673, 1090, 994, 996.

TABLE 14
Minimum Inhibitory Concentration (MIC) of Peptides

| | MIC (ug/mL) | |
|---|---|---|
| SEQ ID NO:/Sequence | E. coli/S. Enteritidis/S. aureus | |
| 12.* GILDTLKQLGKAAVQGLLSKAACKLAKTC | AmMa1 | 2-4/16-32/8 |
| 718. DNKWQNVHFHRSAVTGPTSFSFSHK | AnFI1 | >128/>128/>128 |
| 721.* GILRSLGWIQMPRSRRRHR | AnFI2 | 16-32/128/>128 |
| 695.* GIYTGRLLPVYIPQPRPPHPRLRR | ApCe1 | 4-8/16-32/>128 |
| 729. VKCRVRR | ApMe1 | >128/>128/>128 |
| 782. GAHKEVFKRDTALTKEAAKKAKK | ApMe2 | >128/>128/>128 |
| 746. GWGLINIKIPPVLHKVSVPLVSKR | ApMe3 | >128/>128/>128 |
| 781. KHHHIKLRHERHRRYILKSLI | ApMe4 | >128/>128/>128 |
| 745. SILSTLSHKR | ApMe5 | >128/>128/>128 |
| 770. RARKIRRRRGSLRHCVTIPSTPSGR | ApMe6 | >128/>128/>128 |
| 723. AAGAGKVTKSAQKAQKAK | BoAr1 | >128/>128/>128 |
| 793. ATAAECLKHPWLKIKK | BoAr2 | >128/>128/>128 |
| 797. IIRATAAECLKHPWLKIKK | BoAr3 | >128/>128/>128 |
| 789. SVASLAKNSAWPVSLKR | BoAr4 | >128/>128/>128 |
| 796. VTISIARRVSSHKRG | BoAr5 | >128/>128/>128 |
| 790.* GILRLVTRRFRFSPTNLNRYTVARLVSGVP | BoAr6 | 32/64/64-128 |
| 800. NKIKFINKYVKKVQLKKILVKS | BoCo1 | >128/>128/>128 |
| 815.* RKIIAVSVHKLCRVKR | BoUs1 | 64-128/64/>128 |
| 826.* FACPIGFFRLKR | CaCa1 | 128/>128/>128 |
| 819.* FIKTQVLKHLVAGVRVARGLDWKWR | CaCa2 | 8/>128/64-128 |
| 831. KHHHIKLRHGRHRRSVLRTLV | CaCa3 | >128/>128/>128 |
| 816.* RRFFFATAPCGYSRKFCKITRRKR | CaCa4 | 16-32/32-64/>128 |
| 864.* GAFVLWGPTPRPRRR | DiLo1 | 128/>128/>128 |
| 1100. GIFSLIKGAAK | LeBo1 | >128/>128/>128 |
| 37.* GWLDIAKKVASVVAGIVKR | LiVe1 | 4-8/32/16-32 |
| 40.* GWLDIAKKVASVVAGLGKR | LiVe2 | 4-8/64-128/64 |
| 867. VMLPKFKR | MiDe1 | >128/>128/>128 |
| 881.* RRAIFASIRGYLGLRKR | MyGu1 | 4/8-16/128 |
| 892. TPLSDIFRGQLRSRVSR | NaVi1 | >128/>128/>128 |
| 889. SSLSPLSSSSGLGKKKKRKSKRASR | NaVi2 | >128/>128/>128 |
| 895.* KLFLTLWKLKR | NaVi3 | 8-16/64/128 |
| 906. GSSSRSCRCIRLSRLSSKRT | NaVi4 | >128/>128/>128 |
| 71.* GLLSGILGAGKKIVCGFSGLC | OdMa1 | 8-16/128/64 |
| 64.* GLLRGILGAGKKIVCGLSGLC | OdMa2 | 4/8-16/4-8 |
| 59.* GLLSGLLGAGKKIVCGLSGMC | OdMa3 | 8-16/64/32 |
| 62.* GILSGLLGAGKKIVC | OdMa4 | 8-16/32/>128 |
| 1091.* GILSGLLGAGKKIVCGLSGLC | OdMa5 | 8-16/64-128/8-16 |
| 1092.* GLLSGVLGVGKKIVCGLSGLC | OdMa6 | 8-16/128/16-32 |
| 1093.* GLLSGVLGVGKKVLCGLSGLC | OdMa7 | 8-16/128/16-32 |
| 63. GLISGILGAGKK | OdMa8 | >128/>128/>128 |
| 68.* GLISGILGAGKKVLC | OdMa9 | 64-128/128/>128 |
| 1094.* GLISGILGAGKKVLCGLSGLC | OdMa10 | 16-32/>128/64-128 |
| 69.* GFMDTAKNVAKNVAVTLLYNLKCKITKAC | OdMa12 | 4/64/64-128 |
| 1095.* GFMDTAKNVAKNVAVTLLDNLKCKITKAC | OdMa13 | 64-128/>128/>128 |
| 153.* GILSGLLGAGKKLACGLIGLC | OdTo1 | 8/32-64/8-16 |
| 139.* GIFGGHLKVGKKIACGLSGLC | OdTo2 | 32/>128/>128 |
| 141.* GIFGGLLKEGKKIACGLSGLC | OdTo3 | 32/>128/>128 |
| 190.* KLMIPRKKRGIFGGLLKVGKKIACGLSGLC | OdTo4 | 8-16/16/16 |
| 956.* RPRPQQVPPRPPHPRLRR | PaVa1 | 2-4/4-8/>128 |
| 961. KYHHIKLRHGRHRRTIH | PaVa2 | >128/>128/>128 |
| 955. ITEPVGTKAPTFTSELRGGWLKKR | PaVa3 | >128/>128/>128 |
| 968. WALRWKTR | PaVi1 | >128/>128/>128 |
| 363.* GLLGKVLGVGKKVLCGVTGLC | PeNi1 | 4/8-16/4 |
| 395.* GLLGKVLGVGKKVLCVVSGLC | PeNi2 | 4/16-32/4 |
| 1096.* GIFSLIKGAAKVVAKGLG | PeNi3 | 16-32/64-128/64-128 |
| 424.* GLLGKVLGVGKKVLC | PeNi4 | 8-16/4-8/4-8 |
| 359.* GLLGKVLGVGKKVLCGVTGRERCQ | PeNi5 | 32/64/>128 |
| 1. AGLQFPVGRIHRHLKTR | PeNi6 | >128/>128/>128 |
| 1088.* VIPFVASVAAEMMHHVYCAASKRCKN | PeNi7 | 128/>128/>128 |
| 1097.* GILLNTLKGAAKNVAGVLLDKLKCKITGGC | PeNi8 | 8/64/4-8 |
| 32.* GLLGKILGVGKKVLCGVSGLC | PeNi9 | 2-4/8-16/4-8 |
| 1086.* GLLLDTVKGAAKNVAGILLNKLKCKVTGDC | PeNi10 | 8/64-128/16-32 |
| 1087.* GILTDTLKGAAKNVAGVLLDKLKCKITGGC | PeNi11 | 8-16/128/64 |
| 1101. GAPKGCWTKSYPPKPCSGKR | PeNi12 | >128/>128/>128 |
| 16. KEERGAPKGCWTKSYPPKPCSGKR | PeNi13 | >128/>128/>128 |
| 15.* GLWTTIKEGVKNFSVGVLDKIRCKITGGC | PeNi14 | 4-8/32/16-32 |
| 1102. FLPSSPWNEGTYVLKKLKS | PeNi15 | >128/>128/>128 |
| 200. ATAWKVPPGLQPIRPIRIRPLCGNDKS | PeNi16 | >128/>128/>128 |
| 1103. RMIRRPPGFSPFRVAPASSLKR | PeNi17 | >128/>128/>128 |
| 308. RPRWSHRSRR | PeNi18 | >128/>128/>128 |
| 927. VAAFAIIGCLCCRRPRR | PoRo1 | >128/>128/>128 |
| 936.* ISIKEALEHSFFHTVPRKWCKKH | PoSn1 | 64/>128/>128 |
| 942.* TALKSLSILKKLAKLNM | PoSn2 | 64-128/>128/>128 |
| 1098.* FLPVVAGLAAKVLPSIICAVTKKC | RaCa15 | 8-16/>128/4-8 |
| 632. GLLSGILGAGKK | RaOm1 | >128/>128/>128 |
| 629.* GILSGLLGAGKKIVCGLSGMC | RaOm2 | 64-128/>128/>128 |
| 1099.* GIFSLIKGAAKVVAKGLGK | RaOm3 | 8-16/32-64/64 |
| 628.* GLLGKVLGVGKKVLCGVSGRC | RaOm4 | 4-8/32/64-128 |
| 1089. AGYSRMIRRPPGFSPFRVAPASSLKR | RaOm5 | >128/>128/>128 |
| 2.* GLVGKLVKGGLKLIGHVANG | RaSi1 | 8-16/>128/64-128 |
| 4. FPFPFGRR | RaSi2 | >128/>128/>128 |
| 673.* EEQRFLPVVAGLAAKVLPSIICAVTKKC | RaSy2 | 64/>128/64-128 |
| 1090.* KIKIPWGKVKDFLVGGMKAVGKK | TeBi1 | 1-2/4-8/4-8 |
| 833. VPFGLKPR | TeRu1 | >128/>128/>128 |
| 994.* AFVRILCYCCPRRIKRR | TeRu2 | 64-128/>128/128 |
| 993. AVLSFVHKLFLNFLHVDTSKGKCRATLQ | TeRu3 | >128/>128/>128 |
| 996.* SWLSKSVKKLVNKKNYTRLEKLAKKKLFNE | TeRu4 | 1/2-4/>128 |
| 1022. FILHAKKTRSAK | VeSi1 | >128/>128/>128 |

*Indicates inhibitory activity

### 2.2.2. Antimicrobial Susceptibility Testing and Cytotoxicity

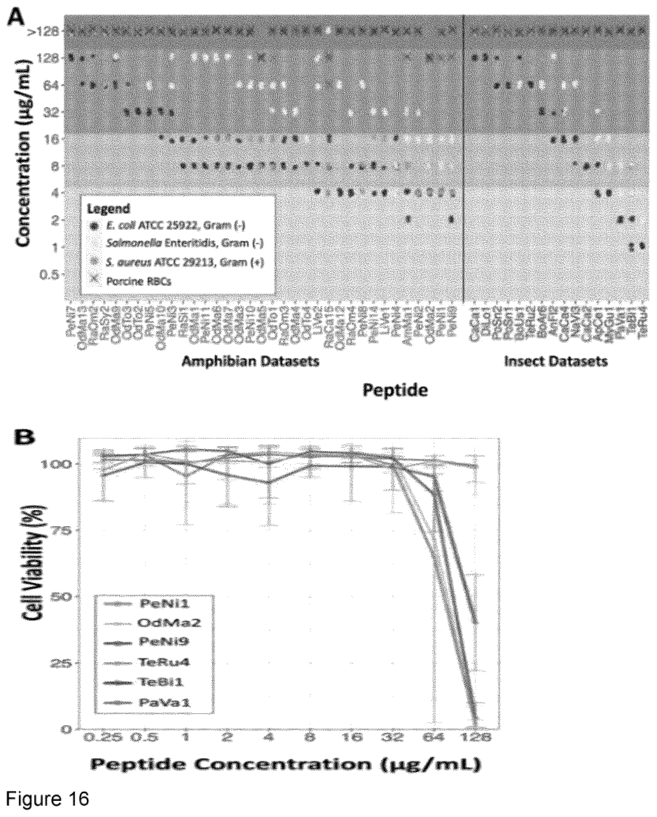

The 88 putative AMPs were tested for their antimicrobial activity using a broth microdilution assay [137]. The panel of bacteria tested included two Gram-negative strains: E. coli ATCC 25922 and Salmonella Enteritidis. Additionally, one Gram-positive strain was tested: S. aureus ATCC 29213. Of the 88 peptides tested, 51 displayed antimicrobial activity (MIC s 128 μg/mL) against at least one of the pathogens, including 35 from amphibian and 16 from insect sources (FIG. 16, part A). All AMPs that only inhibited growth of E. coli (10 peptides) had low activity. Eleven peptides were selective against the Gram-negative bacteria, with no inhibitory activity against S. aureus at the tested concentrations. Further, 30 peptides were active against both Gram-negative and Gram-positive species, with 5 having no inhibitory effect on Salmonella Enteritidis and 25 being active against all three species. Bioactivity quantitative data is displayed in Table 14. Additionally, the hemolytic 50 concentration (HC50, see Methods for definition) of the peptides was determined with porcine red blood cells as a cost-effective initial toxicity assessment of the AMPs. Six of the active peptides displayed low hemolytic activity, with the HC50s ranging from 64-128 μg/mL.

FIG. 16 shows antimicrobial activity and mammalian cell toxicity of peptides discovered. (part A) Minimum inhibitory and hemolytic 50 concentration (MIC and HC50, respectively) of peptides with antimicrobial activity against at least one bacterial strain within a panel composed of E. coli ATCC 25922, Salmonella Enteritidis and S. aureus ATCC 29213. HC50 was determined using porcine red blood cells (RBCs), as described in the Methods. Bioactive peptides identified from amphibian (green text) and insect (brown text) datasets are shown. Peptide bioactivity levels are separated by grey shaded blocks. From top to bottom, the dark grey block indicates no observable activity, the medium-dark grey block includes low activity, the medium grey block designates moderate activity, and the light grey block corresponds to high antimicrobial activity. (part B) Mean relative cellular viability of HEK293 (cultured human kidney cells) after 24 h incubation with the six most active insect and amphibian peptides at concentrations up to 128 μg/mL from three independent experiments. Error bars indicate range of data.

A total of 14 peptides displayed high antimicrobial activity against at least one of the pathogens tested. The three most active amphibian peptides, OdMa2, PeNi1 and PeNi9, were broadly active. OdMa2 and PeNi1 had MICs of 4 μg/mL against E. coli and 8-16 μg/mL against Salmonella Enteritidis. OdMa2 had an MIC of 4-8 μg/mL and PeNi1 had an MIC of 4 μg/mL against S. aureus. PeNi9 had MICs of 2-4 μg/mL against E. coli, 8-16 μg/mL against Salmonella Enteritidis, and 4-8 μg/mL against S. aureus. OdMa2 was slightly hemolytic, having an HC50=128 μg/mL, while PeNi1 and PeNi9 did not show hemolytic activity at the highest concentration tested. The three most active insect peptides were PaVa1, TeBi1 and TeRu4. PaVa1 and TeRu4 were selectively active against Gram-negatives, with MICs of 2-4 μg/mL and 1 μg/mL against E. coli and 4-8 μg/mL and 2-4 μg/mL against Salmonella Enteritidis, respectively. These two peptides did not inhibit growth of S. aureus at the concentrations tested. TeBi1 was broadly active, with an MIC of 1-2 μg/mL against E. coli, 4-8 μg/mL against Salmonella Enteritidis and S. aureus. All top three insect peptides were not hemolytic at the concentrations tested.

The six most active peptides were tested for cytotoxicity against the human embryonic kidney cell line HEK293 using the alamarBlue cell viability assay. PeNi1 and OdMa2 were toxic to less than 10% of cells up until 32 μg/mL, where cell viability decreased to <75% at 64 μg/mL and to approximately 0% at 128 μg/mL (FIG. 16, part B). More variation in cell viability was observed at 64 μg/mL for these peptides compared to other concentrations. Cell viability when exposed to PeNi9 and TeBi1 was, on average, 0% and 40% at 128 μg/mL, respectively. These peptides had low or no cytotoxicity at lower concentrations. TeRu4 and PaVa1 were observed, and found not to be toxic to HEK293 cells at any of the concentrations tested.

### 2.2.3. Sequence and Structural Characterization of AMPs Discovered Using rAMPage

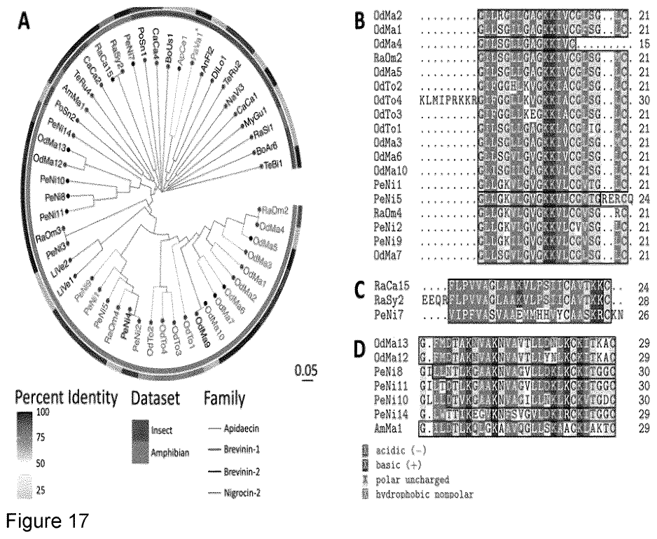

To investigate the sequence diversity of the 51 peptides displaying antimicrobial activity, a multiple sequence alignment of the mature amino acid sequences was performed and a phylogenetic tree was generated. Peptides derived from amphibian/insect datasets mostly clustered with other peptides from the same datasets (FIG. 17, part A). To investigate whether the mature sequences were similar to any known proteins, a local BLASTp [29] search of the validated AMPs was performed. Approximately two thirds of the peptides were novel, with less than 100% sequence identity to their top BLAST hits (FIG. 17, part A). Of these peptides, six amphibian-derived and seven insect-derived AMPs had no significant hits in the non-redundant protein database. CaCa1 and CaCa2 had 100% sequence identity with their top BLAST hits but no antimicrobial activity was indicated in the NCBI entries. OdMa13, PeNi11, OdMa5, OdMa6, OdMa7 and RaCa15 were identical to AMPs seen in other organisms of the same genera. OdMa10, an AMP discovered in the frog Odorrana margaretae, was identical to the AMP nigrosin-MG1 from this species. PeNi8, PeNi10, and PeNi7, derived from the black-spotted frog Pelophylax nigromaculatus, aligned with 100% sequence identity to the mature region of the AMPs pelophylaxin-2, ranatuerin-2N, and nigrocin-6N, respectively, from this species. Of note, it was also discovered the mature sequence of PeNi8 in five other amphibian species, PeNi10 in four other amphibian species, and PeNi7 in six other amphibian species. TeBi1, PeNi3 and RaOm3 also had BLAST hits with 100% sequence identity to precursors of known antimicrobial peptides, however they were not identical to the mature bioactive peptide. TeBi1 included the sequence of the mature AMP bicarinalin from the same species, but at its C-terminus it also contained three amino acids present in the peptide precursor but not in the mature sequence. Of their top BLAST hit esculentin-2N, a 36-residue AMP, PeNi3 and RaOm3 only spanned 18 and 19 residues, respectively. Additionally, PaVa1 had 100% sequence identity to a region of an apidaecin type 14-like isoform; however no mature region was annotated in this result and no in vitro validation of antimicrobial activity was available from public repositories.

Further, the peptide sequences were analyzed with InterProScan to identify protein family domains and classifications. 18 active amphibian peptides were discovered containing the signature for the Ranidae AMP Nigrocin-2 family, three from the Brevinin-1 family, and seven with the Brevinin-2 signature. Additionally, two insect peptides were classified as being a part the Apidaecin family. While PaVa1 was not identified as containing any family signatures by InterProScan, it was classified as an Apidaecin as it possessed the RP . . . PRPPHPRL motif that has been identified as being conserved in Apidaecins [139].

FIG. 17 shows phylogenetic analysis of certain validated AMPs. (part A) Circular phylogenetic tree based on a multiple sequence alignment of active rAMPage peptides with family classifications highlighted (colour-coded AMP names). The origin of AMP (insects in brown and amphibian in green) and BLASTp percent identity to entries in the NCBI non-redundant protein database (on a scale of 0-100%) are indicated by the inner and outer rings, respectively. Tip points of peptides that do not have 100% sequence identity to the bioactive region of known AMPs are highlighted in red. Multiple sequence alignments of the Nigrocin-2 (part B), Brevinin-1 (part C) and Brevinin-2 families (part D), with the outlines indicating the identified family signature. (B) Sequences are: OdMa2 (SEQ ID NO:64), OdMa1 SEQ ID NO:71), OdMa4 (SEQ ID NO:62), RaOm2 (SEQ ID NO:629), OdMa5 (SEQ ID NO: 1091), OdTo2 (SEQ ID NO:139), OdTo4 (SEQ ID NO:190), OdTo3 (SEQ ID NO:141), OdTo1 (SEQ ID NO:153), OdMa3 (SEQ ID NO:59), OdMa6 (SEQ ID NO:1092), OdMa10 (SEQ ID NO:1094), PeNi1 (SEQ ID NO:363), PeNi5 (SEQ ID NO:359), RaOm4 (SEQ ID NO:628), PeNi2 (SEQ ID NO:395), PeNi9 (SEQ ID NO:32), and OdMa7(SEQ ID NO:1093). (C) Sequences are: RaCa15 (SEQ ID NO:1098), RaSy2(SEQ ID NO:673), and PeNi7 (SEQ ID NO:1088). (D) Sequences are: OdMa13 (SEQ ID NO:1095), OdMa12 (SEQ ID NO:69), PeNi8 (SEQ ID NO:1097), PeNi11 (SEQ ID NO:1087), PeNi10 (SEQ ID NO:1086), PeNi14 (SEQ ID NO:15), and AmMa1 (SEQ ID NO:12).

Most Nigrocin-2-like peptides (15/18) were 21 amino acids long (FIG. 17, part B) with all 21 residues identified as part of the family signature. OdMa4, which is 14 amino acids long, aligns with the N-terminal of the other 15 peptides and 100% of its length is identified as containing the signature. In contrast, the two peptides longer than 21 amino acids, OdTo4 and PeNi5, only have part of their sequences containing the signature. The peptides characterized as belonging to the Brevinin-1 family were longer and contained more hydrophobic residues than most of the Nigrocin-2 peptides within the set (FIG. 17 Part C). All peptides classified as being part of the Brevinin-2 family (FIG. 17 Part D) were longer than those in the Nigrocin-2 and Brevinin-1 families, containing 29-30 amino acids and 1-2 negatively charged residues.

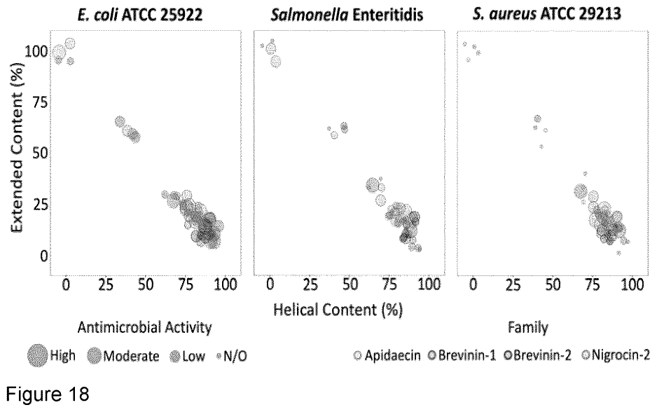

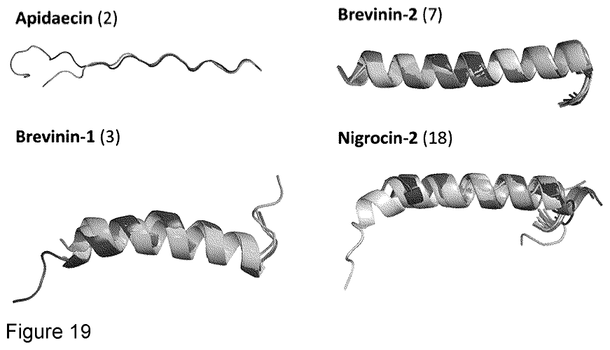

To explore the relationship between predicted helical or extended structure and activity, the distribution of bioactive peptides was investigated for each bacterial species tested in relation to the percentage of residues contained in helical or extended structures. Peptides with higher extended content were active against E. coli and Salmonella Enteritidis, but not S. aureus (FIG. 18). All peptides with activity against S. aureus were predicted to have some helical secondary structure, with the majority having >70% of the peptide assigned as having helical structure. Peptides identified as containing the signatures for the Apidaecin, Nigrocin-2, Brevinin-1 and Brevinin-2 families appeared to possess similar bioactivity profiles and predicted secondary structure content to other members within their families. The two Apidaecin peptides were linear extended and selective to the Gram-negative bacteria tested, with moderate to high activity against both E. coli and Salmonella Enteritidis. The Brevinin-2 and Nigrocin-2 peptides were mostly helical and broadly active against all three bacteria with the exception of OdMa4, which is mainly extended and displayed selective antimicrobial activity against the Gram-negative bacteria tested. All of the Brevinin-1 peptides were mostly helical and active against E. coli, non-inhibitory against Salmonella Enteritidis, and two out of three were active against S. aureus.

FIG. 18 shows predicted structural content and antimicrobial activity of 51 bioactive peptides against E. coli, Salmonella Enteritidis, and S. aureus. Secondary structure content indicated by the percentage of residues assigned to participate in helical vs. linear extended regions. Distribution of AMP family members and level of antimicrobial activity are distinguished by colour and size of circles, respectively.

TABLE 15
Range of Selectivity Indices of Select Bioactive Antimicrobial Peptides

| | Selectivity Index | |
|---|---|---|
| SEQ ID NO:/Sequence | E. coli/S. Enteritidis/S. aureus | |
| 12. GILDTLKQLGKAAVQGLLSKAACKLAKTC | AmMa1 | 32- >64/≥16/4- >8 |
| 721. GILRSLGWIQMPRSRRRHR | AnFI2 | 16-32/128/>128 |
| 695. GIYTGRLLPVYIPQPRPPHPRLRR | ApCe1 | >16- >32/N/O/>4- >8 |
| 790. GILRLVTRRFRFSPTNLNRYTVARLVSGVP | BoAr6 | >4/>1- >2/>2 |
| 815. RKIIAVSVHKLCRVKR | BoUs1 | >1- >2/N/O/>2 |
| 826. FACPIGFFRLKR | CaCa1 | >1/N/O/N/O |
| 819. FIKTQVLKHLVAGVRVARGLDWKWR | CaCa2 | >16/>1- >2/N/O |
| 816. RRFFFATAPCGYSRKFCKITRRKR | CaCa4 | >4- >8/N/O/>2- >4 |
| 864. GAFVLWGPTPRPRRR | DiLo1 | >1/N/O/N/O |
| 37. GWLDIAKKVASVVAGIVKR | LiVe1 | >16- >32/>4- >8/>4 |
| 40. GWLDIAKKVASVVAGLGKR | LiVe2 | >16- >32/>2/>1- >2 |
| 881. RRAIFASIRGYLGLRKR | MyGu1 | >32/>1/>8- >16 |
| 895. KLFLTLWKLKR | NaVi3 | >8- >16/>1/>2 |
| 71. GLLSGILGAGKKIVCGFSGLC | OdMa1 | >8- >16/>2- >4/>1 |
| 64. GLLRGILGAGKKIVCGLSGLC | OdMa2 | 32/16- 32/8- 16 |
| 59. GLLSGLLGAGKKIVCGLSGMC | OdMa3 | >8- >16/>4/>2 |
| 62. GILSGLLGAGKKIVC | OdMa4 | >8- >16/N/O/>4 |
| 1091. GILSGLLGAGKKIVCGLSGLC | OdMa5 | 8- 16/8- 16/1- 2 |
| 1092. GLLSGVLGVGKKIVCGLSGLC | OdMa6 | >8- >16/>4- >8/>1 |
| 1093. GLLSGVLGVGKKVLCGLSGLC | OdMa7 | >8- >16/>4- >8/>1 |
| 68. GLISGILGAGKKVLC | OdMa9 | >1- >2/N/O/>1 |
| 1094. GLISGILGAGKKVLCGLSGLC | OdMa10 | >4/>1- >2/N/O |
| 69. GFMDTAKNVAKNVAVTLLYNLKCKITKAC | OdMa12 | 32/>1- >2/>2 |
| 1095. GFMDTAKNVAKNVAVTLLDNLKCKITKAC | OdMa13 | >1- >2/N/O/> N/O |
| 153. GILSGLLGAGKKLACGLIGLC | OdTo1 | ≥16/8- >16/2- >4 |
| 139. GIFGGHLKVGKKIACGLSGLC | OdTo2 | >4/N/O/N/O |
| 141. GIFGGLLKEGKKIACGLSGLC | OdTo3 | >2- >4/N/O/N/O |
| 190. KLMIPRKKRGIFGGLLKVGKKIACGLSGLC | OdTo4 | >8- >16/>8/>8 |
| 956. RPRPQQVPPRPPHPRLRR | PaVa1 | >32- >64/N/O/>16- >32 |
| 363. GLLGKVLGVGKKVLCGVTGLC | PeNi1 | ≥32/≥32/8- >16 |
| 395. GLLGKVLGVGKKVLCVVSGLC | PeNi2 | >32/>32/>4- >8 |
| 1096. GIFSLIKGAAKVVAKGLG | PeNi3 | >4- >8/>1- >2/>1- >2 |
| 424. GLLGKVLGVGKKVLC | PeNi4 | >8- >16/>16- >32/>16- >32 |
| 359. GLLGKVLGVGKKVLCGVTGRERCQ | PeNi5 | >4/N/O/>2 |
| 1088. VIPFVASVAAEMMHHVYCAASKRCKN | PeNi7 | >1/N/O/>N/O |
| 1097. GILLNTLKGAAKNVAGVLLDKLKCKITGGC | PeNi8 | >16/>16- >32/>2 |
| 32. GLLGKILGVGKKVLCGVSGLC | PeNi9 | 32- >64/16- >32/8- >16 |
| 1086.GLLLDTVKGAAKNVAGILLNKLKCKVTGDC | PeNi10 | >16/>4- >8/>1- >2 |
| 1087. GILTDTLKGAAKNVAGVLLDKLKCKITGGC | PeNi11 | >8- >16/>2- >4/>1 |
| 15. GLWTTIKEGVKNFSVGVLDKIRCKITGGC | PeNi14 | >16- >32/>4- >8/>4 |
| 936. ISIKEALEHSFFHTVPRKWCKKH | PoSn1 | >2/N/O/N/O |
| 942. TALKSLSILKKLAKLNM | PoSn2 | >1- >2/N/O/N/O |
| 1098. FLPVVAGLAAKVLPSIICAVTKKC | RaCa15 | 4- 16/8- 32/N/O |
| 629. GILSGLLGAGKKIVCGLSGMC | RaOm2 | >1- >2/N/O/N/O |
| 1099. GIFSLIKGAAKVVAKGLGK | RaOm3 | >8- >16/>2/>2- >4 |
| 628. GLLGKVLGVGKKVLCGVSGRC | RaOm4 | >16- >32/>1- >2/>4 |
| 2. GLVGKLVKGGLKLIGHVANG | RaSi1 | >8- >16/>1- >2/N/O |
| 673. EEQRFLPVVAGLAAKVLPSIICAVTKKC | RaSy2 | >2/>1- >2/N/O |
| 1090. KIKIPWGKVKDFLVGGMKAVGKK | TeBi1 | >64- >128/>16- >32/>16- >32 |
| 994. AFVRILCYCCPRRIKRR | TeRu2 | >1- >2/N/O >32- >64 |
| 996. SWLSKSVKKLVNKKNYTRLEKLAKKKLENE | TeRu4 | 1/2- 4/>128 |

*N/O indicates no observed antimicrobial activity against the given bacterial species

### 2.3. Discussion

In the present Example, 51 AMP candidates originally derived from a bioinformatics scan of RNA-seq data from amphibians and insects were characterized using rAMPage [119]. Fourteen of these had high antimicrobial activity to at least one of the bacterial species tested. The most bioactive amphibian AMPs, PeNi1 (SEQ ID NO:363), OdMa2 (SEQ ID NO:64) and PeNi9 (SEQ ID NO:32), and the insect peptide TeBi1 (SEQ ID NO: 1090) were active against all three bacteria. TeRu4 (SEQ ID NO:996) and PaVa1 (SEQ ID NO:956) both demonstrated selectivity for the Gram-negative species tested, with TeRu4 (SEQ ID NO:996) having higher antimicrobial activity against both strains.

The AMPs reported herein had low to no toxicity, with only six peptides having hemolytic activity at the highest concentrations tested. This observation is consistent with previous research that suggests that AMPs tend to have selectivity for microbial cells over eukaryotic cells due to differences in membrane composition [140]. Table 15 shows range of selectivity indices. Despite this selectivity, some AMPs may disrupt mammalian membranes and cell processes [141]. In the reported list of AMPs, PeNi1 (SEQ ID NO:363), OdMa2 (SEQ ID NO:64), PeNi9 (SEQ ID NO:32) and TeBi1 (SEQ ID NO: 1090) displayed high levels of cytotoxicity at concentrations higher than their MICs. The ratio of the toxicity to the MIC of the peptides is described using the selectivity index, with a larger selectivity index indicating preference for bacterial cells [142, 143]. TeBi1 (SEQ ID NO: 1090) displayed the most selectivity of these, as it was not toxic to over 50% of cells until 16 to 128-fold its MIC. TeRu4 (SEQ ID NO:996) and PaVa1 (SEQ ID NO:956) were not toxic against either porcine erythrocytes or HEK293 cells. The selectivity indices of TeBi1 (SEQ ID NO: 1090), TeRu4 (SEQ ID NO:996) and PaVa1 (SEQ ID NO:956) make them excellent candidates for therapeutic development.

The AMPs characterized here vary in their amino acid sequences, with 30 active peptides belonging to four AMP families. AMPs from the Ranidae family can be classified into 14 peptide families based on their amino acid sequence, with AMPs from the same peptide family thought to share a common evolutionary origin [144]. AMPs were identified from three of these families: Brevinin-1, Brevinin-2 and Nigrocin-2 using the families' signatures. Brevinin-2 peptides are the longest of the three, with a mature peptide length of 33-34 residues. Brevinin-1 and Nigrocin-2 peptides are shorter, with an approximate length of 24 and 21 amino acids, respectively. However, there is considerable variation among individual family members. AMPs from these three families share some common characteristics: presence of a C-terminal disulfide-bridged cyclic heptapeptide, otherwise known as the ‘Rana box’, and antimicrobial activity against both Gram-negative and Gram-positive bacteria [145]. Additionally, Brevinin-1, Brevinin-2 and Nigrocin-2 peptides have been found to adopt an amphipathic α-helical conformation in membrane-like environments [145,146]. All peptides that were identified as members of these families included the Rana box and were predicted to have a helical or mainly helical structure except for the Nigrocin-2 peptide OdMa4. OdMa4 was the shortest of the Nigrocin-2 peptides and terminated after the first cysteine of the Rana box. Additionally, two peptides were identified as belonging to the Apidaecin AMP family. Apidaecins are short, proline-rich AMPs produced by insects [139]. These peptides do not typically form α-helices or β-strands [139], as was seen with ApCe1 and PaVa1, which were predicted to have linear extended structures. Similar to other Apidaecins, both ApCe1 and PaVa1 showed antimicrobial activity against Gram-negative bacterial species but not Gram-positives [139].

Most of the validated AMPs were predicted to adopt a helical structure by ColabFold, despite helical structures only making up half of the peptide set. In contrast, predicted linear extended peptides made up a smaller proportion of the antimicrobially active peptides compared to their abundance in the overall set. Note that, while there was a tendency of peptides with a predicted helical structure to be bioactive, not all of the helical peptides were active, and vice versa. In addition, it was observed that while there were peptides from all structural categories that were active against Gram-negative species, only peptides that had some predicted helical content were active against both Gram-positive and Gram-negative bacteria. AMPs that form α-helices make up the majority of AMPs with known structure, and it is thought to be the most effective structure rendering antibacterial activity against the bacterial membrane [132]. However, this also biases machine learning algorithms such as the one used to discover the tested peptides in favour of peptides with α-helix structures.

The majority of known AMPs do not have an experimentally validated structure [133]. Determination of peptide structures is often accomplished with NMR, X-ray crystallography or cryo-EM; however, this is time consuming and costly in comparison to in silico techniques. In contrast, one can predict the secondary and tertiary structures in a high-throughput manner based on the amino acid sequences of peptides of interest using a tool like ColabFold [135] prior to synthesis and testing. The association between ColabFold predicted structures and antimicrobial activity identified here can be informative in selecting peptides identified in silico as putative AMPs for in vitro validation. Further, AMPs are often chemically modified to improve their stability and bioavailability. The insights into the predicted structure-function relationships identified here could also be used to investigate how chemical modifications may influence antimicrobial activity.

One of the limitations of using rAMPage for peptide discovery is that the pipeline uses RNA-seq reads, and thus does not detect post-translational modifications such as amidation. Amidation of the C-terminus is a common post-translational modification of AMPs [147], and can impact the antimicrobial activity and toxicity of peptides [148]. PeNi12 for example, has 100% sequence identity to the mature region of ranacyclin-N from P. nigromaculatus, however PeNi12 was non-inhibitory against the panel of bacteria at the concentrations tested. Ranacyclins have amidated C-termini [149], so the non-amidated carboxyl end may have contributed to the lack of antimicrobial activity of PeNi12. TeBi1 has 100% sequence identity to the C-terminus region of the propeptide of bicarinalin and includes the mature sequence of bicarinalin, however the last three amino acids at the C-terminus of bicarinalin precursor are cleaved off and the peptide is amidated [34]. Additionally, as the peptides were tested in vitro, the peptides that did not exhibit particularly acute antimicrobial activity in the test conducted may nevertheless have immunomodulatory properties or act on other bacterial strains not tested in the present study. The AMPs identified here can undergo further testing to determine the extent of their impact on the immune system to combat infections within the host.

While ColabFold determines protein structures in silico with high accuracy, peptides in their biological environments are flexible and may adopt different conformations [131]. AMPs that adopt a helical structure at the membrane are often disordered in aqueous environments [131]. Thus, the predicted structure may not be representative of the 3D structure of the peptide when interacting with specific targets. Additionally, AMPs have diverse sequences and targets. Peptides that act on the bacterial membrane may have different structural characteristics compared to those that mainly interact with intracellular components.

AMPs are a promising alternative to conventional small molecule antibiotics as they represent a diverse group of molecules with a wide variety of physiochemical properties. Herein is presented the discovery and characterization of structurally and functionally diverse AMPs with potent antimicrobial activity and low toxicity. The utility of predicting the structure of AMPs is demonstrated and a significant association between peptides predicted to adopt a helical conformation displayed antimicrobial activity (p=6.6×10⁻⁵). The potential of structural prediction to prioritize putative AMPs is an exciting avenue for discovery of new therapeutics in the fight against bacterial infections.

### 2.4. Materials and Methods

### 2.4.1. Peptide Discovery Using rAMPage

Peptide sequences were discovered using the Rapid Antimicrobial Peptide Annotation and Gene Estimation (rAMPage) pipeline v1.0 [119], which is publicly available on Github (https://github.com/bcgsc/rAMPage). rAMPage is a homology-based pipeline that uses RNA-seq reads as input to generate putative AMPs for downstream in vitro testing. Briefly, rAMPage generates putative AMPs by first processing the input RNA-seq reads using fastp v0.20.0 [60] and assembles the reads into transcripts with RNA-bloom v1.3.1 [61]. Transcripts are subsequently translated by Transdecoder v5.5.0 [63] and a homology search is conducted with HMMER v3.3.1 [64]. Precursor sequences are cleaved with ProP v1.0c [66] and putative mature AMP sequences are prioritized by AMPlify v1.0.3, a deep-learning classifier [27].

The putative AMPs were filtered to retain peptides with a minimum charge of +2 and maximum length of 30 amino acids. Peptides were further characterized with ENTAP v0.10.7 [70], Exonerate v2.4.0 [73] and SABLE v4.0 [74] before being clustered with CD-HIT v4.8.1 [75]. Ninety sequences were prioritized for synthesis from this list using three selection criteria: “Species Count”—peptides identified in two or more species; “Insect Peptide,”—insect-derived peptides chosen using a reduced AMPlify prediction score threshold; and “AMPlify Score”—the top-scoring peptides with the highest net positive charge.

Peptides were purchased from GenScript and provided in lyophilized 0.08 milligram aliquots. Two peptides were unable to be synthesized, resulting in a final experimental set of 88 putative AMPs.

### 2.4.2. Bacterial Isolates

Bacteria were grown overnight at 37° C. in Mueller-Hinton Broth (MHB; Sigma-Aldrich, St. Louis. MO, USA) in a shaking incubator and aliquoted into cryovials with a 50% glycerol solution in a 1:1 ratio. No additives were added to the MHB during the growth of bacterial isolates. Glycerol stocks were stored at −80° C.

### 2.4.3. Antimicrobial Susceptibility Testing (AST)

The antimicrobial activity of the putative AMPs was determined by a broth microdilution assay as described by the Clinical and Laboratory Standards Institute, with the published adaptations for testing of cationic peptides [137]. Escherichia coli 25922 and Staphylococcus aureus 29213 isolates were purchased from the American Type Culture Collection (ATCC; Manassas, VA, USA). A human clinical isolate of Salmonella enterica serovar Enteritidis was provided by the BC Centre for Disease Control. Bacteria from stocks stored at −80° C. were streaked onto nonselective Columbia blood agar with 5% sheep blood (Oxoid) and incubated overnight at 37° C. Next, 2-4 colonies were streaked onto a new agar plate to ensure uniform health of colonies used in the broth microdilution assay. Isolated colonies were suspended in MHB and the optical density was measured with a spectrophotometer to create an initial inoculum concentration of approximately 1×10⁸ cfu/mL. The inoculum was diluted 1:250 to a final concentration of 2-8×10⁵ cfu/mL, which was confirmed by performing a Total Viability Count (TVC). The TVC consisted of plating a 1:1000 dilution of the final inoculum on nonselective media and was also used to confirm the health and purity of the inoculum in each trial.

Lyophilized 80 μg peptide aliquots synthesized by GenScript (Piscataway, NJ, USA) were resuspended to 1.28 mg/mL with UltraPure water (Thermo Fisher Scientific, Waltham, MA, USA) and serially diluted in polypropylene 96-well microtiter plates (Greiner Bio-One #650261, Kremsmunster, Austria) from 128 down to 0.5 μg/mL. Two columns per plate were reserved for growth and sterility controls. Inoculum was added to the wells containing the peptide and the growth control. Plates were incubated for 20-24 h at 37° C. The minimum inhibitory concentration (MIC) was determined as the lowest peptide concentration where there was no visible bacterial growth.

### 2.4.4. Structure Prediction and Clustering

The 3D structures of the mature peptides was predicted using a local installation of ColabFold [135]. Using ColabFold's server, five structures were generated for each peptide with sub-models of AlphaFold. Structural templates were used as input along with the peptide sequences for better accuracy. The five estimated structures were relaxed using the Amber force field, which helps remove stereochemical violations in predictions [134]. The five structures for each sequence were ranked by the per-residue estimate of AlphaFold's confidence in its prediction (pLDDT score). The PDB file of the Amber relaxed rank 1 model for each peptide was used for further analysis.

Similarity of the predicted structures of peptides was determined using mTM-align (version 20180725) [150], which was used to prepare a distance matrix calculated from the pairwise TM-scores of the PDB files produced for each peptide. This distance matrix was clustered using scipy.cluster.hierarchy [151] in python with complete linkage to produce a dendrogram. PDB files were fed into STRIDE [136] and the residue assignments were used to categorize peptides. Peptides were classified into linear extended peptides (only possessing turn secondary structure and/or coils), mainly extended (>50% of the residues were in the extended category), mainly helical (between 50-80% of the residues were helical), helical (>80% of the residues were assigned as participating in an α-, pi- or 310-helical structure), or p-strand containing. The fisher.test( ) function in base R was used to conduct a Fisher's exact test to investigate the independence of the helical or linear extended structure and possession of activity. Only peptides classified as helical (>80% of residues assigned helical, 45 peptides) or linear extended (no residues in helical or p-strand structures, 17 peptides) were included in this statistical test. Peptides were labeled as active if they visually inhibited at least one of the bacterial strains at one of the tested concentrations.

### 2.4.5. Hemolysis Assay

Peptides were evaluated for toxicity using three independent hemolysis experiments. Whole blood from healthy donor pigs, supplemented with Na Citrate, was purchased from Lampire Biological Laboratories (Pipersville, PA, USA). Red blood cells (RBCs) were washed and isolated by three centrifugation cycles using Roswell Park Memorial Institute medium (RPMI; Thermo-Fisher Scientific) to create a 1% (v/v) RBC solution. Lyophilized AMPs were suspended and serially diluted from 128 down to 1 μg/mL using RPMI in a 96-well plate, before being combined with 100 μL of the 1% RBC solution. Following a minimum 30 min incubation at 37° C., plates were centrifuged and % volume from each supernatant was transferred to a new 96-well plate. The absorbance of these wells was measured at 415 nm. To quantify hemolytic activity and determine the AMP concentration that lyses 50% of the RBCs (HC50), absorbance reading from wells containing RBCs treated with 11 μL of a 2% TritonX-100 detergent solution (TX-100) or RPMI (AMP solvent-only) were used to define 100% and 0% hemolysis, respectively. All centrifugation steps were performed at 500× g for five minutes in an Allegra-6R centrifuge (Beckman Coulter, Brea, CA, USA).

### 2.4.6. Cytotoxicity

The human embryonic kidney cell line HEK293 and the corresponding growth media and supplements were purchased from the ATCC. The cells were maintained in Eagle's Minimum Essential Medium (EMEM) supplemented with 10% fetal bovine serum (FBS) and 1% Penicillin-Streptomycin solution and grown in a 5% CO2 incubator at 37° C. Approximately 1×10⁴ cells were distributed to each well of a 96-well flat-bottom cell culture plate (Corning #3595, Corning, NY, USA). Cells were incubated overnight to allow them to adhere. Lyophilized 80 μg peptide aliquots were resuspended to 1.28 mg/mL with UltraPure deionized water and serially diluted in polypropylene 96-well microtiter plates from 128-0.5 μg/mL. TX-100 was diluted to 2% in UltraPure deionized water and added as a positive control. Complete growth medium was added to the peptides and TX-100 containing wells. Spent growth medium of wells with adhered cells was replaced with the contents of wells containing serially diluted peptides or TX-100. Cells were incubated for four hours at 37° C. in 5% CO2 incubator. Growth medium containing peptides or TX-100 was replaced with fresh growth medium containing 10% (v/v) alamarBlue (Bio-Rad Laboratories, Hercules, CA, USA) and incubated for 20 h. Fluorescence was measured at excitation of 540 nm and emission of 590 nm in a Cytation 5 plate reader (Agilent BioTek, Winooski, VT, USA) and results recorded and analyzed with Gen5 software (Agilent BioTek, Winooski, VT, USA). The average fluorescence reads of TX-100 and growth media only wells were used to calculate 0% and 100% cell viability, respectively. The percentage of viable cells at each peptide concentration was determined by Formula 1:

$$Cell viability ( \% ) = 1 - ( \frac{[ {fluorescence}_{( 100 \% viable )} - {fluorescence}_{( peptide )} ]}{[ {fluorescence}_{( 100 \% viable )} - {fluorescence}_{( 0 \% viable )} ]} ) \times 100$$

### 2.4.7. BLAST and Phylogenetic Analysis

Amino acid sequences for the mature peptides tested in vitro were used as the query sequences for BLASTp analysis with NCBI BLAST+v2.13.0 [29]. The sequences were searched against the non-redundant protein database (downloaded on 01/19/2022) [29]. Max target sequences was set to 5. To identify protein family memberships, mature sequences were fed into InterProScan v5.56-89.0 [37] in FASTA format with default parameters. Multiple sequence alignment was performed with the 51 validated AMPs using the Bioconductor R package msa v1.28.0 [152] with the ClustalW option. A distance matrix was created using the multiple sequence alignment and a phylogenetic tree using neighbour-joining tree estimation was created using the ggtree v3.4.2 [153,154,155] and ape v5.6-2 [156]R packages. AMP protein families, origin of AMPs and BLASTp results were annotated on the phylogenetic tree using ggtree [153,154,155].

Reference is made to Richter et al. (2022). Associating Biological Activity and Predicted Structure of Antimicrobial Peptides from Amphibians and Insects. Antibiotics (Basel), Nov. 27, 2022: 11(12), 1710.

### Example 3

### Antimicrobial Peptides as Antibiotic, Antiviral and Anticancer Agents

A bioinformatics approach was implemented to identify peptides having antimicrobial activity, based on data and information derived from and based upon the immune defense systems of higher eukaryotes.

The bioinformatics approach employed herein located antimicrobial peptides using rAMPage: Rapid Antimicrobial Peptide Annotation and Gene Estimation. rAMPage takes RNA sequencing data, uses state-of-the-art open-source bioinformatics tools implemented in a high-throughput pipeline. The present approach using rAMPage is the first time a process has been packaged for easy reproducibility at a high-throughput and cost-effective scale. Using rAMPage, 1,029 AMPs have been identified, as described herein.

The rAMPage process is utilized as described in detail in Example 1. Out of 1,137 putative antimicrobial peptides discovered using rAMPage, 1029 (5+1,024, as listed below) are novel antimicrobial sequences in these aspects: Sequence exists in NCBI nr database, but is uncharacterized; Sequence exists in NCBI nr database, is characterized but not as an antimicrobial peptide; Sequence is a truncation or subsequence of an existing antimicrobial peptide; or Sequence contains mutations differing it from an existing antimicrobial peptide.

The antimicrobial peptide sequences are as follows:

SEQ ID NO: 1
AGLQFPVGRIHRHLKTR.
SEQ ID NO: 2
GLVGKLVKGGLKLIGHVANG.
SEQ ID NO: 3
GLLGKIVGGGLKLIGNHVANHLANG.
SEQ ID NO: 4
FPFPFGRR.
SEQ ID NO: 5
LLICALCPPGAARSLRKR.
SEQ ID NO: 6
HTGLHLCIYCCNCCRNKGCGYCCRT.
SEQ ID NO: 7
SRMIRRPPGFSPFRVAPASSLRR.
SEQ ID NO: 8
AGYSRMIRRPPGFSPFRVAPASSLRR.
SEQ ID NO: 9
SWHGRGGGRRGGSGRRGGRGGGR.
SEQ ID NO: 10
FCSQIGCRIGKRRLSSSVHRERR.
SEQ ID NO: 11
FLPIVGKLLSGLLGK.
SEQ ID NO: 12
GILDTLKQLGKAAVQGLLSKAACKLAKTC.
SEQ ID NO: 13
RDRSLCRRRFPVLLVPELRTRR.
SEQ ID NO: 14
ISEHVEKIKIGYEKHSLSKHFKFVHNK.
SEQ ID NO: 15
GLWTTIKEGVKNFSVGVLDKIRCKITGGC.
SEQ ID NO: 16
KEERGAPKGCWTKSYPPKPCSGKR.
SEQ ID NO: 17
GILTLKYPCRAWYHH.
SEQ ID NO: 18
LLWGHRLQR.
SEQ ID NO: 19
KVWCQIFSMSSQSKR.
SEQ ID NO: 20
LPPPHLWRRAMNCLMGR.
SEQ ID NO: 21
LVWVKK.
SEQ ID NO: 22
GILTLKVSNMIWVIFSRLAL.
SEQ ID NO: 23
HRAKTVQTFLEKRHRR.
SEQ ID NO: 24
GVFDTLKKIGKTVGKVALGVAKNYLNSQK.
SEQ ID NO: 25
GFWSHLKDKFIETGKAIKRVWKNFFSSG.
SEQ ID NO: 26
FLGTALKLGKEKKMRRKRKKMKK.
SEQ ID NO: 27
FLGTALKLGKAFAKTIVPMISDAFHKNQ.
SEQ ID NO: 28
FLGTAIKLGKAFAKTIVPMISDALRKNQ.
SEQ ID NO: 29
GRKKPKLGTFWG.
SEQ ID NO: 30
GVFDTLKKIRKRRMKNFMKR.
SEQ ID NO: 31
GVFDTLKKIGKTVVKVALGVAKNYLNSQK.
SEQ ID NO: 32
GLLGKILGVGKKVLCGVSGLC.
SEQ ID NO: 33
GILLNTLKGAAKNV.
SEQ ID NO: 34
VSMLIPVTSRIRVKR.
SEQ ID NO: 35
SGKTLEKTLAKKNPKLGKTKF.
SEQ ID NO: 36
GWLDIAKNVASIVAGLGKR.
SEQ ID NO: 37
GWLDIAKKVASVVAGIVKR.
SEQ ID NO: 38
GWLDIAKKVASVVAGIGKR.
SEQ ID NO: 39
GLFDLVTSVLGGLGKRR.
SEQ ID NO: 40
GWLDIAKKVASVVAGLGKR.
SEQ ID NO: 41
GWLDIAKKSLQLLQASEKEVKRR.
SEQ ID NO: 42
GLFDLVTGLLGNLGKRR.
SEQ ID NO: 43
RIQEHLRSFSKYVPVPVRR.
SEQ ID NO: 44
SNRRPCTGRNCRPRRPGVFTPIAKPLKK.
SEQ ID NO: 45
RIHRHLKTR.
SEQ ID NO: 46
GWSDIAKKVASAVAGLGKR.
SEQ ID NO: 47
GWSDIAKKVASAVAGLGKRIIFFPG.
SEQ ID NO: 48
GSSRMIRRPPGFSPFRVAPASSLKR.
SEQ ID NO: 49
GMQKNGRCANVNVMCVSVRSNKK.
SEQ ID NO: 50
FFRVLCTSIRRRLGSKDLMCKDKKK.
SEQ ID NO: 51
HTGLHLCIYCCKCCRYKGCGYCCRT.
SEQ ID NO: 52
SNIKGTYRAVAVVKKIKYKRHIP.
SEQ ID NO: 53
GIIDSVMGQVCVKMNYKLPFCARFKPK.
SEQ ID NO: 54
AITRMIRRPPGFTPFRIAPAIV.
SEQ ID NO: 55
GIFGKILGVGKKVLCGLSGLC.
SEQ ID NO: 56
VVGNLISTIVNLVKKLGK.
SEQ ID NO: 57
AAPRGCWTKSIPPKP.
SEQ ID NO: 58
EQERFLPLLAGLAANFLPKLFCKITRKC.
SEQ ID NO: 59
GLLSGLLGAGKKIVCGLSGMC.
SEQ ID NO: 60
GLFTLIKGAAKLIGKTVAKEAGKTVAKEA.
SEQ ID NO: 61
GLLSGILGAGKNRVCGLSGLC.
SEQ ID NO: 62
GILSGLLGAGKKIVC.
SEQ ID NO: 63
GLISGILGAGKK.
SEQ ID NO: 64
GLLRGILGAGKKIVCGLSGLC.
SEQ ID NO: 65
GLFTLIKGAAKLIGKTVAKEAGKTGLEFMF.
SEQ ID NO: 66
ATAWGIPPNGIPPIVAVRIRPLCGTV.
SEQ ID NO: 67
GFMDTAKSVAKNVAVTLLDNLKCKITKAC.
SEQ ID NO: 68
GLISGILGAGKKVLC.
SEQ ID NO: 69
GFMDTAKNVAKNVAVTLLYNLKCKITKAC.
SEQ ID NO: 70
GFMDTAKNVAKKV.
SEQ ID NO: 71
GLLSGILGAGKKIVCGFSGLC.
SEQ ID NO: 72
FLPPWLRPSKK.
SEQ ID NO: 73
FLPPWLRPSKTTKRQLELKQY.
SEQ ID NO: 74
CYSFSFLGPSPYLSVRKSKR.
SEQ ID NO: 75
GIFGKILGVGKKVLC.
SEQ ID NO: 76
GVLGTVKNLLIGAGKSAAQSVR.
SEQ ID NO: 77
AGYTRMIRRPPGFSPFRIAPAIV.
SEQ ID NO: 78
GLLDSFKNLALNAAKRC.
SEQ ID NO: 79
TVWGFRPTNPKPKSPG.
SEQ ID NO: 80
GLRSGILGAGKKVLCGLS.
SEQ ID NO: 81
ATALGLSSRGLLPIGFMFKDTIRCRKD.
SEQ ID NO: 82
AGYTRMIRRPPGFSPFRVAPASSLKR.
SEQ ID NO: 83
FFPLFASLAANFLPKLFCKIARKC.
SEQ ID NO: 84
RPAKSRR.
SEQ ID NO: 85
GVLGTVKNLPGKGLRICSSKGSST.
SEQ ID NO: 86
GLLSGILGAGKKLLKWKR.
SEQ ID NO: 87
GIFTLIKGAAKLIGKTVAKEAGKTGLEFMF.
SEQ ID NO: 88
GLLSGILGAGKR.
SEQ ID NO: 89
VIPFVARVAAEMMQHVYCAASKRC.
SEQ ID NO: 90
DDPQERFLPFLAGLAANFLPKLFCKITRKC.
SEQ ID NO: 91
ERERFLPFLAGLAANFLPKLFCKITRKC.
SEQ ID NO: 92
EQARFLPLLAGLAANFLPKLFCKITR.
SEQ ID NO: 93
KQERCVDMGFSPTGKRPPFCPYPG.
SEQ ID NO: 94
EQERFLPLLAGLAANFLPKIFCKITKKC.
SEQ ID NO: 95
EQERFFPLFASLAANFLPKLFCKIARKC.
SEQ ID NO: 96
EQERFLPFLAGLAANFLPKLFCKITRKC.
SEQ ID NO: 97
KQERWVDTGFSHTGKRPPFCPYPG.
SEQ ID NO: 98
LPHLHPWRR.
SEQ ID NO: 99
IIKGLSPLRKIEFGPGGSIRSKR.
SEQ ID NO: 100
NSHLSICIYCCKCCKRKGCGMCCKT.
SEQ ID NO: 101
FFPLFGSLVANFLPKLICKIARKC.
SEQ ID NO: 102
GIFGGLLKVGKKIACGLSGLC.
SEQ ID NO: 103
GIFGGLLKVGKKIACGSVFTLVKSSLDC.
SEQ ID NO: 104
GLFTIIKGAAKLIGKAVAKEAGKSG.
SEQ ID NO: 105
AVRLRGCWTKSLPPQPCYGKR.
SEQ ID NO: 106
DIFGGLLKVGKKIACGLSGLCQSLN.
SEQ ID NO: 107
GIFGGLLKVGKK.
SEQ ID NO: 108
GIFGGLLKVGKKIPRFISSILATSSSLFS.
SEQ ID NO: 109
GIFGGLLKVGMKIACGLSGLC.
SEQ ID NO: 110
GIFGGLLKVGKKIACGLS.
SEQ ID NO: 111
ATAWDLGPHGLRPIRPIRIRPLCGDRS.
SEQ ID NO: 112
GFLDTAKNAAKNVAKNVAVTLL.
SEQ ID NO: 113
GIFGLLRKKYFLQFLNFQKTVIKKLQL.
SEQ ID NO: 114
GIFGGRGKLFIEHAPPRPL.
SEQ ID NO: 115
ALKFPFRCKARIRR.
SEQ ID NO: 116
AAPLRGCWTKSLPPQPCYGKR.
SEQ ID NO: 117
GIFGGLLKVGKKKEMKKLLKWKR.
SEQ ID NO: 118
ATAWDLGPHGLRPIRPIRIRPLCGKDNS.
SEQ ID NO: 119
GIFGGLLKVGKKLLKWKR.
SEQ ID NO: 120
AGYSRMIRRPPGFSPFRVAPPSSLKR.
SEQ ID NO: 121
GLFTIIKGAAKLIGKAGQRSRQEWA.
SEQ ID NO: 122
GIFPKMFTLKKPLLLIVL.
SEQ ID NO: 123
GFLDTAKNAAKNAAK.
SEQ ID NO: 124
GIFGGLLKMGKKIACGLS.
SEQ ID NO: 125
GIFGGLLKVR.
SEQ ID NO: 126
FKCRIPLVKPLLNNKRQKR.
SEQ ID NO: 127
AGYSRMIRRPPGFTPFRIAPEMG.
SEQ ID NO: 128
GIFGGLLKVGKKIACGLSG.
SEQ ID NO: 129
GIFGGILKVGKKIACGLSGLC.
SEQ ID NO: 130
GIFGGLLKVGKKIACGLTGLC.
SEQ ID NO: 131
GIFGGLLKVGKKIACGLSGL.
SEQ ID NO: 132
SADQTGMNKAALSPIKKKKKSV.
SEQ ID NO: 133
GIFGGLLKVGKKIA.
SEQ ID NO: 134
RWVPFGKR.
SEQ ID NO: 135
WVPFGKR.
SEQ ID NO: 136
RRYKKSNSLGLGIVSSSPCLRKR.
SEQ ID NO: 137
GIFGGLLKVGKKIACGLSGLCQS.
SEQ ID NO: 138
GCSRWIIGSRKKR.
SEQ ID NO: 139
GIFGGHLKVGKKIACGLSGLC.
SEQ ID NO: 140
GIFGGLLKVGKKIACGLNTDVSVIYNMN.
SEQ ID NO: 141
GIFGGLLKEGKKIACGLSGLC.
SEQ ID NO: 142
KLMIPRKKR.
SEQ ID NO: 143
GIFGGLLKVGKKI.
SEQ ID NO: 144
GLLGTVKNLLIGAGKSA.
SEQ ID NO: 145
GILTLKYPRSSL.
SEQ ID NO: 146
GIFGGLLKVGKKIACRLS.
SEQ ID NO: 147
GIFGGLLKVKKKKEMKKLLKWKR.
SEQ ID NO: 148
GILSVFKNALPGIMKFITG.
SEQ ID NO: 149
GIFGGLLKVRKKIACGLSVLC.
SEQ ID NO: 150
APAWGIPPNGIPRMVAVRIRPLCGAV.
SEQ ID NO: 151
GIFGGLLKVGKKIACG.
SEQ ID NO: 152
VIPFVASVAAEMMQHVYCAASRRCKN.
SEQ ID NO: 153
GILSGLLGAGKKLACGLIGLC.
SEQ ID NO: 154
GIFGGLLKVGKKIACGLSTAR.
SEQ ID NO: 155
GILSVIKNALPGIMRFIAG.
SEQ ID NO: 156
GIFGGLLKVGKKIACGLSGQRIGGE.
SEQ ID NO: 157
NAISMIADLARKLLGK.
SEQ ID NO: 158
ALKFPFRCKAAFC.
SEQ ID NO: 159
GIFSKITGKGIKNLLFKGIKNV.
SEQ ID NO: 160
ATAWDLGPHGLRPIRPIRIRPLCGKDRS.
SEQ ID NO: 161
APVWGIPPNGIPRMVAVRIRPLCGAV.
SEQ ID NO: 162
ATAWDLGPHGLRPIRPIRIRPLCGK.
SEQ ID NO: 163
SGYSRMIRRPPGFSPFRVAPASSLKR.
SEQ ID NO: 164
GSLEVQRARCRHPLLRSSLRCRHLAQMRT.
SEQ ID NO: 165
FLPPWLRPPKKTKR.
SEQ ID NO: 166
RRYWILRRLESGVERR.
SEQ ID NO: 167
VIPFVASVAAEMMQNVYCAASRRCKN.
SEQ ID NO: 168
GSLEVQRARCRHPLYRSSLRCRHLAQMRT.
SEQ ID NO: 169
AAPLRGCWTKNLPPQPCYGKR.
SEQ ID NO: 170
SILSVIKNALPGIMKLIAG.
SEQ ID NO: 171
GIFGGLLKVGKKIACGL.
SEQ ID NO: 172
TIWGFRPTNPKPKPPG.
SEQ ID NO: 173
GIFGGLLKKKKK.
SEQ ID NO: 174
SILSVIKNALPGIMKFIAG.
SEQ ID NO: 175
GIFGGLLKVGKKIACFISSILATSSSLFSS.
SEQ ID NO: 176
GIFSKITGKGIKNLLFKGIK.
SEQ ID NO: 177
GLFTIIKGAAKLIGKAVAKR.
SEQ ID NO: 178
EEERFFPLFGSLVANFLPKLICKIARKC.
SEQ ID NO: 179
QIGSRKKRGCSRWIIGINGPVCRD.
SEQ ID NO: 180
SNRGIFGGLLKVGKKIACGLSGLC.
SEQ ID NO: 181
AGYSRMIRRPPGFSPFRVAPASSLKRA.
SEQ ID NO: 182
RWVPFGKRWVPFGKR.
SEQ ID NO: 183
WVPFGKRWVPFGKR.
SEQ ID NO: 184
RWVPFGKRWVPFGKRWVPFGKR.
SEQ ID NO: 185
WPFGKRWVPFGKRWVPFGKR.
SEQ ID NO: 186
VPFGKRWVPFGKRQIE.
SEQ ID NO: 187
WVPFGKRWVPFGKRQIE.
SEQ ID NO: 188
GSRKKRGCSRWIIGINGPVCRD.
SEQ ID NO: 189
AGYSRMIRRPPGFSPFRVAPASSLKRAGYS.
SEQ ID NO: 190
KLMIPRKKRGIFGGLLKVGKKIACGLSGLC.
SEQ ID NO: 191
QDERNAEEEKRGIFGGLLKVGKKIACRLS.
SEQ ID NO: 192
QDGRNAEEEKRGIFGGLLKVGKKIACGLSG.
SEQ ID NO: 193
IPNGCSIRAEPPVPPPKPSLKKR.
SEQ ID NO: 194
SRSGRGGRGGRGGRGGRGGRG.
SEQ ID NO: 195
SSATWLWTLKMKWLLLPLPLPLKR.
SEQ ID NO: 196
GWACSIGVRLLPQWFGLFLR.
SEQ ID NO: 197
QTNLLGKVSKVANDALKILPIQV.
SEQ ID NO: 198
HTVLGLCIYCCKCCRYKGCGYCCRT.
SEQ ID NO: 199
GLWTTIKEGVKNFSVGVLDKIRC.
SEQ ID NO: 200
ATAWKVPPGLQPIRPIRIRPLCGNDKS.
SEQ ID NO: 201
RRPPGFTPFRVAPASSLKR.
SEQ ID NO: 202
KSVMSGRGKGGKSKSSGKVKSRSSR.
SEQ ID NO: 203
GRGKRGLVTNLLSTVL.
SEQ ID NO: 204
GRGKRGIVKNLLSTVL.
SEQ ID NO: 205
HTGLHLCTYCCKCCRNKGCGYCCRT.
SEQ ID NO: 206
DTLFGPCGHIATCSLCSPRVKKC.
SEQ ID NO: 207
FLKKTWKKLKKAAKSILKNTG.
SEQ ID NO: 208
RAKNTFIVESEKTVLSICRKAIKKR.
SEQ ID NO: 209
FLKKTWKKLKKAAKSILKNTGPVTYQHKF.
SEQ ID NO: 210
GLLGKILGVGKKVLCGVS.
SEQ ID NO: 211
GIFSKLAGKIPLFVSLPS.
SEQ ID NO: 212
GIFSKLAGKKIKNPLISGLKNIGKEVGMA.
SEQ ID NO: 213
VILFVASVAAEMMHHVYCAASKRCKN.
SEQ ID NO: 214
GILTDTLKGAAKNLAGVLLDKLKCKITGGC.
SEQ ID NO: 215
GIFSLIKGAAKVVAKGLGKEEVKR.
SEQ ID NO: 216
GIFSKLAGKMIKNLLISGLKNIGK.
SEQ ID NO: 217
GLLGKIPGVGKKVLCGVSGLC.
SEQ ID NO: 218
GIFSKLAGKKIK.
SEQ ID NO: 219
GIFSLIKGAAKVVAKGLGREVQEEEVKRG.
SEQ ID NO: 220
GLLGKILGLGKKVLCGVSGLC.
SEQ ID NO: 221
GLWTTIKEGVKNFSVGVLDKIRCKITGEC.
SEQ ID NO: 222
GLLGKILGVGKKVLCGGSGLC.
SEQ ID NO: 223
GVPLIYKRPGIYVTKRPRGK.
SEQ ID NO: 224
GLLGKILGVRKKEEMSLMKGMLKR.
SEQ ID NO: 225
GILLNTLKGAAKNG.
SEQ ID NO: 226
VIPFVASVAAEMMHRVYCAASKRCKN.
SEQ ID NO: 227
GIFSKLAGKRNKKRYFL.
SEQ ID NO: 228
GLLLDTVKGAAKNVAGKLQMKK.
SEQ ID NO: 229
RLHHNLSRMIRRPPGFTPFRVAPASSLKR.
SEQ ID NO: 230
FDQGYGHFCRQGALQNLLKMASCKLDKTC.
SEQ ID NO: 231
FIPLVSGLFSRLLGK.
SEQ ID NO: 232
GILTDTLKGAAKNVAGVLLDKLKCKITGGY.
SEQ ID NO: 233
GIFSLIKGAAKVVAKGLGKEVKR.
SEQ ID NO: 234
GLLGKILGVGKKVLC.
SEQ ID NO: 235
GIFSLIKGAAKVVAKPLATT.
SEQ ID NO: 236
GIFSLIKGAAKNVAG.
SEQ ID NO: 237
GIFPKLAGKKIKNLLISG.
SEQ ID NO: 238
GILTDTSKGAAKNVAGVLLDKLKCKITGGC.
SEQ ID NO: 239
GRPRWSHRSRR.
SEQ ID NO: 240
GLLGKILGVGKKVLTPHSTFFP.
SEQ ID NO: 241
SNRDFFKVNIFGFGRFSWC.
SEQ ID NO: 242
GIFSKLAGKKRYFL.
SEQ ID NO: 243
GIFSLIKGAAKVVAKGLGKEVGG.
SEQ ID NO: 244
GPLGKILGVGKKVLCGVSGLC.
SEQ ID NO: 24
5GIFSKLAGKKIKKREAKQKEVFSLNWR.
SEQ ID NO: 246
GAPKGCWTKS.
SEQ ID NO: 247
FLPSSPWNEGTYVLKKLKT.
SEQ ID NO: 248
GIFSKLAGKKIKNPLMSRFL.
SEQ ID NO: 249
GIFSKLAGKKIKNL.
SEQ ID NO: 250
GILLNTLKGAAKNGRGF.
SEQ ID NO: 251
GILLNTLKGAAKNVAGLLLDKLKCKITGGC.
SEQ ID NO: 252
FLPLLAGLAANFLPTIFCKISRKC.
SEQ ID NO: 253
GIFSKLAGKKIKNLLISGLKNLLISG.
SEQ ID NO: 254
GIFSKLAGKKIKNLLISGLKNIGAR.
SEQ ID NO: 255
GIFSLIKGAAKVVAKGLGKFGLDLMACKVT.
SEQ ID NO: 256
GIFSKLAGKKIKSL.
SEQ ID NO: 257
GALKGCWTKSYPPKPCSGKR.
SEQ ID NO: 258
GIFSKLAGKKIKNLLISGLKNYPPKDV.
SEQ ID NO: 259
GIFSKLLGKRSRTCS.
SEQ ID NO: 260
GIFKVNIFGRVVLSIFSAL.
SEQ ID NO: 261
GILLNTLKGAAKNVAGDLLDKLKCKITGGC.
SEQ ID NO: 262
GAPKGCWTKSYPPKPCSATLGRTSFYFF.
SEQ ID NO: 263G
IFSKLAGKKMFTLKKPLLLI.
SEQ ID NO: 264
GIFSKLAGKKIKNLLISGLKNIGKEVGKFG.
SEQ ID NO: 265
GIFTKLAGKKIKNLLISGLKNIGRFLIFF.
SEQ ID NO: 266
VLLGKILGVGKK.
SEQ ID NO: 267
TPSWVFPISYCTSVSLKR.
SEQ ID NO: 268
GAPKGCWTKSYPPQPCSGRSKKRCAQ.
SEQ ID NO: 269
GASKGCWTKSYPPKPCSGKR.
SEQ ID NO: 270
GLLGKILGVGKKALTPHSTFFPTPRILPNR.
SEQ ID NO: 271
GLLLDTVKGAAKNVAGVLLDKLKCKITGGC.
SEQ ID NO: 272
GSSRMIRRPPGFTPFRIAPASSLKR.
SEQ ID NO: 273
GIFSKLAGKKIKNLLISGPKNIGKEVGMDV.
SEQ ID NO: 274
GIFSKLAGKKIKNLLISGLKNIGKEAGP.
SEQ ID NO: 275
GIFSKLAGKKIKNLLISGLKNIGLP.
SEQ ID NO: 276
GLLGKILGVEKR.
SEQ ID NO: 277
GIFSKLAGKKIKNLLISGLKNIGAPP.
SEQ ID NO: 278
GIFSKLAGKKIKNLLISGLKNIGPQ.
SEQ ID NO: 279
GLLGKVLGVGKKEEMSLMKGMLKR.
SEQ ID NO: 280
GIFSLIKGAAKVVAKG.
SEQ ID NO: 281
GLLGKVLGVGKK.
SEQ ID NO: 282
GIFSKLAGKKIKNLLISGLKNIGKNLLIS.
SEQ ID NO: 283
GIFSKLAGKKIKNFLAYVLESAYEQV.
SEQ ID NO: 284
GILLYTLKGAAKNVAEVLLDKLKCKITGGC.
SEQ ID NO: 285
GIFSLIKGAAKVVAKGLGKEGKFRRKK.
SEQ ID NO: 286
GLLGKILGVGKR.
SEQ ID NO: 287
GFSPFRIAPASSLKR.
SEQ ID NO: 288
GIFSKLAGKKIKNLLISGQPGPSAPWDV.
SEQ ID NO: 289
GIFSKLAGKKIKNLLISGLKNIGSP.
SEQ ID NO: 290
GIFSKLAGKKIKSLLISGLKNIGE.
SEQ ID NO: 291
FLPSSPWNEGTYVLKKLKRKK.
SEQ ID NO: 292
GLLGKILGVGKKVQCGVSGLC.
SEQ ID NO: 293
GILLNTLKGAAR.
SEQ ID NO: 294
GIFSKLAGKKIKNPLISGLKNIGKEVGMD.
SEQ ID NO: 295
GLLGKILGVGRKVLCGVSGLC.
SEQ ID NO: 296
GIFSLIKGAAKVVAKGLGKEVGKFGQGSGQ.
SEQ ID NO: 297
GLLGKILGVGKKALCGVSGLC.
SEQ ID NO: 298
KWGKPRWASSLKR.
SEQ ID NO: 299
GLLGKILGVGKKVLCGVSGFVKA.
SEQ ID NO: 300
GLLGKILGVGKKVLCGVSGL.
SEQ ID NO: 301
RRCNTKWGKPRWASSLKR.
SEQ ID NO: 302
GILLNTLKGAAKNWPGFC.
SEQ ID NO: 303
GLLGKILGVGKKEEMSLMKGMLKR.
SEQ ID NO: 304
GIFSLIKGAAKVVAKGLGKEVGKFGQGSG.
SEQ ID NO: 305
GIFSLIKGAAKVVAKGLGKEVQEEEVKR.
SEQ ID NO: 306
GTMSTNKGVSKNVAVALLDRLQCKITGTC.
SEQ ID NO: 307
RCNTKWGKPRWASSLKR.
SEQ ID NO: 308
RPRWSHRSRR.
SEQ ID NO: 309
GIFSKLAGKKIKNLLISGLKNMGKEVGKFG.
SEQ ID NO: 310
GIFSKLAGKKIKNLLISGLKNIGKEVGKR.
SEQ ID NO: 311
GLWTTIKEGVKNFSVGVLDKIRCKITG.
SEQ ID NO: 312
GLLGKILGVRKKVLCGVSGLC.
SEQ ID NO: 313
GIFSKLAGKKIKNLLISGLKNK.
SEQ ID NO: 314
GIFSKLAGKK.
SEQ ID NO: 315
GIFSKLARRKR.
SEQ ID NO: 316
GIFSKLAGKKKIKNLLISGLKNIGKE.
SEQ ID NO: 317
VVPGFTPFRIAPASSLKR.
SEQ ID NO: 318
GLLGKVLGVGKKVLTPHSTF.
SEQ ID NO: 319
ATAWKVPPGLQPIRPIRIRPLC.
SEQ ID NO: 320
GIFSKLAGKKIKSLLVSGLKNIGKEVGMD.
SEQ ID NO: 321
GIFSLIKGAAKVV.
SEQ ID NO: 322
GIFSKLAVKKIKNLL.
SEQ ID NO: 323
GIFSLIKGAAKVVAKGLGKEAGKFG.
SEQ ID NO: 324
GIFSKLAGKKIKNLP.
SEQ ID NO: 325
GIFSKLAGKKIKNLPMFLSPLMSR.
SEQ ID NO: 326
GIFSKLAGEAIKNLLISGLKNIGK.
SEQ ID NO: 327
EQKRGLLGKILGVGKKVLCGVSGLC.
SEQ ID NO: 328
LAHRELRRGLLGKILGVGKKVLCGVSGLC.
SEQ ID NO: 329
EQERGLLGKIPGVGKKVLCGVSGLC.
SEQ ID NO: 330
KRSKRVIPIVSGLLSSLLGK.
SEQ ID NO: 331
RKKRVIPIVSGLLSSLLGK.
SEQ ID NO: 332
EQKRGIFSKLAGKKIKNLLISGLKNLLISG.
SEQ ID NO: 333
EQKRGIFSKLAGKKIKNLLISGLKNIG.
SEQ ID NO: 334
EQKRGIFSKLAGKKIKNLLISGLKN.
SEQ ID NO: 335
ELERGLLGKILGVGKKVLCGVSGLC.
SEQ ID NO: 336
KEERGAPKGCWTKSYPPQPCSGRSKKRCAQ.
SEQ ID NO: 337
KEERGASKGCWTKSYPPKPCSGKR.
SEQ ID NO: 338
EQKRGIFSKLAGKKIKNLLISGLKNIGPQ.
SEQ ID NO: 339
EQERGLLGKILGVGKKVLCGVSGLC.
SEQ ID NO: 340
DANGEERRGLLGKILGVGKKVLCGVSGLC.
SEQ ID NO: 341
NEEERRGLLGKILGVGKKVLCGVS.
SEQ ID NO: 342
EQKRGIFSKLAGKKKIKNLLISGLKNIGKE.
SEQ ID NO: 343
GIFSLIKGAAKVVAKGLGKEVDKFGL.
SEQ ID NO: 344
GIFSLIKGAAKVVAKGLGKGGEVQEEEVKR.
SEQ ID NO: 345
VIPIVSGLLSCLLEKKKKILKL.
SEQ ID NO: 346
GILLNTLKGAAKNVAG.
SEQ ID NO: 347
GLWTTIKEGVKNFSVGVLDKIRSKKR.
SEQ ID NO: 348
GIFSLIKGLGKEVGKFGL.
SEQ ID NO: 349
VIPIVSGLLSSLLKKKQKILKL.
SEQ ID NO: 350
GVPLIYKRPGLLVTYIPG.
SEQ ID NO: 351
SLLDSIKSMAISAGKGALQNLLKM.
SEQ ID NO: 352
GIVSLIKGAAKVVAKGLGKEVGKFGLDLM.
SEQ ID NO: 353
GSSRMIRRPPGFTPFRVAPASSLKR.
SEQ ID NO: 354
GIFSLIKGAAKVVAKGLGREVQEEEVKR.
SEQ ID NO: 355
GIFSKLAGKKIKRKYLFLFRFPLLHHH.
SEQ ID NO: 356
GIFSLIKGAAKVVAKGSGGRSEKR.
SEQ ID NO: 357
GIFSLIKGAAKVVAK.
SEQ ID NO: 358
VIPFQHPLHRDHLFFLPHRRLSL.
SEQ ID NO: 359
GLLGKVLGVGKKVLCGVTGRERCQ.
SEQ ID NO: 360
GIFSKLAGKKIKNRQCLSQF.
SEQ ID NO: 361
RPPGFPPFRVAPASSLKR.
SEQ ID NO: 362
GICSLIKGAAKVVAKGLGKEVGKFGLDL.
SEQ ID NO: 363
GLLGKVLGVGKKVLCGVTGLC.
SEQ ID NO: 364
GIFSLIKGAAKVVAKGLGKEVE.
SEQ ID NO: 365
GLLGKVLGVGKR.
SEQ ID NO: 366
GVPLIYKRPGIYVTKRPRGS.
SEQ ID NO: 367
RMIRRPPGFTPFRVAPASSLKR.
SEQ ID NO: 368
RPPGFPPFRIAPASSLKR.
SEQ ID NO: 369
GIFKVNIFGRVVLCNA.
SEQ ID NO: 370
GILLNTLKRCR.
SEQ ID NO: 371
RPPGCSPFRIAPASSLKR.
SEQ ID NO: 372
GIFSLIKGAAKVVAKGLGKEVGKFGLGNTS.
SEQ ID NO: 373
GFSPFRVAPASFLKR.
SEQ ID NO: 374
GIFSLIKGAAKVVAKGLGRGSSGGRSEKR.
SEQ ID NO: 375
GIFSLIKGAAKVVAKSSGGRSEKR.
SEQ ID NO: 376
GIFSLIKGLGKEVGKFGLDLMACKVTNQC.
SEQ ID NO: 377
GIFSLIKGAAKVVAKGLISEKIPLFTSSS.
SEQ ID NO: 378
FLPSSPWNEGTYVLKKLKWKK.
SEQ ID NO: 379
GIFSKLAGKKR.
SEQ ID NO: 380
SIISKLAGKKIKNLLISGLKNVGKEVG.
SEQ ID NO: 381
GLLGKVLGVGNKAQ.
SEQ ID NO: 382
GIFSKLAGKKIKNLAYVLESAYEQ.
SEQ ID NO: 383
GVPLIYKRLGLLVTYIPG.
SEQ ID NO: 384
GLLGKVLGVGKKVLCGVT.
SEQ ID NO: 385
SSCRTLSRYEAKRSLR.
SEQ ID NO: 386
VIPFQHPLHRDHLFFLPHRRL.
SEQ ID NO: 387
PPGFSPFRVAPASFLKR.
SEQ ID NO: 388
GIFSLIKGAAKVVAKGLGKVVAKGLGKEVG.
SEQ ID NO: 389
GIFSLIKGAAKVVAKGLGKEVAKGLGKEVG.
SEQ ID NO: 390
ATAWKVPPGLQPIRPIR.
SEQ ID NO: 391
GIFSLIKGAAKVVPKLAHFLAQTFG.
SEQ ID NO: 392
GLLGKVLGVGKKVL.
SEQ ID NO: 393
GIFSLIKGAAKVVARFGQGSGQVWAGPYGL.
SEQ ID NO: 394
VIPIVSGLLSCLLEKKK.
SEQ ID NO: 395
GLLGKVLGVGKKVLCVVSGLC.
SEQ ID NO: 396
VIPFVASVAAEMMHHMYCAASKRCKN.
SEQ ID NO: 397
EQERVIPIVSGLLSCLLEKKKKILKL.
SEQ ID NO: 398
EQERVIPIVSGLLSSLLKKKQKILKL.
SEQ ID NO: 399
GIFSKLAGKKRGIFSKL.
SEQ ID NO: 400
GIFSPIKGAAKVVAKGLGKEVGKFGLD.
SEQ ID NO: 401
VPPVFTPFRIAPASSLKR.
SEQ ID NO: 402
FLPAIIGMAANVLPTLLCKITKKC.
SEQ ID NO: 403
ILPFLAGLFSKILGK.
SEQ ID NO: 404
FLPALIGMAANVLPTLICKITKKC.
SEQ ID NO: 405
GIFSLIKGAAKVVAKGLGKEVGKFGVD.
SEQ ID NO: 406
GLWTTIKEGVKNVSVGVLDKIRCKITGGC.
SEQ ID NO: 407
GFSPFRVAPASSLKR.
SEQ ID NO: 408
GLWTTIKEGVKNFSVGSYRRSKKR.
SEQ ID NO: 409
GLWTTIKEGVKK.
SEQ ID NO: 410
FLKKTWKKLKKAAKSILKNTGPVTYQQSF.
SEQ ID NO: 411
GILTDTLKGAAKNVAGVLLDKLRCKITGGC.
SEQ ID NO: 412
GIFSLIKGAAKVVAKGLGKEVGKFRRKK.
SEQ ID NO: 413
FLKKTWKKLKKA.
SEQ ID NO: 414
GALKGCWTKSYPPQPCSGKR.
SEQ ID NO: 415
GIFSLIKGAAKVVA.
SEQ ID NO: 416
GLCTTIKEGVNNFSVGVLDKIRCKIT.
SEQ ID NO: 417
FLPAILGMAAKLVPTLLCKITKKC.
SEQ ID NO: 418
GLLGKVLGVGKKMWS.
SEQ ID NO: 419
VIPFVASVATEMMHHVYCAASRRKKR.
SEQ ID NO: 420
RLPGFSPFRVAPASSLKR.
SEQ ID NO: 421
GIFSKLSGKK.
SEQ ID NO: 422
FFPAILRMAAKVVPTVFCQISKKC.
SEQ ID NO: 423
VPPGFTAFRIAPASSLKR.
SEQ ID NO: 424
GLLGKVLGVGKKVLC.
SEQ ID NO: 425
VIPFGASVAAEMMHHVYCAASKRCKK.
SEQ ID NO: 426
GIFSKLAGKKIKDL.
SEQ ID NO: 427
VPPGFTPFRIAPASSLKRVPPGFTPFRIA.
SEQ ID NO: 428
VPPVFTPFRIAPASSLKRVPPGFTPFRIA.
SEQ ID NO: 429
KEERGALKGCWTKSYPPQPCSGKR.
SEQ ID NO: 430
EEERFLPAILGMAAKLVPTLLCKITKKC.
SEQ ID NO: 431
GSSGGRSEKRGIFSLIKGAAKVVAKGL.
SEQ ID NO: 432
VPPGFTPFRIAPASSLKRVPPGFTPFRIAP.
SEQ ID NO: 433
EQKRGIFSKLAGKKIKNLLISGLKNIGKEV.
SEQ ID NO: 434
GLLGKILGVGKRGLLGKILGV.
SEQ ID NO: 435
RNKKRYFLCEQKRGIFSLSLCEQKR.
SEQ ID NO: 436
GLLGKIVGGGLKLIGNHV.
SEQ ID NO: 437
LIDGYMKPLVSVIFSKGCATKY.
SEQ ID NO: 438
GLLTPIGDRGFCKINNSCRN.
SEQ ID NO: 439
HLERMKKSADVIKGIRKY.
SEQ ID NO: 440
IKDHIAPLYKHLTTTALKACGHRA.
SEQ ID NO: 441
CYSFSSLGPSTYLSVRKR.
SEQ ID NO: 442
AFLSTVKNTLTNVAGTMIDTFKCKITGVC.
SEQ ID NO: 443
VPLLPKR.
SEQ ID NO: 444
RRKLLLSAKNKMACFEFAKR.
SEQ ID NO: 445
FISSIASFLGKFLGK.
SEQ ID NO: 446
SLSGCWTKSFPRKPCLRNR.
SEQ ID NO: 447
SAWRRPSRKRRPRR.
SEQ ID NO: 448
SWLRSVPRRQFLRLRR.
SEQ ID NO: 449
FPAIICKVSKN.
SEQ ID NO: 450
KANRKLCGRKRCTSSRDER.
SEQ ID NO: 451
FPAIICKVPKNC.
SEQ ID NO: 452
GLKTHSVSKHFRIHHN.
SEQ ID NO: 453
FFPRVLPLANKFLPTIYCALPKSVGN.
SEQ ID NO: 454
CRMLYVGRTIRALRARFGEHRR.
SEQ ID NO: 455
FPAIICKVSKNC.
SEQ ID NO: 456
CVMCKTSRRRKR.
SEQ ID NO: 457
RDFIGCHTEGVVYILQCSFNLQYVGRTKR.
SEQ ID NO: 458
KIKLFGINSTHRVWRRR.
SEQ ID NO: 459
IRRGKIGPHGCAMGLQKK.
SEQ ID NO: 460
CSGYVPKDILKKKTLKTGKPPWHP.
SEQ ID NO: 461
SLSGCWTKSFPPKPCLRNR.
SEQ ID NO: 462
FLPIIGKILSTVFGKKPRNVETLKMELEII.
SEQ ID NO: 463
GIWDTIKSMGRAFAGRRLENVG.
SEQ ID NO: 464
AGYSRMIRRPPGCTPFRIAPASTLKR.
SEQ ID NO: 465
AISWGRIRRPLWCNPFRIAPASSLKR.
SEQ ID NO: 466
FLGGLIKIAPAMICAVTKKC.
SEQ ID NO: 467
GLKKLESLRPGLKIHT.
SEQ ID NO: 468
GIPFLGKILSPAFGK.
SEQ ID NO: 469
ALLKKLRVKVMEWLCDVMEEWKR.
SEQ ID NO: 470
SLRGGWTKSYPPQPCLGKR.
SEQ ID NO: 471
SLRGCWTKSYP.
SEQ ID NO: 472
FLPFIARLAAKVFPSITCSVTKKC.
SEQ ID NO: 473
ATAWRIPPPGMQPIIPIRIRPLCGKQ.
SEQ ID NO: 474
FLGGLIKIVPAMICAVTKKCTKTNKRPLM.
SEQ ID NO: 475
SILSVLKNLGKVGLGFVACEINKQC.
SEQ ID NO: 476
VESLCRTPSPATISRRKR.
SEQ ID NO: 477
YCRVMQGIAGYCRALQG.
SEQ ID NO: 478
VLLLKILSKR.
SEQ ID NO: 479
CSAQRAGVMGIQSRECR.
SEQ ID NO: 480
GIQGLFDNLKGYYKCGRCRVCRLNSVTSRR.
SEQ ID NO: 481
SFLQNLTGFYRCGRCQVCSLNTNRSRR.
SEQ ID NO: 482
IKDHIAPLYKHLTTTALKACSHRA.
SEQ ID NO: 483
RRLCRFSR.
SEQ ID NO: 484
LRPKSSKR.
SEQ ID NO: 485
GPGTSPPDAGKSARCRLSRKRRPRR.
SEQ ID NO: 486
KTISTFLDRGKVFITALGAKQVKRLRTKR.
SEQ ID NO: 487
FLPFIARLAAEVFPSIICSVTKKC.
SEQ ID NO: 488
GVKTHSVSRHFRLC.
SEQ ID NO: 489
ATIGCALHKSGLYGSMAKRKPLLKESHKK.
SEQ ID NO: 490
HQRLCKRKAYWIFTLGSMTPGGVE.
SEQ ID NO: 491
KLKLKIVDAHKAGYKKIAKR.
SEQ ID NO: 492
GQKWITHKSVYRVQPKITPSSQFKSFLQS.
SEQ ID NO: 493
FSLHPKKWLGQTIKHLQHHRTSPAECD.
SEQ ID NO: 494
ALKRCFTKDNRPIPCFGKR.
SEQ ID NO: 495
GVLQSMGGIAEYGGYCRVRR.
SEQ ID NO: 496
LMVPRKKR.
SEQ ID NO: 497
GLFLDTLKGAAKDVAGKLQEGLKCKITGC.
SEQ ID NO: 498
ATAWRIPPPGMQPIIPIRIRPLCGKR.
SEQ ID NO: 499
GFLDIIKKLGKTFAGHMLDK.
SEQ ID NO: 500
GLKTHYVSKHFRICHNKD.
SEQ ID NO: 501
GIPSLGKILSPAFGK.
SEQ ID NO: 502
GLFLDTLKGAAKDVARKLLEGLK.
SEQ ID NO: 503
LASHLLHIIPVGYNAVFKR.
SEQ ID NO: 504
RLSLLLR.
SEQ ID NO: 505
GSNIVIHCDLVLRASSELNSKRCKR.
SEQ ID NO: 506
SWVWKR.
SEQ ID NO: 507
SLRGCWTKSFPPQPLGKR.
SEQ ID NO: 508
EQERSMLSVLKNLGKVGLGFVACKINKQC.
SEQ ID NO: 509
EEERFLPFIARLAAKVFPSIICSVTKKC.
SEQ ID NO: 510
AMGRVSRAEDRVSRAEKRVKK.
SEQ ID NO: 511
AMDRVSRAEKRVKK.
SEQ ID NO: 512
AGYSRMIRRPPGCTPFRIAPASTLKRAGN.
SEQ ID NO: 513
AISWGRIRRPLWCNPFRIAPASSLKRAGN.
SEQ ID NO: 514
DTSLSMFLSRGIPFLGKILSPAFGK.
SEQ ID NO: 515
DQERSLSGCWTKSFPRKPCLRNR.
SEQ ID NO: 516
EEKRFLPFIARLAAKVFPSIICSVTKKC.
SEQ ID NO: 517
FLFPLITSFLSKFLGK.
SEQ ID NO: 518
SMLSVLKNLGKVGLGFVACKVNKQC.
SEQ ID NO: 519
QPKPR.
SEQ ID NO: 520
ATAWRIPPPGLQPIIPIRIRPLCGKQ.
SEQ ID NO: 521
SLRGCWTKSYPPQKKKKKR.
SEQ ID NO: 522
KKNVWKKKKNVPNPPKR.
SEQ ID NO: 523
FFPRVLPLANKFLPTIYCALPKGVGN.
SEQ ID NO: 524
IRLPHQGHHGKAKAKAVSRSQR.
SEQ ID NO: 525
AGLQFPVGRIHGHLKTR.
SEQ ID NO: 526
GVFLDTLKGLAGKMLGSLKCKIAGCKP.
SEQ ID NO: 527
SSPGMRRRQRWPSPARAQSKR.
SEQ ID NO: 528
AISWGRIRRPPGYTPFRIAPASTLKR.
SEQ ID NO: 529
GVCLDTLKGLAGKMLESLKR.
SEQ ID NO: 530
RLQCMPTVKGALFGGLVKPGSCPIPR.
SEQ ID NO: 531
FLGGLIRIVPAMICAVTKKC.
SEQ ID NO: 532
GLFLDTLKGAAKKRPLFTSSSVIS.
SEQ ID NO: 533
VGMHWSLPTVKVKPGSCPIPR.
SEQ ID NO: 534
GFLDIIKNLGKTFAGHMLDKIRCTIG.
SEQ ID NO: 535
FNRRGWRRR.
SEQ ID NO: 536
ARSWGRIRRPPGCNPFPITPASYLKR.
SEQ ID NO: 537
FLPIIGKILSTVFGKKPRNVETLKMEL.
SEQ ID NO: 538
GIWDTIKSMGRAFAGRRLENV.
SEQ ID NO: 539
AGKPPRRPPVRPPRRPPVRPPRRPRFP.
SEQ ID NO: 540
SLRECWTKSYPPQKKKKKR.
SEQ ID NO: 541
GVFLDTRKGLAGKMLESLKCKIAGCKP.
SEQ ID NO: 542
GKCSSQTSGVPRFATTTIETLWGIVKRKMR.
SEQ ID NO: 543
FFSELTGYFQFRNCPVCSLNGCRSRR.
SEQ ID NO: 544
ILFSAIGNALSRLFGK.
SEQ ID NO: 545
KVSLLKMRAKWRKKR.
SEQ ID NO: 546
AFLSTVKNTLTNVAGTMIDTFKCKVTGVC.
SEQ ID NO: 547
GTPHGQVRPPSRVKPGVCQPPSPECRFIR.
SEQ ID NO: 548
GQCKFYETFAPCQSITRKCHG.
SEQ ID NO: 549
ALPILGGVAAEVLPQVYCRIIRNSKKMN.
SEQ ID NO: 550
TLFRSVCLRKR.
SEQ ID NO: 551
SLRGCWTKSFPPQ.
SEQ ID NO: 552
FLPIIGKILSTVFGKKPRN.
SEQ ID NO: 553
RRWMGRIRR.
SEQ ID NO: 554
GGLQFPVGRIHRHLKTR.
SEQ ID NO: 555
GLFLDTLKGAAKDVALSVSKKRPLFTSS.
SEQ ID NO: 556
FLPFIARLAAKAFPSIICSVTKKC.
SEQ ID NO: 557
AVFSKCSGKAIKNLLIKQL.
SEQ ID NO: 558
SLRGCWTKSYLPQPCLGSCKKITQ.
SEQ ID NO: 559
FKEKKKSKKKWKMEKAWLKPTRKRKRR.
SEQ ID NO: 560
RNAILKR.
SEQ ID NO: 561
FFGGLIKIVPAMICAVTKMLKL.
SEQ ID NO: 562
FLPFIARLAAKVFPSIICSVTKRC.
SEQ ID NO: 563
FLGGLIKIVPAMICAVTKRC.
SEQ ID NO: 564
RRSNIFTGVLLKR.
SEQ ID NO: 565
FLGGLIKIVPAMICAVTKMLKLWKLELEII.
SEQ ID NO: 566
GIPLLGKILSPAFGK.
SEQ ID NO: 567
EQERSMLSVLKNLGKVGLGFVACKVNKQC.
SEQ ID NO: 568
AISWGRIRRPPGYTPFRIAPASTLKRAGN.
SEQ ID NO: 569
ARSWGRIRRPPGCNPFPITPASYLKRAGN.
SEQ ID NO: 570
VARKKRFLGGLIKIVPAMICAVTKKC.
SEQ ID NO: 571
EEERFLPFIARLAAKAFPSIICSVTKKC.
SEQ ID NO: 572
EEERFLPFIARLAAKVFPSIICSVTKRC.
SEQ ID NO: 573
SMMTGGWRKR.
SEQ ID NO: 574
GILTLKYPSLLQCRV.
SEQ ID NO: 575
HPQHLRKGIPKGQFYRIKR.
SEQ ID NO: 576
TKAWFKR.
SEQ ID NO: 577
ILQKELRWIHR.
SEQ ID NO: 578
GILQRNVLHDVRKLGLSRR.
SEQ ID NO: 579
RKWRRGR.
SEQ ID NO: 580
CVYSVINKFKAHGTVANLPRCGRKRKIDKR.
SEQ ID NO: 581
GILEKNVLPCVRKLGLSRR.
SEQ ID NO: 582
GVEAIKGVRKYGKREK.
SEQ ID NO: 583
WGLPKLTLKSLKRFGR.
SEQ ID NO: 584
TGLHFPVGRIHRHFKTR.
SEQ ID NO: 585
SRLWWNRQFLEKYKSNGMIPRGLRVR.
SEQ ID NO: 586
NKFKAHGTVANLPRCGRRRKIDKR.
SEQ ID NO: 587
RGRIQKKLSRR.
SEQ ID NO: 588
HLFHGVLHVKRSVHNKKTQVQ.
SEQ ID NO: 589
KNLKKKLKR.
SEQ ID NO: 590
GTEAIKGVRKYERRVKELTYQVKSSSRHR.
SEQ ID NO: 591
LIASYLKCLIAVVAAKGGPTSY.
SEQ ID NO: 592
GRGKSSPGPLDTRAPPPPKRSPR.
SEQ ID NO: 593
GLLGKVLGVRKKEEMSLMK.
SEQ ID NO: 594
VIPIVSGRLSSLLGK.
SEQ ID NO: 595
GIFSLIKGAAKVVAKGLGKEVGSSGGR.
SEQ ID NO: 596
GLLGKILGVGKKVLCGVSVLC.
SEQ ID NO: 597
VIPFVASVAAEMMHHVYCASSKRCKN.
SEQ ID NO: 598
GLLGKILGVGKKVLCG.
SEQ ID NO: 599
GIFSKLAGKKI.
SEQ ID NO: 600
GLLGKILGVGKKVLCGVSGRC.
SEQ ID NO: 601
GLLGKILGVGKKR.
SEQ ID NO: 602
GILLNTLKGAAKNVAVVLLDKLKCKITGGC.
SEQ ID NO: 603
GLLGKILGVGKKALTPHSTFFP.
SEQ ID NO: 604
GLLGKILGVGKKVLCGGGPPILHP.
SEQ ID NO: 605
GAPKGCWTKSYPPKPCSGIR.
SEQ ID NO: 606
GLWTTIKEGGKLQKK.
SEQ ID NO: 607
GIFSLIKGAAKVVAKPLATTLAA.
SEQ ID NO: 608
GIFSKLAGKKMFTLKKPLLLIVLL.
SEQ ID NO: 609
GLLGKVLGVGKKELCGVS.
SEQ ID NO: 610
GFLSLFSNAAKFLGKTLLKNVGKAGLET.
SEQ ID NO: 611
GIFSKLAGKKIKNLLISETKRGIFSKL.
SEQ ID NO: 612
GLLSGILGAGKKIVC.
SEQ ID NO: 613
GIHLNTLKGAAKNVAG.
SEQ ID NO: 614
HPPGFSPFRVAPASSLKR.
SEQ ID NO: 615
GIFSKLAGKMIKNLLISGLKNIGKEAG.
SEQ ID NO: 616
GIFSKLARKKIKNLLISGLKNIGKEVGMD.
SEQ ID NO: 617
GLLGKVLGVGKKEEMSLMKG.
SEQ ID NO: 618
AIPFVASVAAEMMHHVYCAASKRCKN.
SEQ ID NO: 619
FRFPLLHHHPSLSLCEQKR.
SEQ ID NO: 620
GLLGKILGVGKKVLCGVSG.
SEQ ID NO: 621
GLWTTIKEGVKNFSVGVLDKIRCKI.
SEQ ID NO: 622
DLLGKILGVGKKVLCGV.
SEQ ID NO: 623
VIPFVASVAAEMMHHVYRAASKRCKN.
SEQ ID NO: 624
RLFLFRFPLLHHHPSFAHREIRR.
SEQ ID NO: 625
FISISLKRRRPPGFSPFRVAPASSLKR.
SEQ ID NO: 626
GILLNTLKGAAKNVAGVLIDKLKCKITGGC.
SEQ ID NO: 627
GIPFVASVAAEMMHHVYCAASKRCKN.
SEQ ID NO: 628
GLLGKVLGVGKKVLCGVSGRC.
SEQ ID NO: 629
GILSGLLGAGKKIVCGLSGMC.
SEQ ID NO: 630
GLLDSFKNLALNAAKSAGVSVLNYPSPK.
SEQ ID NO: 631
FFPLLAGLAANFLPKIFCKI.
SEQ ID NO: 632
GLLSGILGAGKK.
SEQ ID NO: 633
KKRSKRVIPIVSGLLSSLLGK.
SEQ ID NO: 634
EQERGLLGKILGVGKKVLCG.
SEQ ID NO: 635
VPPGFTPFRIAPASSLKRVPPGFTPFR.
SEQ ID NO: 636
GILTASHLPISPAPGTPSHHQKNGRRNCR.
SEQ ID NO: 637
GRPRWSHRSRRR.
SEQ ID NO: 638
KEVVAKILKK.
SEQ ID NO: 639
SSESGMTPLAYAAASGHLTIVMLLCKRR.
SEQ ID NO: 640
QSGLSICTYCCKCCQNKQCGYCCRT.
SEQ ID NO: 641
GIPRGQFFRVKR.
SEQ ID NO: 642
GLGKILSAAVKVIGVVYRVLNQ.
SEQ ID NO: 643
GGGSRRGGGGRGGGSRRGGGG.
SEQ ID NO: 644
IKFTCKLSA.
SEQ ID NO: 645
GGGSRRGGGGRGGRASSRSTA.
SEQ ID NO: 646
HSHLSICSFCCNCCKYKGCGWCCLT.
SEQ ID NO: 647
RLALGFRGASVSKR.
SEQ ID NO: 648
GGGSRRGGGGRGGRGGRPGHRCGELPEP.
SEQ ID NO: 649
GGGSRRGGGGRGGRGG.
SEQ ID NO: 650
LIYLMKCRCGAFYVGKTIRPLKKR.
SEQ ID NO: 651
VRKWKTTGTVLVKARSGRPHKILERRRMVR.
SEQ ID NO: 652
FLPGILPAICAATKKC.
SEQ ID NO: 653
WISNIPKSQFCRLRR.
SEQ ID NO: 654
HTKWLTCKPTGPWRPTD.
SEQ ID NO: 655
HTKWRTSRCTGHWRPTETIGALRKMLG.
SEQ ID NO: 656
FIPKLPPLFNKFLPKIYCALPNAVEN.
SEQ ID NO: 657
FLPIFLPLICSVTKKC.
SEQ ID NO: 658
GILTLTNTGKKTQHLNVKL.
SEQ ID NO: 659
ALPILGGVAAEVLPQVYCRIPLKSSGRLHS.
SEQ ID NO: 660
GLGSILKVGAKLLGKTLLKTAGKA.
SEQ ID NO: 661
GGRTPRNSRRGPSREPVSPQLRR.
SEQ ID NO: 662
FLPVVASLATKIRCAVTKKC.
SEQ ID NO: 663
RTLIRCWLNGHRSMRR.
SEQ ID NO: 664
HTIKGIPVGQFLRLKR.
SEQ ID NO: 665
SSKKKKCKFFCKLKKKINSIPIISIPFK.
SEQ ID NO: 666
AVTLLGILKKRLK.
SEQ ID NO: 667
FLPVVASLAAKVLPSIICAVTKKC.
SEQ ID NO: 668
FIPKLPPLFNKFS.
SEQ ID NO: 669
RLNLKLLKR.
SEQ ID NO: 670
GILTLKYPIEHGIITNRGVHCRRCGARSS.
SEQ ID NO: 671
GALRGCWTKSYPPQPCSGKR.
SEQ ID NO: 672
RTLKNKIQKETKKRSAR.
SEQ ID NO: 673
EEQRFLPVVAGLAAKVLPSIICAVTKKC.
SEQ ID NO: 674
EQERGLGSILKVGAKLLGKTLLKTAGKA.
SEQ ID NO: 675
EEERFLPVVASLATKIRCAVTKKC.
SEQ ID NO: 676
DAEEERRFLPVVASLATKIRCAVTKKC.
SEQ ID NO: 677
ALSSALGRPRRIAATCPAILSR.
SEQ ID NO: 678
FIGALVQALSRVLGK.
SEQ ID NO: 679
FWPPFGRR.
SEQ ID NO: 680
FWPFPFGRR.
SEQ ID NO: 681
QSHLSICVHCCNCCKFKGCGKCCLT.
SEQ ID NO: 682
GVGKFLHAAGKFGKALMGEMMKSKR.
SEQ ID NO: 683
AIRGVGKFLHAAGKFGKALMGEMLKSKR.
SEQ ID NO: 684
FVRGMASKAGTIVGKLAKVALNALGRRDS.
SEQ ID NO: 685
GVGKFLHAAGKFGKALMGEMMKSKRWVE.
SEQ ID NO: 686
VIRKSCRR.
SEQ ID NO: 687
HSHLSICVHCCNCCKYKGCGKCCLT.
SEQ ID NO: 688
SSSTGSHGKVVRRILHCCRIS.
SEQ ID NO: 689
HRPGGKETLNDAQPCRR.
SEQ ID NO: 690
GAVPGLPSKGRLPH.
SEQ ID NO: 691
EPEPGNNRPVYIPQPRPPHPRLRREP.
SEQ ID NO: 692
RLIRHKR.
SEQ ID NO: 693
TRPGCGRGRCRWTVCRSDRSRTSCA.
SEQ ID NO: 694
AAGRRWKFGRRRLLGHGVRHT.
SEQ ID NO: 695
GIYTGRLLPVYIPQPRPPHPRLRR.
SEQ ID NO: 696
KNILLKGKR.
SEQ ID NO: 697
EIPHIYKKVKKGKKVKN.
SEQ ID NO: 698
FFFFFFQIIYVPSCNRNLMRIGAIFH.
SEQ ID NO: 699
YARPCAARRPSSRWS.
SEQ ID NO: 700
SSRSVSRTRPGSKR.
SEQ ID NO: 701
ITVTSTFSIFRKCT.
SEQ ID NO: 702
GRQIQRLKLHQSLERARGQR.
SEQ ID NO: 703
YAPWMRKR.
SEQ ID NO: 704
AKIMEKMPRRIKRRRR.
SEQ ID NO: 705
GVVWENLRKIR.
SEQ ID NO: 706
DNKWQNVHFHRSAVTGPSPFSFNHK.
SEQ ID NO: 707
GISTYFVFTRPHGVTLTFHEILKR.
SEQ ID NO: 708
AFVRILCYCCPRKMKRR.
SEQ ID NO: 709
SRSPSKSCKASRKLKSKSPRPR.
SEQ ID NO: 710
KKFPNTPLELPLIRNKLTKR.
SEQ ID NO: 711
IPTTICVCIFIYLRMIYIGTSRSIKR.
SEQ ID NO: 712
GMIHSGGRGR.
SEQ ID NO: 713
GISTYFVFTRPHGIALTFQEILKR.
SEQ ID NO: 714
GTSLVKLISLFLNKSI.
SEQ ID NO: 715
FLSTPKKARMKL.
SEQ ID NO: 716
GFKSQQRGEGREAFKYCVKQEMKAR.
SEQ ID NO: 717
ITFLECVTRVTQAVRRKR.
SEQ ID NO: 718
DNKWQNVHFHRSAVTGPTSFSFSHK.
SEQ ID NO: 719
AVKLRSNTITKKL.
SEQ ID NO: 720
FFGFWKYGEKTAASITLNPPQWRS.
SEQ ID NO: 721
GILRSLGWIQMPRSRRRHR.
SEQ ID NO: 722
DAEPSRPRPQQVPPRPPHPRLRR.
SEQ ID NO: 723
AAGAGKVTKSAQKAQKAK.
SEQ ID NO: 724
RSKRAKKFSGKTLLTG.
SEQ ID NO: 725
RVLTIASRANSRRKKR.
SEQ ID NO: 726
KHHHIKLRHERHRR.
SEQ ID NO: 727
GGGAGGARRSSPPSRRPSSRR.
SEQ ID NO: 728
IQILAPVIRSKR.
SEQ ID NO: 729
VKCRVRR.
SEQ ID NO: 730
ALLKLEKPLKLGGAVQPINLPCIPSTPSGR.
SEQ ID NO: 731
IAFLAVVASAKPYRGFRISLFGTR.
SEQ ID NO: 732
VYLYCLSRGGPSAKPYRGFRISLFDTR.
SEQ ID NO: 733
FLSVVASSLPYRGFRISLFGTR.
SEQ ID NO: 734
IAFLAVVASAKPYRGFRISLFATR.
SEQ ID NO: 735
NIKIPPVLHKVSVPLVSKR.
SEQ ID NO: 736
ISLSSLFKR.
SEQ ID NO: 737
PMAPDISVNIHGSLKKR.
SEQ ID NO: 738
FLAVVASSKPYRGFRISLFATR.
SEQ ID NO: 739
VLSYYKLKSKNIFV.
SEQ ID NO: 740
RAIFTINSLHAIPFAGCTVTGRLTR.
SEQ ID NO: 741
LKLEKPLKLGGAVQPINLPSISSTPSGR.
SEQ ID NO: 742
IQLLAPVIRSKR.
SEQ ID NO: 743
VMLPDKFGNTALHVATRKKR.
SEQ ID NO: 744
KLEKPLKLGGAVQPINLPSIPSTPSGR.
SEQ ID NO: 745
SILSTLSHKR.
SEQ ID NO: 746
GWGLINIKIPPVLHKVSVPLVSKR.
SEQ ID NO: 747
NVKKLPLQAWKRWCTQILSALR.
SEQ ID NO: 748
YPCPVRRIDDRRGHLPHLRPPIL.
SEQ ID NO: 749
VIFYFVTIKFRKKFNLI.
SEQ ID NO: 750
TGWGLTNIKIPPVLHKVSVPLVSKR.
SEQ ID NO: 751
FLAVVASAKPYRGFRIPLFDTR.
SEQ ID NO: 752
FRISLFATR.
SEQ ID NO: 753
FIRTITKCTTTRTSSVSNVSGSGTIQPEH.
SEQ ID NO: 754
RRRTSPGPWTVSLFRR.
SEQ ID NO: 755
IKLTGELPSPMNPPPGCAFNARCRR.
SEQ ID NO: 756
RKRRWNTTFNTCQKMTRFTRRRR.
SEQ ID NO: 757
YRKFIFKR.
SEQ ID NO: 758
KVEHLSKR.
SEQ ID NO: 759
KQKLVKNIIFKYCIKIFIYFLK.
SEQ ID NO: 760
SVSSLAKNSAWPVSLKR.
SEQ ID NO: 761
IPPLIYNLFPNLREMRGRR.
SEQ ID NO: 762
AFVRILCACCPGRVRRR.
SEQ ID NO: 763
IALPTEKLKKFIVRRKWQWPARLRTRGY.
SEQ ID NO: 764
TLVLWRRGFFFCGSSGPASFSRAFYKR.
SEQ ID NO: 765
GGGLTNIKIPPLLHKVSVPLVSKR.
SEQ ID NO: 766
FLSVVASSLHYRGFRISLFDTR.
SEQ ID NO: 767
LKLAKPLKLGGAVQPINLPSIPSTPSGR.
SEQ ID NO: 768
NVKLFLFQLLRGLAYCHRR.
SEQ ID NO: 769
GTKGIINFRIKR.
SEQ ID NO: 770
RARKIRRRRGSLRHCVTIPSTPSGR.
SEQ ID NO: 771
FRISLFGTR.
SEQ ID NO: 772
VRTWAQLVNVEKLLKR.
SEQ ID NO: 773
WMAISKR.
SEQ ID NO: 774
FLSVVASAIHYRGFRISLFDTR.
SEQ ID NO: 775
LKLEKPLKLGGAVQPINLPSIASTPSGR.
SEQ ID NO: 776
GKKLRTLRRHIGMIFQSFNLVKR.
SEQ ID NO: 777
KIRDFRKEVKKVLKR.
SEQ ID NO: 778
KTSSFLTWIGNILPSIPSTPSGR.
SEQ ID NO: 779
GAHKEVFKRDTALTKKASEKPGK.
SEQ ID NO: 780
SRRVKCRVRR.
SEQ ID NO: 781
KHHHIKLRHERHRRYILKSLI.
SEQ ID NO: 782
GAHKEVFKRDTALTKEAAKKAKK.
SEQ ID NO: 783
GAHKEVFKRALAQKLNRNQTKPGKYR.
SEQ ID NO: 784
GAHKEVFKRDTALTKKASQKTGK.
SEQ ID NO: 785
GAHKEVFKRDTALTKQTVKKDNK.
SEQ ID NO: 786
VTISIARRVSSHKR.
SEQ ID NO: 787
PVKLAKCR.
SEQ ID NO: 788
GFKSQQRHKHLQAFTSCVKQEMKSR.
SEQ ID NO: 789
SVASLAKNSAWPVSLKR.
SEQ ID NO: 790
GILRLVTRRFRFSPTNLNRYTVARLVSGVP.
SEQ ID NO: 791
LMWVLSEMILFGKNCKR.
SEQ ID NO: 792
PPHALNTLNSQHHKR.
SEQ ID NO: 793
ATAAECLKHPWLKIKK.
SEQ ID NO: 794
PGSGSGSASRPVYIPPPRPPHPRLRR.
SEQ ID NO: 795
KEVVRVWFCNRRQKEKR.
SEQ ID NO: 796
VTISIARRVSSHKRG.
SEQ ID NO: 797
IIRATAAECLKHPWLKIKK.
SEQ ID NO: 798
RTNIKSFFLSQLRRRR.
SEQ ID NO: 799
GRDKISKR.
SEQ ID NO: 800
NKIKFINKYVKKVQLKKILVKS.
SEQ ID NO: 801
ILLKGKR.
SEQ ID NO: 802
IIGEWRKHKQRKNKLPSKVRVM.
SEQ ID NO: 803
AKMPISVLSKVSDHVKFIKKAAKM.
SEQ ID NO: 804
APEPEPGRPRPQQVPPRPPHPRL.
SEQ ID NO: 805
AKMPISVLSKVSEHVKFIKKAAKM.
SEQ ID NO: 806
GAVHRPYLGVNLRNLSPR.
SEQ ID NO: 807
FAVRDMRQTVAVGVIKSVTFK.
SEQ ID NO: 808
GIFLPGSVILRALSRHGR.
SEQ ID NO: 809
VSSRTICLRVRKKR.
SEQ ID NO: 810
VYIPPPRPPHPRLRR.
SEQ ID NO: 811
SRWLITKKRTRHYSLTRLRNRR.
SEQ ID NO: 812
GFKSQRRHKHLQAFSSCVKQEVKTR.
SEQ ID NO: 813
ITSLSLIRYPADKFVILRKGR.
SEQ ID NO: 814
KLIEWFKVVLQWDPKKR.
SEQ ID NO: 815
RKIIAVSVHKLCRVKR.
SEQ ID NO: 816
RRFFFATAPCGYSRKFCKITRRKR.
SEQ ID NO: 817
FRPPSPKRR.
SEQ ID NO: 818
FFRKPGHGHHKKR.
SEQ ID NO: 819
FIKTQVLKHLVAGVRVARGLDWKWR.
SEQ ID NO: 820
KHHHIKLRHGRHRR.
SEQ ID NO: 821
GRMASGRIIARVLNRHGR.
SEQ ID NO: 822
HIGALARLGWLPSFRAPRFSRSPR.
SEQ ID NO: 823
VGKIPGSFLSGPARRGRR.
SEQ ID NO: 824
DSKWQNVHFHRSAVTGPSPFSFNHK.
SEQ ID NO: 825
IAWSFRFPSMNTLQNSSRRRKR.
SEQ ID NO: 826
FACPIGFFRLKR.
SEQ ID NO: 827
FCKFLRRLGNKKR.
SEQ ID NO: 828
GEFRTVRRQRPIDPRPPRHGPPAKR.
SEQ ID NO: 829
FRPPSPKRRSR.
SEQ ID NO: 830
GVDFTHIRKRMTSWFKR.
SEQ ID NO: 831
KHHHIKLRHGRHRRSVLRTLV.
SEQ ID NO: 832
TVSSLFRRAVNFVGARSRGSRHPSPIRR.
SEQ ID NO: 833
VPFGLKPR.
SEQ ID NO: 834
GLLPLIDPRVCTRSKA.
SEQ ID NO: 835
VALPTDKLKKFIVRRKWQTTKKKSHG.
SEQ ID NO: 836
SPPGKLPRSTRKR.
SEQ ID NO: 837
TPALCAAAKGQLETLKILALHR.
SEQ ID NO: 838
WVPPINELWKKFIKQGTAIWRSGKQ.
SEQ ID NO: 839
RRHAHSYFGEKLKVKR.
SEQ ID NO: 840
YVIKKRWVKAVNTILALQRMGAH.
SEQ ID NO: 841
IWVFLRQKSRRVQFA.
SEQ ID NO: 842
YVIKKRWVKAVNTILALQRMGA.
SEQ ID NO: 843
RSHSLVKR.
SEQ ID NO: 844
IIPIQLGKCFKWGTHDKTGNPRSSR.
SEQ ID NO: 845
GIVFFTLLSKTRRKR.
SEQ ID NO: 846
IVVCYARIFYIVRKTAKRSRR.
SEQ ID NO: 847
LKRHKKQSKKKPSKK.
SEQ ID NO: 848
VCDYPRRAKCEGIGGSGHAKA.
SEQ ID NO: 849
ITICDWAAAVIHKVRSKK.
SEQ ID NO: 850
VALPTDKLKKFIVRRKWQTRKTR.
SEQ ID NO: 851
FPLEQRLKK.
SEQ ID NO: 852
GMPGSNSRTKR.
SEQ ID NO: 853
IAAIFHKR.
SEQ ID NO: 854
VPMRLLSRR.
SEQ ID NO: 855
GIFFFGMSNSLVNPLIYGAFHLWPRRRR.
SEQ ID NO: 856
SMVKLLLSRR.
SEQ ID NO: 857
RSHALVKR.
SEQ ID NO: 858
FFRFPLEQRLKK.
SEQ ID NO: 859
TTRGRRSGFS.
SEQ ID NO: 860
SRSPRKSRSPKLLKSLRKHR.
SEQ ID NO: 861
KASLSLMLGQKSSKK.
SEQ ID NO: 862
PPPKTHQSRIDRPESPRR.
SEQ ID NO: 863
LSALIWLLKRKR.
SEQ ID NO: 864
GAFVLWGPTPRPRRR.
SEQ ID NO: 865
EMMLNNHMKIQRIILEEAAESRLKCRNTR.
SEQ ID NO: 866
STRSRSPRKSRSPKLLKSLRKHR.
SEQ ID NO: 867
VMLPKFKR.
SEQ ID NO: 868
GSKPTTPTGPSPSLPASPAPSRR.
SEQ ID NO: 869
QLWIYWKYGTPRNHLLPR.
SEQ ID NO: 870
GEKRPRAVPAGAGFHLKF.
SEQ ID NO: 871
HKRSKRAVPAGAGFHLKF.
SEQ ID NO: 872
HKRGEKRPRAVPAGAGFHLKF.
SEQ ID NO: 873
HKRAVPAGAGFHLKFW.
SEQ ID NO: 874
GEKRPRAVPAGAGFHLKFW.
SEQ ID NO: 875
HKRSKRAVPAGAGFHLKFW.
SEQ ID NO: 876
HKRGEKRPRAVPAGAGFHLKFW.
SEQ ID NO: 877
GRPNPVNTKPTPYPRLRR.
SEQ ID NO: 878
VIAEIQHITYKEWLPNFLGRRYTR.
SEQ ID NO: 879
AFRHRRTLRTWAASRCGLRACYK.
SEQ ID NO: 880
NKSTSKKKPKKPKKLKK.
SEQ ID NO: 881
RRAIFASIRGYLGLRKR.
SEQ ID NO: 882
KIVKR.
SEQ ID NO: 883
TILTPKRHFSQINVFAKLLKL.
SEQ ID NO: 884
FIKTQVLRHLVAGVRVARGLDWKWR.
SEQ ID NO: 885
SLVGCPRPNFLRSWIRCKFFCKNNKPMCRK.
SEQ ID NO: 886
GFGSGGRCNFHRKRKLIVRPGISKR.
SEQ ID NO: 887
WPAWVYCTRYSDRPSSGRSPRTRRPKR.
SEQ ID NO: 888
RPRKHR.
SEQ ID NO: 889
SSLSPLSSSSGLGKKKKRKSKRASR.
SEQ ID NO: 890
RVSRLRNTLCSWVYRHFVDLRKKRR.
SEQ ID NO: 891
NIGLCITCHNRNFLRR.
SEQ ID NO: 892
TPLSDIFRGQLRSRVSR.
SEQ ID NO: 893
SSKTLPFGKRKSKQR.
SEQ ID NO: 894
DMSSSLIAPPAPPKRLSHKR.
SEQ ID NO: 895
KLFLTLWKLKR.
SEQ ID NO: 896
TIKIIVGTLISKKHVLTAAHCTHD.
SEQ ID NO: 897
IYMYSYCKKGFTGKILIGCAC.
SEQ ID NO: 898
GGTLISKKHVLTAAHCTHDWIMQRRDKR.
SEQ ID NO: 899
GVGRRMGVRHYM.
SEQ ID NO: 900
SKPILKR.
SEQ ID NO: 901
RRLKPR.
SEQ ID NO: 902
VKGFRRFFVKHVFLTSSFA.
SEQ ID NO: 903
NIRPRPTARPPYINRPPNPFKPRWKR.
SEQ ID NO: 904
HIFQVLRGLNFCHNNHIMHR.
SEQ ID NO: 905
LLCGGLRHRR.
SEQ ID NO: 906
GSSSRSCRCIRLSRLSSKRT.
SEQ ID NO: 907
GGGGARHGRQTIWGNIRRNKR.
SEQ ID NO: 908
ERKGVGRRMGVRHYM.
SEQ ID NO: 909
RYHHVKLRHDRHRRSLFSFL.
SEQ ID NO: 910
TRVKGFRRFFVKHVFLTSSFA.
SEQ ID NO: 911
GGGGARHGRGLLAYC.
SEQ ID NO: 912
FLSFLKNKPVTPPPSPCYCSCGLRNEESR.
SEQ ID NO: 913
CRQQCLQPGRSRESCSLPR.
SEQ ID NO: 914
RVSRLRNTLCSWAYRHFVDLRKKRR.
SEQ ID NO: 915
CRQQCLQPGRSRESCSLPRSLFSFL.
SEQ ID NO: 916
AAKLRSNAITKKL.
SEQ ID NO: 917
SKRHLLIAKRRKDLLKSLVEKKLKR.
SEQ ID NO: 918
VRDGTLLKVVHVVRCAESSPR.
SEQ ID NO: 919
GSRRCREAIPRRSASRRAAPLRHFVSDPR.
SEQ ID NO: 920
RTLRFLSVRENLLRR.
SEQ ID NO: 921
AMPYPGTPTNRILQLLKSGYRMERPPNCGR.
SEQ ID NO: 922
ALPLMVGLYRTKQLKKRRR.
SEQ ID NO: 923
SWLARKLLFGAC.
SEQ ID NO: 924
STQSTWGITGGFRFKR.
SEQ ID NO: 925
GRPQFPGYGTFNPKGKLPVPFPKPF.
SEQ ID NO: 926
NGHFASFPHYARARVDTRIFPRRRCCTTT.
SEQ ID NO: 927
VAAFAIIGCLCCRRPRR.
SEQ ID NO: 928
RRPLVSYRCVRRRRRRRR.
SEQ ID NO: 929
TVCKKFTT.
SEQ ID NO: 930
ILLHLLLPSVLPLLLTAIRR.
SEQ ID NO: 931
GVIRYSSYTLKHFLP.
SEQ ID NO: 932
FNRAKRPASRALYAAKIMRKRR.
SEQ ID NO: 933
DVPKLRNHRRAVVRKVVKNHRSKR.
SEQ ID NO: 934
AVLPPFRIPRLRHHR.
SEQ ID NO: 935
FTEPIPHLHQSYSKRRGKR.
SEQ ID NO: 936
ISIKEALEHSFFHTVPRKWCKKH.
SEQ ID NO: 937
HIGSLARQGLLPSIRAARFSR.
SEQ ID NO: 938
GRLWDFIMKNYKLCDHSFKR.
SEQ ID NO: 939
KKKKKKKKKKKKKKQKR.
SEQ ID NO: 940
IKVWFQNRRTKIKKQLTQQ.
SEQ ID NO: 941
FITIIPLFGLLVMNRLSNAFNK.
SEQ ID NO: 942
TALKSLSILKKLAKLNM.
SEQ ID NO: 943
WMLYSRYQNRKALLRRR.
SEQ ID NO: 944
KFYQHLHMLYPNVTRVHVLLLKK.
SEQ ID NO: 945
KTISTRLKR.
SEQ ID NO: 946
GIMFAGRVVQPGVPSVAPGRSF.
SEQ ID NO: 947
FLCTRRENEGRRRRFRR.
SEQ ID NO: 948
GFPKFRGIWN.
SEQ ID NO: 949
VIVVSPRKRAKR.
SEQ ID NO: 950
KYHHIKLRHGRHRR.
SEQ ID NO: 951
IPHPHHLVKR.
SEQ ID NO: 952
FTEPIPHLHQSSSKRRGKR.
SEQ ID NO: 953
LEGSGSASRRRPQQVPPRPPHPRLRR.
SEQ ID NO: 954
SLPKPPQASPRPRIRR.
SEQ ID NO: 955
ITEPVGTKAPTFTSELRGGWLKKR.
SEQ ID NO: 956
RPRPQQVPPRPPHPRLRR.
SEQ ID NO: 957
RNRDHGLASYNDFRKYCHLKR.
SEQ ID NO: 958
ICQIVCIMNIMLILFFFKSFSHSQLKNYLH.
SEQ ID NO: 959
VALPTDKLKKFIMPMKIRMQG.
SEQ ID NO: 960
RPPHPRLRR.
SEQ ID NO: 961
KYHHIKLRHGRHRRTIH.
SEQ ID NO: 962
KYHHIKLRHGRHRRDACFLPKTIH.
SEQ ID NO: 963
HPKKKFDPYVPVRNKTSIRFS.
SEQ ID NO: 964
ATKLVNSAPAKKI.
SEQ ID NO: 965
RRIKR.
SEQ ID NO: 966
SPKKKGGHVSVNVQQEVCLPSC.
SEQ ID NO: 967
FWGARIRR.
SEQ ID NO: 968
WALRWKTR.
SEQ ID NO: 969
SPKKKGGHVSVNVQQDGRHTSW.
SEQ ID NO: 970
LIFSMLPCIVQRTSHHSTRR.
SEQ ID NO: 971
SPKKKGGHVSVNKISHEILRQ.
SEQ ID NO: 972
RAPSLFRGASGVCLISRCSCSHPR.
SEQ ID NO: 973
FSVWALRWKTR.
SEQ ID NO: 974
IKLHEVEKGVVVLKLDLRLFSSR.
SEQ ID NO: 975
TSLLVKR.
SEQ ID NO: 976
AVKLRTSAATKKL.
SEQ ID NO: 977
AALLWSLKKRNRKR.
SEQ ID NO: 978
LSVSHRRPASLPGIALRASR.
SEQ ID NO: 979
FTLQQVKRHPWTICRHPR.
SEQ ID NO: 980
GFKSQQRREGWPAFTFCVKQEMKAP.
SEQ ID NO: 981
AAFGKR.
SEQ ID NO: 982
GTSTVAGSTSSSTPSGRASSKR.
SEQ ID NO: 983
WRASRATIGLQYSLYFFRTLIALTNRRY.
SEQ ID NO: 984
QNMGLRRRSSILSSVTGVRISFWW.
SEQ ID NO: 985
TVLQKR.
SEQ ID NO: 986
FHAVAKIKIPWGKVKDFLVGGMKAVGKK.
SEQ ID NO: 987
FDAVAKIKIPWGKVKDFLVGGMKAVGKK.
SEQ ID NO: 988
HSIGHSIRHSFGHSFGHSFSFRR.
SEQ ID NO: 989
GCRLPRYFWYHRWRLLRDTR.
SEQ ID NO: 990
AAIGRAGRRGTRGGAASRIPR.
SEQ ID NO: 991
FFFFCNFYKDNFKADAIISATRRRSPR.
SEQ ID NO: 992
TLPLMVGRYRAKRRLREKRR.
SEQ ID NO: 993
AVLSFVHKLFLNFLHVDTSKGKCRATLQ.
SEQ ID NO: 994
AFVRILCYCCPRRIKRR.
SEQ ID NO: 995
VPEHARVRGR.
SEQ ID NO: 996
SWLSKSVKKLVNKKNYTRLEKLAKKKLFNE.
SEQ ID NO: 997
VGTLIWILRNRSRRR.
SEQ ID NO: 998
VWSLRKR.
SEQ ID NO: 999
NIGLCIICHNRNFLRR.
SEQ ID NO: 1000
SIKLLPKP.
SEQ ID NO: 1001
LFYKTVLNSSKNIHNRMFSRLVR.
SEQ ID NO: 1002
IFPHTTRFFTFRPLASRSLYSS.
SEQ ID NO: 1003
RHRSLLCVRARTYIDCVRSCEWGHWHRR.
SEQ ID NO: 1004
RNMALAVSRLSRR.
SEQ ID NO: 1005
VIPLFLRVHILPKMKRHFHPALYLYP.
SEQ ID NO: 1006
RRLHKR.
SEQ ID NO: 1007
DIFRSLIWMQMPRSRRRHR.
SEQ ID NO: 1008
VLKLASSKK.
SEQ ID NO: 1009
EADPGSQKPRPPQVPPRPPHPRLRR.
SEQ ID NO: 1010
VTLKPVKR.
SEQ ID NO: 1011
PPHPRLRR.
SEQ ID NO: 1012
RNMALAVSRLSRRGFKVSSN.
SEQ ID NO: 1013
VPSHQSHPRLRRSQIPIPTPVPIRPRPRR.
SEQ ID NO: 1014
RPPHPRLRREAVTES.
SEQ ID NO: 1015
VPSHQSHPRLRRSLGCGGRG.
SEQ ID NO: 1016
GNPGPQPPAKSMNTLVIKKRKKKKRKCFIR.
SEQ ID NO: 1017
VVGQLLGQPKLRHSF.
SEQ ID NO: 1018
GISTYFVFTRPHGITLTFQEILKR.
SEQ ID NO: 1019
VIPLCLRVHILPKMKRHFLPALYLHA.
SEQ ID NO: 1020
STPGKIFKF.
SEQ ID NO: 1021
NKKKIRRRRRRNRRRSRR.
SEQ ID NO: 1022
FILHAKKTRSAK.
SEQ ID NO: 1023
SIRKSRKKKKNYISRKFNLFNSIEYKW.
SEQ ID NO: 1024
DIFRSLIWIQMPRSRRRHR.
SEQ ID NO: 1086
GLLLDTVKGAAKNVAGILLNKLKCKVTGDC.
SEQ ID NO: 1087
GILTDTLKGAAKNVAGVLLDKLKCKITGGC.
SEQ ID NO: 1088
VIPFVASVAAEMMHHVYCAASKRCKN.
SEQ ID NO: 1089
AGYSRMIRRPPGFSPFRVAPASSLKR.
SEQ ID NO: 1090
KIKIPWGKVKDFLVGGMKAVGKK.
SEQ ID NO: 1091
GILSGLLGAGKKIVCGLSGLC.
SEQ ID NO: 1092
GLLSGVLGVGKKIVCGLSGLC.
SEQ ID NO: 1093
GLLSGVLGVGKKVLCGLSGLC.
SEQ ID NO: 1094
GLISGILGAGKKVLCGLSGLC.
SEQ ID NO: 1095
GFMDTAKNVAKNVAVTLLDNLKCKITKAC.
SEQ ID NO: 1096
GIFSLIKGAAKVVAKGLG.
SEQ ID NO: 1097
GILLNTLKGAAKNVAGVLLDKLKCKITGGC.
SEQ ID NO: 1098
FLPVVAGLAAKVLPSIICAVTKKC.
SEQ ID NO: 1099
GIFSLIKGAAKVVAKGLGK.
SEQ ID NO: 1100
GIFSLIKGAAK.
SEQ ID NO: 1101
GAPKGCWTKSYPPKPCSGKR.
SEQ ID NO: 1102
FLPSSPWNEGTYVLKKLKS.
SEQ ID NO: 1103
RMIRRPPGFSPFRVAPASSLKR.

The discovered AMPs have the potential to reduce the morbidity and mortality caused by bacterial infections (especially by antibiotic resistant strains), viral diseases, and cancer. This direct relevance to public health makes the present bioinformatics approach and the resulting discoveries relevant to industrial players. The future applications of the discovery pipeline may expand into the analysis of genomic resources from other studies, including on species from environmental and forestry genomics studies, as well as from metagenomics surveys.

Developing AMPs into therapeutics can involve a variety of delivery modes and forms such as oral, injectable, rectal, topical, transdermal, nasal, or ocular delivery. Using a high throughput methodology to identify a large number of AMPs with therapeutic potential may permit the development of combination compositions. The antimicrobial peptides can be used in combination with more than one providing antimicrobial activity, or in a composition that provides a type of “cocktail” approach, together with other active ingredients.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

The following publications are incorporated by reference herein.
1. Hede K. Antibiotic Resistance: An Infectious Arms Race. Nature. 2014; 509:S2-S3. doi: 10.1038/509S2a.
2. Koo H. B., Seo J. Antimicrobial Peptides under Clinical Investigation. Pept. Sci. 2019; 111:e24122. doi: 10.1002/pep2.24122.
3. Zhang L., Gallo R. L. Antimicrobial Peptides. Curr. Biol. 2016; 26:R14-R19. doi: 10.1016/j.cub.2015.11.017.
4. Andersson D. I., Hughes D., Kubicek-Sutherland J. Z. Mechanisms and Consequences of Bacterial Resistance to Antimicrobial Peptides. Drug Resist. Updat. 2016; 26:43-57. doi: 10.1016/j.drup.2016.04.002.
5. Brandenburg K., Heinbockel L., Correa W., Lohner K. Peptides with Dual Mode of Action: Killing Bacteria and Preventing Endotoxin-Induced Sepsis. Biochim. Biophys. Acta BBA-Biomembr. 2016; 1858:971-979. doi: 10.1016/j.bbamem.2016.01.011.
6. Klotman M. E., Chang T. L. Defensins in Innate Antiviral Immunity. Nat. Rev. Immunol. 2006; 6:447-456. doi: 10.1038/nri1860.
7. De Lucca A. J., Walsh T. J. Antifungal Peptides: Novel Therapeutic Compounds against Emerging Pathogens. Antimicrob. Agents Chemother. 1999; 43:1-11. doi: 10.1128/AAC.43.1.1.
8. Hancock R. E. W., Sahl H. G. Antimicrobial and Host-Defense Peptides as New Anti-Infective Therapeutic Strategies. Nat. Biotechnol. 2006; 24:1551-1557. doi: 10.1038/nbt1267.
9. Moravej H., Moravej Z., Yazdanparast M., Heiat M., Mirhosseini A., Moosazadeh Moghaddam M., Mirnejad R. Antimicrobial Peptides: Features, Action, and Their Resistance Mechanisms in Bacteria. Microb. Drug Resist. 2018; 24:747-767. doi: 10.1089/mdr.2017.0392.
10. Vanhoye D., Bruston F., Nicolas P., Amiche M. Antimicrobial Peptides from Hylid and Ranin Frogs Originated from a 150-Million-Year-Old Ancestral Precursor with a Conserved Signal Peptide but a Hypermutable Antimicrobial Domain. Eur. J. Biochem. 2003; 270:2068-2081. doi: 10.1046/j.1432-1033.2003.03584.x.
11. Helbing C. C., Hammond S. A., Jackman S. H., Houston S., Warren R. L., Cameron C. E., Birol I. Antimicrobial Peptides from Rana [Lithobates] Catesbeiana: Gene Structure and Bioinformatic Identification of Novel Forms from Tadpoles. Sci. Rep. 2019; 9:1529. doi: 10.1038/s41598-018-38442-1.
12. Conlon J. M., Mechkarska M. Host-Defense Peptides with Therapeutic Potential from Skin Secretions of Frogs from the Family Pipidae. Pharmaceuticals. 2014; 7:58-77. doi: 10.3390/ph7010058.
13. Wu Q., Patoc̆ka J., Kuc̆a K. Insect Antimicrobial Peptides, a Mini Review. Toxins. 2018; 10:461. doi: 10.3390/toxins10110461.
14. Sheehan G., Farrell G., Kavanagh K. Immune Priming: The Secret Weapon of the Insect World. Virulence. 2020; 11:238-246. doi: 10.1080/21505594.2020.1731137.
15. Novković M., Simunić J., Bojović V., Tossi A., Juretić D. DADP: The Database of Anuran Defense Peptides. Bioinformatics. 2012; 28:1406-1407. doi: 10.1093/bioinformatics/bts141.
16. Wang G., Li X., Wang Z. APD3: The Antimicrobial Peptide Database as a Tool for Research and Education. Nucleic Acids Res. 2016; 44:D1087-D1093. doi: 10.1093/nar/gkv1278.
17. Zhang R. W., Liu W. T., Geng L. L., Chen X. H., Bi K. S. Quantitative Analysis of a Novel Antimicrobial Peptide in Rat Plasma by Ultra Performance Liquid Chromatography-Tandem Mass Spectrometry. J. Pharm. Anal. 2011; 1:191-196. doi: 10.1016/j.jpha.2011.04.001.
18. Shen W., Chen Y., Yao H., Du C., Luan N., Yan X. A Novel Defensin-like Antimicrobial Peptide from the Skin Secretions of the Tree Frog, Theloderma Kwangsiensis. Gene. 2016; 576:136-140. doi: 10.1016/j.gene.2015.09.086.
19. Pei J., Feng Z., Ren T., Sun H., Han H., Jin W., Dang J., Tao Y. Purification, Characterization and Application of a Novel Antimicrobial Peptide from Andrias Davidianus Blood. Lett. Appl. Microbiol. 2018; 66:38-43. doi: 10.1111/lam.12823.
20. Chen W., Hwang Y. Y., Gleaton J. W., Titus J. K., Hamlin N.J. Optimization of a Peptide Extraction and LC-MS Protocol for Quantitative Analysis of Antimicrobial Peptides. Future Sci. OA. 2019; 5:FS0348. doi: 10.4155/fsoa-2018-0073.
21. Chowdhury T., Mandal S. M., Kumari R., Ghosh A. K. Purification and Characterization of a Novel Antimicrobial Peptide (QAK) from the Hemolymph of Antheraea Mylitta. Biochem. Biophys. Res. Commun. 2020; 527:411-417. doi: 10.1016/j.bbrc.2020.04.050.
22. Amaral A. C., Silva O. N., Mundim N. C. C. R., de Carvalho M. J. A., Migliolo L., Leite J. R. S. A., Prates M. V., Bocca A. L., Franco O. L., Felipe M. S. S. Predicting Antimicrobial Peptides from Eukaryotic Genomes: In Silico Strategies to Develop Antibiotics. Peptides. 2012; 37:301-308. doi: 10.1016/j.peptides.2012.07.021.
23. Prichula J., Primon-Barros M., Luz R. C. Z., Castro í. M. S., Paim T. G. S., Tavares M., Ligabue-Braun R., d'Azevedo P. A., Frazzon J., Frazzon A. P. G., et al. Genome Mining for Antimicrobial Compounds in Wild Marine Animals-Associated Enterococci. Mar. Drugs. 2021; 19:328. doi: 10.3390/md19060328.
24. De la Lastra J. M. P., Garrido-Orduna C., Borges A. A., Jimenez-Arias D., Garcia-Machado F. J., Hernendez M., Gonzelez C., Boto A. Bioinformatics discovery of vertebrate cathelicidins from the mining of available genomes. In: Bobbarala V., editor. Drug Discovery—Concepts to Market. InTech; London, UK: 2018.
25. Tomazou M., Oulas A., Anagnostopoulos A. K., Tsangaris G. T., Spyrou G. M. In Silico Identification of Antimicrobial Peptides in the Proteomes of Goat and Sheep Milk and Feta Cheese. Proteomes. 2019; 7:32. doi: 10.3390/proteomes7040032.
26. Mhade S., Panse S., Tendulkar G., Awate R., Kadam S., Kaushik K. S. AMPing Up the Search: A Structural and Functional Repository of Antimicrobial Peptides for Biofilm Studies, and a Case Study of Its Application to Corynebacterium striatum, an Emerging Pathogen. Front. Cell. Infect. Microbiol. 2021; 11:803774. doi: 10.3389/fcimb.2021.803774.
27. Li C., Sutherland D., Hammond S. A., Yang C., Taho F., Bergman L., Houston S., Warren R. L., Wong T., Hoang L. M. N., et al. AMPlify: Attentive Deep Learning Model for Discovery of Novel Antimicrobial Peptides Effective against WHO Priority Pathogens. BMC Genom. 2022; 23:77. doi: 10.1186/s12864-022-08310-4.
28. Muir P., Li S., Lou S., Wang D., Spakowicz D. J., Salichos L., Zhang J., Weinstock G. M., Isaacs F., Rozowsky J., et al. The Real Cost of Sequencing: Scaling Computation to Keep Pace with Data Generation. Genome Biol. 2016; 17:53. doi: 10.1186/s13059-016-0917-0.
29. NCBI Resource Coordinators Database Resources of the National Center for Biotechnology Information. Nucleic Acids Res. 2016; 44:D7-D19. doi: 10.1093/nar/gkv1290.
30. Guo R., Chen D., Diao Q., Xiong C., Zheng Y., Hou C. Transcriptomic Investigation of Immune Responses of the Apis Cerana Cerana Larval Gut Infected by Ascosphaera Apis. J. Invertebr. Pathol. 2019; 166:107210. doi: 10.1016/j.jip.2019.107210.
31. Li J., Xu X., Xu C., Zhou W., Zhang K., Yu H., Zhang Y., Zheng Y., Rees H. H., Lai R., et al. Anti-Infection Peptidomics of Amphibian Skin. Mol. Cell. Proteom. 2007; 6:882-894. doi: 10.1074/mcp.M600334-MCP200.
32. Song Y., Ji S., Liu W., Yu X., Meng Q., Lai R. Different Expression Profiles of Bioactive Peptides in Pelophylax Nigromaculatus from Distinct Regions. Biosci. Biotechnol. Biochem. 2013; 77:1075-1079. doi: 10.1271/bbb.130044.
33. Wang X., Ren S., Guo C., Zhang W., Zhang X., Zhang B., Li S., Ren J., Hu Y., Wang H. Identification and Functional Analyses of Novel Antioxidant Peptides and Antimicrobial Peptides from Skin Secretions of Four East Asian Frog Species. Acta Biochim. Biophys. Sin. 2017; 49:550-559. doi: 10.1093/abbs/gmx032.
34. Rifflet A., Gavalda S., Tene N., Orivel J., Leprince J., Guilhaudis L., Genin E., Vetillard A., Treilhou M. Identification and Characterization of a Novel Antimicrobial Peptide from the Venom of the Ant Tetramorium Bicarinatum. Peptides. 2012; 38:363-370. doi: 10.1016/j.peptides.2012.08.018.
35. Téné N., Bonnafé E., Berger F., Rifflet A., Guilhaudis L., Ségalas-Milazzo I., Pipy B., Coste A., Leprince J., Treilhou M. Biochemical and Biophysical Combined Study of Bicarinalin, an Ant Venom Antimicrobial Peptide. Peptides. 2016; 79:103-113. doi: 10.1016/j.peptides.2016.04.001.
36. Kaur R., Stoldt M., Jongepier E., Feldmeyer B., Menzel F., Bornberg-Bauer E., Foitzik S. Ant Behaviour and Brain Gene Expression of Defending Hosts Depend on the Ecological Success of the Intruding Social Parasite. Philos. Trans. R. Soc. Lond. B Biol. Sci. 2019; 374:20180192. doi: 10.1098/rstb.2018.0192.
37. Jones P., Binns D., Chang H. Y., Fraser M., Li W., McAnulla C., McWilliam H., Maslen J., Mitchell A., Nuka G., et al. InterProScan 5: Genome-Scale Protein Function Classification. Bioinformatics. 2014; 30:1236-1240. doi: 10.1093/bioinformatics/btu031.
38. Finn R. D., Bateman A., Clements J., Coggill P., Eberhardt R. Y., Eddy S. R., Heger A., Hetherington K., Holm L., Mistry J., et al. Pfam: The Protein Families Database. Nucleic Acids Res. 2014; 42:D222-D230. doi: 10.1093/nar/gkt1223.
39. Sarkar N. Polyadenylation of MRNA in Prokaryotes. Annu. Rev. Biochem. 1997; 66:173-197. doi: 10.1146/annurev.biochem.66.1.173.
40. Wangsanuwat C., Heom K. A., Liu E., O'Malley M. A., Dey S. S. Efficient and Cost-Effective Bacterial MRNA Sequencing from Low Input Samples through Ribosomal RNA Depletion. BMC Genom. 2020; 21:717. doi: 10.1186/s12864-020-07134-4.
41. Sievers F., Wilm A., Dineen D., Gibson T. J., Karplus K., Li W., Lopez R., McWilliam H., Remmert M., Söding J., et al. Fast, Scalable Generation of High-Quality Protein Multiple Sequence Alignments Using Clustal Omega. Mol. Syst. Biol. 2011; 7:539. doi: 10.1038/msb.2011.75.
42. Meher P. K., Sahu T. K., Saini V., Rao A. R. Predicting Antimicrobial Peptides with Improved Accuracy by Incorporating the Compositional, Physico-Chemical and Structural Features into Chou's General PseAAC. Sci. Rep. 2017; 7:42362. doi: 10.1038/srep42362.
43. Xiao X., Wang P., Lin W. Z., Jia J. H., Chou K. C. IAMP-2L: A Two-Level Multi-Label Classifier for Identifying Antimicrobial Peptides and Their Functional Types. Anal. Biochem. 2013; 436:168-177. doi: 10.1016/j.ab.2013.01.019.
44. Veltri D., Kamath U., Shehu A. Deep Learning Improves Antimicrobial Peptide Recognition. Bioinformatics. 2018; 34:2740-2747. doi: 10.1093/bioinformatics/bty179.
45. Das P., Wadhawan K., Chang O., Sercu T., Santos C. D., Riemer M., Chenthamarakshan V., Padhi I., Mojsilovic A. PepCVAE: Semi-Supervised Targeted Design of Antimicrobial Peptide Sequences. arXiv. 2018 doi: 10.48550/ARXIV.1810.07743.1810.07743.
46. Dean S. N., Alvarez J. A. E., Zabetakis D., Walper S. A., Malanoski A. P. PepVAE: Variational Autoencoder Framework for Antimicrobial Peptide Generation and Activity Prediction. Front. Microbiol. 2021; 12:725727. doi: 10.3389/fmicb.2021.725727.
47. Szymczak P., Mozejko M., Grzegorzek T., Bauer M., Neubauer D., Michalski M., Sroka J., Setny P., Kamysz W., Szczurek E. HydrAMP: A Deep Generative Model for Antimicrobial Peptide Discovery. bioRxiv. 2022 doi: 10.1101/2022.01.27.478054.
48. Porto W. F., Pires A. S., Franco O. L. Computational Tools for Exploring Sequence Databases as a Resource for Antimicrobial Peptides. Biotechnol. Adv. 2017; 35:337-349. doi: 10.1016/j.biotechadv.2017.02.001.
49. Ramazi S., Mohammadi N., Allahverdi A., Khalili E., Abdolmaleki P. A Review on Antimicrobial Peptides Databases and the Computational Tools. Database. 2022; 2022:baac011. doi: 10.1093/database/baac011.
50. Aronica P. G. A., Reid L. M., Desai N., Li J., Fox S. J., Yadahalli S., Essex J. W., Verma C. S. Computational Methods and Tools in Antimicrobial Peptide Research. J. Chem. Inf. Model. 2021; 61:3172-3196. doi: 10.1021/acs.jcim.1c00175.
51. Cho J. H., Sung B. H., Kim S. C. Buforins: Histone H2A-Derived Antimicrobial Peptides from Toad Stomach. Biochim. Biophys. Acta BBA-Biomembr. 2009; 1788:1564-1569. doi: 10.1016/j.bbamem.2008.10.025.
52. De Gregorio E., Spellman P. T., Tzou P., Rubin G. M., Lemaitre B. The Toll and Imd Pathways Are the Major Regulators of the Immune Response in Drosophila. EMBO J. 2002; 21:2568-2579. doi: 10.1093/emboj/21.11.2568.
53. Guilhelmelli F., Vilela N., Albuquerque P., Derengowski L. D. S., Silva-Pereira I., Kyaw C. M. Antibiotic Development Challenges: The Various Mechanisms of Action of Antimicrobial Peptides and of Bacterial Resistance. Front. Microbiol. 2013; 4:353. doi: 10.3389/fmicb.2013.00353.
54. Rodriguez-Rojas A., Baeder D. Y., Johnston P., Regoes R. R., Rolff J. Bacteria Primed by Antimicrobial Peptides Develop Tolerance and Persist. PLoS Pathog. 2021; 17:e1009443. doi: 10.1371/journal.ppat.1009443.
55. da Cunha N. B., Cobacho N. B., Viana J. F. C., Lima L. A., Sampaio K. B. O., Dohms S. S. M., Ferreira A. C. R., de la Fuente-Nunez C., Costa F. F., Franco O. L., et al. The next Generation of Antimicrobial Peptides (AMPs) as Molecular Therapeutic Tools for the Treatment of Diseases with Social and Economic Impacts. Drug Discov. Today. 2017; 22:234-248. doi: 10.1016/j.drudis.2016.10.017.
56. Cao J., de la Fuente-Nunez C., Ou R. W., Torres M. D. T., Pande S. G., Sinskey A. J., Lu T. K. Yeast-Based Synthetic Biology Platform for Antimicrobial Peptide Production. ACS Synth. Biol. 2018; 7:896-902. doi: 10.1021/acssynbio.7b00396.
57. Hazam P. K., Goyal R., Ramakrishnan V. Peptide Based Antimicrobials: Design Strategies and Therapeutic Potential. Prog. Biophys. Mol. Biol. 2019; 142:10-22. doi: 10.1016/j.pbiomolbio.2018.08.006.
58. Hirano M., Saito C., Goto C., Yokoo H., Kawano R., Misawa T., Demizu Y. Rational Design of Helix-Stabilized Antimicrobial Peptide Foldamers Containing α,α-Disubstituted AAs or Side-Chain Stapling. ChemPlusChem. 2020; 85:2731-2736. doi: 10.1002/cplu.202000749.
59. Leinonen R., Sugawara H., Shumway M., On behalf of the International Nucleotide Sequence Database Collaboration Sequence Read Archive. Nucleic Acids Res. 2011; 39:D19-D21. doi: 10.1093/nar/gkq1019.
60. Chen S., Zhou Y., Chen Y., Gu J. Fastp: An Ultra-Fast All-in-One FASTQ Preprocessor. Bioinformatics. 2018; 34:i884-i890. doi: 10.1093/bioinformatics/bty560.
61. Nip K. M., Chiu R., Yang C., Chu J., Mohamadi H., Warren R. L., Birol I. RNA-Bloom Enables Reference-Free and Reference-Guided Sequence Assembly for Single-Cell Transcriptomes. Genome Res. 2020; 30:1191-1200. doi: 10.1101/gr.260174.119.
62. Patro R., Duggal G., Love M. I., Irizarry R. A., Kingsford C. Salmon Provides Fast and Bias-Aware Quantification of Transcript Expression. Nat. Methods. 2017; 14:417-419. doi: 10.1038/nmeth.4197.
63. Haas B. J., Papanicolaou A., Yassour M., Grabherr M., Blood P. D., Bowden J., Couger M. B., Eccles D., Li B., Lieber M., et al. De Novo Transcript Sequence Reconstruction from RNA-Seq Using the Trinity Platform for Reference Generation and Analysis. Nat. Protoc. 2013; 8:1494-1512. doi: 10.1038/nprot.2013.084.
64. Johnson L. S., Eddy S. R., Portugaly E. Hidden Markov Model Speed Heuristic and Iterative HMM Search Procedure. BMC Bioinform. 2010; 11:431. doi: 10.1186/1471-2105-11-431.
65. Finn R. D., Clements J., Eddy S. R. HMMER Web Server: Interactive Sequence Similarity Searching. Nucleic Acids Res. 2011; 39:W29-W37. doi: 10.1093/nar/gkr367.
66. Duckert P., Brunak S., Blom N. Prediction of Proprotein Convertase Cleavage Sites. Protein Eng. Des. Sel. 2004; 17:107-112. doi: 10.1093/protein/gzh013.
67. Wang X., Song Y., Li J., Liu H., Xu X., Lai R., Zhang K. A New Family of Antimicrobial Peptides from Skin Secretions of Rana Pleuraden. Peptides. 2007; 28:2069-2074. doi: 10.1016/j.peptides.2007.07.020.
68. Yi H. Y., Chowdhury M., Huang Y. D., Yu X. Q. Insect Antimicrobial Peptides and Their Applications. Appl. Microbiol. Biotechnol. 2014; 98:5807-5822. doi: 10.1007/s00253-014-5792-6.
69. Jiang Z., Vasil A. I., Hale J. D., Hancock R. E. W., Vasil M. L., Hodges R. S. Effects of Net Charge and the Number of Positively Charged Residues on the Biological Activity of Amphipathic Alpha-Helical Cationic Antimicrobial Peptides. Biopolymers. 2008; 90:369-383. doi: 10.1002/bip.20911.
70. Hart A. J., Ginzburg S., Xu M., Fisher C. R., Rahmatpour N., Mitton J. B., Paul R., Wegrzyn J. L. EnTAP: Bringing Faster and Smarter Functional Annotation to Non-model Eukaryotic Transcriptomes. Mol. Ecol. Resour. 2020; 20:591-604. doi: 10.1111/1755-0998.13106.
71. The UniProt Consortium. Bateman A., Martin M. J., Orchard S., Magrane M., Agivetova R., Ahmad S., Alpi E., Bowler-Barnett E. H., Britto R., et al. UniProt: The Universal Protein Knowledgebase in 2021. Nucleic Acids Res. 2021; 49:D480-D489. doi: 10.1093/nar/gkaa 1100.
72. O'Leary N. A., Wright M. W., Brister J. R., Ciufo S., Haddad D., McVeigh R., Rajput B., Robbertse B., Smith-White B., Ako-Adjei D., et al. Reference Sequence (RefSeq) Database at NCBI: Current Status, Taxonomic Expansion, and Functional Annotation. Nucleic Acids Res. 2016; 44:D733-D745. doi: 10.1093/nar/gkv1189.
73. Slater G., Birney E. Automated Generation of Heuristics for Biological Sequence Comparison. BMC Bioinform. 2005; 6:31. doi: 10.1186/1471-2105-6-31.
74. Adamczak R., Porollo A., Meller J. Combining Prediction of Secondary Structure and Solvent Accessibility in Proteins. Proteins. 2005; 59:467-475. doi: 10.1002/prot.20441.
75. Fu L., Niu B., Zhu Z., Wu S., Li W. CD-HIT: Accelerated for Clustering the next-Generation Sequencing Data. Bioinformatics. 2012; 28:3150-3152. doi: 10.1093/bioinformatics/bts565.
76. Wiegand I., Hilpert K., Hancock R. E. W. Agar and Broth Dilution Methods to Determine the Minimal Inhibitory Concentration (MIC) of Antimicrobial Substances. Nat. Protoc. 2008; 3:163-175. doi: 10.1038/nprot.2007.521.
77. Sanchez E., Rodriguez A., Grau J. H., Lötters S., Kunzel S., Saporito R. A., Ringler E., Schulz S., Wollenberg Valero K. C., Vences M. Transcriptomic Signatures of Experimental Alkaloid Consumption in a Poison Frog. Genes. 2019; 10:733. doi: 10.3390/genes10100733.
78. Siu-Ting K., Torres-Senchez M., San Mauro D., Wilcockson D., Wilkinson M., Pisani D., O'Connell M. J., Creevey C. J. Inadvertent Paralog Inclusion Drives Artifactual Topologies and Timetree Estimates in Phylogenomics. Mol. Biol. Evol. 2019; 36:1344-1356. doi: 10.1093/molbev/msz067.
79. Xia Y., Luo W., Yuan S., Zheng Y., Zeng X. Microsatellite Development from Genome Skimming and Transcriptome Sequencing: Comparison of Strategies and Lessons from Frog Species. BMC Genom. 2018; 19:886. doi: 10.1186/s12864-018-5329-y.
80. Fan W., Jiang Y., Zhang M., Yang D., Chen Z., Sun H., Lan X., Yan F., Xu J., Yuan W. Comparative Transcriptome Analyses Reveal the Genetic Basis Underlying the Immune Function of Three Amphibians' Skin. PLoS ONE. 2017; 12:e0190023. doi: 10.1371/journal.pone.0190023.
81. Reilly B. D., Schlipalius D. I., Cramp R. L., Ebert P. R., Franklin C. E. Frogs and Estivation: Transcriptional Insights into Metabolism and Cell Survival in a Natural Model of Extended Muscle Disuse. Physiol. Genom. 2013; 45:377-388. doi: 10.1152/physiolgenomics.00163.2012.
82. Liscano Martinez Y., Arenas Gómez C. M., Smith J., Delgado J. P. A Tree Frog (Boana Pugnax) Dataset of Skin Transcriptome for the Identification of Biomolecules with Potential Antimicrobial Activities. Data Brief. 2020; 32:106084. doi: 10.1016/j.dib.2020.106084.
83. Grogan L. F., Mulvenna J., Gummer J. P. A., Scheele B. C., Berger L., Cashins S. D., McFadden M. S., Harlow P., Hunter D. A., Trengove R. D., et al. Survival, Gene and Metabolite Responses of Litoria Verreauxii Alpina Frogs to Fungal Disease Chytridiomycosis. Sci. Data. 2018; 5:180033. doi: 10.1038/sdata.2018.33.
84. Qiao L., Yang W., Fu J., Song Z. Transcriptome Profile of the Green Odorous Frog (Odorrana Margaretae) PLoS ONE. 2013; 8:e75211. doi: 10.1371/journal.pone.0075211.
85. Chang L., Zhu W., Shi S., Zhang M., Jiang J., Li C., Xie F., Wang B. Plateau Grass and Greenhouse Flower?Distinct Genetic Basis of Closely Related Toad Tadpoles Respectively Adapted to High Altitude and Karst Caves. Genes. 2020; 11:123. doi: 10.3390/genes 11020123.
86. Caty S. N., Alvarez-Buylla A., Byrd G. D., Vidoudez C., Roland A. B., Tapia E. E., Budnik B., Trauger S. A., Coloma L. A., O'Connell L. A. Molecular Physiology of Chemical Defenses in a Poison Frog. J. Exp. Biol. 2019; 222:jeb.204149. doi: 10.1242/jeb.204149.
87. Shu Y., Xia J., Yu Q., Wang G., Zhang J., He J., Wang H., Zhang L., Wu H. Integrated Analysis of MRNA and MiRNA Expression Profiles Reveals Muscle Growth Differences between Adult Female and Male Chinese Concave-Eared Frogs (Odorrana Tormota) Gene. 2018; 678:241-251. doi: 10.1016/j.gene.2018.08.007.
88. ( ) Yoshida N., Kaito C. Dataset for de Novo Transcriptome Assembly of the African Bullfrog Pyxicephalus Adspersus. Data Brief. 2020; 30:105388. doi: 10.1016/j.dib.2020.105388.
89. Bossuyt F., Schulte L. M., Maex M., Janssenswillen S., Novikova P. Y., Biju S. D., Van de Peer Y., Matthijs S., Roelants K., Martel A., et al. Multiple Independent Recruitment of Sodefrin Precursor-Like Factors in Anuran Sexually Dimorphic Glands. Mol. Biol. Evol. 2019; 36:1921-1930. doi: 10.1093/molbev/msz115.
90. Zhang Y., Li Y., Qin Z., Wang H., Li J. A Screening Assay for Thyroid Hormone Signaling Disruption Based on Thyroid Hormone-Response Gene Expression Analysis in the Frog Pelophylax Nigromaculatus. J. Environ. Sci. 2015; 34:143-154. doi: 10.1016/j.jes.2015.01.028.
91. Eskew E. A., Shock B. C., LaDouceur E. E. B., Keel K., Miller M. R., Foley J. E., Todd B. D. Gene Expression Differs in Susceptible and Resistant Amphibians Exposed to Batrachochytrium Dendrobatidis. R. Soc. Open sci. 2018; 5:170910. doi: 10.1098/rsos.170910.
92. Stuckert A. M. M., Chouteau M., McClure M., LaPolice T. M., Linderoth T., Nielsen R., Summers K., MacManes M. D. The Genomics of Mimicry: Gene Expression throughout Development Provides Insights into Convergent and Divergent Phenotypes in a Müllerian Mimicry System. Mol. Ecol. 2021; 30:4039-4061. doi: 10.1111/mec.16024.
93. Christenson M. K., Trease A. J., Potluri L. P., Jezewski A. J., Davis V. M., Knight L. A., Kolok A. S., Davis P. H. De Novo Assembly and Analysis of the Northern Leopard Frog Rana Pipiens Transcriptome. J. Genom. 2014; 2:141-149. doi: 10.7150/jgen.9760.
94. Price S. J., Garner T. W. J., Balloux F., Ruis C., Paszkiewicz K. H., Moore K., Griffiths A. G. F. A de Novo Assembly of the Common Frog (Rana Temporaria) Transcriptome and Comparison of Transcription Following Exposure to Ranavirus and Batrachochytrium Dendrobatidis. PLoS ONE. 2015; 10:e0130500. doi: 10.1371/journal.pone.0130500.
95. Furman B. L. S., Evans B. J. Sequential Turnovers of Sex Chromosomes in African Clawed Frogs (Xenopus) Suggest Some Genomic Regions Are Good at Sex Determination. G3 (Bethesda) 2016; 6:3625-3633. doi: 10.1534/g3.116.033423.
96. Birol I., Behsaz B., Hammond S. A., Kucuk E., Veldhoen N., Helbing C. C. De Novo Transcriptome Assemblies of Rana (Lithobates) Catesbeiana and Xenopus Laevis Tadpole Livers for Comparative Genomics without Reference Genomes. PLoS ONE. 2015; 10:e0130720. doi: 10.1371/journal.pone.0130720.
97. Barbosa-Morais N. L., Irimia M., Pan Q., Xiong H. Y., Gueroussov S., Lee L. J., Slobodeniuc V., Kutter C., Watt S., Colak R., et al. The Evolutionary Landscape of Alternative Splicing in Vertebrate Species. Science. 2012; 338:1587-1593. doi: 10.1126/science.1230612.
98. Arvidson R., Kaiser M., Lee S. S., Urenda J. P., Dail C., Mohammed H., Nolan C., Pan S., Stajich J. E., Libersat F., et al. Parasitoid Jewel Wasp Mounts Multipronged Neurochemical Attack to Hijack a Host Brain. Mol. Cell. Proteom. 2019; 18:99-114. doi: 10.1074/mcp.RA118.000908.
99. Yek S. H., Boomsma J. J., Schiott M. Differential Gene Expression in Acromyrmex Leaf-Cutting Ants after Challenges with Two Fungal Pathogens. Mol. Ecol. 2013; 22:2173-2187. doi: 10.1111/mec.12255.
100. Yoon K. A., Kim K., Kim W. J., Bang W. Y., Ahn N. H., Bae C. H., Yeo J. H., Lee S. H. Characterization of Venom Components and Their Phylogenetic Properties in Some Aculeate Bumblebees and Wasps. Toxins. 2020; 12:47. doi: 10.3390/toxins12010047.
101. McNamara-Bordewick N. K., McKinstry M., Snow J. W. Robust Transcriptional Response to Heat Shock Impacting Diverse Cellular Processes despite Lack of Heat Shock Factor in Microsporidia. mSphere. 2019; 4:e00219-19. doi: 10.1128/mSphere.00219-19.
102. Becchimanzi A., Avolio M., Bostan H., Colantuono C., Cozzolino F., Mancini D., Chiusano M. L., Pucci P., Caccia S., Pennacchio F. Venomics of the Ectoparasitoid Wasp Bracon Nigricans. BMC Genom. 2020; 21:34. doi: 10.1186/s12864-019-6396-4.
103. de Bekker C., Ohm R. A., Loreto R. G., Sebastian A., Albert I., Merrow M., Brachmann A., Hughes D. P. Gene Expression during Zombie Ant Biting Behavior Reflects the Complexity Underlying Fungal Parasitic Behavioral Manipulation. BMC Genom. 2015; 16:620. doi: 10.1186/s12864-015-1812-x.
104. von Wyschetzki K., Lowack H., Heinze J. Transcriptomic Response to Injury Sheds Light on the Physiological Costs of Reproduction in Ant Queens. Mol. Ecol. 2016; 25:1972-1985. doi: 10.1111/mec.13588.
105. Zhao W., Shi M., Ye X., Li F., Wang X., Chen X. Comparative Transcriptome Analysis of Venom Glands from Cotesia Vestalis and Diadromus Collaris, Two Endoparasitoids of the Host Plutella Xylostella. Sci. Rep. 2017; 7:1298. doi: 10.1038/s41598-017-01383-2.
106. Coffman K. A., Harrell T. C., Burke G. R. A Mutualistic Poxvirus Exhibits Convergent Evolution with Other Heritable Viruses in Parasitoid Wasps. J. Virol. 2020; 94:e02059-19. doi: 10.1128/JVI.02059-19.
107. Burke G. R., Strand M. R. Systematic Analysis of a Wasp Parasitism Arsenal. Mol. Ecol. 2014; 23:890-901. doi: 10.1111/mec.12648.
108. Robinson S. D., Mueller A., Clayton D., Starobova H., Hamilton B. R., Payne R. J., Vetter I., King G. F., Undheim E. A. B. A Comprehensive Portrait of the Venom of the Giant Red Bull Ant, Myrmecia Gulosa, Reveals a Hyperdiverse Hymenopteran Toxin Gene Family. Sci. Adv. 2018; 4:eaau4640. doi: 10.1126/sciadv.aau4640.
109. Martinson E. O., Mrinalini, Kelkar Y. D., Chang C. H., Werren J. H. The Evolution of Venom by Co-Option of Single-Copy Genes. Curr. Biol. 2017; 27:2007-2013. doi: 10.1016/j.cub.2017.05.032.
110. Cook N., Boulton R. A., Green J., Trivedi U., Tauber E., Pannebakker B. A., Ritchie M. G., Shuker D. M. Differential Gene Expression Is Not Required for Facultative Sex Allocation: A Transcriptome Analysis of Brain Tissue in the Parasitoid Wasp Nasonia vitripennis. R. Soc. Open sci. 2018; 5:171718. doi: 10.1098/rsos.171718.
111. Sim A. D., Wheeler D. The Venom Gland Transcriptome of the Parasitoid Wasp Nasonia Vitripennis Highlights the Importance of Novel Genes in Venom Function. BMC Genom. 2016; 17:571. doi: 10.1186/s12864-016-2924-7.
112. Kazuma K., Masuko K., Konno K., Inagaki H. Combined Venom Gland Transcriptomic and Venom Peptidomic Analysis of the Predatory Ant Odontomachus Monticola. Toxins. 2017; 9:323. doi: 10.3390/toxins9100323.
113. Smith C. R., Helms Cahan S., Kemena C., Brady S. G., Yang W., Bornberg-Bauer E., Eriksson T., Gadau J., Helmkampf M., Gotzek D., et al. How Do Genomes Create Novel Phenotypes?Insights from the Loss of the Worker Caste in Ant Social Parasites. Mol. Biol. Evol. 2015; 32:2919-2931. doi: 10.1093/molbev/msv165.
114. Özbek R., Wielsch N., Vogel H., Lochnit G., Foerster F., Vilcinskas A., von Reumont B. M. Proteo-Transcriptomic Characterization of the Venom from the Endoparasitoid Wasp Pimpla Turionellae with Aspects on Its Biology and Evolution. Toxins. 2019; 11:721. doi: 10.3390/toxins11120721.
115. Yang L., Yang Y., Liu M. M., Yan Z. C., Qiu L. M., Fang Q., Wang F., Werren J. H., Ye G. Y. Identification and Comparative Analysis of Venom Proteins in a Pupal Ectoparasitoid, Pachycrepoideus Vindemmiae. Front. Physiol. 2020; 11:9. doi: 10.3389/fphys.2020.00009.
116. Bouzid W., Verdenaud M., Klopp C., Ducancel F., Noirot C., Vetillard A. De Novo Sequencing and Transcriptome Analysis for Tetramorium Bicarinatum: A Comprehensive Venom Gland Transcriptome Analysis from an Ant Species. BMC Genom. 2014; 15:987. doi: 10.1186/1471-2164-15-987.
117. Negroni M. A., Foitzik S., Feldmeyer B. Long-Lived Temnothorax Ant Queens Switch from Investment in Immunity to Antioxidant Production with Age. Sci. Rep. 2019; 9:7270. doi: 10.1038/s41598-019-43796-1.
118. Johnson, M.; Zaretskaya, I.; Raytselis, Y.; Merezhuk, Y.; McGinnis, S.; Madden, T. L. NCBI BLAST: A Better Web Interface. Nucleic Acids Research 2008, 36, W5-W9, doi:10.1093/nar/gkn201.
119. Lin D., Sutherland D., Aninta S. I., Louie N., Nip K. M., Li C., Yanai A., Coombe L., Warren R. L., Helbing C. C., et al., Mining Amphibian and Insect Transcriptomes for Antimicrobial Peptide Sequences with rAMPage, Antibiotics (Basel), 2022 Jul 15; 11(7):952. doi: 10.3390/antibiotics11070952.
120. Murray C. J., Ikuta K. S., Sharara F., Swetschinski L., Robles Aguilar G., Gray A., Han C., Bisignano C., Rao P., Wool E., et al. Global Burden of Bacterial Antimicrobial Resistance in 2019: A Systematic Analysis. Lancet. 2022; 399:629-655. doi: 10.1016/S0140-6736(21)02724-0.
121. Gillings M. R., Paulsen I. T., Tetu S. G. Genomics and the Evolution of Antibiotic Resistance: Genomics and Antibiotic Resistance. Ann. N.Y. Acad. Sci. 2017; 1388:92-107. doi: 10.1111/nyas.13268.
122. Llor C., Bjerrum L. Antimicrobial Resistance: Risk Associated with Antibiotic Overuse and Initiatives to Reduce the Problem. Ther. Adv. Drug Saf. 2014; 5:229-241. doi: 10.1177/2042098614554919.
123. Durand G. A., Raoult D., Dubourg G. Antibiotic Discovery: History, Methods and Perspectives. Int. J. Antimicrob. Agents. 2019; 53:371-382. doi: 10.1016/j.ijantimicag.2018.11.010.
124. Cruz J., Ortiz C., Guzmen F., Fernendez-Lafuente R., Torres R. Antimicrobial Peptides: Promising Compounds against Pathogenic Microorganisms. CMC. 2014; 21:2299-2321. doi: 10.2174/0929867321666140217110155.
125. Zasloff M. Antimicrobial Peptides of Multicellular Organisms. Nature. 2002; 415:389-395. doi: 10.1038/415389a.
126. Petchiappan A., Chatterji D. Antibiotic Resistance: Current Perspectives. ACS Omega. 2017; 2:7400-7409. doi: 10.1021/acsomega.7b01368.
127. Rima M., Rima M., Fajloun Z., Sabatier J. M., Bechinger B., Naas T. Antimicrobial Peptides: A Potent Alternative to Antibiotics. Antibiotics. 2021; 10:1095. doi: 10.3390/antibiotics10091095.
128. Yu G., Baeder D. Y., Regoes R. R., Rolff J. Predicting Drug Resistance Evolution: Insights from Antimicrobial Peptides and Antibiotics. Proc. R. Soc. B. 2018; 285:20172687. doi: 10.1098/rspb.2017.2687.
129. Meylan S., Andrews I. W., Collins J. J. Targeting Antibiotic Tolerance, Pathogen by Pathogen. Cell. 2018; 172:1228-1238. doi: 10.1016/j.cell.2018.01.037.
130. Koehbach J., Craik D. J. The Vast Structural Diversity of Antimicrobial Peptides. Trends Pharmacol. Sci. 2019; 40:517-528. doi: 10.1016/j.tips.2019.04.012.
131. Mahlapuu M., Hakansson J., Ringstad L., Bjorn C. Antimicrobial Peptides: An Emerging Category of Therapeutic Agents. Front. Cell. Infect. Microbiol. 2016; 6:194. doi: 10.3389/fcimb.2016.00194.
132. Tossi A., Sandri L., Giangaspero A. Amphipathic, α-Helical Antimicrobial Peptides. Biopolymers. 2000; 55:4-30. doi: 10.1002/1097-0282(2000)55:1<4::AID-BIP30>3.0.CO; 2-M.
133. Chen C. H., Bepler T., Pepper K., Fu D., Lu T. K. Synthetic Molecular Evolution of Antimicrobial Peptides. Curr. Opin. Biotechnol. 2022; 75:102718. doi: 10.1016/j.copbio.2022.102718.
134. Jumper J., Evans R., Pritzel A., Green T., Figurnov M., Ronneberger O., Tunyasuvunakool K., Bates R., Z̆idek A., Potapenko A., et al. Highly Accurate Protein Structure Prediction with AlphaFold. Nature. 2021; 596:583-589. doi: 10.1038/s41586-021-03819-2.
135. Mirdita M., SchQtze K., Moriwaki Y., Heo L., Ovchinnikov S., Steinegger M. ColabFold: Making Protein Folding Accessible to All. Nat. Methods. 2022; 19:679-682. doi: 10.1038/s41592-022-01488-1.
136. Frishman D., Argos P. Knowledge-Based Protein Secondary Structure Assignment. Proteins. 1995; 23:566-579. doi: 10.1002/prot.340230412.
137. Wiegand I., Hilpert K., Hancock R. E. W. Agar and Broth Dilution Methods to Determine the Minimal Inhibitory Concentration (MIC) of Antimicrobial Substances. Nat. Protoc. 2008; 3:163-175. doi: 10.1038/nprot.2007.521.
138. NCBI Resource Coordinators Database Resources of the National Center for Biotechnology Information. Nucleic Acids Res. 2016; 44:D7-D19.
139. Li W. F., Ma G. X., Zhou X. X. Apidaecin-Type Peptides: Biodiversity, Structure-Function Relationships and Mode of Action. Peptides. 2006; 27:2350-2359. doi: 10.1016/j.peptides.2006.03.016.
140. Greco I., Molchanova N., Holmedal E., Jenssen H., Hummel B. D., Watts J. L., Hakansson J., Hansen P. R., Svenson J. Correlation between Hemolytic Activity, Cytotoxicity and Systemic in Vivo Toxicity of Synthetic Antimicrobial Peptides. Sci. Rep. 2020; 10:13206. doi: 10.1038/s41598-020-69995-9.
141. Maher S., McClean S. Investigation of the Cytotoxicity of Eukaryotic and Prokaryotic Antimicrobial Peptides in Intestinal Epithelial Cells in Vitro. Biochem. Pharmacol. 2006; 71:1289-1298. doi: 10.1016/j.bcp.2006.01.012.
142. Maturana P., Martinez M., Noguera M. E., Santos N.C., Disalvo E. A., Semorile L., Maffia P. C., Hollmann A. Lipid Selectivity in Novel Antimicrobial Peptides: Implication on Antimicrobial and Hemolytic Activity. Colloids Surf. B Biointerfaces. 2017; 153:152-159. doi: 10.1016/j.colsurfb.2017.02.003.
143. Ilid N., Novkovid M., Guida F., Xhindoli D., Benincasa M., Tossi A., Juretid D. Selective Antimicrobial Activity and Mode of Action of Adepantins, Glycine-Rich Peptide Antibiotics Based on Anuran Antimicrobial Peptide Sequences. Biochim. Biophys. Acta (BBA)-Biomembr. 2013; 1828:1004-1012. doi: 10.1016/j.bbamem.2012.11.017.
144. Conlon J. M. Reflections on a Systematic Nomenclature for Antimicrobial Peptides from the Skins of Frogs of the Family Ranidae. Peptides. 2008; 29:1815-1819. doi: 10.1016/j.peptides.2008.05.029.
145. Park S., Park S. H., Ahn H. C., Kim S., Kim S. S., Lee B. J., Lee B. J. Structural Study of Novel Antimicrobial Peptides, Nigrocins, Isolated from Rana Nigromaculata. FEBS Lett. 2001; 507:95-100. doi: 10.1016/S0014-5793(01)02956-8.
146. Conlon J. M., Ahmed E., Condamine E. Antimicrobial Properties of Brevinin-2-Related Peptide and Its Analogs: Efficacy Against Multidrug-Resistant Acinetobacter Baumannii. Chem. Biol. Drug Des. 2009; 74:488-493. doi: 10.1111/j.1747-0285.2009.00882.x.
147. Wang G. Post-Translational Modifications of Natural Antimicrobial Peptides and Strategies for Peptide Engineering. CBIOT. 2012; 1:72-79. doi: 10.2174/2211550111201010072.
148. Strandberg E., Tiltak D., leronimo M., Kanithasen N., Wadhwani P., Ulrich A. S. Influence of C-Terminal Amidation on the Antimicrobial and Hemolytic Activities of Cationic α-Helical Peptides. Pure Appl. Chem. 2007; 79:717-728. doi: 10.1351/pac200779040717.
149. Mangoni M. L., Papo N., Mignogna G., Andreu D., Shai Y., Barra D., Simmaco M. Ranacyclins, a New Family of Short Cyclic Antimicrobial Peptides: Biological Function, Mode of Action, and Parameters Involved in Target Specificity. Biochemistry. 2003; 42:14023-14035. doi: 10.1021/bi0345211.
150. Dong R., Peng Z., Zhang Y., Yang J. MTM-Align: An Algorithm for Fast and Accurate Multiple Protein Structure Alignment. Bioinformatics. 2018; 34:1719-1725. doi: 10.1093/bioinformatics/btx828.
151. Virtanen P., Gommers R., Oliphant T. E., Haberland M., Reddy T., Cournapeau D., Burovski E., Peterson P., Weckesser W., Bright J., et al. SciPy 1.0: Fundamental Algorithms for Scientific Computing in Python. Nat. Methods. 2020; 17:261-272. doi: 10.1038/s41592-019-0686-2.
152. Palme J., Hochreiter S., Bodenhofer U. KeBABS: An R Package for Kernel-Based Analysis of Biological Sequences. Bioinformatics. 2015; 31:2574-2576. doi: 10.1093/bioinformatics/btv176.
153. Yu G., Smith D. K., Zhu H., Guan Y., Lam T. T. ggtree: An r Package for Visualization and Annotation of Phylogenetic Trees with Their Covariates and Other Associated Data. Methods Ecol. Evol. 2017; 8:28-36. doi: 10.1111/2041-210λ.12628.
154. Yu G., Lam T. T. Y., Zhu H., Guan Y. Two Methods for Mapping and Visualizing Associated Data on Phylogeny Using Ggtree. Mol. Biol. Evol. 2018; 35:3041-3043. doi: 10.1093/molbev/msy194.
155. Yu G. Using Ggtree to Visualize Data on Tree-Like Structures. Curr. Protoc. Bioinform. 2020; 69:e96. doi: 10.1002/cpbi.96.
156. Paradis E., Schliep K. Ape 5.0: An Environment for Modern Phylogenetics and Evolutionary Analyses in R. Bioinformatics. 2019; 35:526-528. doi: 10.1093/bioinformatics/bty633.
157. Richter, A., Sutherland, D., Ebrahimikondori, H., Babcock, A., Louie, N., Li, C., Coombe, L., Lin, D., Warren, R. L., Yanai, A., Kotkoff, M., Helbing, C. C., Hof, F., Hoang, L. M. N., & Birol, I. (2022). Associating Biological Activity and Predicted Structure of Antimicrobial Peptides from Amphibians and Insects. Antibiotics (Basel), Nov. 27, 2022: 11(12), 1710. https://doi.org/10.3390/antibiotics11121710.
158. WO 2020/118427 (Jun. 18, 2020) Antimicrobial Peptides (Birol et al.)
159. Lin, D. (Published: Oct. 31, 2022) M.Sc. Thesis High throughput in silico discovery of antimicrobial peptides in amphibian and insect transcriptomes (T). University of British Columbia. https://open.library.ubc.ca/collections/ubctheses/24/items/1.0402476.

What is claimed is:

1. An antimicrobial peptide consisting of the amino acid sequence according to any one of SEQ ID NO:1090, SEQ ID NO:1-SEQ ID NO:1024, SEQ ID NO:1086-SEQ ID NO:1089, and SEQ ID NO:1091-SEQ ID NO: 1103, or having at least 95% at least 96%, at least 97%, or at least 98% amino acid sequence identity thereto.

2. The antimicrobial peptide of claim 1, consisting of the amino acid sequence according to SEQ ID NO:1090.

3. (canceled)

4. (canceled)

5. A composition comprising the antimicrobial peptide according to claim 1, and a pharmaceutically acceptable excipient.

6. (canceled)

7. (canceled)

8. (canceled)

9. The composition of claim 5, wherein the composition is formulated for oral, injectable, rectal, topical, transdermal, nasal, or ocular delivery.

10. The composition of claim 5, wherein the composition is lyophilized.

11. (canceled)

12. (canceled)

13. (canceled)

14. (canceled)

15. (canceled)

16. A method of treating a bacterial infection in a subject in need thereof, wherein the method comprises administering to the subject a therapeutically effective amount of the antimicrobial peptide according to claim 1.

17. The method of claim 16, wherein the bacteria is Gram-negative bacteria.

18. The method of claim 16, wherein the bacteria is Escherichia coli, Salmonella enterica, Staphylococcus aureus, Pseudomonas aeruginosa, Streptococcus pyogenes, Mycobacterium smegmatis, Methicillin-resistant Staphylococcus aureus (MRSA), Salmonella enteritidis, or Salmonella Heidelberq.

19. (canceled)

20. The method of claim 16, wherein the subject is a human, a livestock animal, or a pet.

21. A lipid vesicle comprising the antimicrobial peptide of claim 1.

22. A nucleic acid molecule encoding the antimicrobial peptide of claim 1.

23. A vector comprising the nucleic acid molecule of claim 22.

24. (canceled)

25. (canceled)

26. (canceled)

27. (canceled)

28. (canceled)

29. (canceled)

30. A kit for identifying a target molecule associated with an infectious agent, said kit comprising the antimicrobial peptide of claim 1 together with instructions for conducting a method of identifying a target molecule associated with an infectious agent, wherein said target molecule binds to the antimicrobial peptide,
said method comprising the step of screening a library of candidate target molecules associated with the infectious agent, for a molecule that binds to the antimicrobial peptide;
wherein said infectious agent is Gram-negative bacteria, Gram-positive bacteria, acid fast bacteria, or a drug resistant bacteria.

31. A kit for identifying a target molecule for modulating biological activity, said kit comprising the antimicrobial peptide of claim 1 together with instructions for conducting a method of identifying a target molecule for modulating biological activity, wherein said target molecule binds to the antimicrobial peptide,
said method comprising the step of screening a library of candidate target molecules for a molecule that binds to the antimicrobial peptide.

32. The method of claim 16, wherein the bacteria is Gram-positive bacteria.
